(19) United States
(12) Patent Application Publication
(10) Pub. No.: US 2009/0246276 A1
(43) Pub. Date: Oct. 1, 2009

(54) Pharmaceutical Compositions

(76) Inventors: Graham Jackson, Atlantic Shores (BB); Edwin Walsh, Marino (IE); Werner Oberegger, Waterdown (CA); Xiaopin Jin, Mississauga (CA)

Correspondence Address: Lovells LLP, 590 Madison Avenue, New York, NY, 10222, US

(21) Appl. No.: 12/360,673

(22) Filed: Jan. 27, 2009

Related U.S. Application Data
(60) Provisional application No. 61/023,951, filed on Jan. 28, 2008.

(51) Int. Cl. A61K 9/22; A61K 9/00; A61K 31/343

(52) U.S. Cl. 424/465; 424/400; 514/468

(57) ABSTRACT

The present invention relates to a pharmaceutical composition comprising a tablet core comprising a combination of actives selected from the group consisting of bupropion hydrochloride and escitalopram oxalate, bupropion hydrobromide and citalopram hydrochloride, bupropion hydrobromide and escitalopram oxalate, and bupropion hydrobromide and quetiapine fumarate, and at least one pharmaceutically acceptable excipient, and a control-releasing coat surrounding the tablet core, wherein said composition surprisingly provides for a synchronous release of the combination of active agents across in-vitro. The once-daily pharmaceutical composition surprisingly also provides for enhanced absorption of bupropion hydrobromide when administered to a subject in need of such administration.

### RELATED APPLICATIONS

This application claims priority to U.S. provisional patent application Ser. No. 61/023,951 filed Jan. 28, 2008, the contents of which are hereby incorporated by reference in its entirety.

### FIELD OF THE INVENTION

The present invention relates to novel once daily pharmaceutical compositions comprising combinations of escitalopram and bupropion or citalopram and bupropion and their use for the treatment of central nervous system disorders, such as for example mood disorders (e.g., major depressive disorder (MDD)—also known as major depression, unipolar depression, unipolar disorder, or clinical depression) and anxiety disorders (general anxiety disorder, social anxiety disorder, post traumatic stress disorder, or panic disorder). The present invention also relates to novel once daily pharmaceutical compositions comprising a combination of bupropion and quetiapine fumarate.

### BACKGROUND OF THE INVENTION

The burden of mental illness and neurological disorders worldwide is significant. According to the World Health Organization, neuropsychiatric disorders account for 31% of the disability in the world, affecting both rich and poor nations alike (World Health Report 2001: Mental Health. New Understanding, New Hope. World Health Organization (WHO). January 2001. Geneva, Switzerland). It is estimated that the incidence of major depression in the general population is around 5% and its lifetime prevalence is about 20% (Weissman, M. M., et al. (1996) Am Med Ass 276: 293-299). Similarly, the incidence of anxiety disorders in the general population is widespread and high, with lifetime prevalence rates ranging between about 14% and 29% in Western countries (Michael, T et al. (2007). Psychiatry, 6 (4): 136-142). This study also found co-morbidity among individuals with an anxiety disorder to be high, with three out of four individuals with a lifetime anxiety disorder experiencing at least one other mental disorder. These FIG.ures, together with the conclusion that unipolar depression accounted for the fourth leading cause of worldwide Disability Adjusted Life Years (Murray C. J. et al. (1997). Lancet 349: 1436-1442), makes neuropsychiatric disorders, particularly MDD, a significant global health issue, which needs to be treated.

Today, physicians have about 20 FDA approved medications as effective options for treating depressed patients. However, no one treatment is universally effective. While some patients respond to one antidepressant, others respond to another, and some patients may require a combination of medications.

The Sequenced Treatment Alternatives to Relieve Depression (STAR*D) study, the largest and longest study conducted to date, assessed the effectiveness of antidepressant treatments in patients diagnosed with MDD (Rush A. J. et al. (2006). Am J Psychiatry 163: 1905-1917). STAR*D was divided into four levels, each of which assessed the effectiveness of a different medication or combination of medications.

Patients who did not become symptom free in one level of treatment moved on to the next level. Level 1 evaluated the effectiveness of the antidepressant citalopram alone. Citalopram and escitalopram (the s-enantiomer of citalopram) currently marketed in the United States as Celexa® and Lexapro® respectively, belong to the class of antidepressants known as selective serotonin reuptake inhibitors (SSRIs). Citalopram was chosen as the first line of treatment because of its superior efficacy, ease of administration (once-a-day), and safety profile in older patients. Patients who did not become symptom free over a 12 to 14 week period after being treated with Celexa® moved on to level 2.

Level 2 patients had the option of switching to a different medication or adding on to the existing Celexa® treatment. Patients who opted for the “add-on” group were prescribed either bupropion-SR (Wellbutrin® SR), or buspirone (BuSpar®). Buspirone itself is not an antidepressant, but enhances the action of antidepressants. Bupropion, on the other hand, is an antidepressant belonging to the chemical class of aminoketones. Bupropion, marketed in the U.S. as Wellbutrin®, Wellbutrin® SR, or Wellbutrin® XL, is classified as an atypical antidepressant. Bupropion was chosen as the antidepressant of choice in Level 2 possibly for several reasons. For one, clinical studies have confirmed the efficacy of bupropion for MDD. (Fava M. et al. (2005). Prim Care Companion J Clin Psychiatry 7(3): 106-113). For another, bupropion, in contrast to nearly all other antidepressants, does not cause weight gain or sexual dysfunction (Zimmerman M. et al. (2005). J Clin Psychiatry 66(10): 1336-1339; Clayton A H. (2003). Primary Psychiatry 10(1): 55-61) and is more effective than SSRIs at improving symptoms of hypersomnia and fatigue in depressed patients (Baldwin et al. (2006). J Clin Psychiatry 67 (suppl 6): 9-15). Further, a survey of clinicians found bupropion to be the preferred “add-on” antidepressant for patients not responding to SSRIs as the first line of treatment (Zisook S et al. (2006). Biol Psychiatry 59(3): 203-210, Lam R. W. (2004). J Clin Psychiatry 65(3): 337-340).

One of the conclusions of the STAR*D study is that patients resistant to citalopram and subsequently treated with a combination of citalopram and bupropion faired much better than patients on citalopram or bupropion alone. The study also indicated a possibility of a higher remission rate in citalopram non-responders when bupropion was added on to the existing citalopram treatment rather than when switched to bupropion (30% vs. 20%).

One of the major problems in treating depression with SSRIs is the two to three week delay in onset of their action. This is believed to be a consequence of the mechanism of action of SSRIs (Piñeyro G. and Blier P. (1999) Pharmacol Rev 51(3): 533-591). Citalopram, primarily through its S-enantiomer, escitalopram, mediates its antidepressant effects by inhibiting re-uptake of serotonin (5-hydroxytryptamine [5-HT]) released into the synaptic cleft (Broestrup C. and Sanchez C. (2004). Int J Psychiatry Clin Practice 8 (suppl 1): 11-13). A result of the inhibition of this uptake is that 5-HT persists in the synaptic cleft thereby stimulating receptors of postsynaptic neurons for an extended period in patients suffering from MDD.

Current research suggests that early in the SSRI antidepressant response, 5-HT_{1A} autoreceptors, located at the cell body of neurons, exert a negative feedback response on the firing activity of serotonergic (5-HT) neurons by binding to excess 5-HT. These autoreceptors, after a period of time of treatment with the SSRI, become desensitized and allow 5-HT neurons to regain their normal firing rate in the presence of sustained reuptake inhibition (Blier, P. (2003) European Neuropsychopharmacology 13: 57-66). The time taken to desensitize 5-HT_{1A} autoreceptors, about two to three weeks, is believed to represent the delay in onset of action of SSRIs. Several lines of evidence suggest, however, that the time to desensitization of 5-HT_{1A} autoreceptors can be reduced, and hence the delay in the onset of action of SSRIs can be shortened, when SSRIs are administered in combination with other antidepressants, like mirtazapine and bupropion.

The mechanism of action of bupropion is not clearly understood. Bupropion has the ability to increase synaptic availability of norepinephrine (NE) and differentially effect dopamine (DA) release in various parts of the brain (Dong J and Blier P. (2001). Psychopharmacology 155: 52-57; Mansari M. E. et al. (2008). Neuropharmacology 55: 1191-1198). It is believed that this enhanced NE release results in an attenuation of firing of NE neurons due to an increased activation of inhibitory somatodendritic α₂-adrenoceptors located on NE neurons rather than due to the re-uptake inhibition of NE as previously thought. NE neurons gradually re-initiate firing to normal levels over a two-week period of bupropion administration as the α₂-adrenoceptors become desensitized.

Although SSRIs and bupropion exert their action via different neuronal systems it appears that these systems work in concert in the antidepressant response. In fact, 5-HT and NE neurons have reciprocal connections. Mansari et al. have recently demonstrated that bupropion leads to a rapid and sustained increase in the firing rate of 5-HT neurons and conclude that this is a result of the desensitization of the 5-HT_{1A} autoreceptors after only two days of administration (Mansari et al. (2008). Neuropharmacology 55: 1191-1198). Additionally, long-term SSRI administration has been shown to gradually attenuate NE neuronal firing (Szabo S. T. et al. (2000). Int J Neuropsychopharmacol 3: 1-11). Given this interplay between SSRI and bupropion, it has been postulated that a combination of an SSRI, such as citalopram, and bupropion should exert a synergistic effect on both 5-HT and NE systems. The rapid desensitization of the 5-HT_{1A} autoreceptors by bupropion should override the two to three week suppression of serotonergic neurons induced by SSRIs in as little as two days. Additionally, the enhanced NE releasing action by bupropion should counteract the decreased firing rate of NE neurons produced by long-term administration of SSRIs. Intuitively then, treatment methods that affect both 5-HT and NE neuronal systems might be expected to benefit depressed patients regardless of whether their depression is a result of 5-HT and/or NE deficiency.

Preliminary clinical evidence has provided support for the efficacy of combining bupropion and escitalopram for depressed patients (CITATION). Patients were treated for 8 weeks with a combination of escitalopram and bupropion in escalating doses up to 40 mg/d and 450 mg/d respectively. This study did not conduct a head-to-head comparison of combination versus monotherapy. Nevertheless, of 43 patients who took part in the study, 34% remitted within 2 weeks and 61% remitted after 8 weeks. Historically, according to the study, 10% of patients remit after two weeks and 41% remit after 2 weeks on monotherapy. Further support for a combination of SSRI and bupropion was recently demonstrated in an animal model. Prica et al. evaluated the effects of co-administration of bupropion and SSRIs in mice using the forced swimming test, which is predictive of the antidepressant activity of drugs (Prica et al. Behav. Brain Res. (2008). 194: 92-99). The results suggest that bupropion might enhance the effectiveness of SSRIs and SNRIs but not NRIs. Their results also suggest that bupropion enhances only the serotonergic system, which is in agreement with the pre-clinical studies presented above.

Taken together, basic research and preliminary clinical studies support the notion that a combination of antidepressant drugs having complementary mechanisms of action on 5-HT and NE systems, such as citalopram and escitalopram in combination with bupropion, have the potential of producing a more robust and/or rapid response, and hence a more efficacious outcome with possibly a lower side-effect profile. It is further reasonable to assume that the synergistic effects of these drugs can be maximized if both these drugs can be administered such that the drugs can act on the 5-HT/NE neuronal network at or about the same time. It would therefore be a significant advance in the art if a pharmaceutical composition can be manufactured such that both citalopram or escitalopram and bupropion can be formulated into a single composition, which provides for the release of both drugs such that the drugs might be able to act on the 5-HT and NE neuronal systems at or about the same time to maximize the expected synergistic antidepressant outcome.

Pharmaceutical compositions for the delivery of combinations of drugs are not new in the art of drug delivery. For example, U.S. Pat. No. 4,449,983, (the '983 application) refers to ‘an osmotic device for delivering two beneficial drugs to an environment of use’. The patent refers to a tri-layer tablet coated with a semi-permeable membrane with two separate orifices to allow for drug release. The semi-permeable membrane is substantially impermeable to the drugs. The first tablet layer contains the first active ingredient, the second tablet layer forms a swellable (hydrogel) partition barrier and the third tablet layer contains the second drug. The tablet is then coated with a semi-permeable membrane to form two drug-containing compartments in one tablet. Two separate orifices are then formed across the membrane to communicate each drug containing compartment with the environment such that drug is delivered separately from each compartment. The swellable (hydrogel) partition layer acts as a ‘driving’ layer. As the partition layer hydrates it expands and reduces the volume of each drug-containing compartment. The rate of drug release from this device is controlled by an osmotic pressure gradient within each drug-containing compartment.

U.S. Pat. No. 4,455,143, refers to a similar osmotic device to that described in the '983 patent, the difference being the composition of the tablet partition layer. Instead of a swellable (hydrogel) layer, the partition layer is made of a material ‘selected from the group consisting essentially of semi-permeable, microporous and impermeable materials’. The function of the partition layer is to ‘maintain the integrity of the first and second compartments’, (i.e. the drug containing compartments). The rate of drug release is controlled by the osmotic pressure within the drug compartment.

U.S. Pat. No. 4,601,894, refers to a matrix tablet composition for the controlled release of the triple drug combination of acetaminophen, pseudoephedrine sulfate and dexbrompheniramine maleate. The matrix composition contains the three actives but a choice of polymers (preferably hydroxypropyl methylcellulose (HPMC) ethers and ethylcellulose. The patent refers to a simple combination dosage form with unexpected release rates (based on very different drug solubilities) specific to three actives, ‘acetaminophen, pseudoephedrine or a pharmaceutically acceptable salt thereof and dexbrompheniramine or a pharmaceutically acceptable salt thereof.’ The matrix tablet composition referred to is an uncoated matrix tablet, with drug release controlled by a combination of drug diffusion and polymer erosion.

U.S. Pat. No. 4,662,880, refers to an osmotic device for the controlled delivery of the two pharmaceutical actives pseudoephedrine and brompheniramine. Both actives are formulated in one tablet core; a semi-permeable membrane, which is substantially impermeable to the passage of drug, is applied followed by an immediate release active coat containing both pharmaceutical actives.

U.S. Pat. No. 4,844,907, refers to a ‘multiphase (especially a bi-layered, optionally coated) tablet’ composition for the delivery of a combination of a narcotic analgesic and a non-steroidal anti-inflammatory. The patent refers to a bi-layer tablet consisting of two separate controlled release matrix layers, each layer containing one of the actives individually. There is no partition layer between the two active layers. U.S. Pat. No. 5,866,164, refers to a similar method for the controlled delivery of an opioid and an opioid antagonist.

U.S. Pat. Nos. 4,814,181, and 4,915,954 refer to an osmotic pump dosage form for delivering actives at two different rates. The patents refer to a bi-layer tablet core coated with a semi-permeable membrane with a single passageway for osmotic drug release. The semi-permeable membrane is substantially impermeable to the passage of the drug. The first drug layer (closest to the passageway) releases drug rapidly while the second drug layer releases active over a prolonged period of time.

U.S. patent application Ser. No. 11/355,315 refers to an osmotic dual delivery technology containing a bi-layered core. The application purports to teach a dual controlled release of both drugs from a controlled release bi-layered core osmotic device. The arrangement of the layers of the bi-layer core can be stacked or the second layer can surround the first. The application refers to a first and second drug which can be released sequentially or in an overlapping manner when the osmotic device is exposed to an aqueous environment in a timed, targeted, pseudo-first order, first order, pseudo-zero order, zero-order, and/or delayed release profile.

PCT International Application Number PCT/US2007/011186 (WO 2007/133583) refers to a solid dosage form for delivery of water-soluble pharmaceutical agents. The solid dosage form comprises a matrix core containing the pharmaceutical agent and a hydrophobic material, and a coating containing a hydrophilic pore-forming agent and a hydrophobic polymer. The dosage form exhibits a zero-order release profile upon dissolution.

U.S. patent application Ser. Nos. 11/582,164 (the '164 application) and 11/549,714 both refer to stable once-a-day oral dosage forms containing escitalopram or pharmaceutically acceptable salt thereof and bupropion and pharmaceutically acceptable salt thereof. The '164 application teaches that the escitalopram and bupropion are preferably physically separated. The applicants teach that commercial escitalopram oxalate compositions, which are normally stable up to about 12 months, degrade significantly more rapidly when stored in intimate contact with bupropion hydrochloride such that each drug degrades by more than 10% in potency after just one month of storage at 40° C. and 75% relative humidity. Accordingly, the applicants teach that compositions comprising the drugs may be separated into separate discrete zones such as separate layers or the compositions may take the form of a plurality of escitalopram beads or tablets and a plurality of bupropion tablets or beads, where ate least one or both of the bead or tablet populations are coated.

U.S. Pat. No. 7,241,805 (the '805 patent) refers to combinations of bupropion hydrobromide with a second drug, which may be citalopram or escitalopram. In particular, the '805 patent refers to controlled release microparticulate compositions wherein combination products can be made by providing an overcoat comprising a second drug substantially surrounding a control-releasing coat of each microparticle core comprising bupropion hydrobromide. In certain embodiments, a pulsatile release of at least one other drug is achieved from the coated microparticles. The overcoat can be an immediate release overcoat that includes at least one other drug. As such, this composition can provide an immediate release of at least one other drug from the overcoat in a first phase of drug release, and then a subsequent controlled release of the bupropion hydrobromide from the control-releasing coated microparticle in a second phase of drug release.

While the above referenced prior art refers to pharmaceutical compositions suitable for the delivery of combinations of drugs, they are either complicated or costly to manufacture. Further, the ability of the above referenced pharmaceutical compositions to release two drugs, such as bupropion and citalopram or escitalopram, at about the same rate to maximize the expected synergistic antidepressant outcome, is further hampered by the significantly different physicochemical characteristics of the two drugs, which vary across the physiological pH range. Accordingly, there is a need to develop a once-a-day pharmaceutical composition capable of delivering bupropion and citalopram or escitalopram at about the same rate independent of the pH of the environment of use.

### SUMMARY OF THE INVENTION

The present invention relates to a once-daily pharmaceutical composition comprising a tablet core comprising a combination of actives selected from the group consisting of bupropion hydrochloride and escitalopram oxalate, bupropion hydrobromide and citalopram hydrochloride, bupropion hydrobromide and escitalopram oxalate, and bupropion hydrobromide and quetiapine fumarate, optionally a stabilizer in an effective stabilizing amount, and at least one pharmaceutically acceptable excipient, and a control-releasing coat surrounding the tablet core, wherein said composition surprisingly provides for a synchronous release of the combination of active agents across the pH range i.e., 0.1N HCl, pH 4.5 acetate buffer, and pH 6.8 phosphate buffer in-vitro. The once daily pharmaceutical composition surprisingly also provides for enhanced absorption of bupropion hydrobromide when administered to a subject in need of such administration. The once-daily pharmaceutical composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate.

The synchronous release of the combination of actives comprising the once-daily pharmaceutical compositions of the present invention is particularly surprising when one considers that the differing physicochemical characteristics of the active ingredients and the likely differences in the permeability coefficients for the combination of active drugs would result in a differing rate and extent of drug release for each of the drugs chosen to be part of the combination. Accordingly, it was expected that it would be difficult to optimize the release kinetics of the combination of drugs contemplated without one drug potentially negatively influencing the release kinetics of the other drug of the combination. However, it was surprisingly found that despite the differing physicochemical characteristics (shown below) for the actives used in the combinations described herein, the in-vitro rate and extent of drug release was substantially synchronous across the pH range.

Bupropion Physichochemistry

| | Bupropion HCl | Bupropion HBr |
|---|---|---|
| Molecular Weight | 276.2 | 320.7 |
| pKa | 7.8 | 7.8 |
| Solubility (37° C.) | | |
| 0.1N HCl | 416 | 190 |
| pH 4.5 | 437 | 180 |
| pH 6.8 | 437 | 200 |

Escitalopram Physicochemistry

| | Escitalopram Oxalate |
|---|---|
| Molecular Weight | 414.4 |
| pKa | 9.5 |
| Solubility (37° C.) | |
| 0.1N HCl | 183 |
| pH 4.5 | 111 |
| pH 6.8 | 121 |

Quetiapine Fumarate Physicochemistry

| | Quetiapine Fumarate |
|---|---|
| Molecular Weight | 883.11 |
| pKa | 3.3, 6.87 |
| Solubility (37° C.) | |
| 0.1N HCl | 41 |
| pH 4.5 | 3 |
| pH 6.8 | 0.3 |

Accordingly, at least one embodiment of the present invention provides for a once-daily pharmaceutical composition comprising a homogenous core comprising a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer in an effective stabilizing amount, and at least one pharmaceutically acceptable excipient, and a control-releasing coating surrounding said core, said coating comprising a water-insoluble water-permeable film-forming polymer, a water-soluble polymer and at least one plasticizer; wherein said composition provides for a synchronous release of the combination of active agents.

In at least one embodiment of the present invention, the pharmaceutical compositions provide for a synchronous release of the combination of actives in 0.1N HCl, pH 4.5 acetate buffer, pH 6.8 phosphate buffer when measured in 900 ml of each aqueous solution at 37° C. using USP1 apparatus at 75 rpm.

In at least one embodiment of the invention, the stabilizer comprises at least one suitable pharmaceutically acceptable inorganic acid, at least one suitablem pharmaceutically acceptable organic acid, at least one suitable pharmaceutically acceptable salt of an organic base, at least one suitable pharmaceutically acceptable salt of an inorganic acid, at least one suitable pharmaceutically acceptable acid salt of an amino acid, potassium metabisulfite, sodium bisulfite, or at least one suitable pharmaceutically acceptable phenylated antioxidant, or any combination thereof.

In at least one embodiment of the present invention, stabilizer comprises at least one suitable inorganic acid, which at a concentration of about 0.31% w/w/ forms an aqueous solution having a pH of from about 0.5 to about 0.4.

In at least one embodiment of the present invention, the stabilizer comprises hydrochloric acid, phosphoric acid, nitric acid, or sulfuric acid, or any combination thereof.

In at least one embodiment of the present invention, the stabilizer comprises at least one suitable organic acid that has a solubility in water at 20° C. of less than about 10 g/100 g water and that at a concentration of about 60% w/w forms an aqueous suspension having a pH of from about 0.9 to about 4.0.

In at least one embodiment of the present invention, the stabilizer comprises at least one suitable dicarboxylic acid that has a solubility in water at 20° C. of less than about 10 g/100 g water and that at a concentration of about 60% w/w forms an aqueous suspension having a pH of from about 0.9 to about 4.0.

In at least one embodiment of the present invention, the stabilizer comprises hydrochloric acid, phosphoric acid, nitric acid, and sulfuric acid, or any combination thereof.

In at least one embodiment of the present invention, the stabilizer comprises at least one suitable pharmaceutically acceptable salt of an organic base having an aqueous pH of from about 2.70 to about 3.10 at a concentration of about 10% w/w.

In at least one embodiment of the present invention, the stabilizer comprises creatinine hydrochloride.

In at least one embodiment of the present invention, the stabilizer comprises at least one suitable pharmaceutically acceptable salt of an organic base having an aqueous pH of from about 2.95 to about 3.05, at a concentration of about 20% w/w.

In at least one embodiment of the present invention, the stabilizer comprises thiamine hydrochloride.

In at least one embodiment of the present invention, the stabilizer comprises sat least one salt of an organic base having an aqueous pH of from about 2.70 to about 2.72, at a concentration of about 20% w/w.

In at least one embodiment of the present invention, the stabilizer comprises thiamine hydrochloride.

In at least one embodiment of the invention, the stabilizer is citric acid.

In at least one embodiment of the present invention, the stabilizer comprises at least one suitable pharmaceutically acceptable salt of an inorganic acid having an aqueous pH of from about 4.20 to about 4.30 at a concentration of about 10 w/w.

In at least one embodiment of the present invention, the stabilizer comprises potassium phosphate monobasic.

In at least one embodiment of the present invention, the stabilizer comprises at least one suitable pharmaceutically acceptable acid salt of an amino acid.

In at least one embodiment of the present invention, the stabilizer comprises L-cysteine hydrochloride, L-cystine dihydrochloride, glycine hydrochloride or any combination thereof.

In at least one embodiment of the present invention, the stabilizer comprises potassium metabisulfite, sodium bisulfite, or any combination thereof.

In at least one embodiment of the invention, the stabilizer comprises at least one suitable pharmaceutically acceptable phenylated antioxidant.

In at least one embodiment of the invention, the stabilizer comprises butlylated hydroxytoluene (BHT), butylated hydroxyanisole (BHA), or any combination thereof.

In at least one embodiment of the present invention, the stabilizer comprises butylated hydroxytoluene.

In at least one embodiment of the present invention, the stabilizer comprises a combination of citric acid and butylated hydroxytoluene.

In at least one embodiment of the present invention, the once-daily pharmaceutical composition comprises at least one pharmaceutically acceptable excipient selected from the group consisting of a binder, a lubricant, a filler, a glidant, or any combinations thereof.

In at least one embodiment of the present invention, the water-insoluble water-permeable film-forming polymer comprises at least one cellulose ether, cellulose ester, methacrylic acid derivative, aqueous ethylcellulose dispersion, aqueous acrylic enteric system, or polyvinyl derivative, or any combination thereof.

In at least one embodiment of the present invention, the water-soluble polymer comprising the control-releasing coat comprises at least one methylcellulose, hydroxypropylcellulose, hydroxypropylmethylcellulose, hydroxyethylcellulose, polyvinyl alcohol, or polyvinylpyrrolidone, or any combination thereof.

In at least one embodiment of the present invention, the at least one plasticizer comprises a combination of two plasticizers.

In at least one embodiment of the present invention, the at least one plasticizer comprises at least one ester, or a polyalkylene glycol, or any combination thereof.

In at least one embodiment of the invention, the plastizer is a combination of polyethylene glycol 3350 and dibutyl sebacate.

In at least one embodiment of the invention, the once-daily pharmaceutical composition is in the form of a tablet.

In at least one embodiment, the once-daily pharmaceutical composition when administered to a subject in need of such administration can provide an about 15-25% increase in the bioavailability of bupropion when compared to co-administration of single active agent pharmaceutical compositions of bupropion hydrobromide and citalopram hydrochloride or bupropion hydrobromide and escitalopram oxalate.

At least one embodiment of the present invention provides for a method of treating a mood and/or anxiety disorder in a subject in need of such treatment comprising administering once daily to said subject any one of the pharmaceutical compositions of the invention.

At least one embodiment of the present invention provides for a method of treating a mood and/or anxiety disorder in a subject in need of such treatment comprising administering a once daily pharmaceutical composition comprising a homogenous core comprising a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer in an effective stabilizing amount, and at least one pharmaceutically acceptable excipient, and a control-releasing coating surrounding said core, said coating comprising a water-insoluble water-permeable film-forming polymer, a water-soluble polymer and at least one plasticizer.

At least one embodiment of the present invention provides for a method of treating a mood and/or anxiety disorder in a subject in need of such treatment comprising administering a once daily pharmaceutical composition comprising a homogenous core comprising a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer in an effective stabilizing amount, and at least one pharmaceutically acceptable excipient, and a control-releasing coating surrounding said core, said coating comprising a water-insoluble water-permeable film-forming polymer, a water-soluble polymer and at least one plasticizer, wherein said composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate.

At least one embodiment of the present invention provides for a method of treating a mood and/or anxiety disorder in a subject in need of such treatment comprising administering a once daily pharmaceutical composition comprising a homogenous core comprising a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer in an effective stabilizing amount, and at least one pharmaceutically acceptable excipient, and a control-releasing coating surrounding said core, said coating comprising a water-insoluble water-permeable film-forming polymer, a water-soluble polymer and at least one plasticizer; wherein said composition provides for a synchronous release of the combination of active agents.

At least one embodiment of the present invention provides for a pharmaceutical composition comprising a controlled release matrix core, said controlled release matrix core comprising at least one hydrophilic control-releasing polymer present in a control-releasing amount, a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer, and at least one pharmaceutically acceptable excipient; wherein said pharmaceutical composition provides for a synchronous release of the combination of active agents.

In at least one embodiment of the present invention, the at least one hydrophilic control-releasing polymer comprising the controlled-release matrix core comprises at least one hydrophilic cellulose, ethylcellulose, polysaccharide, polyvinylpyrrolidone, polymethacrylate, or a mixture of polyvinyl acetate and polyvinylpyrrolidone, or any combination thereof.

At least one embodiment of the present invention provides for a method of treating a mood and/or anxiety disorder in a subject in need of such treatment comprising administering once daily to said subject a pharmaceutical composition comprising a controlled release matrix core, said controlled release matrix core comprising at least one hydrophilic control-releasing polymer present in a control-releasing amount, a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer, and at least one pharmaceutically acceptable excipient; wherein said composition provides for a synchronous release of the combination of actives.

At least one embodiment of the present invention provides for a method of treating a mood and/or anxiety disorder in a subject in need of such treatment comprising administering once daily to said subject a pharmaceutical composition comprising a controlled release matrix core, said controlled release matrix core comprising at least one hydrophilic control-releasing polymer present in a control-releasing amount, a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer, and at least one pharmaceutically acceptable excipient.

At least one embodiment of the present invention provides for a method of treating a mood and/or anxiety disorder in a subject in need of such treatment comprising administering once daily to said subject a pharmaceutical composition comprising a controlled release matrix core, said controlled release matrix core comprising at least one hydrophilic control-releasing polymer present in a control-releasing amount, a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer, and at least one pharmaceutically acceptable excipient, wherein said composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate.

At least one embodiment provides for a pharmaceutical composition comprising a core comprising a first immediate release layer comprising a therapeutically effective amount of an active agent selected from the group consisting of bupropion hydrochloride and bupropion hydrobromide, optionally a stabilizer and at least one pharmaceutically acceptable excipient in direct contact with a second immediate release layer comprising an active agent selected from the group consisting of citalopram hydrochloride and escitalopram oxalate, optionally a stabilizer, and at least one pharmaceutically acceptable excipient, and a control-releasing coating surrounding said core, said coating comprising a water-insoluble water-permeable film-forming polymer, a water-soluble polymer and at least one plasticizer, wherein said composition provides for a synchronous release of the active agents.

At least one embodiment of the present invention provides for a method of treating a mood and/or anxiety disorder in a subject in need of such treatment comprising administering once daily to said subject a pharmaceutical composition comprising a core comprising a first immediate release layer comprising a therapeutically effective amount of an active agent selected from the group consisting of bupropion hydrochloride and bupropion hydrobromide, optionally a stabilizer and at least one pharmaceutically acceptable excipient in direct contact with a second immediate release layer comprising an active agent selected from the group consisting of citalopram hydrochloride and escitalopram oxalate, optionally a stabilizer, and at least one pharmaceutically acceptable excipient, and a control-releasing coating surrounding said core, said coating comprising a water-insoluble water-permeable film-forming polymer, a water-soluble polymer and at least one plasticizer, wherein said composition provides for a synchronous release of the active agents.

At least one embodiment of the present invention provides for a method of treating a mood and/or anxiety disorder in a subject in need of such treatment comprising administering once daily to said subject a pharmaceutical composition comprising administering once daily to said subject a pharmaceutical composition comprising a core comprising a first immediate release layer comprising a therapeutically effective amount of an active agent selected from the group consisting of bupropion hydrochloride and bupropion hydrobromide, optionally a stabilizer and at least one pharmaceutically acceptable excipient in direct contact with a second immediate release layer comprising an active agent selected from the group consisting of citalopram hydrochloride and escitalopram oxalate, optionally a stabilizer, and at least one pharmaceutically acceptable excipient, and a control-releasing coating surrounding said core, said coating comprising a water-insoluble water-permeable film-forming polymer, a water-soluble polymer and at least one plasticizer.

At least one embodiment of the present invention provides for a method of treating a mood and/or anxiety disorder in a subject in need of such treatment comprising administering once daily to said subject a pharmaceutical composition comprising administering once daily to said subject a pharmaceutical composition comprising a core comprising a first immediate release layer comprising a therapeutically effective amount of an active agent selected from the group consisting of bupropion hydrochloride and bupropion hydrobromide, optionally a stabilizer and at least one pharmaceutically acceptable excipient in direct contact with a second immediate release layer comprising an active agent selected from the group consisting of citalopram hydrochloride and escitalopram oxalate, optionally a stabilizer, and at least one pharmaceutically acceptable excipient, and a control-releasing coating surrounding said core, said coating comprising a water-insoluble water-permeable film-forming polymer, a water-soluble polymer and at least one plasticizer, wherein said composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate.

In at least one embodiment of the present invention, the once-daily pharmaceutical compositions of the invention avoid dose dumping of the combination of actives in the presence of food and/or alcohol.

In at least one embodiment of the present invention, the once-daily pharmaceutical compositions of the invention are free of food-effect.

At least one embodiment of the present invention provides for a method of treating a mood and/or anxiety disorder in a subject in need of such treatment comprising administering a once daily pharmaceutical composition comprising a homogenous core comprising a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer in an effective stabilizing amount, and at least one pharmaceutically acceptable excipient, and a control-releasing coating surrounding said core, said coating comprising a water-insoluble water-permeable film-forming polymer, a water-soluble polymer and at least one plasticizer, wherein said composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate and is free of food effect.

At least one embodiment of the present invention provides for a method of treating a mood and/or anxiety disorder in a subject in need of such treatment comprising administering once daily to said subject a pharmaceutical composition comprising a controlled release matrix core, said controlled release matrix core comprising at least one hydrophilic control-releasing polymer present in a control-releasing amount, a therapeutically effective combination of active agents selected from the group consisting of bupropion hydrochloride and escitalopram oxalate, bupropion hydrobromide and citalopram hydrochloride, and bupropion hydrobromide and escitalopram oxalate, a stabilizer, and at least one pharmaceutically acceptable excipient, wherein said composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate and is free of food effect.

At least one embodiment of the present invention provides for a method of treating a mood and/or anxiety disorder in a subject in need of such treatment comprising administering once daily to said subject a pharmaceutical composition comprising administering once daily to said subject a pharmaceutical composition comprising a core comprising a first immediate release layer comprising a therapeutically effective amount of an active agent selected from the group consisting of bupropion hydrochloride and bupropion hydrobromide, optionally a stabilizer and at least one pharmaceutically acceptable excipient in direct contact with a second immediate release layer comprising an active agent selected from the group consisting of citalopram hydrochloride and escitalopram oxalate, optionally a stabilizer, and at least one pharmaceutically acceptable excipient, and a control-releasing coating surrounding said core, said coating comprising a water-insoluble water-permeable film-forming polymer, a water-soluble polymer and at least one plasticizer, wherein said composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate and is free of food effect.

At least one embodiment of the present invention provides for a method of manufacturing a pharmaceutical composition, said method comprising the steps of: a) granulating an active agent selected from the group consisting of bupropion hydrobromide and bupropion hydrochloride by homogenously blending with a solution of at least one suitable binder and optionally a suitable stabilizer; b) drying said granules comprising either bupropion hydrobromide or bupropion hydrochloride and retaining said granules of a size between about 355 μm and about 800 μm; c) granulating an active agent selected from the group consisting of citalopram hydrochloride, escitalopram oxalate, and quetiapine fumarate by homogenously blending with a solution of at least one suitable binder and optionally at least one suitable stabilizer; d) drying said granules comprising either citalopram hydrochloride, escitalopram oxalate, and quetiapine fumarate and retaining said granules of a size between about 355 μm and about 800 μm; e) homogenously blending the granules in (b) and (d) in an amount equivalent to the desired dosage strength of each of the actives selected in (a) and (c) with at least one suitable lubricant; (f) compressing the homogenously blended mixture obtained in (e) into a homogenously blended tablet core; and coating said homogenously blended tablet core with a control-releasing coat comprising a water-insoluble water-permeable film-forming polymer, a water-soluble polymer and at least one plasticizer; wherein said pharmaceutical composition provides for a synchronous release of the actives selected in (a) and (b).

At least one embodiment of the present invention provides for a method of manufacturing a pharmaceutical composition comprising the steps of: a) granulating a first active selected from the group consisting of bupropion hydrochloride and bupropion hydrobromide with a second active selected from the group consisting of citalopram hydrochloride, escitalopram oxalate and quetiapine fumarate, in an amount equivalent to the desired dosage strength the first and second active by homogenously blending with a solution of at least one suitable binder and optionally at least suitable stabilizer; b) drying the granules obtained in (a) and retaining granules of <1.00 μm) homogenously blending the granules obtained in (b) with at least one suitable lubricant; d) compressing the homogenously blended mixture obtained in (c) into a homogenous tablet core; and d) coating said homogenously blended tablet core with a control-releasing coat comprising a water-insoluble water-permeable film-forming polymer, a water-soluble polymer and at least one plasticizer; wherein said pharmaceutical composition provides for a synchronous release of the first and second actives.

In certain embodiments of the present invention, the amount of bupropion hydrobromide present is at least about 10% less than a single active agent pharmaceutical composition comprising bupropion hydrobromide.

In certain embodiments of the present invention, the amount of bupropion hydrobromide present is at least about 10% less than a single active agent pharmaceutical composition comprising 348 mg bupropion hydrobromide.

### BRIEF DESCRIPTION OF THE DRAWINGS

The present invention will be further understood from the following detailed description with references to the following drawings.

FIG. 1A is a graph depicting the dissolution profile in 900 ml of 0.1N HCl using USP Apparatus 1 at 75 rpm at 37° C. of the composition described in Example 1.

FIG. 1B is a graph depicting the dissolution profile in 900 ml of pH 4.5 acetate buffer using USP Apparatus 1 at 75 rpm at 37° C. of the composition described in Example 1.

FIG. 1C is a graph depicting the dissolution profile in 900 ml of pH 6.8 phosphate buffer using USP Apparatus 1 at 75 rpm at 37° C. of the composition described in Example 1.

FIG. 2A is a graph depicting the dissolution profile in 900 ml of 0.1N HCl using USP Apparatus 1 at 75 rpm at 37° C. of the composition described in Example 2.

FIG. 2B is a graph depicting the dissolution profile in 900 ml of pH 4.5 acetate buffer using USP Apparatus 1 at 75 rpm at 37° C. of the composition described in Example 2.

FIG. 2C is a graph depicting the dissolution profile in 900 ml of pH 6.8 phosphate buffer using USP Apparatus 1 at 75 rpm at 37° C. of the composition described in Example 2.

FIG. 3A is a graph depicting the dissolution profile in 900 ml of 0.1N HCl using USP Apparatus 1 at 75 rpm at 37° C. of the composition described in Example 3.

FIG. 3B is a graph depicting the dissolution profile in 900 ml of pH 4.5 acetate buffer using USP Apparatus 1 at 75 rpm at 37° C. of the composition described in Example 3.

FIG. 3C is a graph depicting the dissolution profile in 900 ml of pH 6.8 phosphate buffer using USP Apparatus 1 at 75 rpm at 37° C. of the composition described in Example 3.

FIG. 4 is a graph depicting the dissolution profile in 900 ml of 0.1N HCl using USP Apparatus 1 at 75 rpm at 37° C. of the composition described in Example 4.

FIG. 5 is a graph depicting the dissolution profile in 900 ml of 0.1N HCl using USP Apparatus 1 at 75 rpm at 37° C. of the composition described in Example 5.

FIG. 6 is a graph depicting the dissolution profile in 900 ml of 0.1N HCl using USP Apparatus 1 at 75 rpm at 37° C. of the composition described in Example 6.

FIG. 7 is a graph depicting the dissolution profile in 900 ml of 0.1N HCl using USP Apparatus 1 at 75 rpm at 37° C. of the composition described in Example 7.

FIG. 8 is a graph depicting the dissolution profile in 900 ml of 0.1N HCl using USP Apparatus 1 at 75 rpm at 37° C. of the composition described in Example 8.

FIG. 9 is a graph depicting the dissolution profile in 900 ml of 0.1N HCl using USP Apparatus 1 at 75 rpm at 37° C. of the composition described in Example 9.

FIG. 10 is a graph depicting the dissolution profile in 900 ml of 0.1N HCl using USP Apparatus 1 at 75 rpm at 37° C. of the composition described in Example 10.

FIG. 11 is a graph depicting the dissolution profile in 900 ml of 0.1N HCl using USP Apparatus 1 at 75 rpm at 37° C. of the composition described in Example 11.

FIG. 12 is a graph depicting the dissolution profile in 900 ml of 0.1N HCl using USP Apparatus 1 at 75 rpm at 37° C. of the composition described in Example 12.

FIG. 13 is a graph depicting the dissolution profile in 900 ml of 0.1N HCl using USP Apparatus 1 at 75 rpm at 37° C. of the composition described in Example 13.

FIG. 14 is a graph depicting the dissolution profile in 900 ml of 0.1N HCl using USP Apparatus 1 at 75 rpm at 37° C. of the composition described in Example 14.

FIG. 15 is a graph depicting the dissolution profile in 900 ml of 0.1N HCl using USP Apparatus 1 at 75 rpm at 37° C. of the composition described in Example 15.

FIG. 16 is a graph depicting the dissolution profile in 900 ml of 0.1N HCl using USP Apparatus 1 at 75 rpm at 37° C. of the composition described in Example 16.

FIG. 17A is a graph depicting the dissolution profile in 900 ml of 0.1N HCl using USP Apparatus 1 at 75 rpm at 37° C. of the composition described in Example 17.

FIG. 17B is a graph depicting the dissolution profile in 900 ml of pH 6.8 phosphate buffer using USP Apparatus 1 at 75 rpm at 37° C. of the composition described in Example 17.

FIG. 18 is a graph depicting the dissolution profile in 900 ml of 0.1N HCl using USP Apparatus 1 at 75 rpm at 37° C. of the composition described in Example 18.

FIG. 19 is a graph depicting the dissolution profile in 900 ml of 0.1N HCl using USP Apparatus 1 at 75 rpm at 37° C. of the composition described in Example 19.

FIG. 20A is a graph depicting the dissolution profile in 900 ml of 0.1N HCl using USP Apparatus 1 at 75 rpm at 37° C. of the composition described in Example 20.

FIG. 20B is a graph depicting the dissolution profile in 900 ml of pH 6.8 phosphate buffer using USP Apparatus 1 at 75 rpm at 37° C. of the composition described in Example 20.

FIG. 21A is a graph depicting the mean plasma bupropion concentration versus time profile for the study described in Example 21 (linear scale (n=23)).

FIG. 21B is a graph depicting the mean plasma hydroxybupropion concentration versus time profile for the study described in Example 21 (linear scale (n=23)).

FIG. 21C is a graph depicting the mean plasma bupropion threoamino alcohol concentration versus time profile for the study described in Example 21 (linear scale (n=23)).

FIG. 21D is a graph depicting the mean plasma bupropion erythroamino alcohol concentration versus time profile for the study described in Example 21 (linear scale (n=23)).

FIG. 21E is a graph depicting the mean plasma PAWC concentration versus time profile for the study described in Example 21 (linear scale (n=23)).

FIG. 22A is a graph depicting the mean concentration-time profiles of citalopram during steady-state dosing of Celexa® 20 mg alone and Celexa 20 mg plus Wellbutrin XL® 300 mg for the study described in Example 22 (linear scales (n=26)).

FIG. 22B is a graph depicting the mean concentration-time profile of desmethylcitalopram during steady-state dosing of Celexa® 20 mg alone and Celexa® 20 mg plus Wellbutrin XL® 300 mg for the study described in Example 22 (linear scales (n=26)).

FIG. 22C is a graph depicting the mean concentration time profile for didesmethylcitalopram during steady-state dosing of Celexa® 20 mg alone and Celexa® 20 mg plus Wellbutrin XL® 300 mg for the study described in Example 22 (linear scales (n=26)).

FIG. 22D is a graph depicting the mean concentration time for bupropion, rrythro-hydrobupropion, hydroxybupropion, and threo-hydrobupropion during steady-state dosing of Celexa® 20 mg plus Wellbutrin XL® 300 mg for the study described in Example 22 (linear scales (n=26)).

FIG. 23A is a graph depicting the mean plasma bupropion concentration versus time profile for the study described in Example 23 (linear scale, Group 1 (n=11)).

FIG. 23B is a graph depicting the mean plasma hydroxybupropion concentration versus time profile for the study described in Example 23 (linear scale, Group 1 (n=11)).

FIG. 23C is a graph depicting the mean plasma bupropion threoamino alcohol concentration versus time profile for the study described in Example 23 (linear scale, Group 1 (n=11)).

FIG. 23D is a graph depicting the mean plasma bupropion erythroamino alcohol concentration versus time profile for the study described in Example 23 (linear scale, Group 1 (n=11)).

FIG. 23E is a graph depicting the mean plasma PAWC concentration versus time profile for the study described in Example 23 (linear scale, Group 1 (n=11)).

FIG. 23F is a graph depicting the mean plasma citalopram concentration versus time profile for the study described in Example 23 (linear scale, Group 1 (n=11)).

FIG. 23G is a graph depicting the mean plasma demethylcitalopram concentration versus time profile for the study described in Example 23 (linear scale, Group 1 (n=11)).

FIG. 23H is a graph depicting the mean plasma didemethylcitalopram concentration versus time profile for the study described in Example 23 (linear scale, Group 1 (n=11)).

FIG. 23I is a graph depicting the mean plasma bupropion concentration versus time profile for the study described in Example 23 (linear scale, Group 2 (n=10)).

FIG. 23J is a graph depicting the mean plasma hydroxybupropion concentration versus time profile for the study described in Example 23 (linear scale, Group 2 (n=10)).

FIG. 23K is a graph depicting the mean plasma bupropion threoamino alcohol concentration versus time profile for the study described in Example 23 (linear scale, Group 2 (n=10)).

FIG. 23L is a graph depicting the mean plasma bupropion erythroamino alcohol concentration versus time profile for the study described in Example 23 (linear scale, Group 2 (n=10)).

FIG. 23M is a graph depicting the mean plasma PAWC concentration versus time profile for the study described in Example 23 (linear scale, Group 2 (n=10)).

FIG. 23N is a graph depicting the mean plasma citalopram concentration versus time profile for the study described in Example 23 (linear scale, Group 2 (n=10)).

FIG. 23O is a graph depicting the mean plasma demethylcitalopram concentration versus time profile for the study described in Example 23 (linear scale, Group 2 (n=10)).

FIG. 23P is a graph depicting the mean plasma didemethylcitalopram concentration versus time profile for the study described in Example 23 (linear scale, Group 2 (n=10)).

FIG. 24A is a graph depicting the mean plasma bupropion concentration versus time profile for the study described in Example 24 (linear scale, Group 1 (n=13)).

FIG. 24B is a graph depicting the mean plasma hydroxybupropion concentration versus time profile for the study described in Example 24 (linear scale, Group 1 (n=13)).

FIG. 24C is a graph depicting the mean plasma bupropion threoamino alcohol concentration versus time profile for the study described in Example 24 (linear scale, Group 1 (n=13)).

FIG. 24D is a graph depicting the mean plasma bupropion erythroamino alcohol concentration versus time profile for the study described in Example 24 (linear scale, Group 1 (n=13)).

FIG. 24E is a graph depicting the mean plasma PAWC concentration versus time profile for the study described in Example 24 (linear scale, Group 1 (n=13)).

FIG. 24F is a graph depicting the mean plasma citalopram concentration versus time profile for the study described in Example 24 (linear scale, Group 1 (n=13)).

FIG. 24G is a graph depicting the mean plasma demethylcitalopram concentration versus time profile for the study described in Example 24 (linear scale, Group 1 (n=13)).

FIG. 24H is a graph depicting the mean plasma didemethylcitalopram concentration versus time profile for the study described in Example 24 (linear scale, Group 1 (n=13)).

FIG. 24I is a graph depicting the mean plasma bupropion concentration versus time profile for the study described in Example 24 (linear scale, Group 2 (n=13)).

FIG. 24J is a graph depicting the mean plasma hydroxybupropion concentration versus time profile for the study described in Example 24 (linear scale, Group 2 (n=13)).

FIG. 24K is a graph depicting the mean plasma bupropion threoamino alcohol concentration versus time profile for the study described in Example 24 (linear scale, Group 2 (n=13)).

FIG. 24L is a graph depicting the mean plasma bupropion erythroamino alcohol concentration versus time profile for the study described in Example 24 (linear scale, Group 2 (n=13)).

FIG. 24M is a graph depicting the mean plasma PAWC concentration versus time profile for the study described in Example 24 (linear scale, Group 2 (n=13)).

FIG. 24N is a graph depicting the mean plasma citalopram concentration versus time profile for the study described in Example 24 (linear scale, Group 2 (n=13)).

FIG. 24O is a graph depicting the mean plasma demethylcitalopram concentration versus time profile for the study described in Example 24 (linear scale, Group 2 (n=13)).

FIG. 24P is a graph depicting the mean plasma didemethylcitalopram concentration versus time profile for the study described in Example 24 (linear scale, Group 2 (n=13)).

FIG. 25A is a graph depicting the mean plasma bupropion concentration versus time profile for the study described in Example 25 (linear scale, n=14).

FIG. 25B is a graph depicting the mean plasma hydroxybupropion concentration versus time profile for the study described in Example 25 (linear scale, n=14).

FIG. 25C is a graph depicting the mean plasma bupropion threoamino alcohol concentration versus time profile for the study described in Example 25 (linear scale, n=14).

FIG. 25D is a graph depicting the mean plasma bupropion erythroamino alcohol concentration versus time profile for the study described in Example 25 (linear scale, n=14).

FIG. 25E is a graph depicting the mean plasma PAWC concentration versus time profile for the study described in Example 25 (linear scale, n=14).

FIG. 25F is a graph depicting the mean plasma escitalopram concentration versus time profile for the study described in Example 25 (linear scale, n=13).

FIG. 25G is a graph depicting the mean plasma S-demethylcitalopram concentration versus time profile for the study described in Example 25 (linear scale, n=13).

FIG. 25H is a graph depicting the mean plasma S-didemethylcitalopram concentration versus time profile for the study described in Example 25 (linear scale, n=13).

FIG. 26A is a graph depicting the mean plasma bupropion concentration versus time profile for the study described in Example 26 (linear scale, n=13).

FIG. 26B is a graph depicting the mean plasma hydroxybupropion concentration versus time profile for the study described in Example 26 (linear scale, n=13).

FIG. 26C is a graph depicting the mean plasma bupropion threoamino alcohol concentration versus time profile for the study described in Example 26 (linear scale, n=13).

FIG. 26D is a graph depicting the mean plasma bupropion erythroamino alcohol concentration versus time profile for the study described in Example 26 (linear scale, n=13).

FIG. 26E is a graph depicting the mean plasma PAWC concentration versus time profile for the study described in Example 26 (linear scale, n=13).

FIG. 26F is a graph depicting the mean plasma escitalopram concentration versus time profile for the study described in Example 26 (linear scale, n=14).

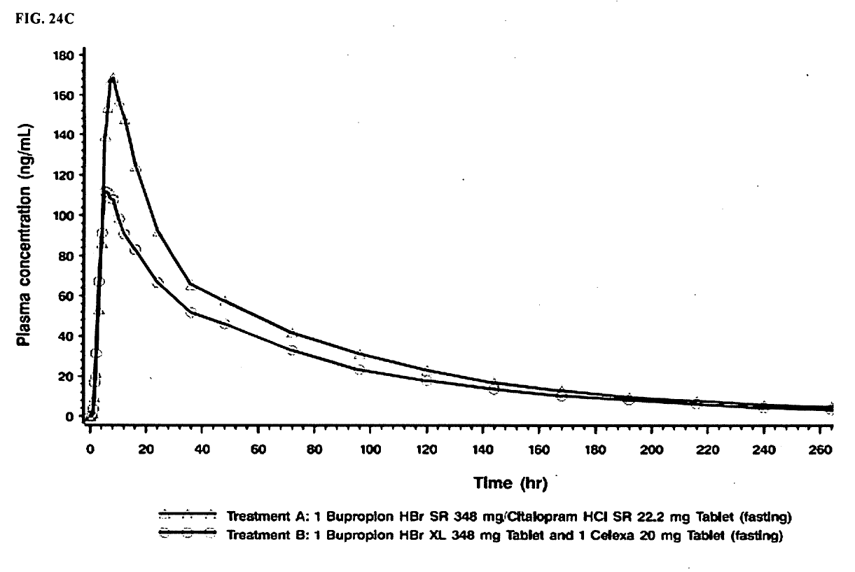

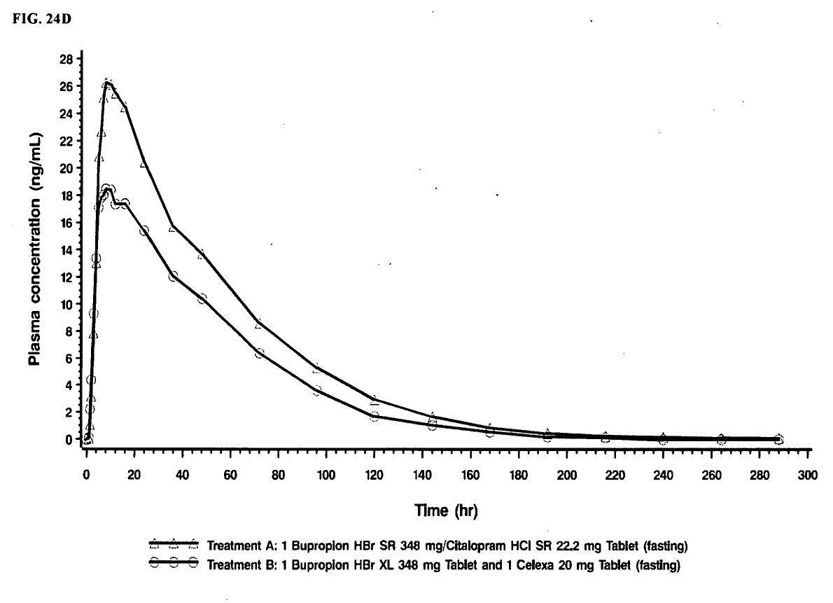

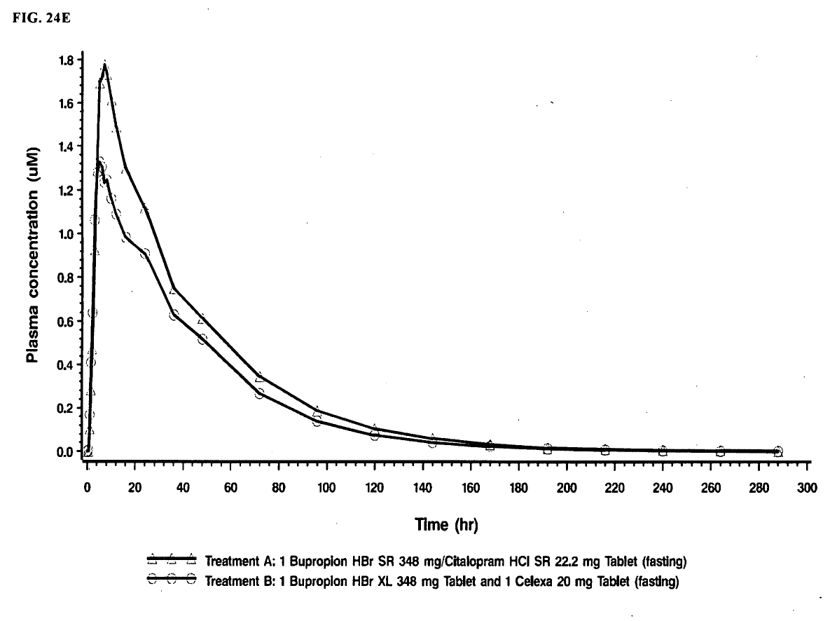

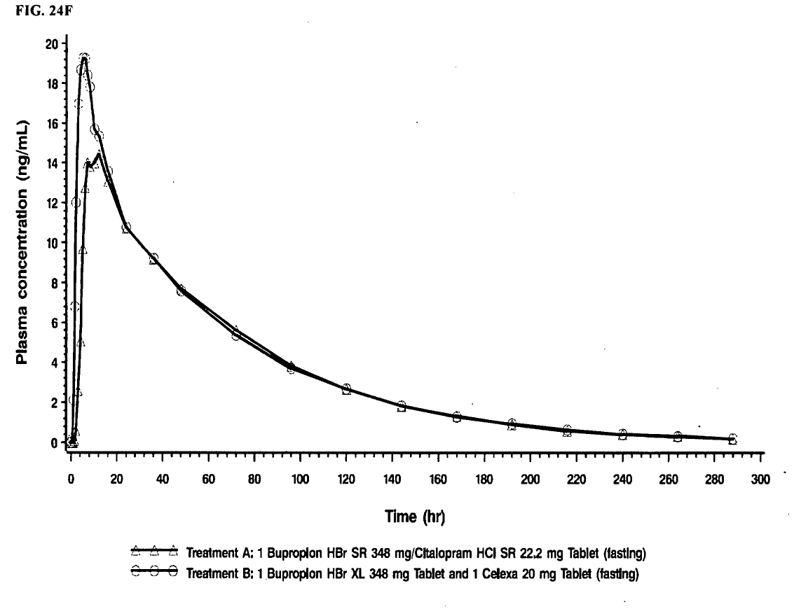

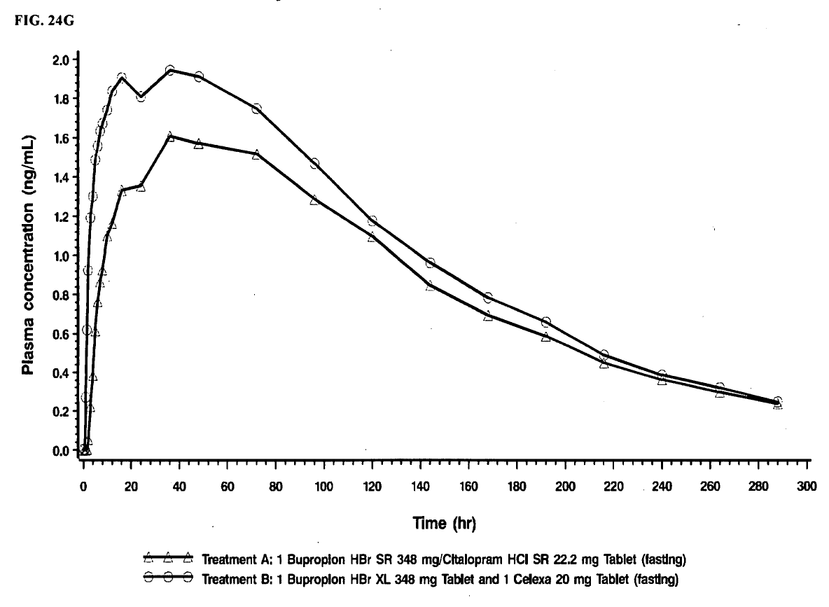

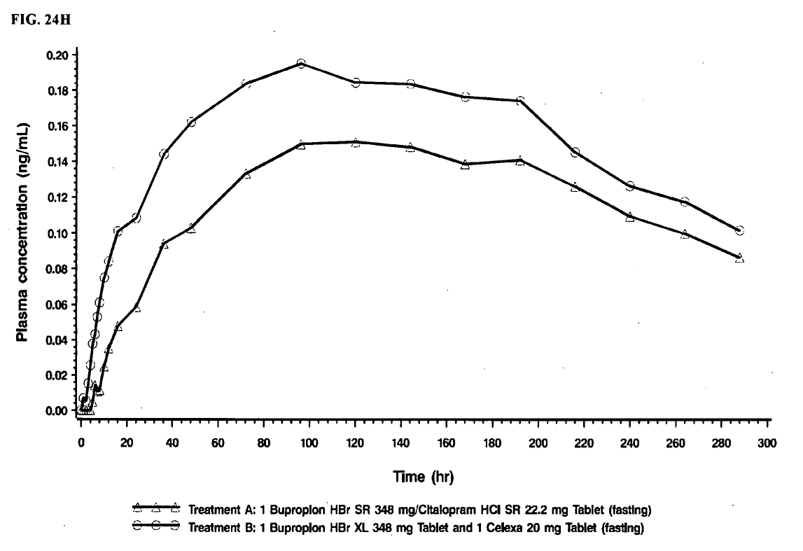

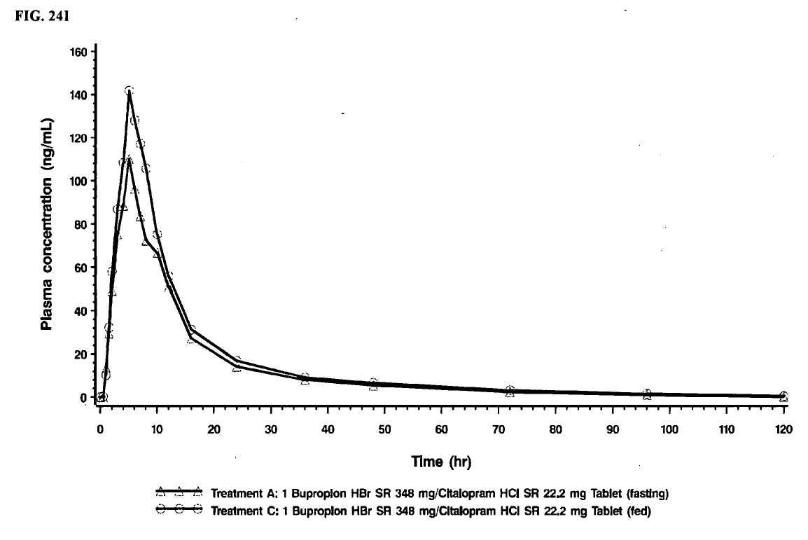

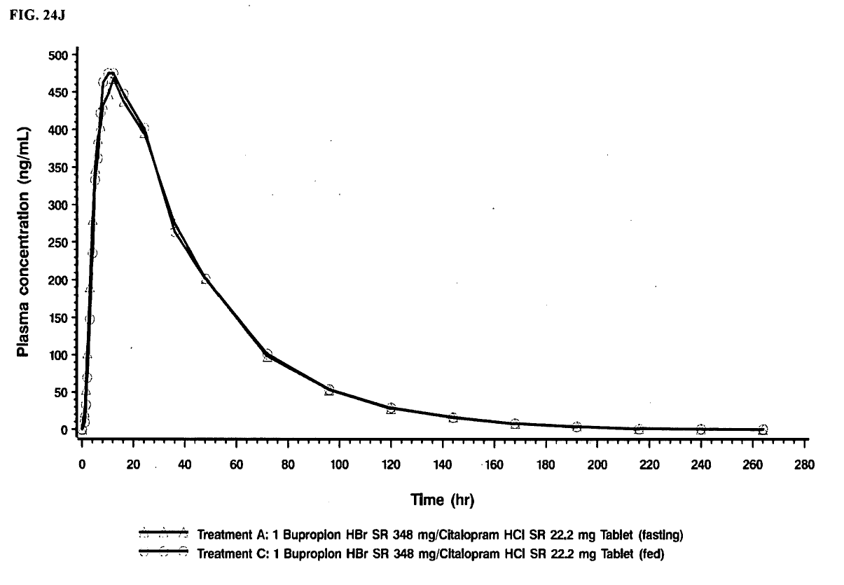

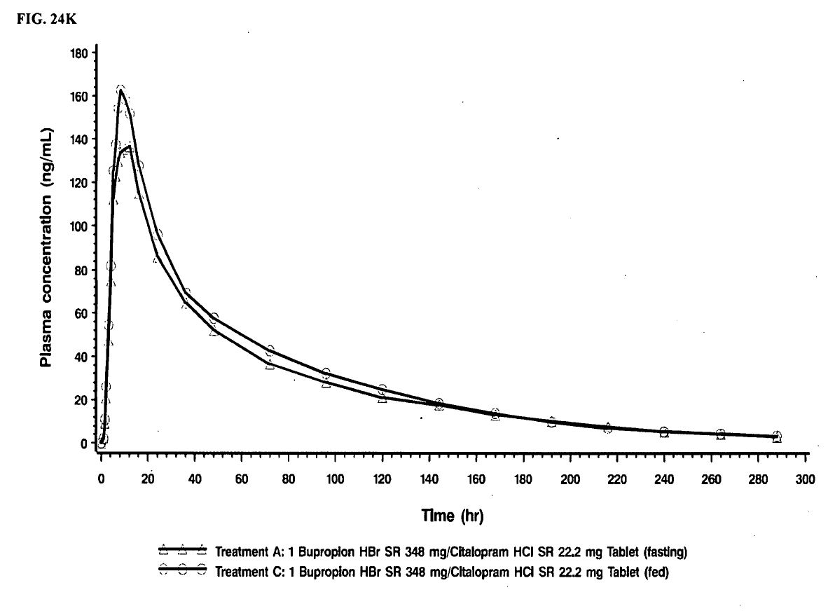

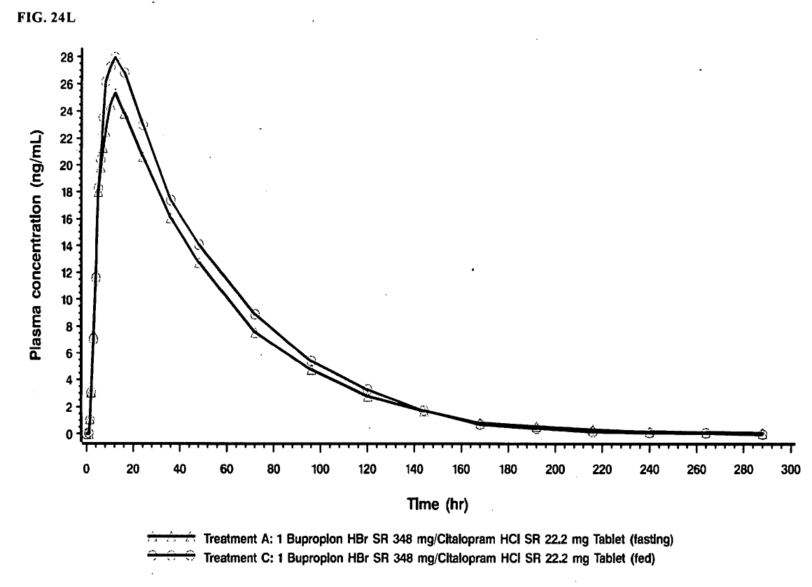

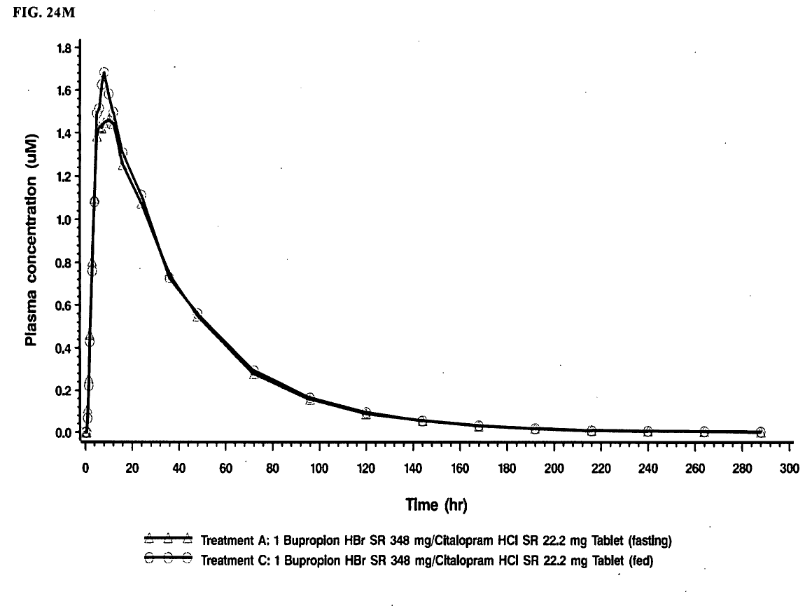

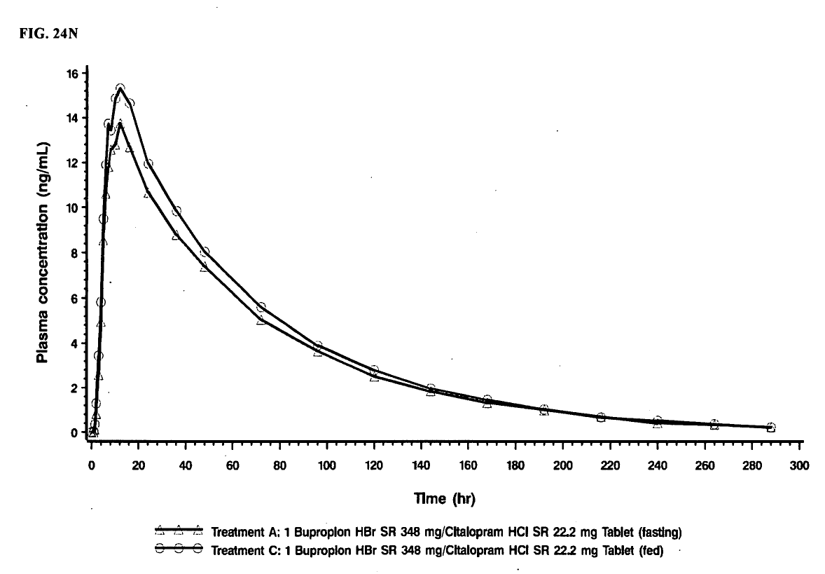

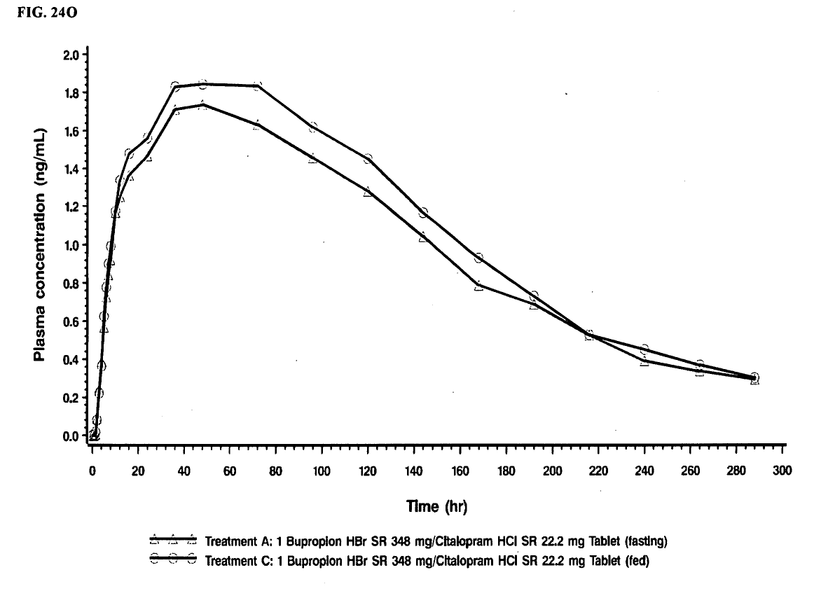

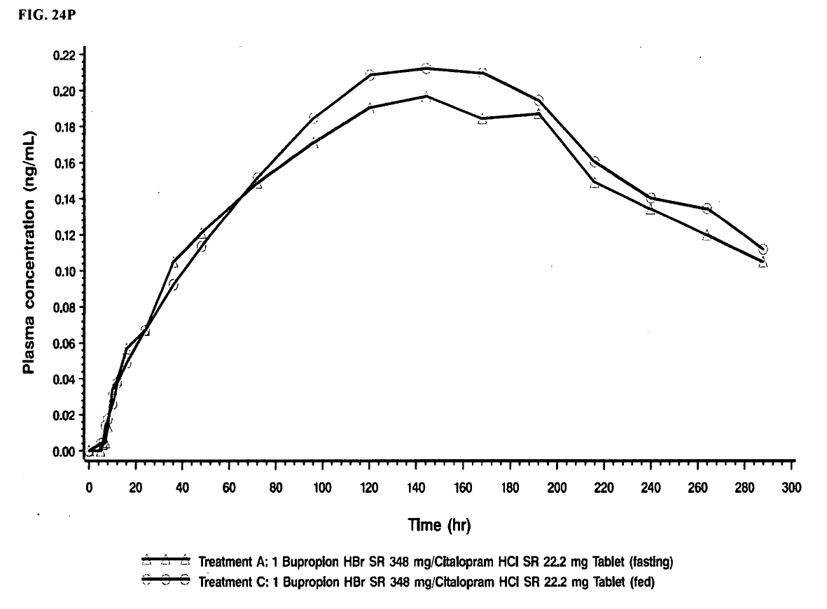

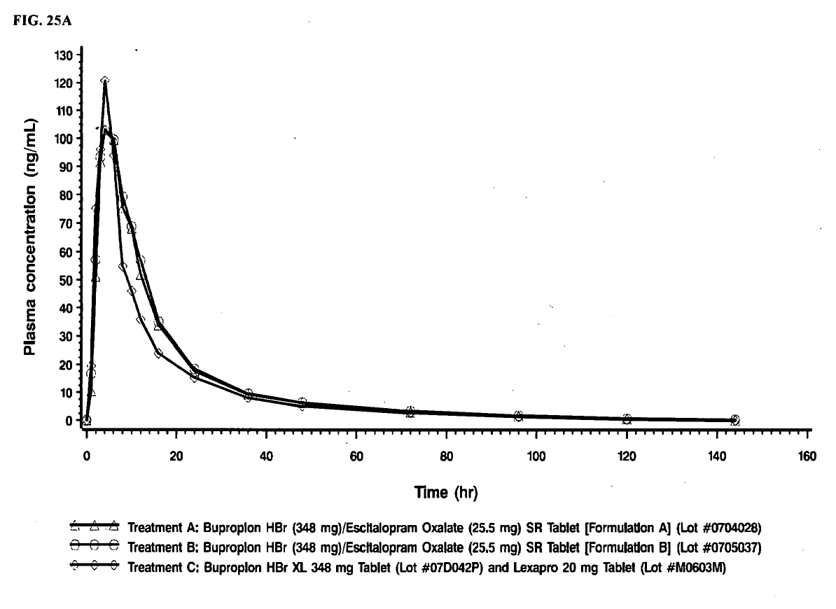

FIG. 24G is a graph depicting the mean plasma S-demethylcitalopram concentration versus time profile for the study described in Example 26 (linear scale, n=14).

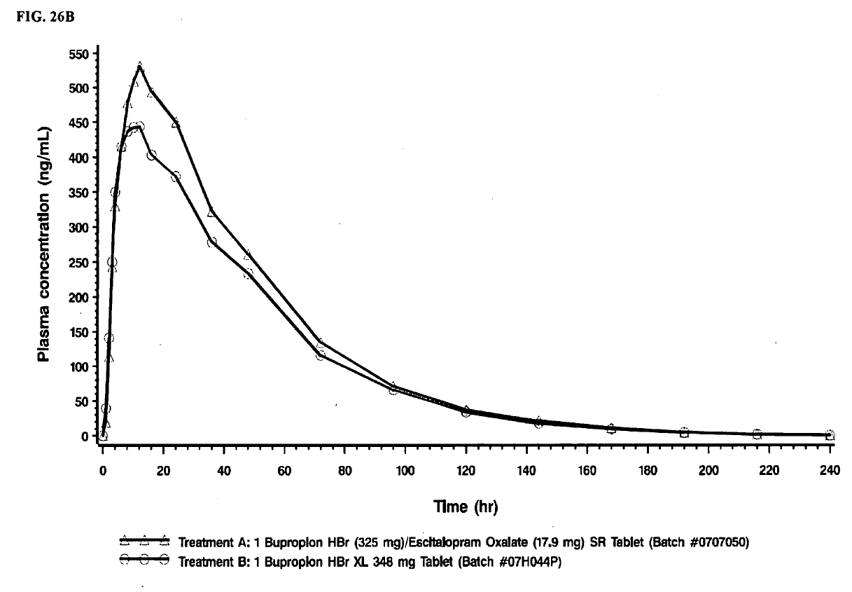

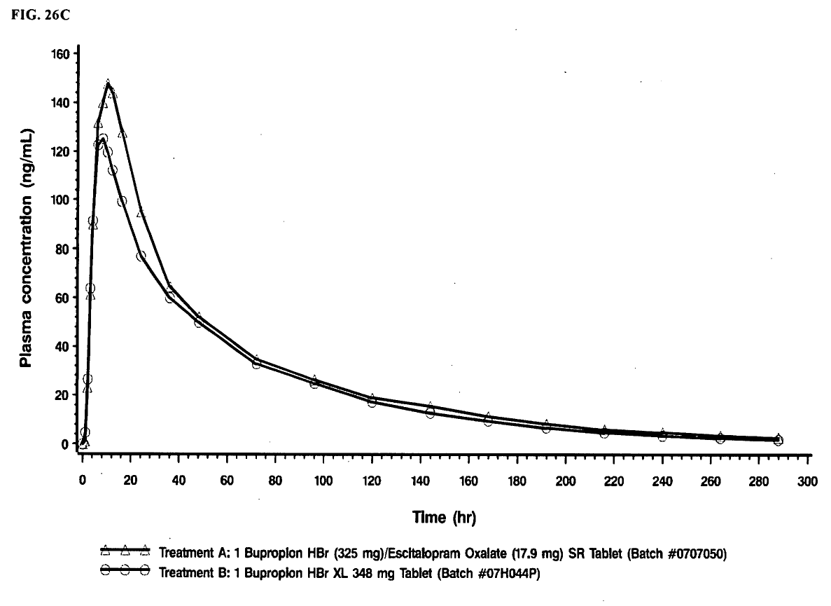

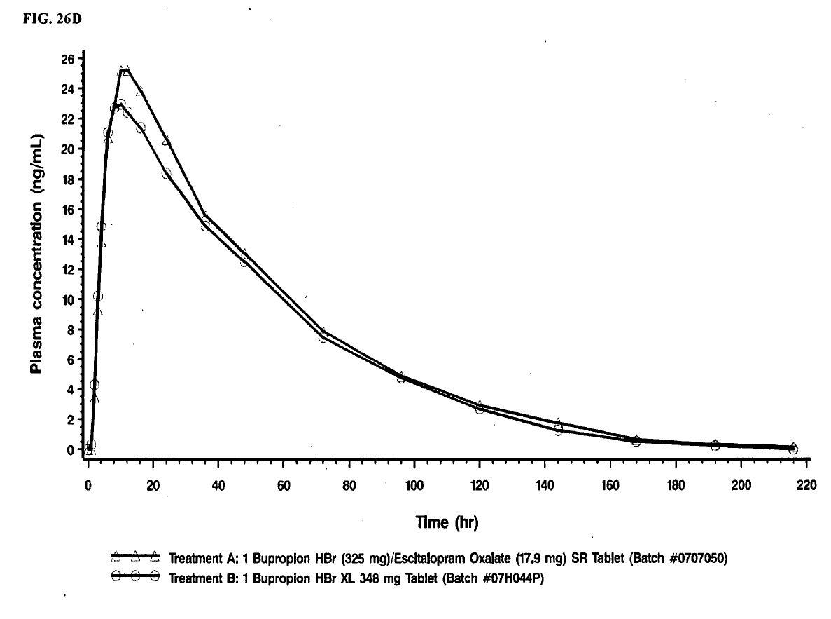

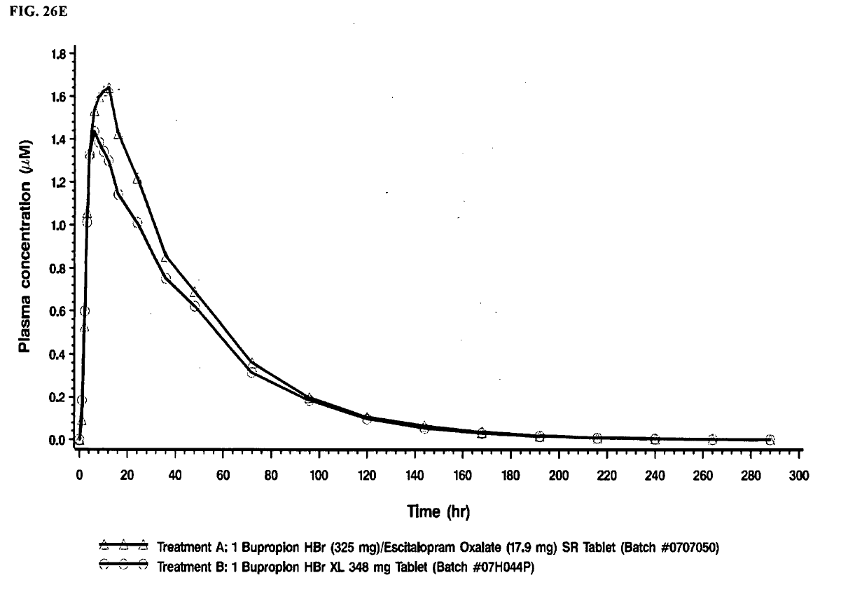

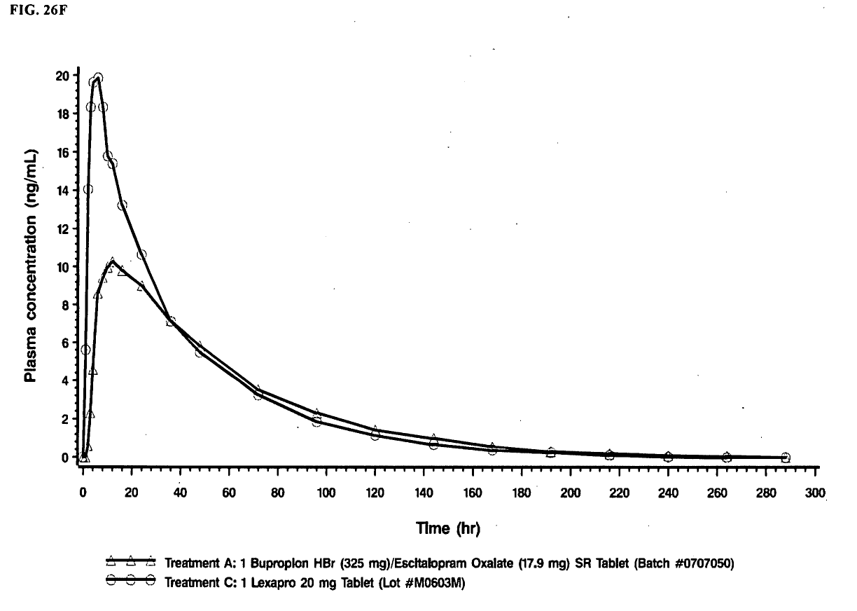

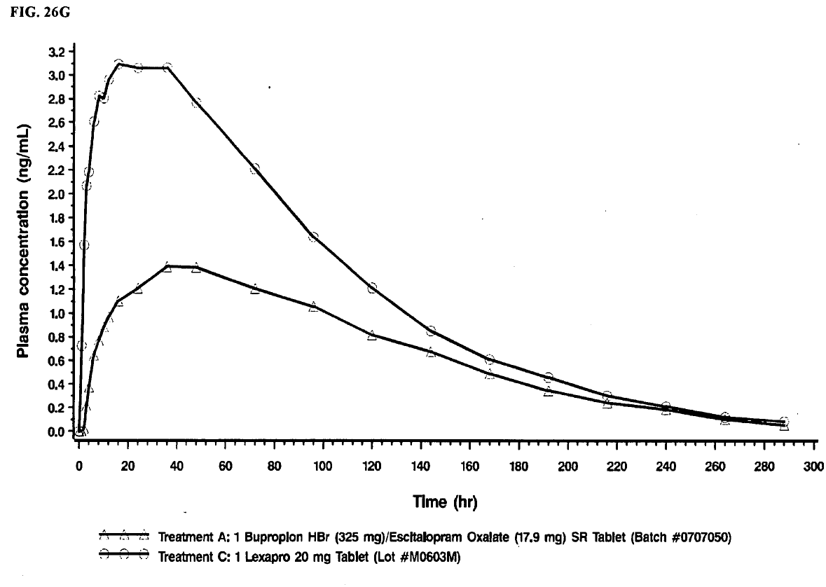

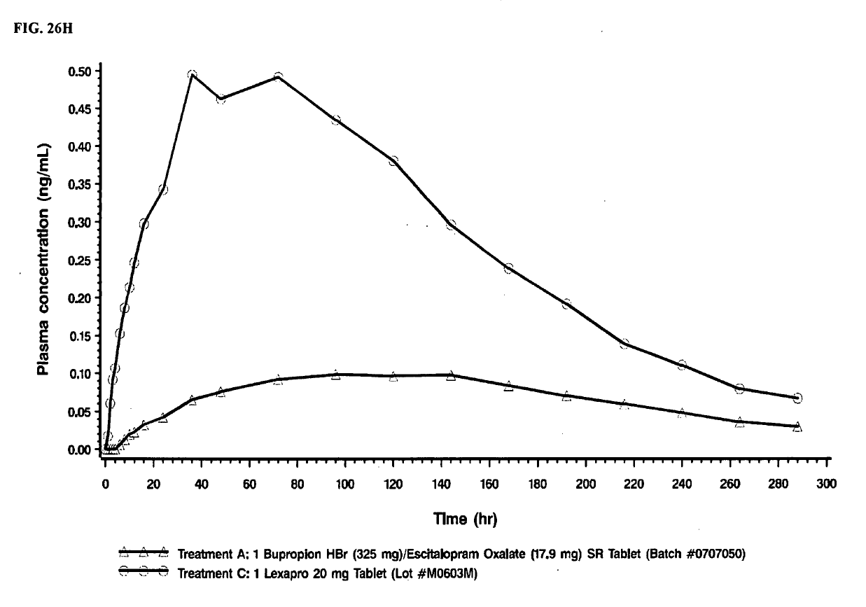

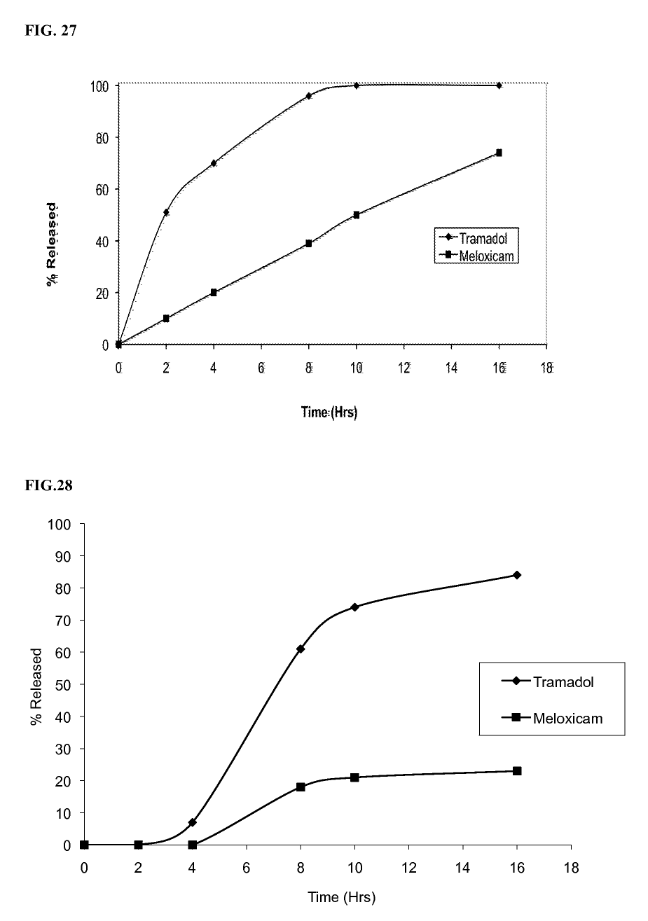

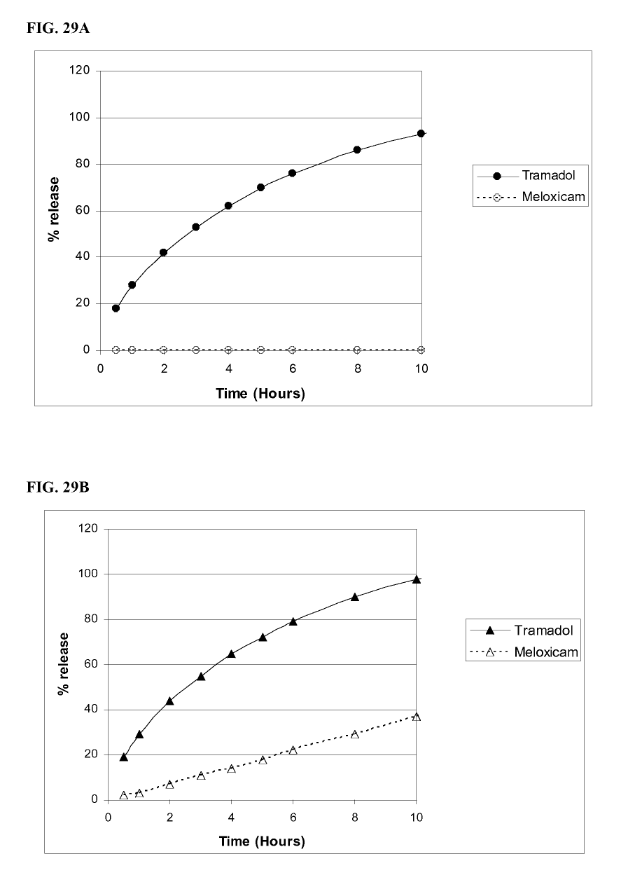

FIG. 26H is a graph depicting the mean plasma S-didemethylcitalopram concentration versus time profile for the study described in Example 26 (linear scale, n=14).

FIG. 27 is a graph depicting the dissolution profile in 900 ml of pH 7.5 phosphate buffer using USP Apparatus 1 at 75 rpm at 37° C. of the composition described in Example 27.

FIG. 28 is a graph depicting the dissolution profile in 900 ml of pH 7.5 phosphate buffer using USP Apparatus 1 at 75 rpm at 37° C. of the composition described in Example 28.

FIG. 29A is a graph depicting the dissolution profile in 900 ml of 0.1N HCl using USP Apparatus 1 at 75 rpm at 37° C. of the composition described in Example 29.

FIG. 29B is a graph depicting the dissolution profile in 900 ml of pH 6.8 phosphate buffer using USP Apparatus 1 at 75 rpm at 37° C. of the composition described in Example 29.

### DEFINITIONS

The term “a” or “an” as used herein means “one” or “one or more”.

The term “about” or “approximately” as used herein means within an acceptable range for the particular value as determined by one of ordinary skill in the art. An accetable range may depend on how the value is measured or determined, i.e., the limitations of the measurement system or on the desired properties sought to be obtained by the present invention.

The term “active”, “active agent”, “active pharmaceutical agent”, “active drug” or “drug” as used herein means the active pharmaceutical ingredient (“API”), which can be either bupropion hydrobromide, bupropion hydrochloride, citalopram hydrochloride, escitalopram oxalate, or quetiapine fumarate alone or in combination. The terms also include the anhydrous, hydrated, solvated forms, prodrugs, as well as polymorphs of the API.

The term “tablet core” as used herein refers to the part of the once-daily pharmaceutical composition comprising the active agents, at least one pharmaceutically acceptable excipient, and optionally at least one stabilizer minus the control-releasing coat. More specifically, a tablet core can be a homogenous core, a controlled-release matrix core, or a bi-layered core.

The term “homogenous core” as used herein refers to a composition in which the combination of active agents selected from the group consisting of bupropion hydrochloride (bupropion HCl) and escitalopram oxalate (escitalopram Ox), bupropion hydrobromide (bupropion HBr) and citalopram hydrochloride (citalopram HCl), bupropion HBr and escitalopram Ox, or bupropion hydrobromide and quetiapine fumarate are blended together with at least one other pharmaceutically acceptable excipient to form a homogenous solid core which has a uniform structure or composition throughout and is free of discreet zones or layers of the active agent combinations. The homogenous core does not have any controlled-release properties and hence can also be referred to as “non-controlled release matrix homogenous cores”. The homogenous core is preferably manufactured into a unitary core.

The term “controlled release matrix core” as used herein refers to a composition comprising at least one hydrophilic control-releasing polymer present in a control-releasing amount, a combination of active agents selected from the group consisting of bupropion HCl and escitalopram Ox, bupropion HBr and citalopram HCl, bupropion HBr and escitalopram Ox, or bupropion hydrobromide and quetiapine fumarate, and at least one pharmaceutically acceptable excipient. Examples of such control-releasing polymers can include, for example, hydrophilic celluloses, ethylcellulose, polysaccharides, polyvinylpyrrolidone, zein, ethylcellulose, polymethacrylates, and mixtures of polyvinyl acetate and polyvinylpyrrolidone, commercially available as Kollidon® SR. The controlled release matrix core may comprise at least one other pharmaceutically acceptable excipient present in amounts that do not contribute to the control-release of the combination of actives, but are present for the ease of manufacture of the controlled release matrix core. The ingredients are blended together to form a homogenous solid core, which has a uniform structure or composition throughout and is free of discreet zones or layers of the active agent combinations.

The terms “therapeutically effective”, “pharmaceutically effective”, or “effective amount” as used herein refers to the amount or quantity of the combination of active agents enough for the required or desired therapeutic response or the amount which is sufficient to elicit an appreciable biological response, when administered to a patient in need of administration of the combination of drugs. The exact amount of the combination of active agents required will vary from subject to subject, depending on age, general condition of the subject, the severity of the condition being treated, and the particular combination of drugs administered. Thus, it is not possible to specify and exact “therapeutically effective” amount. As is well known, the specific dosage for a given patient under specific conditions and for a specific disease will routinely vary, but determination of the optimum amount in each case can readily be accomplished by simple routine procedures. Thus, an appropriate “therapeutically effective” amount in any individual case may be determined by one of ordinary skill in the art using routine experimentation.

The term “dose dumping” as used herein refers to the unintended rapid release of the entire amount or a significant fraction of the active agents in a short period of time from a controlled release or modified-release dosage form in a fixed time relative to the release of the active agents that occurs when the same controlled release or modified-release dosage form is not subject to conditions which induces dose dumping. Conditions that may induce dose dumping include for concomitant ingestion of alcohol or food.

The term “control-releasing coating” or “sustained release coating” as used herein refers to a functional coating which when applied onto a core comprising an active or combination of actives does not result in the immediate release of the active or combination of actives. The coating is permeable to the active or combination of actives in the absence of any monomeric pore forming agents and is free of any pre-formed pores. The coating when applied onto a core comprising an active or combination of actives, modifies or controls the release of the active agents when compared to an uncoated core comprising the same active or combination of actives. The term “controlled release” includes any nonimmediate release pharmaceutical composition. A “controlled release” or “sustained release” pharmaceutical composition, when administered orally or when placed in dissolution media, does not result in the immediate release of the active or combination of actives from the once-daily pharmaceutical composition.

The term “synchronous release” as used herein refers to the substantially similar rate of release of the combination of active agents from the once-daily pharmaceutical composition in dissolution media in-vitro regardless of pH.

The term “plasticizer” as used herein includes any compound or combination of compounds capable of plasticizing or softening a polymer or binder used in the present invention, The use of plasticizers is optional, and can be included in the dosage form to modify the properties of and characteristics of the polymers used in the control-releasing coating for convenient processing of the coat during manufacture of the coated pharmaceutical composition. Once the coated, plasticized pharmaceutical composition has been manufactured, the plasticizer can function to increase the hydrophilicity of the coat in the environment of use. During manufacture of the coated, plasticized pharmaceutical composition, the plasticizer(s) can lower the melting temperature or glass transition temperature (softening point temperature) of the polymer or combination of polymers used in the manufacture of the control-releasing coat. The plasticizer(s) can also broaden the average molecular weight of a polymer or combination of polymers used in the manufacture of the control-releasing coat, thereby also lowering the glass transition temperature of the control-releasing coat. Plasticizers can also reduce the viscosity of a polymer or combinations of polymers for convenient processing of the coat solution when manufacturing the control-releasing coat.

The term “tablet” as used herein refers to a single dosage form comprising the combination of active agents to be administered to a patient in need of such administration. The term “tablet” also includes a tablet that may be a combination of one or more minitablets.

The term “single active agent pharmaceutical compositions” as used herein refers to pharmaceutical compositions comprising only one active agent. For example, a single active agent pharmaceutical composition of bupropion HBr contains only bupropion HBr and no other active agent. A single active agent pharmaceutical composition of bupropion HCl contains only bupropion HCl and no other active agent. The single active agent pharmaceutical composition of bupropion HCl described herein is commercially available as Wellbutrin® XL in 150 mg and 300 mg dosage strengths in the US. Similarly, the single active agent pharmaceutical composition of citalopram HCl contains only citalopram HCl and no other active agent. The single active agent pharmaceutical composition of citalopram HCl described herein is commercially available as Celexa® in the US and is available in dosage strengths of 10 mg, 20 mg, and 40 mg of the base. The single active agent pharmaceutical composition of escitalopram Ox described herein contains escitalopram Ox as the sole active agent and is commercially available as Lexapro® in the US in dosage strengths of 5 mg, 10 mg, and 20 mg of the base.

The term “co-administration” as used herein refers to administering to a patient in need of such administration a first single active agent pharmaceutical composition together with a second single active agent pharmaceutical composition which may containing the same single active agent as the first single active agent pharmaceutical composition or a different single active agent pharmaceutical composition simultaneously. For example, co-administration of 300 mg Wellbutrin® XL and 20 mg Lexapro® means that one 300 mg Wellbutrin® XL tablet and one 20 mg Lexapro® tablet are administered to a patient in need of such administration at the same time.

The term “immediate-release coat” as used herein is defined to mean a coat, which has substantially no influence on the rate of release of an active or combination of actives from the once-daily pharmaceutical composition in-vitro or in-vivo when compared to a pharmaceutical composition comprising the same active or combination of actives. The excipients comprising the immediate release coat have no substantial controlled release, swelling, erosion, or erosion and swelling properties, which could lead to the non-immediate release of the active or combination of actives from the once-daily pharmaceutical composition. The immediate release coat can enhance the chemical, biological, physical stability, or the physical appearance of the once-daily pharmaceutical composition.

The term “immediate release core” or “immediate release layer” as used herein refers to a core or immediate release layer within a core, which has substantially no influence on the rate of release of an active or combination of actives from the once-daily pharmaceutical composition in-vitro or in-vivo when compared to a controlled release matrix core comprising the same active or combination of actives. The excipients comprising the immediate release core or immediate release layer within a core have no substantial controlled release, swelling, erosion, or erosion and swelling properties, which could lead to the non-immediate release of the active or combination of actives from the immediate release core or immediate release layer within a core.

“Stabilizer”, as the term is used herein, means a compound when present in an effective stabilizing amount inhibits or prevents the degradation of the active agents, so that the stabilizer can be used in the once-daily pharmaceutical composition while retaining much of the active agents' potency over time. Stabilizers useful in accordance with the present invention retain at least about 80% of the potency of the active agents and preferably over 90% of potency after one year of storage at room temperature (59-77° C.) at 35-60% humidity. When used herein, the term “potency” means the weight of the active agent remaining in a pharmaceutical composition after a period of time has elapsed, for example about a year under ambient conditions or about 12 weeks at about 40° C. and about 75% relative humidity, expressed as a percentage of the initial weight of the active agents in the composition. The weight is measured by suitable quantitative analytical techniques known to one of ordinary skill in the art, such as for example an HPLC.

“Free of food effect” as used herein means that the bioavailability of the desired combination of drug actives when administered using the once-daily pharmaceutical compositions of the present invention is not statistically significantly different between a fed and fasted study as described in the Guidance for Industry:Food-Effect Bioavailability and Fed Bioequivalence Studies, U.S. Department of Health and Human Services, Food and Drug Administration, Center for Drug Evaluation and Research (CDER), December 2002.

### DETAILED DESCRIPTION OF THE INVENTION

The present invention relates to a pharmaceutical composition comprising a tablet core comprising a combination of actives selected from the group consisting of bupropion hydrochloride and escitalopram oxalate, bupropion hydrobromide and citalopram hydrochloride, bupropion hydrobromide and escitalopram oxalate, and bupropion hydrobromide and quetiapine fumarate, and at least one pharmaceutically acceptable excipient, and a control-releasing coat surrounding the tablet core, wherein said composition surprisingly provides for a synchronous release of the combination of active agents across the pH range i.e., 0.1N HCl, pH 4.5 acetate buffer, and pH 6.8 phosphate buffer in-vitro. The once-daily pharmaceutical composition surprisingly also provides for enhanced absorption of bupropion hydrobromide when administered to a subject in need of such administration. The once-daily pharmaceutical composition provides an about 15-25% increase in the bioavailability of bupropion when compared to co-administration of single active agent pharmaceutical compositions of bupropion hydrobromide and citalopram hydrochloride or bupropion hydrobromide and escitalopram oxalate.

The Tablet Cores

In certain embodiments of the present invention, the tablet core comprises a combination of actives selected from the group consisting of a therapeutically effective combination of active agents selected from the group consisting of bupropion hydrochloride and escitalopram oxalate, bupropion hydrobromide and citalopram hydrochloride, bupropion hydrobromide and escitalopram oxalate, bupropion hydrobromide and quetiapine fumarate, and optionally a stabilizer, and at least one pharmaceutically acceptable excipient.

In embodiments where the tablet core comprises a combination of bupropion hydrochloride and escitalopram oxalate, the amount of bupropion hydrochloride present in the homogenous tablet core can be about 10%, about 20%, about 30%, about 40%, about 50%, about 60%, about 70%, about 80%, about 90%, or about 99% w/w of the dry tablet core weight, and the amount of escitalopram oxalate can be present at about 0.1%, about 0.2%, about 0.4%, about 0.6%, about 0.8%, about 1%, about 2%, about 4%, about 6%, about 8%, about 10%, about 20%, about 30%, about 40%, or about 50% w/w of the dry tablet core weight. In at least one embodiment of the present invention, the amount of bupropion hydrochloride is about 300 mg and the amount of escitalopram oxalate is about 20 mg (16 mg escitalopram free base).

In embodiments where the tablet core comprises a combination of bupropion hydrobromide and escitalopram oxalate, the amount of bupropion hydrobromide present in the homogenous tablet core can be about 10%, about 20%, about 30%, about 40%, about 50%, about 60%, about 70%, about 80%, about 90%, or about 99% w/w of the dry tablet core weight, and the amount of escitalopram oxalate can be present at about 0.1%, about 0.2%, about 0.4%, about 0.6%, about 0.8%, about 1%, about 2%, about 4%, about 6%, about 8%, about 10%, about 20%, about 30%, about 40%, or about 50% w/w of the dry tablet core weight. In at least one embodiment of the present invention, the amount of bupropion hydrobromide is about 325 mg and the amount of escitalopram oxalate is about 16 mg. In at least one other embodiment of the present invention, the amount of bupropion hydrobromide is about 156 mg and the amount of escitalopram oxalate is about 8 mg.

In embodiments where the tablet core comprises a combination of bupropion hydrobromide and citalopram hydrochloride, the amount of bupropion hydrobromide present in the homogenous tablet core can be about 10%, about 20%, about 30%, about 40%, about 50%, about 60%, about 70%, about 80%, about 90%, or about 99% w/w of the dry tablet core weight, and the amount of escitalopram oxalate can be present at about 0.1%, about 0.2%, about 0.4%, about 0.6%, about 0.8%, about 1%, about 2%, about 4%, about 6%, about 8%, about 10%, about 20%, about 30%, about 40%, or about 50% w/w of the dry tablet core weight. In at least one embodiment of the present invention, the amount of bupropion hydrobromide is about 348 mg and the amount of citalopram hydrochloride is about 22.2 mg.

In embodiments where the tablet core comprises a combination of bupropion hydrobromide and quetiapine fumarate, the amount of bupropion hydrobromide present in the homogenous tablet core can be about 10%, about 20%, about 30%, about 40%, about 50%, about 60%, about 70%, about 80%, about 90%, or about 99% w/w of the dry tablet core weight, and the amount of quetiapine fumarate can be present at about 1%, about 2%, about 4%, about 6%, about 8%, about 10%, about 20%, about 30%, about 40%, about 50%, about 60%, about 70%, about 80%, or about 90% w/w of the dry tablet core weight. In at least one embodiment of the present invention, the amount of bupropion hydrobromide is about 348 mg and the amount of quetiapine fumarate is about 23 mg.

In certain embodiments of the present invention, the tablet core can comprise a pharmaceutically acceptable suitable stabilizer. Stabilizers are used to inhibit degradation of the combination of active agents, thereby maintaining their potency over time (at least 12-months) and increasing shelf life of the finished pharmaceutical compositions of the invention. Stabilizers for bupropion hydrochloride or bupropion hydrobromide are optional. Stabilizers suitable for inhibiting degradation of bupropion hydrochloride or bupropion hydrobromide may be chosen based on the stabilizer's ability to provide an acidic environment in the once-daily pharmaceutical composition. Stabilizers suitable for inhibiting degradation of bupropion hydrochloride or bupropion hydrobromide, in embodiments where such stabilizers are utilized include, for example, pharmaceutically acceptable inorganic acids, which at a concentration of about 0.31% w/w/ form an aqueous solution having a pH of from about 0.5 to about 0.4. Examples of such inorganic acids include, but are not limited to, hydrochloric acid, phosphoric acid, nitric acid, and sulfuric acid, or combinations thereof. Pharmaceutically acceptable suitable organic acids that have a solubility in water at 20° C. of less than about 10 g/100 g water and that at a concentration of about 60% w/w form an aqueous suspension having a pH of from about 0.9 to about 4.0 can also function as suitable stabilizers. Examples of such organic acids include, but are not limited to, dicarboxylic acids, such as for example, lactic, formic, acetic, oxalic, succinic, adipic, fumaric, and phthalic acid, or combinations thereof. Citric acid is another example of a suitable organic acid hat can be used as an effective stabilizer. Other non-limiting examples of suitable stabilizers include salts of organic bases such as, creatinine hydrochloride, preferably having an aqueous pH of from about 2.70 to about 3.10 at a concentration of about 10% w/w, thiamine hydrochloride, preferably having an aqueous pH of from about 2.95 to about 3.05, at a concentration of about 20% w/w, pyridoxine hydrochloride, preferably having an aqueous pH of from about 2.70 to about 2.72, at a concentration of about 20% w/w, or combinations thereof. Suitable salts of inorganic acids can also function as stabilizers. An example of such a salt includes, but is not limited to potassium phosphate monobasic, preferably having an aqueous pH of from about 4.20 to about 4.30 at a concentration of about 10 w/w. Other stabilizers suitable for use include acid salts of amino acids such as L-cysteine hydrochloride, L-cystine dihydrochloride and glycine hydrochloride, or combinations thereof and sulfites such as potassium metabisulfite and sodium bisulfite, or combinations thereof. The amount of stabilizer appropriate for inhibiting degradation of bupropion hydrochloride or bupropion hydrobromide can be about 0.1%, about 0.2%, about 0.4%, about 0.6%, about 0.8%, about 1%, about 2%, about 4%, about 6%, about 8%, about 10%, about 15%, about 20%, about 25%, or about 30% w/w of the dry tablet core weight. In at least one embodiment of the present invention, the amount of stabilizer appropriate for inhibiting degradation of bupropion hydrochloride or bupropion hydrobromide can be about 5% w/w of the dry tablet core. Stabilizers for citalopram hydrochloride and escitalopram oxalate are optional. Pharmaceutically acceptable suitable stabilizers can be added to stabilize the citalopram hydrochloride or escitalopram oxalate when the citalopram hydrochloride or escitalopram oxalate is in intimate contact with either bupropion hydrochloride or bupropion hydrobromide. In embodiments of the present invention that utilize stabilizers for citalopram hydrochloride or escitalopram oxalate, the suitable stabilizers can be selected from the class of phenylated antioxidants. Non-limiting examples of such phenylated antioxidants include butlylated hydroxytoluene (BHT), butylated hydroxyanisole (BHA), or combinations thereof. In certain embodiments of the present invention, BHT is the preferred stabilizer and can be present at about 0.01%, about 0.02%, about 0.04%, about 0.06%, about 0.08%, about 1%, about 1.5%, about 2%, about 2.5%, about 3%, about 3.5%, about 4%, about 4.5%, or about 5% w/w of the dry tablet core weight. In at least one embodiment of the present invention, BHT comprises about 0.1% w/w of the dry tablet core weight.

In certain embodiments of the present invention, the tablet core can comprise at least one pharmaceutically acceptable excipient conventional in the pharmaceutical arts. Such pharmaceutically acceptable excipients include spheronization aids, solubility enhancers, disintegrating agents, diluents, lubricants, binders, fillers, glidants, etc. Depending on the intended main function, excipients to be used in formulating compositions are subcategorized into different groups. However, one excipient can affect the properties of a composition in a series of ways, and many excipients used in compositions can thus be described as being multifunctional.

In certain embodiments of the present invention, the tablet cores can comprise at least one diluent. Any suitable diluent conventional in the pharmaceutical art can be used. Non-limiting examples of diluents suitable for use in the present invention include, lactose, microcrystalline cellulose, mannitol, and combinations thereof. In embodiments comprising lactose, the lactose can be lactose anhydrous (direct tabletting). In embodiments comprising microcrystalline cellulose, the microcrystalline cellulose can be, for example, AVICEL®, such as AVICEL® PH101 or AVICEL® PH 102.

In certain embodiments of the present invention, the tablet cores can comprise at least one binder. Any suitable binder conventional in the pharmaceutical art can be used. A binder (also sometimes called adhesive) can be added to a drug-filler mixture to increase the mechanical strength of the tablet cores. Binders can be added to the formulation in different ways: (1) as a dry powder, which is mixed with other ingredients before wet agglomeration, (2) as a solution, which is used as agglomeration liquid during wet agglomeration, and is referred to as a solution binder, and (3) as a dry powder, which is mixed with the other ingredients before compaction. In this form the binder is referred to as a dry binder. Solution binders are a common way of incorporating a binder into granules. In certain embodiments, the binder used in the tablet cores is in the form of a solution binder. Non-limiting examples of binders useful for the tablet cores include hydrogenated vegetable oil, castor oil, paraffin, higher aliphatic alcohols, higher alphatic acids, long chain fatty acids, fatty acid esters, wax-like materials such as fatty alcohols, fatty acid esters, fatty acid glycerides, hydrogenated fats, hydrocarbons, normal waxes, stearic acid, stearyl alcohol, hydrophobic and hydrophilic polymers having hydrocarbon backbones, and mixtures thereof. Specific examples of water-soluble polymer binders include modified starch, gelatin, polyvinylpyrrolidone, cellulose derivatives (such as for example hydroxypropyl methylcellulose (HPMC) and hydroxypropyl cellulose (HPC)), polyvinyl alcohol and mixtures thereof. In certain embodiments of the invention the binder is polyvinylpyrrolidone (KOLLIDON® 90F, KOLLIDON® K29/32, or combinations thereof). The amount of binder present can be present at about 0.1%, about 0.2%, about 0.4%, about 0.6%, about 0.8%, about 1%, about 2%, about 4%, about 6%, about 8%, about 10%, about 12%, about 14%, about 16%, about 18%, or about 20% w/w of the dry tablet core weight. In at least one embodiment, the binder is present at about 3% w/w of the tablet dry weight.

Certain embodiments of the present invention can comprise at least one lubricant. Any suitable lubricant conventional in the pharmaceutical art may be used. Non-limiting examples of lubricants useful for the tablet cores include glyceryl behenate, stearic acid, hydrogenated vegetable oils (such as hydrogenated cottonseed oil (STEROTEX®), hydrogenated soybean oil (STEROTEX® HM) and hydrogenated soybean oil & castor wax (STEROTEX® K), stearyl alcohol, leucine, polyethylene glycol (MW 1450, suitably 4000, and higher), magnesium stearate, glyceryl monostearate, stearic acid. polyethylene glycol, ethylene oxide polymers (CARBOWAX®), sodium lauryl sulfate, magnesium lauryl sulfate, sodium oleate, sodium stearyl fumarate, DL-leucine, colloidal silica, mixtures thereof and others as known in the art. In certain embodiments of the present invention, the lubricant can be glyceryl behenate (for example, COMPRITOL® 888 ATO). The amount of lubricant present can be about 0.1%, about 0.2%, about 0.4%, about 0.6%, about 0.8%, about 1%, about 2%, about 4%, about 6%, about 8%, or about 10% w/w of the dry tablet core weight. In at least one embodiment, the lubricant is present at about 3% w/w of the tablet dry weight.

In certain embodiments, one or both active agents may be granulated for use in this invention to manufacture the tablet core. Well known granulation methods can be used to manufacture the tablet core, including wet mass granulation (such as high shear and top-spray granulation), dry granulation (such as roller compaction and slugging) and hot-melt granulation. In certain embodiments, the active agents can be granulated individually and then combined, in order to be compressed into a tablet core or they can be co-granulated (both actives granulated together into the one granule) for incorporation into a tablet core.

If the characteristics of both active agents are suitable and granulation is not required, both pharmaceutical actives may be incorporated directly into the tablet blend. Alternatively, one active may need to be granulated (as described above) while the second active is added directly to the tablet blend. If both actives are granulated, the active granules (either dispensed separately or as a co-granule) are incorporated into the tablet blend. The tablet blend is made using conventional tablet blend technologies (e.g. low shear blending using v-blenders or bowl blenders or high-shear blending). The actives are combined with a tablet lubricant. The tablet blend is compressed to the required shape, weight and hardness using a standard tablet press.

In embodiments where the active agents are granulated separately, the bupropion hydrochloride or bupropion hydrochloride is uniformly granulated by spraying the active agents with an aqueous mixture comprising a binder, such as for example polyvinyl alcohol, and optionally a stabilizer, such as for example citric acid in a fluid bed processor or other suitable apparatus known in the art. The bupropion hydrochloride or bupropion hydrobromide granules thus formed are then dried and screened for granules between about 355 μm and about 800 μm. These appropriately seized bupropion hydrochloride or bupropion hydrobromide granules are retained for manufacture of the tablet core. Similarly, the citalopram hydrochloride or escitalopram oxalate is uniformly granulated by spraying the active agents with solvent based mixture comprising a binder, such as for example polyvinylpyrrolidone, and optionally a stabilizer, such as for example BHT in a fluid bed processor or other suitable apparatus known in the art. The citalopram hydrochloride or citalopram hydrobromide granules thus formed are then dried and screened for granules between about 355 μm and about 800 μm. In certain embodiments comprising quetiapine fumarate, the quetiapine fumarate can be granulated by spraying with an aqueous solution of polyvinyl alcohol in a suitable granulating apparatus and subsequently dried. The resulting granules are screened and granules between about 355 μm about 800 μm are retained for use in the tablet core.

In embodiments where a homogenous tablet core is desired, an appropriate amount of each of the sized granulated active agents, equivalent to the dosage strength desired, for the combination is mixed uniformly with a lubricant, such as for example glyceryl behenate, to obtain a homogenous mixture of granules of the two actives and lubricant. The homogenous mixture is then compressed into a homogenous tablet core using a tablet press to a hardness of about 130N using 9 mm round normal concave shaped tablet tooling. The resulting immediate release homogenous tablet core is ready to be coated with a control-releasing coat.

In embodiments where the combination of actives are co-granulated, the granulation solution is first prepared by combining an aqueous solution of a binder, such as for example, polyvinyl alcohol and optionally a stabilizer, such as for example, citric acid together with a solvent based solution comprising a binder, such as for example, polyvinylpyrrolidone and optionally a stabilizer, such as for example BHT. When combining the aqueous based and solvent-based solutions together, the BHT becomes finely dispersed in the PVA/citric acid solution. The combination of active agents is charged to the granulation chamber of a suitable apparatus in the required ratio to give the desired final dosage strengths of each active. The combination of active agents are then sprayed and simultaneously uniformly mixed for a period of time with the granulation solution to obtain a homogenously mixed co-granulate of the combination of active agents. The granules thus obtained are screened through a 1.00 mm screen, and the material <1.00 mm retained for use in the tablet core.

In embodiments where a homogenous tablet core using a co-granulated combination of actives is desired, the dried co-granules obtained by the above described co-granulation method, are then uniformly combined with a lubricant (e.g., glyceryl behenate) to obtain a homogenous tablet core which is then compressed to a target tablet hardness of 130N using 9 mm round normal concave shaped tablet tooling. The resulting immediate release homogenous tablet core is ready to be coated with a control-releasing coat.

In certain embodiments of the present invention, the control-releasing coated homogenous tablet cores of the present invention can avoid the dose dumping of the combination of active agents in the presence of food and/or alcohol regardless of whether the homogenous tablet core is manufactured by the separate granulation or co-granulation methods described herein.

In certain embodiments, the tablet core comprises a controlled-release matrix core. A controlled release matrix core is provided from which the kinetics of drug release from the matrix core are dependent at least in part upon the diffusion and/or erosion properties of excipients within the tablet core. In embodiments where the pharmaceutical composition comprises a tablet core comprising a controlled-release matrix core, the controlled release matrix core comprises a therapeutically effective amount of a combination of bupropion hydrochloride or bupropion hydrobromide and citalopram hydrochloride or escitalopram oxalate, bupropion hydrochloride, or bupropion hydrobromide and quetiapine fumarate, optionally a stabilizer, and at least one pharmaceutically acceptable excipient. The amount of the bupropion salt present in the controlled release matrix can be about 10%, about 20%, about 30%, about 40%, about 50%, about 60%, about 70%, or about 80% w/w of the dry controlled-release matrix core. The amount of the citalopram hydrochloride or escitalopram oxalate present in the controlled release matrix can be about 0.1%, about 0.2%, about 0.4%, about 0.6%, about 0.8%, about 1%, about 2%, about 4%, about 6%, about 8%, about 10%, about 20%, about 30%, about 40%, or about 50% w/w of the dry controlled-release matrix core. The amount of quetiapine fumarate present in the controlled release matrix can be about 0.1%, about 0.2%, about 0.4%, about 0.6%, about 0.8%, about 1%, about 2%, about 4%, about 6%, about 8%, about 10%, about 20%, about 30%, about 40%, or about 50% w/w of the dry controlled-release matrix core. The controlled release matrix is preferably uniparticulate, and can be uncoated or further coated with at least one control-releasing or non-functional coating. Functional coatings can include, by way of example, controlled release polymeric coatings, enteric polymeric coatings, and the like. Non-functional coatings are coatings that do not affect drug release but which affect other properties (e.g., they may enhance the chemical, biological, or the physical appearance of the controlled release formulation). Those skilled in the pharmaceutical art and the design of medicaments are well aware of controlled release matrices conventionally used in oral pharmaceutical compositions adopted for controlled release and means for their preparation. Examples of controlled release matrices are described in U.S. Pat. Nos. 6,326,027; 6,340,475; 6,905,709; 6,645,527; 6,576,260; 6,326,027; 6,254,887; 6,306,438; 6,129,933; 5,891,471; 5,849,240; 5,965,163; 6,162,467; 5,567,439; 5,552,159; 5,510,114; 5,476,528; 5,453,283; 5,443,846; 5,403,593; 5,378,462; 5,350,584; 5,283,065; 5,273,758; 5,266,331; 5,202,128; 5,183,690; 5,178,868; 5,126,145; 5,073,379; 5,023,089; 5,007,790; 4,970,075; 4,959,208; 4,59,208; 4,861,598; 4,844,909; 4,834,984; 4,828,836; 4,806,337; 4,801,460; 4,764,378; 4,421,736; 4,344,431; 4,343,789; 4,346,709; 4,230,687; 4,132,753; 5,591,452; 5,965,161; 5,958,452; 6,254,887; 6,156,342; 5,395,626; 5,474,786; and 5,919,826.

Suitable excipient materials for use in such controlled release matrices include, by way of example, release-resistant or controlled release materials such as hydrophobic polymers, hydrophilic polymers, lipophilic materials and mixtures thereof. Non-limiting examples of hydrophobic, or lipophilic components include glyceryl monostearate, mixtures of glyceryl monostearate and glyceryl monopalmitate (Myvaplex, Eastman Fine Chemical Company), glycerylmonooleate, a mixture of mono, di and tri-glycerides (ATMUL 84S), glycerylmonolaurate, paraffin, white wax, long chain carboxylic acids, long chain carboxylic acid esters, long chain carboxylic acid alcohols, and mixtures thereof. The long chain carboxylic acids can contain from 6 to 30 carbon atoms; in certain embodiments at least 12 carbon atoms, and in other embodiments from 12 to 22 carbon atoms. In some embodiments this carbon chain is fully saturated and unbranched, while others contain one or more double bonds. In at least one embodiment the long chain carboxylic acids contain 3-carbon rings or hydroxyl groups. Non-limiting examples of saturated straight chain acids include n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids. Non-limiting examples of these include oleic acid, gadoleic acid and erucic acid. Also useful are unsaturated (polyolefinic) straight chain monocaboxyic acids. Non-limiting examples of these include linoleic acid, linolenic acid, arachidonic acid and behenolic acid. Useful branched acids include, for example, diacetyl tartaric acid. Non-limiting examples of long chain carboxylic acid esters include glyceryl monostearates; glyceryl monopalmitates; mixtures of glyceryl monostearate and glyceryl monopalmitate (Myvaplex 600, Eastman Fine Chemical Company); glyceryl monolinoleate; glyceryl monooleate; mixtures of glyceryl monopalmitate, glyceryl monostearate glyceryl monooleate and glyceryl monolinoleate (Myverol 18-92, Eastman Fine Chemical Company); glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate (Myverol 18-99, Eastman Fine Chemical Company); acetylated glycerides such as distilled acetylated monoglycerides (Myvacet 5-07, 7-07 and 9-45, Eastman Fine Chemical Company); mixtures of propylene glycol monoesters, distilled monoglycerides, sodium stearoyl lactylate and silicon dioxide (Myvatex TL, Eastman Fine Chemical Company); mixtures of propylene glycol monoesters, distilled monoglycerides, sodium stearoyl lactylate and silicon dioxide (Myvatex TL, Eastman Fine Chemical Company) d-alpha tocopherol polyethylene glycol 1000 succinate (Vitamin E TPGS, Eastman Chemical Company); mixtures of mono- and diglyceride esters such as Atmul (Humko Chemical Division of Witco Chemical); calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; cetearyl octanoate; C10-C30 cholesterol/lavosterol esters; sucrose long chain carboxylic acid esters; and mixtures thereof.

In certain embodiments, alcohols useful as excipient materials for controlled release matrices can include the hydroxyl forms of the carboxylic acids exemplified above and also cetearyl alcohol.

In certain other embodiments, waxes can be useful alone or in combination with the materials listed above, as excipient materials for the controlled release matrix embodiments of the present invention. Non-limiting examples of these include white wax, paraffin, microcrystalline wax, carnauba wax, and mixtures thereof.

The lipophilic agent can be present in an amount of from 5% to 90% by weight of the controlled release matrix dosage form. For example, in certain embodiments the lipophilic agent is present in an amount of from 10% to 85%, and in other embodiments from 30% to 60% by weight of the controlled release matrix dosage form.

Non-limiting examples of hydrophilic polymers that can be used in certain embodiments of the controlled release matrix dosage form include hydroxypropylmethylcel lulose (HPMC), hydroxypropylcellulose (HPC), hydroxyethylceflulose (HEC), carboxymethylcellulose (CMC) or other cellulose ethers, polyoxyethylene, alginic acid, acrylic acid derivatives such as polyacmylic acid, Carbopol (B. F. Goodrich, Cleveland, Ohio). polymethacrylate polymer such as EUDRAGIT® RL, RS, R. S, NE and E (Rhome Pharma, Darmstadt, Germany), acrylic acid polymer, methacrylic acid polymer, hydroyethyl methacrylic acid (HEMA) polymer, hydroxymethyl methacrylic acid (HMMA) polymer, polyvinyl alcohols. In at least one embodiment of the present invention, the control-release matrix core is uncoated and comprises hydroxypropyl cellulose as the hydrophilic polymer.

The hydrophilic polymer can be present in an amount of from 10% to 90% by weight of the controlled release matrix tablet core. For example, in certain embodiments the hydrophilic polymer can be present in an amount of from 20% to 75%, and in other embodiments from 30% to 60% by weight of the controlled release matrix tablet core.

In certain embodiments, the controlled release matrix tablet core can comprise hydroxypropylmethylcellulose (HPMC). Non-limiting examples of hydroxypropyl methylcelluloses that are commercially available include METHOCEL® E (USP type 2910), METHOCEL® F (USP type 2906), METHOCEL® J (USP type 1828), METHOCEL® K (USP type 2201), and METHOCEL® 310 Series, products of The Dow Chemical Company, Midland, Mich., USA. The average degree of methoxyl substitution in these products can range from 1.3 to 1.9 (of the three positions on each unit of the cellulose polymer that are available for substitution) while the average degree of hydroxypropyl substitution per unit expressed in molar terms can range from 0.13 to 0.82. The controlled release matrix tablet core can comprise different HPMC grades having different viscosities. The size of a HPMC polymer is expressed not as molecular weight but instead in terms of its viscosity as a 2% solution by weight in water. Different HPMC grades can be combined to achieve the desired viscosity characteristics. For example, the at least one pharmaceutically acceptable polymer can comprise two HPMC polymers such as for example METHOCEL® K3 LV (which has a viscosity of 3 cps) and METHOCEL® K100M CR (which has a viscosity of 100,000 cps). In addition, the polymer can comprise two hydroxypropylcellulose forms such as KLUCEL® LF and KLUCEL® EF. In addition, the at least one polymer can comprise a mixture of a KLUCEL® and a METHOCEL®.

In certain embodiments the controlled release matrix tablet core can comprise a polyethylene oxide (PEO). PEO is a linear polymer of unsubstituted ethylene oxide. In certain embodiments poly(ethylene oxide) polymers having viscosity-average molecular weights of 100,000 daltons and higher can be used. Non-limiting examples of poly(ethylene oxide)s that are commercially available include: POLYOX® NF, grade WSR Coagulant, molecular weight 5 million; POLYOX® grade WSR 301, molecular weight 4 million; POLYOX® grade WSR 303, molecular weight 7 million; POLYOX® grade WSR N-60 K, molecular weight 2 million; and mixtures thereof. These particular polymers are products of Dow Chemical Company, Midland, Mich., USA. Other examples of polyethylene oxides exist and can likewise be used. The required molecular weight for the PEO can be obtained by mixing PEO of differing molecular weights that are available commercially.

In certain embodiments of the controlled release matrix tablet core, PEO and HPMC can be combined within the same controlled release matrix. In certain embodiments, the polyethylene oxides can have molecular weights ranging from 2,000,000 to 10,000,000 Da. For example, in certain embodiments the polyethylene oxides can have molecular weights ranging from 4,000,000 to 7,000,000 Da. In certain embodiments the HPMC polymers have a viscosity within the range of 4,000 centipoise to 200,000 centipoise. For example, in certain embodiments, the HPMC polymers can have a viscosity of from 50,000 centipoise to 200,000 centipoise, and in other embodiments from 80,000 centipoise to 120,000 centipoise. The relative amounts of PEO and HPMC within the controlled release matrix tablet core can vary within the scope of the invention. In at least one embodiment the PEO:HPMC weight ratio can be from about 1:3 to about 3:1. For example, in certain embodiments the PEO:HPMC weight ratio is from about 1:2 to about 2:1. As for the total amount of polymer relative to the entire controlled release matrix tablet core, this can vary as well and can depend on the desired drug loading. In at least one embodiment the total amount of polymer in the controlled release matrix tablet core can constitute from 15% to 90% by weight of the controlled release matrix tablet core. For example, in certain embodiments the total amount of polymer in the controlled release matrix tablet core can be from 20% to 75%, in other embodiments from 30% to 60%, and in still other embodiments from 10% to 20% by weight of the controlled release matrix tablet core.

In certain embodiments of the invention the controlled release matrix tablet core can comprise a hydrophobic polymer such as ethylcellulose. The viscosity of ethylcellulose can be selected in order to influence of rate the drug release. In certain embodiments the ethylcellulose has a viscosity from 7 to 100 cP (when measured as a 5% solution at 25.degree. C. in an Ubbelohde viscometer, using a 80:20 toluene:ethanol solvent.) In certain embodiments the hydrophobic polymer can constitute from 10% to 90% by weight of the controlled release matrix core. For example, in certain embodiments the hydrophobic polymer can constitutes from 20% to 75%, and in other embodiments from 30% to 60% by weight of the controlled release matrix dosage tablet core.

In certain embodiments of the invention the controlled release matrix tablet core can comprise at least one lubricant. Non-limiting examples of lubricants include stearic acid, hydrogenated vegetable oils (such as hydrogenated cottonseed oil (STEROTEX®), hydrogenated soybean oil (STEROTEX® HM) and hydrogenated soybean oil & castor wax (STEROTEX®K)) stearyl alcohol, leucine, polyethylene glycol (MW 1450, suitably 4000, and higher), magnesium stearate, glyceryl monostearate, stearic acid, glycerylbehenate, polyethylene glycol, ethylene oxide polymers (for example, available under the registered trademark CARBOWAX® from Union Carbide, Inc., Danbury, Conn.), sodium lauryl sulfate, magnesium lauryl sulfate, sodium oleate, sodium stearyl fumarate, DL-leucine, colloidal silica, and mixtures thereof. A lubricant can be present in an amount of from 0 to 4% by weight of the compressed uncoated matrix. For example, in certain embodiments the lubricant can be present in an amount of from 0 to 2.5% by weight of the controlled release matrix tablet core.

In certain embodiments of the invention the controlled release matrix tablet core comprises a plasticizer. Non-limiting examples of plasticizers include dibutyl sebacate, diethyl phthalate, triethyl citrate, tributyl citrate, triacetin, citric acid esters such as triethyl citrate NF XVI, tributyl citrate, dibutyl phthalate, 1,2-propylene glycol, polyethylene glycols, propylene glycol, diethyl phthalate, castor oil, acetylated monoglycerides, phthalate esters, and mixtures thereof. In certain embodiments of the invention, the plasticizer can be present in an amount of from 1% to 70% by weight of the controlled release polymer in the controlled release matrix tablet core. For example, in certain embodiments the plasticizer can be present in an amount of from 5% to 50%, and in other embodiments from 10% to 40% by weight of the controlled release polymer in the controlled release matrix tablet core.

In certain embodiments of the invention the controlled release matrix tablet core can comprise at least one diluent, non-limiting examples of which include dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol, sucralfate, calcium hydroxyl-apatite, calcium phosphates and fatty acid salts such as magnesium stearate. In certain embodiments the diluent can be added in an amount so that the combination of the diluent and the combination of active agent comprises up to 60%, and in other embodiments up to 50%, by weight of the composition.

In certain embodiments of the invention the controlled release matrix tablet core can comprise a solubilizer. The solubilizer can act to increase the instantaneous solubility of the bupropion salt. The solubilizer can be selected from hydrophilic surfactants or lipophilic surfactants or mixtures thereof. The surfactants can be anionic, nonionic, cationic, and zwitterionic surfactants. The hydrophilic non-ionic surfactants can be selected from the group comprised of, but not limited to: polyethylene glycol sorbitan fatty acid esters and hydrophilic transesterification products of a polyol with at least one member of the group from triglycerides, vegetable oils, and hydrogenated vegetable oils such as glycerol, ethylene glycol, polyethylene glycol, sorbitol, propylene glycol, pentaerythritol, or a saccharide, d-α-iocopheryl polyethylene glycol 1000 succinate. The ionic surfactants can be selected from the group comprised of, but not limited to: alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof; camitine fatty acid ester salts; salts of alkylsulfates ; fatty acid salts; sodium docusate; acyl lactylates; mono- and di-acetylated tartaric acid esters of mono- and di-glycericles; succinylated mono- and di-glycerides; citric acid esters of mono- and di-glycerides; and mixtures thereof. The lipophilic surfactants can be selected from the group comprised of, but not limited to: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives: polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group from glycerides, vegetable oils, hydrogenated vegetable oils, fatty acids and sterols; oil-soluble vitamins/vitamin derivatives; PEG sorbitan fatty acid esters, PEG glycerol fatty acid esters, polyglycerized fatty acid, polyoxycihylene-polyoxypropylene block copolymers, sorbitan fatty acid esters; and mixtures thereof. In certain embodiments, the solubilizer can be selected from: PEG-20-glyceryl stearate, PEG-40 hydrogenated castor oil, PEG 6 corn oil, lauryl macrogol-32 glyceride, stearoyl macrogol glyceride, polyglyceryl-10 mono dioleate, propylene glycol olcate, Propylene glycol dioctanoate, Propylene glycol caprylate/caprate, Glyceryl monooleate, Glycerol monolinoleate, Glycerol monostearate, PEG-20 sorbitan monolaurate, PEG-4 lauryl ether, Sucrose distearate, Sucrose monopalmitate, polyoxyethylene-polyoxypropylene block copolymer, polyethylene glycol 660 hydroxystearate, Sodium lauryl sulfate, Sodium dodecyl sulphate, Dioctyl suphosuccinate, L-hydroxypropyl cellulose, hydroxylethylcellulose, hydroxylpropylcellulose, Propylene glycol alginate, sodium taurochol ate, sodium glycocholate, sodium deoxycholate, betains, polyethylene glycol, d-a-tocopheryl polyethylene glycol 1000 succinate, and mixtures thereof. In at least one other embodiment the solubilizer can be selected from PEG-40 hydrogenated castor oil, lauryl macrogol-32 glyceride, stearoyl macrogol glyceride, PEG-20 sorbitan monolaurate, PEG-4 lauryl ether, polyoxyethylene-polyoxypropylene block copolymer, Sodium lauryl sulphate, Sodium dodecyl sulphate, polyethylene glycol, and mixtures thereof.

In certain embodiments of the invention the controlled release matrix tablet core comprises a swelling enhancer. Swelling enhancers are members of a special category of excipients that swell rapidly to a large extent resulting in an increase in the size of the tablet. At lower concentrations, these excipients can be used as superdisintegrants; however at concentrations above 5% w/w these agents can function as swelling enhancers and help increase the size of the controlled release matrix tablet core. According to certain embodiments of the controlled release matrix tablet core of the invention, examples of swelling enhancers include but are not limited to: low-substituted hydroxypropyl cellulose, microcrystalline cellulose, cross-linked sodium or calcium carboxymethyl cellulose, cellulose fiber, cross-linked polyvinyl pyrrolidone, cross-linked polyacrylic acid, cross-linked amberlite resin, alginates, colloidal magnesium-aluminum silicate, corn starch granules, rice starch granules, potato starch granules, pregelatinised starch, sodium carboxymethyl starch and mixtures thereof. In certain embodiments comprising the controlled release matrix tablet core, the swelling enhancer is cross-linked polyvinylpyrrolidone. The amount of the swelling enhancer can be from 5% to 90% by weight of the controlled release matrix tablet core. For example, in certain embodiments the swelling enhancer is present in an amount of from 10% to 70%, and in other embodiments from 15% to 50% by weight of the controlled release matrix tablet core.

In certain embodiments of the invention, the controlled release matrix tablet core comprises additives for allowing water to penetrate into the core of the preparation (hereinafter referred to as “hydrophilic base”). In certain embodiments, the amount of water required to dissolve 1 g of the hydrophilic base is not more than 5 ml, and in other embodiments is not more than 4 ml at the temperature of 20° C.±5° C. The higher the solubility of the hydrophilic base in water, the more effective is the base in allowing water into the core of the preparation. The hydrophilic base includes, inter alia, hydrophilic polymers such as polyethylene glycol (PEG); (e.g. PEG400, PEG1500, PEG4000, PEG6000 and PEG20000, produced by Nippon Oils and Fats Co.) and polyvinylpyrrolidone (PVP); (e.g. PVP K30, of BASF), sugar alcohols such as D-sorbitol, xylitol, or the like, sugars such as sucrose, anhydrous maltose, D-fructose, dextran (e.g. dextran 40), glucose or the like, surfactants such as polyoxyethylene-hydrogenated castor oil (HCO; e.g. Cremophor RH40 produced by BASF, HCO-40 and HCO-60 produced by Nikko Chemicals Co.), polyoxyethylene-polyoxypropylene glycol (e.g. Pluronic F68 produced by Asahi Denka Kogyo K.K.), polyoxyethylene-sorbitan high molecular fatty acid ester (Tween; e.g. Tween 80 produced by Kanto Kagaku K.K.), or the like; salts such as sodium chloride, magnesium chloride, or the like; organic acids such as citric acid, tartaric acid, or the like; amino acids such as glycine, β-alanine, lysine hydrochloride, or the like; and amino sugars such as meglumine. In at least one embodiment the hydrophilic base is PEG6000, PVP, D-sorbitol, or mixtures thereof.

The controlled release matrix tablet core of the present invention can further contain one or more pharmaceutically acceptable excipients such as, granulating aids or agents, colorants, flavorants, pH adjusters, anti-adherents, glidants and like excipients conventionally used in pharmaceutical compositions.

In at least one other embodiment of the invention there is provided a controlled release matrix tablet core comprising the combination of actives incorporated within the homogeneous controlled release matrix tablet core, which include effective amounts of at least two polymers having opposing wettability characteristics, wherein at least one polymer is selected which demonstrates a stronger tendency towards hydrophobicity and the other polymer(s) is selected such that it demonstrates a stronger tendency towards hydrophilicity. In at least one embodiment the polymer demonstrating a stronger tendency towards hydrophobicity can be ethylcellulose (EC) whereas the polymer demonstrating a stronger tendency towards hydrophilicity can be hydroxyethylcellulose (HEC) and/or hydroxypropyl methylcellulose (HPMC).

In certain embodiments, the pharmaceutical composition of the present invention can be provided as a controlled release matrix tablet core, which can be optionally encased in the controlled-release coating described herein. Such coated controlled-release matrix core tablets avoid the dose dumping of the combination of active agents in the presence of food and/or alcohol. Certain embodiments of the present invention provide for a method for preparing the controlled release of the combination of actives, the method comprising blending the combination of actives with 5% to 25% by weight of hydrophillic polymer, and 1% to 25% by weight of hydrophobic polymer, adding suitable pharmaceutical excipients, surface active agents and lubricants, granulating the mixture with solvents such as isopropyl alcohol, drying the granular mixture, milling the dried mixture, adding from 5% to 70% by weight of ethylcellulose, adding a lubricant and optionally a glidant and compressing the granules into matrices. The controlled-release matrices can optionally be encased in a gastrointestinal resistant coat or a pharmaceutically acceptable film coat.

In certain embodiments of the present invention, a swellable controlled release matrix tablet core is provided in which the combination of actives is dispersed in a polymeric matrix that is water-swellable rather than merely hydrophilic, that has an erosion rate that is substantially slower than its swelling rate, and that releases the combination of actives primarily by diffusion. The rate of diffusion of the combination of actives out of the swellable matrix can be slowed by increasing the drug particle size, by the choice of polymer used in the matrix, and/or by the choice of molecular weight of the polymer. The swellable matrix can comprise a relatively high molecular weight polymer that swells upon ingestion. In at least one embodiment the swellable matrix swells upon ingestion to a size that is at least twice its unswelled volume, and that promotes gastric retention during the fed mode. Upon swelling, the swellable matrix can also convert over a prolonged period of time from a glassy polymer to a polymer that is rubbery in consistency, or from a crystalline polymer to a rubbery one. The penetrating fluid then causes release of the combination of actives in a gradual and prolonged manner by the process of solution diffusion, i.e., dissolution of the combination of actives in the penetrating fluid and diffusion of the dissolved combination of actives back out of the swellable matrix. The swellable matrix itself is solid prior to administration and, once administered, remains undissolved in (i.e., is not eroded by) the environment of use for a period of time sufficient to permit the majority of the combination of actives to be released by the solution diffusion process during the fed mode. The rate-limiting factor in the release of the combination of actives from the swellable matrix is therefore controlled diffusion of the combination of actives from the swellable matrix rather than erosion, dissolving or chemical decomposition of the swellable matrix.

The combination of actives in the swellable matrix can be present in a therapeutically effective amount of from 0.1% to 99% by weight of the dried controlled release matrix tablet core in a ratio according to the desired dosage strengths. In certain other embodiments the combination of actives is present in the swellable matrix in an amount of from 5% to 90%, in still other embodiments from 10% to 80%, and in even still other embodiments from 25% to 80% by weight of the swellable matrix in a ratio according to the desired dosage strengths.

The water-swellable polymer forming the swellable matrix tablet core in accordance with the embodiments of the present invention can be any polymer that is non-toxic, that swells in a dimensionally unrestricted manner upon imbibition of water, and that provides for a synchronous release of the combination of actives. Non-limiting examples of polymers suitable for use in the swellable matrix include cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, and crosslinked polyacrylic acids and their derivatives, and mixtures thereof. Further examples include copolymers of the polymers listed in the preceding sentence, including block copolymers and grafted polymers. Specific examples of copolymers include PLURONIC® and TECTONIC® which are polyethylene oxide-polypropylene oxide block copolymers available from BASF Corporation, Chemicals Div., Wyandotte, Mich., USA.

The controlled release matrices of the present invention can be manufactured by methods known in the art such as those described in the patents listed above (e.g. U.S. Pat. No. 5,965,161). Other examples of methods of manufacturing controlled release matrices include wet granulation, dry granulation (e.g. slugging, roller compaction), direct compression, melt granulation, and rotary granulation.

In certain embodiments of the present invention the controlled release matrix tablet cores can optionally be coated with the control-releasing coat or a non-functional aesthetic coat using well-known coating methods.

In certain embodiments of the present invention, the tablet core can comprise a bi-layer tablet core, wherein the first layer comprises an immediate release composition comprising one drug of the desired combination, optionally a stabilizer and at least one pharmaceutically acceptable excipient and the second layer comprises an immediate release composition comprising the second drug of the desired combination, optionally a stabilizer, and at least one pharmaceutically acceptable excipient. The two layers are in direct contact. This bi-layer tablet core can subsequently be coated with a control-releasing coat such as the one described herein. The layers can be manufactured according to the separately granulated method described herein and subsequently compressed into a bi-layer tablet core using methods and equipment well known in the art (e.g., using a bi-layer press). Alternatively, each layer can be directly compressed using methods well known in the art and subsequently compressed to form the bi-layer tablet core using methods and equipment well known in the art. The immediate release bi-layer tablet core is subsequently coated with a control-releasing coat. The pharmaceutical compositions comprising a bi-layer tablet core avoid dose dumping of the combination of active agents in the presence of food and/or alcohol regardless of whether the homogenous tablet core is manufactured by the separate granulation or co-granulation methods described herein.

The Control-Releasing Coat

In at least one embodiment, the control-releasing coat is a semipermeable coat, which comprises a water-insoluble water-permeable film forming polymer, a water-soluble polymer and at least one plasticizer. The coat is permeable to both the passage of the actives and water and is free of any preformed pores.

In certain embodiments, the water-insoluble water-permeable film-forming polymer can include, a cellulose ether, such as for example, ethylcellulose; a cellulose ester, such as for example, cellulose acetate; methacrylic acid derivatives, such as for example EUDRAGIT® NE30D or NE40D; aqueous ethylcellulose dispersions, such as for example, Surelease®; aqueous acrylic enteric systems, such as for example, Acryl-EZE®, Kollicoat® MAE30DP, and Kollicoat® MAE100P; and polyvinyl derivatives, such as for example, Kollidon® SR, Kollicoat® SR30D, and Kollicoat® EMM30D. Combinations of these water-insoluble water-permeable film-forming polymers can also be used. In at least one embodiment, ethylcellulose is used as the water-insoluble, water-permeable film-forming polymer. Ethylcelluloses of a variety of viscosities can be utilized. Non-limiting examples of the ethylcellulose that can be used include, for example, ETHOCEL™ Standard Premium 4, 7, 10, 20, 45 and 100 or ETHOCEL™ Standard FP Premium 7, 10, and 100. Any combination of these ethylcelluloses can be used. In at least one embodiment of the invention, ETHOCEL™ Standard FP Premium 100 is the water-insoluble water-permeable film-forming polymer. Depending on the viscosity of the water-insoluble water-permeable film-forming polymer used, the amount of the water-insoluble, water-permeable film-forming polymer can be present at about 10%, about 20%, about 30%, about 40%, about 50%, about 60%, about 70%, about 80%, or about 90% w/w of the dry coat weight. In at least one embodiment of the present invention, the amount of water-insoluble water-permeable film-forming polymer, such as for example ETHOCEL™ Standard FP Premium 100, used can be about 50%, about 50.5%, about 51%, about 51.5%, or about 52% w/w of the dry coat weight. In at least one embodiment of the present invention, the amount of ETHOCEL™ Standard FP Premium 100, used is about 51.2% w/w of the dry coat weight.

In certain embodiments, the water-soluble polymer can be a partially or substantially water-soluble hydrophilic substance intended to modulate film permeability to both the medium and the actives in the environment of use. Non-limiting examples of the water-soluble polymers can be water-soluble cellulose ethers, vinylic polymers and combinations thereof. Non-limiting examples of the water-soluble cellulose ethers that can be used in the manufacture of the control-releasing coat can include, for example, cellulose ethers such as methylcellulose, hydroxypropylmethylcellulose, non-ionic water-soluble cellulose ethers, and any combinations thereof. Non-limiting examples of the vinylic polymers that can be used in the manufacture of the control-releasing coat include, for example, polyvinyl alcohol, polyvinylpyrrolidone, and any combinations thereof. The amount of the water-soluble polymer present can be about 1%, about 2%, about 4%, about 6%, about 8%, about 10%, about 20%, about 30%, about 40%, about 50% or about 60% w/w of the dry coat weight. In at least one embodiment of the present invention, the preferred water-soluble polymer is polyvinylpyrrolidone, such as for example, KOLLIDON® as supplied by BASF and is present at about 32% w/w of the dry coat weight. Similar polyvinylpyrrolidones are also available from other suppliers.

Plasticizers are generally added to film coatings to modify the physical properties of a polymer or polymer combinations used during manufacture of a particular coating system. The amount and choice of the plasticizer contributes to the hardness of the tablet and can even affect its dissolution or disintegration characteristics, as well as the chemical and physical stability of the coated tablet. Certain plasticizers can increase the elasticity and/or pliability of a coat, thereby decreasing the coat's brittleness. Once a pharmaceutical composition or dosage form is manufactured, certain plasticizers can function to increase the hydrophilicity of the coat in the environment of use. Therefore, plasticizers can function to enhance processing of coating formulations during manufacture as well as affect release characteristics of a coating system. Non-limiting examples of plasticizers that can be used in the control-releasing coat described herein include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, polyols (e.g. polyethylene glycol) of various molecular weights, and mixtures thereof. It is contemplated and within the scope of the invention, that a combination of plasticizers can be used in the present composition. In certain embodiments of the invention, the plastizer is a combination of polyethylene glycol 3350 and dibutyl sebacate. In certain other embodiments, the plasticizer is dibutyl sebacate. The amount of plasticizer can be present at about 0.1%, about 0.2%, about 0.4%, about 0.6%, about 0.8%, about 1%, about 2%, about 4%, about 6%, about 8%, about 10%, about 15%, about 20%, about 25%, about 30%, about 35%, or about 40% w/w of the dry coat weight. In at least one embodiment of the present invention where the plasticizer used is a combination of polyethylene glycol 3350 and dibutyl sebacate, the amount of plasticizer used can be present at about 3%, about 5%, about 7%, about 9%, about 11%, about 12%, or about 15% of the dry coat weight in a ratio of about 2:1 (polyethylene glycol 3350:dibutyl sebacate). In embodiments where the plasticizer is dibutyl sebabcate, the amount of dibutyl sebacate is about 5% w/w of the dry coat weight.

It will be readily apparent to the skilled artisan that the permeability of the control-releasing coating will affect the release profile of the actives from the immediate release tablet cores described herein. This is true even if a controlled-releasing coat is applied onto the controlled-release matrix core described herein. Not only can the relative proportions of the preferred polymer coat ingredients, notably the ratio of the water-insoluble water-permeable film-forming polymer:plasticizer:water-soluble polymer, be varied to alter the permeability of the control-releasing coat, but so can the type and viscosity of the polymers used as well as the thickness of coating applied. Thus, if a more controlled i.e., a slower release is desired, the ratio of water-insoluble water-permeable film-forming polymer:water-soluble polymer and/or the amount of coating applied would be increased. Also, if the polymers being used are of a lower viscosity, the amount of coating to be applied can be increased to obtain the desired release profile when compared to a control-release coating formulation with a higher viscosity polymer. Addition of other excipients to the tablet core can also alter the permeability of the control-releasing coat. For example, if it is desired that the tablet cores described herein further comprise an expanding agent, the amount of plasticizer in the control-releasing coat should be increased to make the coat more pliable as the pressure exerted on a less pliable control-releasing coat by the expanding agent would rupture the coat. Other excipients such as taste masking agents and pigments can also be added to the control-releasing coat.

Depending on the desired in-vitro or in-vivo release profile of the actives, the weight gained after coating the tablet cores with the control-releasing coat can be about 4%, about 6%, about 8%, about 10%, about 12%, about 14%, about 16%, about 18%, about 20%, about 22%, about 24%, or about 25% w/w of the dry tablet core. In at least one embodiment of the present invention, the weight gained is about 5% of the dry coat weight.

Preparation and application of the control-releasing coat are well known in the art. Nevertheless, an exemplary process for preparing the coating solution can be as follows. The water-insoluble water-permeable film-forming polymer (e.g. ethylcellulose), and the plasticizer or plasticizers (e.g. PEG 3350 or PEG 3350 and dibutyl sebacate), can be dissolved in an organic solvent (e.g. ethyl alcohol) or a mixture of organic solvents and water (e.g. ethyl alcohol, propanol, and water). The water-soluble polymer (e.g. polyvinylpyrrolidone) is added next until a homogenous mixture is achieved. The resulting solution can be, if necessary, homogenized by passing it through a high-pressure homogenizer. The coating solution is then spray coated onto the tablet cores using a tablet coater, fluidized bed apparatus, or any other suitable coating apparatus known in the art until the desired weight gain is achieved. The tablet cores coated with the control-releasing mixture are subsequently dried.

The controlled-release pharmaceutical formulations prepared by the process described above surprisingly exhibit a synchronous release of the combination of actives at least in-vitro. In certain embodiments the synchronous release is observed in 900 ml of 0.1N HCl, pH4.5 acetate buffer, or pH 6.8 phosphate buffer using a USP Apparatus 1 at 75 rpm at 37° C. In certain embodiments, the control-release pharmaceutical compositions of the invention surprisingly also provide an about 15% to about 26% enhanced absorption of bupropion hydrobromide in the plasma when compared to co-administration of single active agent pharmaceutical compositions of bupropion hydrobromide and citalopram hydrochloride or bupropion hydrobromide and escitalopram oxalate.

In certain embodiments of the present invention, the pharmaceutical compositions of the invention can avoid the dose-dumping phenomenon when the compositions are administered with food and/or alcohol.

Examples for manufacturing the pharmaceutical compositions of the invention are described below. It should be understood that these examples are intended to be exemplary and that the specific constituents, amounts thereof, and methods described may be varied therefrom by the skilled artisan based on his/her skill and knowledge in the art of drug delivery without undue experimentation in order to achieve the desired in-vitro dissolution and pharmacokinetic parameters described and claimed herein.

The embodiments of the invention and variations thereof relating to the pharmaceutical compositions of the invention will be apparent to those versed in the drug delivery art from the above description and the following examples taken in conjunction with the accompanying claims.

### Example 1

### Homogenous Tablet Core Composition and Method of Manufacture

### Separate Granulation Method

A. Bupropion HBr Granulation:

Bupropion HBr Granule Composition

| Component | % w/w |
|---|---|
| Bupropion HBr | 91.72 |
| Polyvinyl Alcohol | 3.24 |
| Citric Acid | 5.04 |

The pharmaceutical active, bupropion HBr, was top-spray granulated using a Glatt GPCG 1 (6 inch chamber). The theoretical batch size was 3270.6 g. An aqueous (purified water) solution of polyvinyl alcohol (PVA) (4.82% of solution) and citric acid (7.5% of solution) was sprayed onto 3 kg of bupropion HBr to a weight gain of 9.02% to produce a granule comprising of 91.73% bupropion HBr, 3.23% PVA, and 5.04% citric acid. The powder bed temperature was maintained between 38-45° C., and the liquid spray rate maintained between 5-7 g/min throughout the granulation process. When spraying of the granulation solution was stopped, the granules were fluid bed dried to an LOD (loss on drying) level of <1%. The granules were screened and the granules with a particle size of between about 355 μm and about 800 μm were retained for manufacture of the homogenous tablet core.

B. Citalopram HCl Granulation:

Citalopram HCl Granule Composition

| Component | % w/w |
|---|---|
| Citalopram HCl | 93.28 |
| Kollidon ® 90F | 5.60 |
| Butylated hydroxytoluene | 1.12 |

The pharmaceutical active, citalopram HCl, was top-spray granulated using a Glatt GPCG 1 (6 inch chamber). The theoretical batch size was 3216 g. An organic solvent (2-propanol) solution of Kollidon® 90F (10% of solution) and butylated hydroxytoluene (BHT) (2% of solution) was sprayed onto about 3 kg of citalopram HCl to a weight gain of about 7.2% to produce a granule comprising of about 93.28% citalopram HCl, 5.60% Kollidon® 90F, and 1.12% BHT. The powder bed temperature was maintained between 20-35° C., and the liquid spray rate maintained between 13-17 g/min throughout the granulation process. When spraying of the granulation solution was stopped, the granules were fluid bed dried to an LOD (loss on drying) level of <1%. The granules were screened and the granules with a particle size of between about 355 μm and about 800 μm were retained for manufacture of the homogenous tablet core.

C. Homogenous Tablet Core Composition and Method of Manufacture

To manufacture the homogenous tablet core comprising about 348 mg of bupropion HBr (equivalent to about 260 mg of bupropion base) and about 22.2 mg of citalopram HCl (equivalent to about 20 mg citalopram base), a blend with the following composition was prepared: about 91.3% bupropion HBr granules (manufactured as described above), about 5.8% citalopram HCl granules (manufactured as described above) and about 2.9% Compritol 888 ATO (screened through a 500 μm screen). A homogenous blend of about 1500 g was manufactured by dispensing about 1369.5 g of bupropion HBr granules, about 87.0 g of citalopram HCl granules, and about 43.5 g of the screened Compritol 888 ATO. The material was added to a v-blender (15 litre shell of a Pharmatech AB-050 v-blender) in the following order:

1. about ½ of the bupropion HBr granules

2. All of the citalopram HCl granules

3. All of the Compritol 888 ATO

4. The remaining bupropion HBr granules

The tablet core components were homogenously blended for about 10 minutes, with the v-shell speed set to 25 rpm and the intensifier bar turned off. The homogenous blend was discharged from the v-shell and charged to a tablet press (Riva Picolla 10 station rotary tablet press) and compressed to a target tablet weight of about 416 mg and a target tablet hardness of about 130N using 9 mm round normal concave shaped tablet tooling. The resulting product comprises the homogenous tablet core, which at this point is an immediate release core having the following composition:

Homogenous Tablet Core Composition

| Component | mg/tablet | % w/w |
|---|---|---|
| Bupropion HBr | 348.36 | 83.74 |
| Citalopram HCl | 22.51 | 5.41 |
| PVA | 12.31 | 2.96 |
| Citric Acid | 19.14 | 4.60 |
| BHT | 0.27 | 0.06 |
| Kollidon ® 90F | 1.35 | 0.32 |
| Compritol 888 ATO | 12.06 | 2.90 |

Tablet Coat Composition and Method of Manufacture

Tablet Control-Releasing Coat Composition

| Component | % w/w |
|---|---|
| Ethocel 100 FP Premium | 4.61 |
| Kollidon ® 90F | 2.86 |
| PEG 3350 | 1.03 |
| Dibutylsebacate | 0.50 |
| 2-Propanol | 8.68 |
| Absolute Ethanol | 81.86 |
| Purified water | 0.46 |

The homogenous IR tablet cores were coated with an ethylcellulose based film by preparing an organic solvent solution consisting of about 4.61% ethocel standard 100FP premium, about 2.86% Kollidon® 90F, about 1.03% carbowax sentry polyethylene glycol 3350 granular NF FCC grade, about 0.5% dibutyl sebacate NF, about 8.68% 2-propanol, about 81.86% absolute ethanol, and about 0.46% purified water. The plasticized polymer solution was applied to about 1 kg of the final tablet cores using an O'Hara Labcoat I tablet coating machine (12″ pan) until about a 10% weight gain was obtained. The product temperature was maintained between about 38-44° C., and the liquid spray rate was maintained between about 7-9 g/min throughout the coating process. The controlled release coated tablets were then cured for about 10 minutes (inlet air is set at about 35° C., pan speed set at 5 rpm).

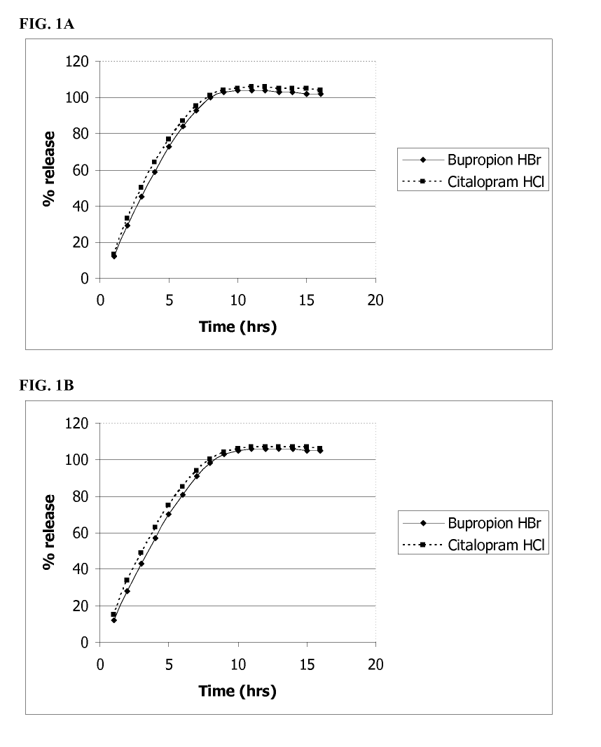

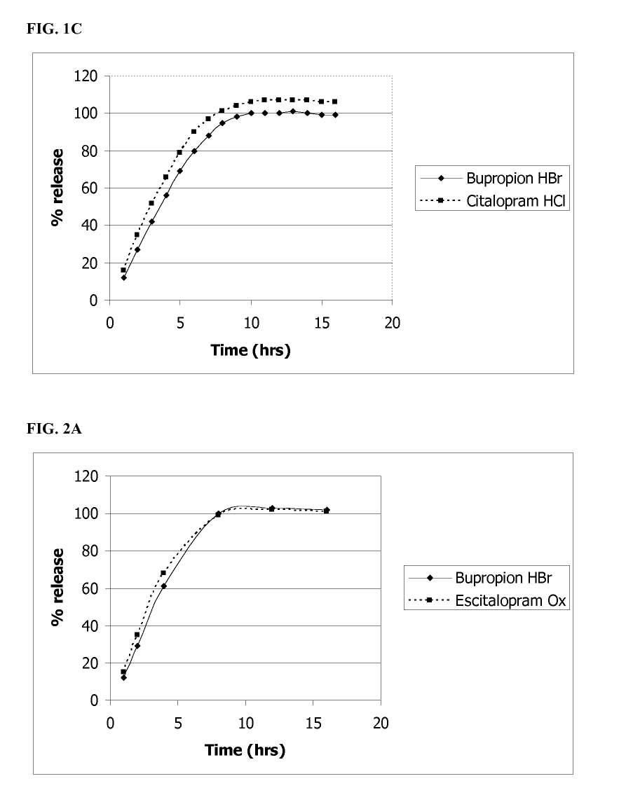

The dissolution profile of the above pharmaceutical composition was determined under the dissolution conditions described below in Tables 1A, 1B, and 1C. The results of the dissolution testing are presented as a % of the total bupropion HBr and citalopram HCl in the controlled release tablet (batch no. 0612087) and are also depicted in FIGS. 1A, 1B, and 1C.

TABLE 1A
Dissolution Conditions: 900 ml 0.1N HCl,
USP Apparatus 1, 75 rpm, 37° C.

% Released

| Time (hr) | Bupropion HBr | Citalopram HCl |
|---|---|---|
| 1 | 12 | 13 |
| 2 | 29 | 33 |
| 3 | 45 | 50 |
| 4 | 59 | 64 |
| 5 | 73 | 77 |
| 6 | 84 | 87 |
| 7 | 93 | 95 |
| 8 | 100 | 101 |
| 9 | 103 | 104 |
| 10 | 104 | 105 |
| 11 | 104 | 106 |
| 12 | 104 | 106 |
| 13 | 103 | 105 |
| 14 | 103 | 105 |
| 15 | 102 | 105 |
| 16 | 102 | 104 |

TABLE 1B
Dissolution Conditions: 900 ml pH 4.5 acetate
buffer, USP Apparatus 1, 75 rpm, 37° C.

% Released

| Time (hr) | Bupropion HBr | Citalopram HCl |
|---|---|---|
| 1 | 12 | 15 |
| 2 | 28 | 34 |
| 3 | 43 | 49 |
| 4 | 57 | 63 |
| 5 | 70 | 75 |
| 6 | 81 | 85 |
| 7 | 91 | 94 |
| 8 | 98 | 100 |
| 9 | 103 | 104 |
| 10 | 105 | 106 |
| 11 | 106 | 107 |
| 12 | 106 | 107 |
| 13 | 106 | 107 |
| 14 | 106 | 107 |
| 15 | 105 | 107 |
| 16 | 105 | 106 |

TABLE 1C
Dissolution Conditions: 900 ml pH 6.8 phosphate
buffer, USP Apparatus 1, 75 rpm, 37° C.

% Released

| Time (hr) | Bupropion HBr | Citalopram HCl |
|---|---|---|
| 1 | 12 | 16 |
| 2 | 27 | 35 |
| 3 | 42 | 52 |
| 4 | 56 | 66 |
| 5 | 69 | 79 |
| 6 | 80 | 90 |
| 7 | 88 | 97 |
| 8 | 95 | 101 |
| 9 | 98 | 104 |
| 10 | 100 | 106 |
| 11 | 100 | 107 |
| 12 | 100 | 107 |
| 13 | 101 | 107 |
| 14 | 100 | 107 |
| 15 | 99 | 106 |
| 16 | 99 | 106 |

### Example 2

### Homogenous Tablet Core Composition and Method of Manufacture

### Separate Granulation Method

A. Bupropion HBr Granulation:

The bupropion HBr granules were prepared as described in Example 1.

B. Escitalopram Ox Granulation:

Escitalopram Ox Granule Composition

| Component | % w/w |
|---|---|
| Escitalopram Ox | 91.74 |
| Kollidon ® 90F | 6.88 |
| Butylated hydroxytoluene | 1.38 |

The pharmaceutical active, escitalopram Ox, was top-spray granulated using a Glatt GPCG 1 (6 inch chamber). The theoretical batch size was 2725 g. An organic solvent (2-propanol) solution of Kollidon® 90F (6% of solution) and butylated hydroxytoluene (BHT) (1.2% of solution) was sprayed onto 2.5 kg of escitalopram Ox to a weight gain of 9% to produce a granule comprising of 91.74% escitalopram oxalate, 6.88% Kollidon® 90F, and 1.38% BHT. The powder bed temperature was maintained between 15-30° C., and the liquid spray rate maintained between 15-19 g/min throughout the granulation process. When spraying of the granulation solution was stopped, the granules were fluid bed dried to an LOD (loss on drying) level of <1%. The granules were screened and the granules with a particle size of between about 355 μm and about 800 μm were retained for use to manufacture the homogenous tablet core.

C. Homogenous Tablet Core Composition and Method of Manufacture

To manufacture the homogenous tablet core comprising 348 mg of bupropion HBr (equivalent to 260 mg of bupropion base) and 25.5 mg of escitalopram Ox (equivalent to 20 mg escitalopram base), a homogenous tablet blend with the following composition was prepared; 90.46% bupropion HBr granules (manufactured as outlined in Example 1), 6.63% escitalopram Ox granules (manufactured as outlined above) and 2.91% Compritol 888 ATO (screened through a 500 μm screen). A homogenous tablet blend of 1500 g was manufactured by dispensing 1356.90 g of bupropion HBr granules, 99.45 g of escitalopram Ox granules, and 43.65 g of the screened Compritol 888 ATO. The material was added to a v-blender (15 litre shell of a Pharmatech AB-050 v-blender) in the following order:

1. ˜½ of the bupropion HBr granules

2. All of the escitalopram Ox granules

3. All of the Compritol 888 ATO

4. The remaining bupropion HBr granules

The tablet components were blended for 10 minutes, with the v-shell speed set to 25 rpm and the intensifier bar turned off. The homogenous tablet blend was discharged from the v-shell and charged to a tablet press (Riva Bilayer 11 station rotary tablet press) and compressed to a target tablet weight of 419.4 mg and a target tablet hardness of 100N using 9 mm round normal concave shaped tablet tooling. The resulting product comprises the homogenous tablet core, which at this point is an immediate release core having the following composition:

Homogenous Tablet Core Composition

| Component | mg/tablet | % w/w |
|---|---|---|
| Bupropion HBr | 347.98 | 82.97 |
| Escitalopram Ox | 25.51 | 6.08 |
| PVA | 12.29 | 2.93 |
| Citric Acid | 19.12 | 4.56 |
| BHT | 0.38 | 0.09 |
| Kollidon ® 90F | 1.91 | 0.46 |
| Compritol 888 ATO | 12.21 | 2.91 |

Tablet Coat Composition and Method of Manufacture

The homogenous IR tablet cores were next coated with the control-releasing coat as described in Example 1.

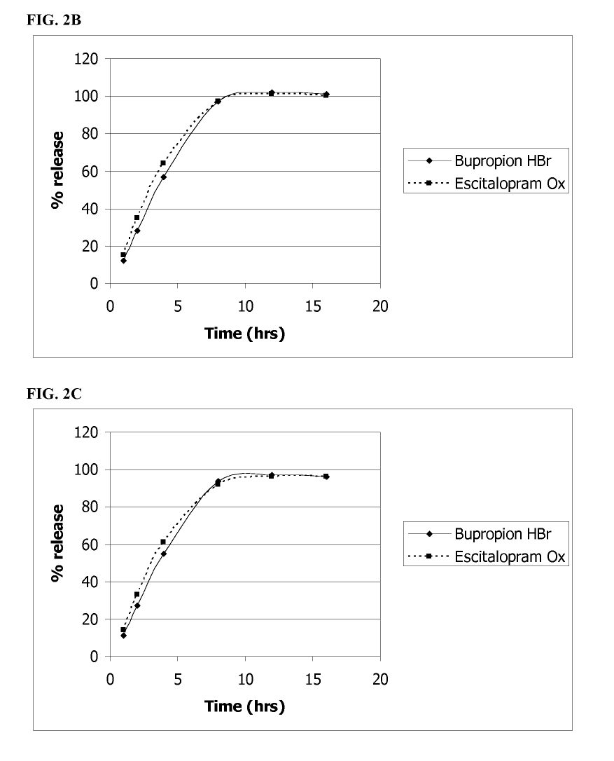

The dissolution profile of the above pharmaceutical composition was determined under the dissolution conditions described below in Tables 2A, 2B, and 2C. The results of the dissolution testing are presented as a % of the total bupropion HBr and escitalopram Ox in the controlled release tablet (batch no. 0705037) and are also depicted in FIGS. 2A, 2B, and 2C.

TABLE 2A
Dissolution Conditions: 900 ml 0.1N HCl,
USP Apparatus 1, 75 rpm, 37° C.

% Released

| Time (hr) | Bupropion HBr | escitalopram Ox |
|---|---|---|
| 1 | 12 | 15 |
| 2 | 29 | 35 |
| 4 | 61 | 68 |
| 8 | 100 | 99 |
| 12 | 103 | 102 |
| 16 | 102 | 101 |

TABLE 2B
Dissolution Conditions: 900 ml pH 4.5 acetate
buffer, USP Apparatus 1, 75 rpm, 37° C.

% Released

| Time (hr) | Bupropion HBr | escitalopram Ox |
|---|---|---|
| 1 | 12 | 15 |
| 2 | 28 | 35 |
| 4 | 57 | 64 |
| 8 | 97 | 97 |
| 12 | 102 | 101 |
| 16 | 101 | 100 |

TABLE 2C
Dissolution Conditions: 900 ml pH 6.8 phosphate
buffer, USP Apparatus 1, 75 rpm, 37° C.

% Released

| Time (hr) | Bupropion HBr | escitalopram Ox |
|---|---|---|
| 1 | 11 | 14 |
| 2 | 27 | 33 |
| 4 | 55 | 61 |
| 8 | 94 | 92 |
| 12 | 97 | 96 |
| 16 | 96 | 96 |

### Example 3

### Homogenous Tablet Core Composition and Method of Manufacture

### Co-Granulation Method

A. Bupropion HBr/Escitalopram Ox Co-Granulation:

Bupropion HBr/Escitalopram Oxalate
Co-Granulation Composition

| Component | % w/w |
|---|---|
| Bupropion HBr | 85.40 |
| Escitalopram Ox | 6.25 |
| Butylatedhydroxytoluene | 0.09 |
| PVA | 3.23 |
| Citric Acid | 5.03 |

The pharmaceutical actives, bupropion HBr and escitalopram Ox, were top-spray granulated using a Glatt GPCG 1 (6 inch chamber). The theoretical batch size was 3273.9 g. The granulation solution was prepared in 3 steps:
1.) an aqueous (purified water) solution of polyvinyl alcohol (PVA) (4.91% of solution) and citric acid (7.64% of solution) was prepared
2.) a solvent (2-propanol) solution of BHT (7.73% of solution) was prepared
3.) the BHT solvent solution was added to the aqueous PVA/citric acid solution and mixed for a minimum of 20 minutes. The BHT became finely dispersed in the PVA/citric acid solution. The final composition of the granulation suspension was 4.82% PVA, 7.50% citric acid, 1.67% 2-propanol, 0.14% BHT, 85.87% purified water.

The buproion HBR and escitalopram Ox were charged to the granulation chamber in the required ratio to give the desired final dosage strengths of each active. For a 348 mg dose of bupropion HBr and a 25.5 mg dose of escitalopram Ox the actives were dispensed such that the material in the granulation chamber consisted of 93.2% bupropion HBr and 6.8% escitalopram Ox. In this example 3 kg of material was granulated, 2796 g of bupropion HBr mixed with 204 g of escitalopram Ox. Approximately half of the bupropion HBr was charged to the granulation chamber, followed by all of the escitalopram Ox, and the remainder of the bupropion HBr. The granulation suspension was sprayed onto the 3 kg mix of bupropion HBr to a weight gain of 9.13% to produce a granule comprising of 85.40% bupropion HBr, 6.23% escitalopam Ox, 3.24% PVA, 5.04% citric acid, and 0.09% BHT. The powder bed temperature was maintained between 40-45° C., and the liquid spray rate maintained between 5-7 g/min throughout the granulation process. When spraying of the granulation solution was stopped, the granules were fluid bed dried to an LOD (loss on drying) level of <1%. The granules were screened through a 1.00 mm screen, and the material <1.00 mm retained for manufacture of the homogenous tablet core.

B. Homogenous Tablet Core Composition and Method of Manufacture

To manufacture a homogenous tablet core comprising 348 mg of bupropion HBr (equivalent to 260 mg of bupropion base) and 25.5 mg of escitalopram Ox (equivalent to 20 mg escitalopram base), a homogenous tablet blend with the following composition was prepared; 97.1% bupropion HBr/escitalopram Ox co-granules (manufactured as outlined above), and 2.9% Compritol 888 ATO (screened through a 500 μm screen). A homogenous tablet blend of 1500 g was manufactured by dispensing 1456.5 g of bupropion HBr/escitalopram Ox co-granules, and 43.5 g of the screened Compritol 888 ATO. The material was added to a v-blender (15 litre shell of a Pharmatech AB-050 v-blender) in the following order:

1. ˜half of the bupropion HBr/escitalopram Ox co-granules

2. All of the Compritol 888 ATO

3. The remaining bupropion HBr/escitalopram Ox co-granules

The tablet components were blended for 10 minutes, with the v-shell speed set to 25 rpm and the intensifier bar turned off. The homogenous tablet blend was discharged from the v-shell and charged to a tablet press (Riva Bilayer 11 station rotary tablet press) and compressed to a target tablet weight of 420 mg and a target tablet hardness of 130N using 9 mm round normal concave shaped tablet tooling. The resulting product comprises the homogenous tablet core, which at this point is an immediate release core has the following composition:

Homogenous Tablet Core Composition

| Component | mg/tablet | % w/w |
|---|---|---|
| Bupropion HBr | 348.28 | 82.92 |
| Escitalopram Ox | 25.49 | 6.07 |
| PVA | 13.17 | 3.14 |
| Citric Acid | 20.51 | 4.88 |
| BHT | 0.37 | 0.09 |
| Compritol 888 ATO | 12.18 | 2.90 |

Tablet Coat Composition and Method of Manufacture

The homogenous IR tablet cores were next coated with the control-releasing coat as described in Example 1.

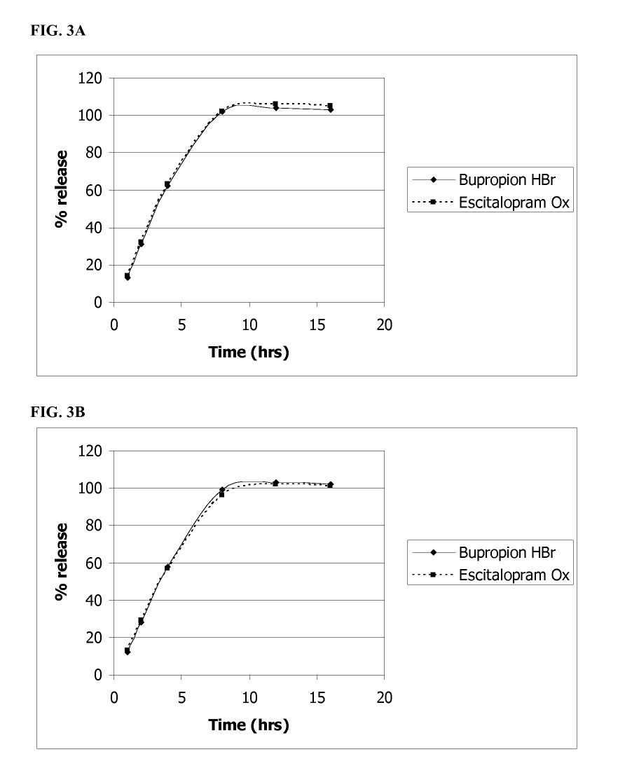

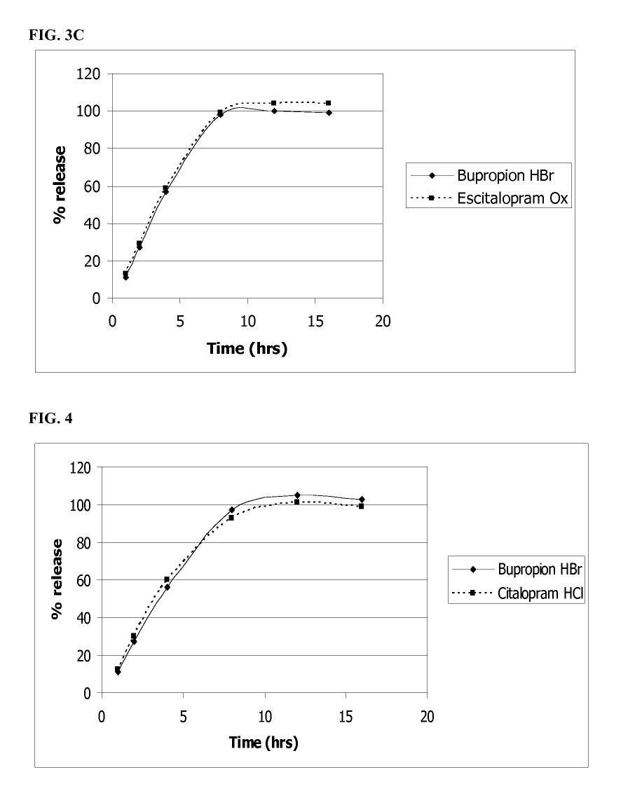

The dissolution profile of the above pharmaceutical composition was determined under the dissolution conditions described below in Tables 3A, 3B, and 3C. The results of the dissolution testing are presented as a % of the total bupropion HBr and escitalopram Ox in the controlled release tablet (batch no. 0704028) and are also depicted in FIGS. 3A, 3B, and 3C.

TABLE 3A
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 13 | 14 |
| 2 | 31 | 32 |
| 4 | 62 | 63 |
| 8 | 102 | 102 |
| 12 | 104 | 106 |
| 16 | 103 | 105 |

TABLE 3B
Dissolution Conditions: 900 ml pH 4.5 acetate
buffer, USP Apparatus 1, 75 rpm, 37° C.

% Released

| Time (hr) | Bupropion HBr | Escitalopram Ox |
|---|---|---|
| 1 | 12 | 13 |
| 2 | 28 | 29 |
| 4 | 58 | 57 |
| 8 | 99 | 96 |
| 12 | 103 | 102 |
| 16 | 102 | 101 |

TABLE 3C
Dissolution Conditions: 900 ml pH 6.8 phosphate
buffer, USP Apparatus 1, 75 rpm, 37° C.

% Released

| Time (hr) | Bupropion HBr | escitalopram Ox |
|---|---|---|
| 1 | 11 | 13 |
| 2 | 27 | 29 |
| 4 | 57 | 59 |
| 8 | 98 | 99 |
| 12 | 100 | 104 |
| 16 | 99 | 104 |

### Example 4

### Homogenous Tablet Core Composition and Method of Manufacture

A. Homogenous Tablet Core Composition and Method of Manufacture

The homogenous tablet core composition comprises a mixture of bupriopion HBr (348 mg) and citalopram HCl (22.2 mg). The homogenous tablet core was made according to the co-granulation method described in Example 3 with citalopram HCl instead of escitalopram Ox. The resulting product comprises the homogenous tablet core, which at this point is an immediate release tablet core has the following composition:

Homogenous Tablet Core Composition

| Component | mg/tablet | % w/w |
|---|---|---|
| Bupropion HBr | 348.36 | 83.74 |
| Citalopram HCl | 22.51 | 5.41 |
| PVA | 12.31 | 2.96 |
| Citric Acid | 19.14 | 4.60 |
| BHT | 0.27 | 0.06 |
| Kollidon ® 90F | 1.35 | 0.32 |
| Compritol 888 ATO | 12.06 | 2.90 |

Tablet Coat Composition and Method of Manufacture

The control-releasing coat was manufactured according to the method described in Example 1 in the absence of the plasticizer PEG 3350. The resulting control-releasing coat has the following composition:

Control-releasing Coat Composition Minus PEG 3500

| Component | % w/w |
|---|---|
| Ethocel 100 FP Premium | 4.61 |
| Kollidon 90F | 2.86 |
| Dibutylsebacate | 0.50 |
| 2-Propanol | 8.68 |
| Absolute Ethanol | 82.89 |
| Purified water | 0.46 |

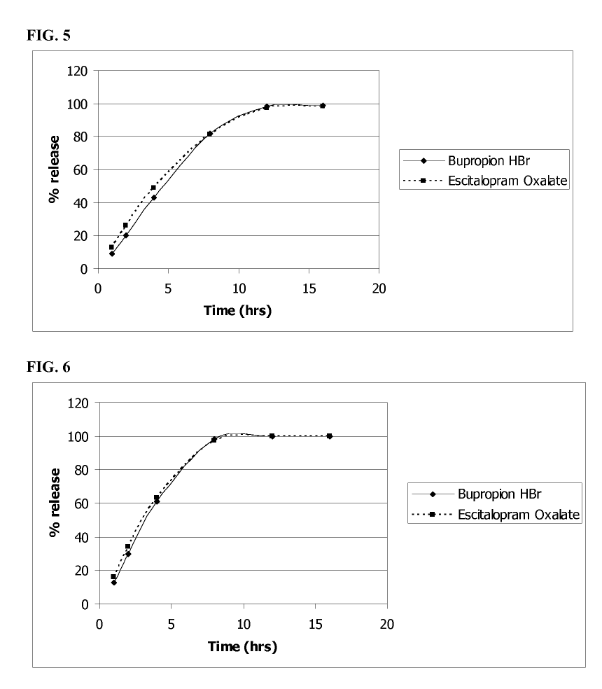

The dissolution profile of the above pharmaceutical composition (batch no. E2240) was determined under the dissolution conditions described below in Table 4. The result of the dissolution testing is presented as a % of the total bupropion HBr and citalopram HCl in the controlled release tablet and is also depicted in FIG. 4.

TABLE 4
Dissolution Conditions: 900 ml 0.1N HCl,
USP Apparatus 1, 75 rpm, 37° C.

% Released

| Time (hr) | Bupropion HBr | Citalopram HCl |
|---|---|---|
| 1 | 11 | 12 |
| 2 | 27 | 30 |
| 4 | 56 | 60 |
| 8 | 97 | 93 |
| 12 | 105 | 101 |
| 16 | 103 | 99 |

### Example 5

### Homogenous Tablet Core Composition and Method of Manufacture

The homogenous tablet core was manufactured according to the method described in Example 3 with the following bupropion HBr/escitalopram Ox co-granule and homogenous tablet core composition:

Bupropion HBr/Escitalopram Oxalate
Co-Granulation Composition

| Component | % w/w |
|---|---|
| Bupropion HBr | 86.88 |
| Escitalopram Ox | 4.78 |
| Butylatedhydroxytoluene | 0.07 |
| PVA | 3.24 |
| Citric Acid | 5.03 |

Homogenous Tablet Core Composition

| Component | mg/tablet | % w/w |
|---|---|---|
| Bupropion HBr | 325.02 | 84.36 |
| Escitalopram Ox | 17.88 | 4.64 |
| PVA | 12.12 | 3.15 |
| Citric Acid | 18.82 | 4.88 |
| BHT | 0.26 | 0.07 |
| Compritol 888 ATO | 11.17 | 2.90 |

Tablet Coat Composition and Method of Manufacture

The control-releasing coat was manufactured according to the method described in Example 1 accept that the polyvinylpyrrolidone (Kollidon® 90F) was replaced with the lower viscosity Kollidon® K29/32 at the same % w/w. The resulting control-releasing coat has the following composition:

Control-releasing Coat Composition with a lower
viscosity PVP (Kollidon ® K29/32)

| Component | % w/w |
|---|---|
| Ethocel 100 FP Premium | 4.61 |
| Kollidon ® K29/32 | 2.86 |
| PEG 3350 | 1.03 |
| Dibutylsebacate | 0.50 |
| 2-Propanol | 8.68 |
| Absolute Ethanol | 81.86 |
| Purified water | 0.46 |

The dissolution profile of the above pharmaceutical composition was determined under the dissolution conditions described below in Table 5. The result of the dissolution testing is presented as a % of the total bupropion HBr and escitalopram Ox in the controlled release tablet and is also depicted in FIG. 5.

TABLE 5
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 9 | 13 |
| 2 | 20 | 26 |
| 4 | 43 | 49 |
| 8 | 82 | 81 |
| 12 | 98 | 97 |
| 16 | 99 | 98 |

### Example 6

### Homogenous Tablet Core Composition and Method of Manufacture

The homogenous tablet core was manufactured according to the method described in Example 3 with the following co-granule and homogenous tablet core composition:

Bupropion HBr/Escitalopram Oxalate
Co-Granulation Composition

| Component | % w/w |
|---|---|
| Bupropion HBr | 86.88 |
| Escitalopram Ox | 4.78 |
| Butylatedhydroxytoluene | 0.07 |
| PVA | 3.24 |
| Citric Acid | 5.03 |

Homogenous Tablet Core Composition

| Component | mg/tablet | % w/w |
|---|---|---|
| Bupropion HBr | 325.02 | 84.36 |
| Escitalopram Ox | 17.88 | 4.64 |
| PVA | 12.12 | 3.15 |
| Citric Acid | 18.82 | 4.88 |
| BHT | 0.26 | 0.07 |
| Compritol 888 ATO | 11.17 | 2.90 |

Tablet Coat Composition and Method of Manufacture

The control-releasing coat was manufactured according to the method described in Example 1 accept that the grade of ethylcellulose polymer was changed from Ethocel Std 100 PREM (Dow Chemical Company) to the lower viscosity grade, Ethocel Std 10 PREM. The resulting control-releasing coat has the following composition:

Control-releasing Coat Composition with a lower viscosity
grade Ethylcellulose (Ethocel Std 10 PREM)

| Component | % w/w |
|---|---|
| Ethocel Std 10 FP Premium | 4.61 |
| Kollidon ® K29/32 | 2.86 |
| PEG 3350 | 1.03 |
| Dibutylsebacate | 0.50 |
| 2-Propanol | 8.68 |
| Absolute Ethanol | 81.86 |
| Purified water | 0.46 |

The dissolution profile of the above pharmaceutical composition was determined under the dissolution conditions described below in Table 6. The result of the dissolution testing is presented as a % of the total bupropion HBr and escitalopram Ox in the controlled release tablet (batch no. E2828) and is also depicted in FIG. 6.

TABLE 6
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 13 | 16 |
| 2 | 30 | 34 |
| 4 | 61 | 63 |
| 8 | 98 | 97 |
| 12 | 100 | 100 |
| 16 | 100 | 100 |

### Example 7

### Homogenous Tablet Core Composition and Method of Manufacture

The homogenous tablet core was manufactured according to the method and composition described in Example 2.

Tablet Coat Composition and Method of Manufacture

The homogenous IR tablet cores were coated with a polymethacrylate based film by preparing an aqueous suspension consisting of about 26.97% aqueous disperion of Eudragit NE30D, 5.3% Talc, 2.47% Hydroxypropyl Methylcellulose, 2.25% PEG4000, 0.31% Somethicone C, 0.23% Tween 80 and 62.47% purified water. Approximately 60% of the required water was heated to approximately 65° C. using a paddle mixer, to which the hydroxypropyl methylcellulose, Tween® 80 and Simethicone® C were added. Talc was added to the remaining water in a separate vessel under conditions of high shear, and the suspension mixed under high shear for approximately 20 minutes, following which the talc suspension was added to the other vessel. Upon cooling, the NE30D dispersion was added directly to the coating suspension, and the entire suspension mixed for approximately 30 minutes, following which the suspension was sieved through a 150 μm screen to remove lumps. The plasticized polymer solution was applied to about 1 kg of the final tablet cores using an O'Hara Labcoat I tablet coating machine (12″ pan) until about a 10% weight gain was obtained. The product temperature was maintained between about 30° C., and the liquid spray rate maintained between about 7-9 g/min throughout the coating process. The controlled release coated tablets were then cured for about 24 hours at 40° C. in a conventional tray drying oven

The control-releasing coating formulation has the following composition:

| Component | % w/w |
|---|---|
| Eudragit NE 30 D | 26.97 |
| Talc 1889 | 5.30 |
| Hydroxypropyl Methylcellulose | 2.47 |
| (Pharmacoat 606) | |
| PEG 4000 | 2.25 |
| Simethicone C | 0.31 |
| Tween 80 | 0.23 |
| Water | 62.47 |

The dissolution profile of the above pharmaceutical composition was determined under the dissolution conditions described below in Table 7. The result of the dissolution testing is presented as a % of the total bupropion HBr and escitalopram oxalate in the controlled release tablet is also depicted in FIG. 7.

TABLE 7
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 2 | 2 |
| 2 | 5 | 7 |
| 4 | 14 | 19 |
| 8 | 36 | 44 |
| 12 | 57 | 63 |
| 16 | 74 | 77 |

### Example 8

### Bupropion HBr (325 Mg)/Escitalopram Oxalate (17.9 Mg) Pharmaceutical Composition

Homogenous Tablet Core Composition and Method of Manufacture.

The homogenous tablet core was manufactured according to the method and composition described in Example 5.

Tablet Coat Composition and Method of Manufacture

The control-releasing coat was manufactured according to the method described in Example 1 accept that the polyvinylpyrrolidone (Kollidon® 90F) was reduced by 50%. The resulting control-releasing coat has the following composition:

Control-releasing Coat Composition with
50% less PVP (Kollidon ® 90F)

| Component | % w/w |
|---|---|
| Ethocel 100 FP Premium | 4.61 |
| Kollidon ® 90F | 1.43 |
| PEG 3350 | 1.03 |
| Dibutylsebacate | 0.50 |
| 2-Propanol | 8.68 |
| Absolute Ethanol | 83.29 |
| Purified water | 0.46 |

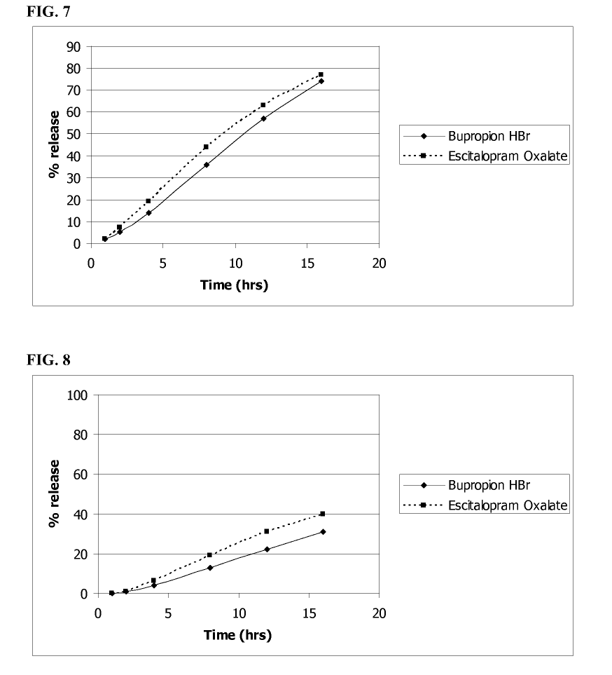

The dissolution profile of the above pharmaceutical composition was determined under the dissolution conditions described below in Table 8. The result of the dissolution testing is presented as a % of the total bupropion HBr and escitalopram oxalate in the controlled release tablet and is also depicted in FIG. 8.

TABLE 8
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 0 | 0 |
| 2 | 1 | 1 |
| 4 | 4 | 6 |
| 8 | 13 | 19 |
| 12 | 22 | 31 |
| 16 | 31 | 40 |

### Example 9

### Homogenous Tablet Core Composition and Method of Manufacture

A. Bupropion HBr/Escitalopram Oxalate Co-Granulation:

Bupropion HBr/Escitalopram Oxalate
Co-Granulation Composition

| Component | % w/w |
|---|---|
| Bupropion HBr | 86.0 |
| Escitalopram Oxalate | 5.6 |
| Butylatedhydroxytoluene | 0.1 |
| PVA | 3.3 |
| Citric Acid | 5.0 |

The pharmaceutical actives, bupropion HBr and escitalopram oxalate, were top-spray granulated using a Glatt GPCG 1 (6 inch chamber). The theoretical batch size was 3273.9 g. The granulation solution was prepared in 3 steps:
4.) an aqueous (purified water) solution of polyvinyl alcohol (PVA) (5.6% of solution) and citric acid (8.7% of solution) was prepared
5.) a solvent (2-propanol) solution of BHT (5.9% of solution) was prepared
6.) the BHT solvent solution was added to the aqueous PVA/citric acid solution and mixed for a minimum of 20 minutes. The BHT became finely dispersed in the PVA/citric acid solution. The final composition of the granulation suspension was 4.82% PVA, 7.50% citric acid, 1.67% 2-propanol, 0.1% BHT, 85.91% purified water.

The bupropion HBR and escitalopram oxalate were charged to the granulation chamber in the required ratio to give the desired final dosage strengths of each active. For a 312 mg dose of bupropion HBr and a 16 mg dose of escitalopram oxalate the actives were dispensed such that the material in the granulation chamber consisted of 93.6% bupropion HBr and 6.4% escitalopram oxalate. In this example 3 kg of material was granulated, 2815.5 g of bupropion HBr mixed with 193.73 g of escitalopram oxalate. Approximately half of the bupropion HBr was charged to the granulation chamber, followed by all of the escitalopram oxalate, and next the remainder of the bupropion HBr was charged to the chamber. The granulation suspension was sprayed onto the 3 kg mix of bupropion HBr to a weight gain of 9.1% to produce a granule comprising of 86% bupropion HBr, 5.6% escitalopam oxalate, 3.3% PVA, 5.0% citric acid, and 0.1% BHT. The powder bed temperature was maintained between 40-45° C., and the liquid spray rate maintained between 5-7 g/min throughout the granulation process. When spraying of the granulation solution was stopped, the granules were fluid bed dried to an LOD (loss on drying) level of <1%. The granules were screened through a 1.00 mm screen, and the material <1.00 mm retained for manufacture of the homogenous tablet core.

B. Homogenous Tablet Core Composition and Method of Manufacture

To manufacture a homogenous tablet core comprising 312 mg of bupropion HBr (equivalent to 234 mg of bupropion base) and 16 mg of escitalopram oxalate (equivalent to 12.5 mg escitalopram base), a homogenous tablet blend with the following composition was prepared; 97% bupropion HBr/escitalopram oxalate co-granules (manufactured as outlined above), and 3% Compritol 888 ATO (screened through a 500 μm screen). A homogenous tablet blend of 1500 g was manufactured by dispensing 1455 g of bupropion HBr/escitalopram oxalate co-granules, and 45 g of the screened Compritol 888 ATO. The material was added to a v-blender (15 litre shell of a Pharmatech AB-050 v-blender) in the following order:

4. ˜half of the bupropion HBr/escitalopram oxalate co-granules

5. All of the Compritol 888 ATO

6. The remaining bupropion HBr/escitalopram oxalate co-granules

The tablet components were blended for 10 minutes, with the v-shell speed set to 25 rpm and the intensifier bar turned off. The homogenous tablet blend was discharged from the v-shell and charged to a tablet press (Riva Bilayer 11 station rotary tablet press) and compressed to a target tablet weight of 373.5 mg and a target tablet hardness of 130N using 9 mm round normal concave shaped tablet tooling. The resulting product comprises the homogenous tablet core, which at this point is an immediate release core having the following composition:

Homogenous Tablet Core Composition

| Component | mg/tablet | % w/w |
|---|---|---|
| Bupropion HBr | 312 | 84 |
| Escitalopram Oxalate | 20.4 | 5 |
| PVA | 11.68 | 3 |
| Citric Acid | 18.21 | 5 |
| BHT | 0.36 | 0.09 |
| Compritol 888 ATO | 10.83 | 3 |

Tablet Coat Composition and Method of Manufacture

The homogenous IR tablet cores were coated with Kollicoat SR30D (a polyvinyl acetate dispersion with 27% polyvinyl acetate, 2.7% povidone and 0.3% sodium lauryl sulfate) by preparing an aqueous suspension consisting of about 50% aqueous disperion of Kollidon SR30D, 3.5% Talc, 1.5% Triethyl citrate and 45% purified water. Talc was added to approximately 80% of the water and the dispersion mixed with high shear for approximately 15 minutes. In a separate vessel triethyl citrate was added to the Kollicoat SR30D, and the suspension mixed, following which the Talc dispersion was added to the other vessel and the coating suspension made up to volume with the remaining water. The entire suspension was mixed overnight prior to use in coating. The plasticized polymer solution was applied to about 1 kg of the final tablet cores using an O'Hara Labcoat I tablet coating machine (12″ pan) until about a 12% weight gain was obtained. The product temperature was maintained between about 30° C., and the liquid spray rate was maintained between about 3-5 g/min throughout the coating process.

The resulting control-releasing coat has the following composition:

Control-releasing Coat Composition with Kollicoat ® SR30D

| Component | % w/w |
|---|---|
| Kollicoat ® SR30D | 50 |
| Triethyl Citrate | 1.5 |
| Talc | 2.5 |
| Purified water | 45 |

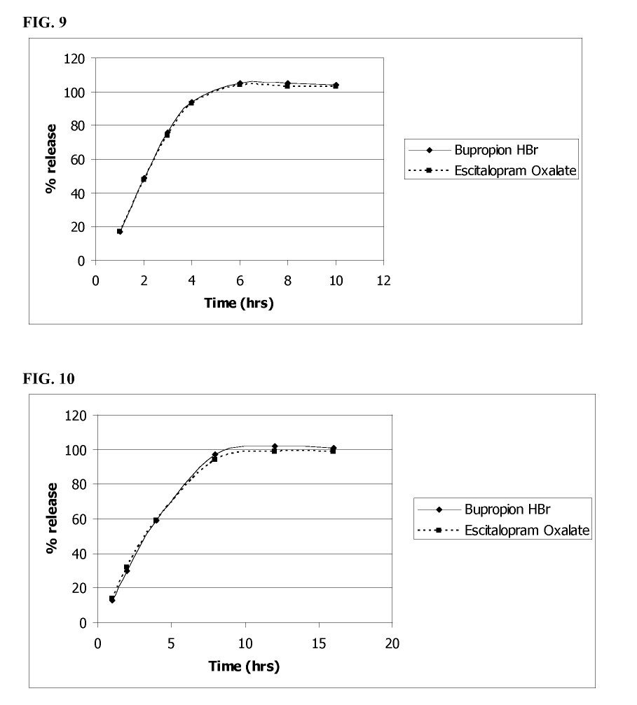

The dissolution profile of the above pharmaceutical composition was determined under the dissolution conditions described below in Table 9. The result of the dissolution testing is presented as a % of the total bupropion HBr and escitalopram oxalate in the controlled release tablet and is also depicted in FIG. 9.

TABLE 9
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 17 | 17 |
| 2 | 49 | 48 |
| 3 | 76 | 74 |
| 4 | 94 | 93 |
| 6 | 105 | 104 |
| 8 | 105 | 103 |
| 10 | 104 | 103 |

### Example 10

### Homogenous Tablet Core Composition and Method of Manufacture

The homogenous tablet core was manufactured according to the method described in Example 3 with the following co-granule and homogenous tablet core composition:

Bupropion HBr (325 mg)/Escitalopram Oxalate
(17.9 mg) Co-Granule Composition

| Component | % w/w |
|---|---|
| Bupropion HBr | 86.88 |
| Escitalopram Ox | 4.78 |
| Butylatedhydroxytoluene | 0.07 |
| PVA | 3.24 |
| Citric Acid | 5.03 |

Homogenous Tablet Core Composition

| Component | mg/tablet | % w/w |
|---|---|---|
| Bupropion HBr | 325.02 | 84.36 |
| Escitalopram Ox | 17.88 | 4.64 |
| PVA | 12.12 | 3.15 |
| Citric Acid | 18.82 | 4.88 |
| BHT | 0.26 | 0.07 |
| Compritol 888 ATO | 11.17 | 2.90 |

Tablet Coat Composition and Method of Manufacture

The control-releasing coat was manufactured according to the method and composition described in Example 1.

The dissolution profile of the above pharmaceutical composition was determined under the dissolution conditions described below in Table 10. The result of the dissolution testing is presented as a % of the total bupropion HBr and escitalopram Ox in the controlled release tablet and is also depicted in FIG. 10.

TABLE 10
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 0 | | |
| 1 | 13 | 14 |
| 2 | 30 | 32 |
| 4 | 59 | 59 |
| 8 | 97 | 94 |
| 12 | 102 | 99 |
| 16 | 101 | 99 |

### Example 11

### Homogenous Tablet Core Composition and Method of Manufacture

The homogenous tablet core was manufactured according to the method described in Example 3 with the following co-granule and homogenous tablet core composition:

Bupropion HBr (400 mg)/Escitalopram Oxalate
(19.2 mg) Co-Granule Composition

| Component | % w/w |
|---|---|
| Bupropion HBr | 87.45 |
| Escitalopram Ox | 4.20 |
| Butylatedhydroxytoluene | 0.08 |
| PVA | 3.24 |
| Citric Acid | 5.03 |

Homogenous Tablet Core Composition

| Component | mg/tablet | % w/w |
|---|---|---|
| Bupropion HBr | 400.03 | 84.92 |
| Escitalopram Ox | 19.21 | 4.07 |
| PVA | 14.82 | 3.15 |
| Citric Acid | 23.00 | 4.88 |
| BHT | 0.37 | 0.08 |
| Compritol 888 ATO | 13.66 | 2.90 |

Tablet Coat Composition and Method of Manufacture

The control-releasing coat was manufactured according to the method and composition described in Example 1.

The dissolution profile of the above pharmaceutical composition was determined under the dissolution conditions described below in Table 11. The result of the dissolution testing is presented as a % of the total bupropion HBr and escitalopram Ox in the controlled release tablet and is also depicted in FIG. 11.

TABLE 11
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 15 | 16 |
| 2 | 30 | 33 |
| 4 | 55 | 56 |
| 8 | 93 | 88 |
| 12 | 103 | 97 |
| 16 | 102 | 97 |

### Example 12

### Homogenous Tablet Core Composition and Method of Manufacture

The homogenous tablet core was manufactured according to the method described in Example 3 with the following co-granule and homogenous tablet core composition:

Bupropion HBr (171.3 mg)/Escitalopram Oxalate
(171.3 mg) Co-Granule Composition

| Component | % w/w |
|---|---|
| Bupropion HBr | 45.78 |
| Escitalopram Ox | 45.78 |
| Butylatedhydroxytoluene | 0.10 |
| PVA | 3.26 |
| Citric Acid | 5.08 |

Homogenous Tablet Core Composition

| Component | mg/tablet | % w/w |
|---|---|---|
| Bupropion HBr | 171.32 | 44.45 |
| Escitalopram Ox | 171.32 | 44.45 |
| PVA | 12.20 | 3.17 |
| Citric Acid | 19.01 | 4.93 |
| BHT | 0.37 | 0.10 |
| Compritol 888 ATO | 11.18 | 2.90 |

Tablet Coat Composition and Method of Manufacture

The control-releasing coat was manufactured according to the method and composition described in Example 1.

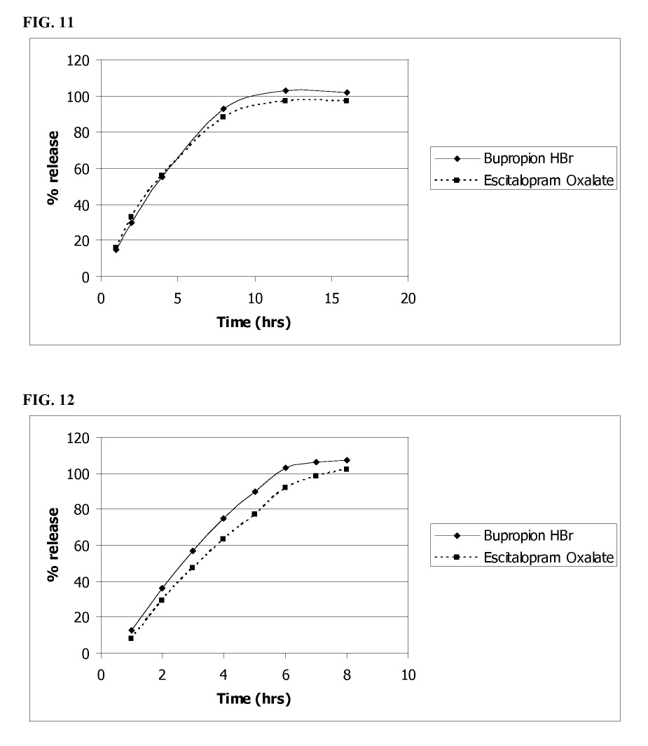

The dissolution profile of the above pharmaceutical composition was determined under the dissolution conditions described below in Table 12. The result of the dissolution testing is presented as a % of the total bupropion HBr and escitalopram Ox in the controlled release tablet and is also depicted in FIG. 12.

TABLE 12
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 13 | 8 |
| 2 | 36 | 29 |
| 3 | 57 | 47 |
| 4 | 75 | 63 |
| 5 | 90 | 77 |
| 6 | 103 | 92 |
| 7 | 106 | 98 |
| 8 | 107 | 102 |

### Example 13

### Homogenous Tablet Core Composition and Method of Manufacture

The homogenous tablet core was manufactured according to the method described in Example 3 with the following co-granule and homogenous tablet core composition:

Bupropion HBr (156 mg)/Escitalopram
(8 mg) Co-Granule Composition

| Component | % w/w |
|---|---|
| Bupropion HBr | 86.03 |
| Escitalopram Ox | 5.63 |
| Butylatedhydroxytoluene | 0.1 |
| PVA | 3.22 |
| Citric Acid | 5.02 |

Homogenous Tablet Core Composition

| Component | mg/tablet | % w/w |
|---|---|---|
| Bupropion HBr | 156.0 | 83.5 |
| Escitalopram Ox | 10.2 | 5.46 |
| PVA | 5.84 | 3.12 |
| Citric Acid | 9.11 | 4.88 |
| BHT | 0.18 | 0.9 |
| Compritol 888 ATO | 5.42 | 3.0 |

Tablet Coat Composition and Method of Manufacture

The control-releasing coat was manufactured according to the method and composition described in Example 1, except that the plasticized polymer solution was applied until about a 18% weight gain was obtained.

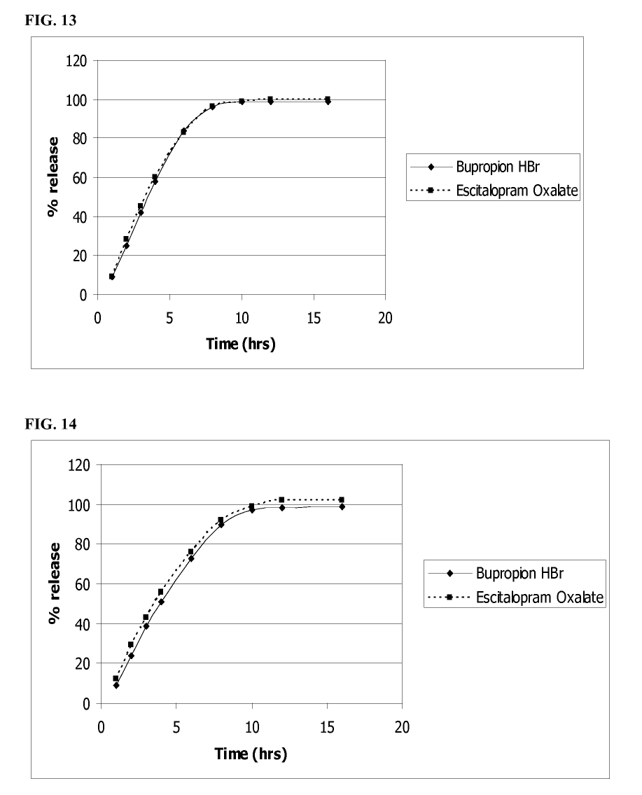

The dissolution profile of the above pharmaceutical composition was determined under the dissolution conditions described below in Table 13. The result of the dissolution testing is presented as a % of the total bupropion HBr and escitalopram Ox in the controlled release tablet and is also depicted in FIG. 13.

TABLE 13
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 9 | 9 |
| 2 | 25 | 28 |
| 3 | 42 | 45 |
| 4 | 58 | 60 |
| 6 | 84 | 83 |
| 8 | 96 | 96 |
| 10 | 99 | 99 |
| 12 | 99 | 100 |
| 16 | 99 | 100 |

### Example 14

### Homogenous Tablet Core Composition and Method of Manufacture

The homogenous tablet core was manufactured according to the method described in Example 3 with the following co-granule and homogenous tablet core composition:

Bupropion HBr (312 mg)/Escitalopram Oxalate
(16 mg) Co-Granule Composition

| Component | % w/w |
|---|---|
| Bupropion HBr | 86.03 |
| Escitalopram Ox | 5.63 |
| Butylatedhydroxytoluene | 0.1 |
| PVA | 3.22 |
| Citric Acid | 5.02 |

Homogenous Tablet Core Composition

| Component | mg/tablet | % w/w |
|---|---|---|
| Bupropion HBr | 312.0 | 83.5 |
| Escitalopram Ox | 20.4 | 5.46 |
| PVA | 11.68 | 3.12 |
| Citric Acid | 18.21 | 4.88 |
| BHT | 0.36 | 0.96 |
| Compritol 888 ATO | 10.83 | 3.0 |

Tablet Coat Composition and Method of Manufacture

The control-releasing coat was manufactured according to the method and composition described in Example 1, except that the plasticized polymer solution was applied until about a 12% weight gain was obtained.

The dissolution profile of the above pharmaceutical composition was determined under the dissolution conditions described below in Table 14. The result of the dissolution testing is presented as a % of the total bupropion HBr and escitalopram Ox in the controlled release tablet and is also depicted in FIG. 14.

TABLE 14
Dissolution Conditions: 900 ml 0.1N HCl,
USP Apparatus 1, 75 rpm, 37° C.

% Released

| Time (hr) | Bupropion HBr | Escitalopram Ox |
|---|---|---|
| 1 | 9 | 12 |
| 2 | 24 | 29 |
| 3 | 39 | 43 |
| 4 | 51 | 56 |
| 6 | 73 | 76 |
| 8 | 90 | 92 |
| 10 | 97 | 99 |
| 12 | 98 | 102 |
| 16 | 99 | 102 |

### Example 15

Pharmaceutical compositions comprising a homogenous controlled-release matrix comprising about 300 mg of bupropion HCl (equivalent to about 260 mg of bupropion base) and about 25.5 mg of escitalopram oxalate (equivalent to about 20 mg escitalopram base) were prepared by direct blending of Bupropion HCl granule fines (initially manufactured as granules described in Example 18 and screened through a 45 mesh screen. Particles <355 μm were retained for the manufacturing the controlled-release matrix pharmaceutical composition) with escitalopram oxalate powder and other excipients with the following composition.

Homogenous Controlled-Release Matrix Composition

| Component | mg/tablet | % w/w |
|---|---|---|
| Bupropion HCl | 300.0 | 39.56 |
| Escitalopram Oxalate | 25.5 | 3.36 |
| Polyvinyl Alcohol | 10.6 | 1.40 |
| Hydroxypropyl cellulose | 200.0 | 26.37 |
| Lactose DT | 125.0 | 16.48 |
| Microcrystalline cellulose | 72.2 | 9.52 |
| Silicon dioxide | 2.5 | 0.33 |
| Magnesium stearate | 22.5 | 2.97 |

A homogenous blend of about 150 g was manufactured by dispensing about 61.44 g of bupropion HCl granule fines, about 5.04 g of escitalopram oxalate, about 39.56 g of hydroxypropyl cellulose, about 24.73 g of lactose, about 14.28 g of microcrystalline cellulose, about 0.49 g of silicon dioxide, and about 4.45 g of magnesium stearate. All the excipients were pre-screened through the 30 mesh screen prior to dispensing. Manual bag mixing was applied according to the following order.
1. Pre-mix active escitalopram oxalate with a small portion of hydroxypropyl cellulose, lactose and microcrystalline cellulose.
2. Add approximately half of the Bupropion HCl granules and mix
3. Add approximately half of the remaining hydroxypropyl cellulose, lactose and microcrystalline cellulose, and mix
4. Add the remaining Bupropion HCl granules, and mix
5. Add the remaining hydroxypropyl cellulose, lactose and microcrystalline cellulose, and mix
6. Add silicon dioxide and magnesium stearate, and mix for 2 minutes.

The homogenous blend was further compressed using Natoli Single Station Press equipped with 0.706″×0.329″ capsule shaped tablet tooling. The target tablet weight was 758.3 mg. The hardness of the table was about 210N. 2.25 tons of compression force was applied.

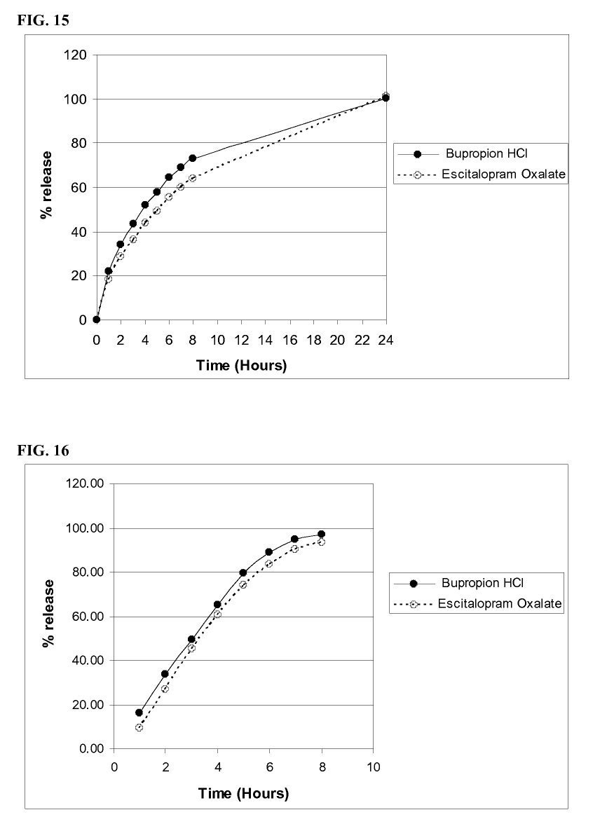

The dissolution results of the homogenous controlled-release matrix pharmaceutical composition (batch no. BUPHCL/ESC-300/25.5-03-07) was determined under the dissolution conditions described below in Table 15. The result of the dissolution testing is presented as a % of the total bupropion HCl and escitalopram oxalate in the controlled release composition. The dissolution profile determined under the dissolution conditions is also depicted in FIG. 15.

TABLE 15
Dissolution Conditions: 900 ml 0.1N HCl,
USP Apparatus 1, 75 rpm, 37° C.

% Released

| | | Escitalopram |
|---|---|---|
| Time (hr) | Bupropion HCl | Oxalate |
| 1 | 21.88 | 18.19 |
| 2 | 34.15 | 28.44 |
| 3 | 43.59 | 36.45 |
| 4 | 52.04 | 44.01 |
| 5 | 57.92 | 49.35 |
| 6 | 64.57 | 55.56 |
| 7 | 69.02 | 59.87 |
| 8 | 73.09 | 63.96 |

### Example 16

### Bi-Layer Tablet Core Composition and Method of Manufacture

To manufacture the bi-layer tablet core comprising about 150 mg of bupropion HCl (equivalent to about 130 mg of bupropion base) and about 150 mg of escitalopram oxalate (equivalent to about 118 mg escitalopram base), two separate blends with the following compositions were prepared separately: 1) about 47.23% bupropion HCl granules (manufactured according to the separate granulation method) with about 1.52% Compritol 888 ATO (screened through a 500 μm screen); 2) about 49.73% escitalopram oxalate granules (manufactured according to the separate granulation method, potency of the escitalopram oxalate granules is 90.1%) with about 1.52% Compritol 888 ATO (screened through a 500 μm screen). The theoretical batch size was about 100 g. About 47.2 g of bupropion HCl granules were lubricated with about 1.52 g of the screened Compritol 888 ATO by bag mixing for 2 minutes, and about 49.7 g of escitalopram oxalate granules were lubricated with about 1.52 g of the screened Compritol 888 ATO by bag mixing for 2 minutes.

The bi-layer tablet cores were prepared using Natoli Single Station Press equipped with 9 mm round concave shaped tablet tooling by pre-compressing the lubricated escitalopram oxalate granules as the first layer and followed by compressing the lubricated bupropion HCl granules as second layer. The target tablet weight was 332 mg and the target tablet hardness was about 175N. 0.5 tons of pre-compression force and 3 tons of compression force was applied. The resulting product comprises the bi-layer tablet core (batch no. BUPHCL/ESC-150-150-01-07), which at this point is an immediate release core having the following composition:

Bi-Layer Tablet Core Composition

| Component | mg/tablet | % w/w |
|---|---|---|
| Bupropion HCl | 150.0 | 45.62 |
| Escitalopram oxalate | 150.0 | 45.62 |
| PVA | 5.3 | 1.61 |
| BHT | 2.3 | 0.70 |
| Kollidon ® 90F | 11.2 | 3.41 |
| Compritol 888 ATO | 10.0 | 3.04 |

Tablet Coat Composition and Method of Manufacture

Tablet Control-releasing Coat Composition

| Component | % w/w |
|---|---|
| Ethocel 100 FP Premium | 4.61 |
| Kollidon ® 90F | 2.86 |
| PEG 3350 | 1.03 |
| Dibutylsebacate | 0.50 |
| 2-Propanol | 8.68 |
| Absolute Ethanol | 81.86 |
| Purified water | 0.46 |

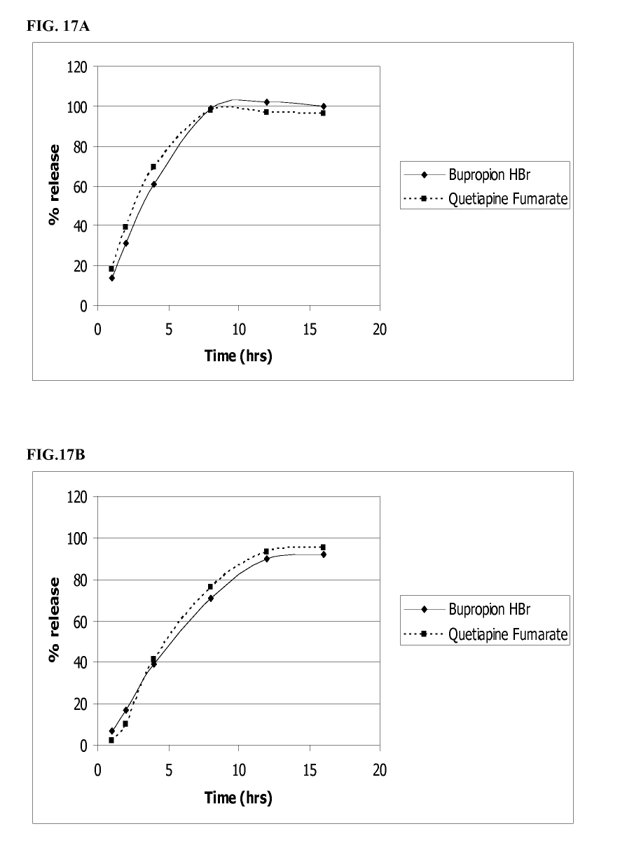

The bi-layer tablet cores were coated separately with an ethylcellulose based film by preparing an organic solvent solution consisting of about 4.61% ethocel standard 100FP premium, about 2.86% Kollidon® 90F, about 1.03% carbowax sentry polyethylene glycol 3350 granular NF FCC grade, about 0.5% dibutyl sebacate NF, about 8.68% 2-propanol, about 81.86% absolute ethanol, and about 0.46% purified water. The plasticized polymer solution is applied to about 1.53 kg of the tablet cores, including about 35 g of active tablet cores and about 1.5 kg placebo tablets (comprising 69% lactose monohydrate, 30% microcrystalline cellulose and 1% magnesium stearate), using an O'Hara Labcoat II-X tablet coater (15″ pan) until about a 10% weight gain is obtained. The product temperature is maintained between about 30-33° C., and the liquid spray rate is maintained between about 20-22 g/min throughout the coating process. The controlled release coated tablets are then cured for about 25 minutes (inlet air is set at about 50° C., pan speed set at 3 rpm). The dissolution profile of the controlled release coated bi-layer tablets (batch no. BUPHCL/ESC-150-150-02-07) was determined under the dissolution conditions described below in Table 16. The result of the dissolution testing is presented as a % of the total bupropion HCl and escitalopram oxalate in the controlled release tablet and is also depicted in FIG. 16.

TABLE 16
Dissolution Conditions: 900 ml 0.1N HCl,
USP Apparatus 1, 75 rpm, 37° C.

% Released
(Bi-layer Tablets)

| | | Escitalopram |
|---|---|---|
| Time (hr) | Bupropion HCl | Oxalate |
| 1 | 16.36 | 9.47 |
| 2 | 33.56 | 27.06 |
| 3 | 49.54 | 45.32 |
| 4 | 65.00 | 60.81 |
| 5 | 79.63 | 74.22 |
| 6 | 88.97 | 83.48 |
| 7 | 94.82 | 90.11 |
| 8 | 97.06 | 93.47 |

### Example 17

### Homogenous Tablet Core Composition and Method of Manufacture

A. Bupropion HBr Granulation:

Bupropion HBr granules were manufactured as described in example 1.

B. Quetiapine Fumarate Granulation:

Quetiapine Fumarate Granule Composition

| Component | % w/w |
|---|---|
| Quetiapine Fumarate | 96.62 |
| Polyvinyl Alcohol | 3.38 |

The pharmaceutical active, Quetiapine fumarate, was top-spray granulated using a Glatt GPCG 1 (6 inch chamber). The theoretical batch size was 2070 g. An aqueous (purified water) solution of polyvinyl alcohol (PVA) (4.8% of solution) was sprayed onto 2 kg of Quetiapine fumarate to a weight gain of 3.5% to produce a granule comprising of 96.62% Quetiapine fumarate, and 3.38% PVA. The powder bed temperature was maintained between 38-45° C., and the liquid spray rate maintained between 5-10 g/min throughout the granulation process. When spraying of the granulation solution was stopped, the granules were fluid bed dried for 10 minutes with a product temperature of ˜45° C. The granules were screened and the granules with a particle size below 800 μm and above 355 μm are retained for use in the tablet core.

C. Homogenous Tablet Core Composition and Method of Manufacture

To manufacture a tablet core containing 348 mg of Bupropion HBr (equivalent to 260 mg of Bupropion base) and 23 mg of Quetiapine fumarate (equivalent to 20 mg quetiapine base), a homogenous tablet blend with the following composition was prepared; 90.79% Bupropion HBr granules (manufactured as outlined in Example 1), 5.71% Quetiapine fumarate granules (manufactured as outlined above), 3.40% Compritol 888 ATO, and 0.1% lake green blend. A homogenous tablet blend of 400 g was manufactured by dispensing 363.16 g of Bupropion HBr granules, 22.84 g of Quetiapine fumarate granules, 13.60 g of Compritol 888 ATO, and 0.40 g of lake green blend. The material was added to a v-blender (4 quart shell of a PK labmaster v-blender) in the following order:

1. ˜half of the Bupropion HBr granules

2. All of the Quetiapine fumarate granules

3. All of the Compritol 888 ATO

4. All of the lake green blend

5. The remaining Bupropion HBr granules

The tablet components were blended for 10 minutes, with the intensifier bar turned off.
The homogenous tablet blend was discharged from the v-shell and charged to a tablet press (Riva Bilayer 11 station rotary tablet press) and compressed to a target tablet weight of 418 mg and a target tablet hardness of 120N using 9 mm round deep concave shaped tablet tooling. The resulting product comprises the homogenous tablet core, which at this point is an immediate release core having the following composition:

Homogenous Tablet Core Composition

| Component | mg/tablet | % w/w |
|---|---|---|
| Bupropion HBr | 348.08 | 83.27 |
| Quetiapine Fumarate | 23.06 | 5.52 |
| PVA | 13.10 | 3.13 |
| Citric Acid | 19.13 | 4.58 |
| Lake Green Blend | 0.42 | 0.10 |
| Compritol 888 ATO | 14.21 | 3.40 |

Tablet Coat Composition and Method of Manufacture

The homogenous IR tablet cores were next coated with the control-releasing coat as described in Example 1.

The dissolution profile of the above pharmaceutical composition was determined under the dissolution conditions described below in Tables 17A and 17B. The results of the dissolution testing are presented as a % of the total bupropion HBr and quetiapine fumarate in the controlled release tablet and are also depicted in FIGS. 17A and 17B.

TABLE 17A
Dissolution Conditions: 900 ml 0.1N HCl,
USP Apparatus 1, 75 rpm, 37° C.

% Released

| Time (hr) | Bupropion HBr | Quetiapine Fumarate |
|---|---|---|
| 1 | 14 | 18 |
| 2 | 31 | 39 |
| 4 | 61 | 69 |
| 8 | 99 | 98 |
| 12 | 102 | 97 |
| 16 | 100 | 96 |

TABLE 17B
Dissolution Conditions: 900 ml pH 6.8 phosphate
buffer, USP Apparatus 1, 75 rpm, 37° C.

% Released

| Time (hr) | Bupropion HBr | Quetiapine Fumarate |
|---|---|---|
| 1 | 7 | 2 |
| 2 | 17 | 10 |
| 4 | 39 | 41 |
| 8 | 71 | 76 |
| 12 | 90 | 93 |
| 16 | 92 | 95 |

### Example 18

### Homogenous IR Tablet Core Composition and Method of Manufacture—Separate Granulation Method

A. Bupropion HCl Granulation:

Bupropion HCl Granule Composition

| Component | % w/w |
|---|---|
| Bupropion HCl | 96.59 |
| Polyvinyl Alcohol | 3.41 |

The pharmaceutical active, bupropion HCl, was top-spray granulated using an Aeromatic MP8 Fluid Bed. The theoretical batch size was 310.6 kg. An aqueous (purified water) solution of polyvinyl alcohol (PVA) (4.82% of solution) was sprayed onto 300.0 kg of bupropion HCl to a weight gain of 3.41% to produce a granule comprising of 96.59% bupropion HCl, 3.41% PVA. The powder bed temperature was maintained between 48-52° C., and the liquid spray rate maintained between 1500 g/min throughout the granulation process. When spraying of the granulation solution was stopped, the granules were fluid bed dried to a LOD (loss on drying) level of <1%. The granules were screened and the granules with a particle size of between about 355 μm and about 850 μm were retained for manufacture of the homogenous tablet core.

B. Escitalopram Oxalate Granulation:

Citalopram HCl Granule Composition

| Component | % w/w |
|---|---|
| Escitalopram Oxalate | 91.74 |
| Kollidon ® 90F | 6.88 |
| Butylated hydroxytoluene | 1.38 |

The pharmaceutical active, escitalopram oxalate, was top-spray granulated using an Aeromatic fluid bed MP1. The theoretical batch size was 1943.5 g. An organic solvent (2-propanol) solution of Kollidon® 90F (6.0% of solution) and butylated hydroxytoluene (BHT) (1.2% of solution) was sprayed onto about 1783.0 g of escitalopram oxalate to a weight gain of about 8.3% to produce a granule comprising of about 91.74% citalopram HCl, 6.88% Kollidon® 90F, and 1.38% BHT. The powder bed temperature was maintained between 35-45° C., and the liquid spray rate maintained between 13-17 g/min throughout the granulation process. When spraying of the granulation solution was stopped, the granules are fluid bed dried to a LOD (loss on drying) level of <1%. The granules were screened and the granules with a particle size of between about 355 μm and about 850 μm were retained for manufacture of the homogenous tablet core.

C. Homogenous IR Tablet Core Composition and Method of Manufacture

To manufacture the homogenous tablet core comprising about 300 mg of bupropion HCl (equivalent to about 260 mg of bupropion base) and about 25.5 mg of escitalopram oxalate (equivalent to about 20 mg escitalopram base), a blend with the following composition was prepared: about 88.9% bupropion HCl granules (manufactured as described above), about 8.1% escitalopram oxalate granules (manufactured as described above, potency of the escitalopram oxalate granules is 90.1%) and about 3.0% Compritol 888 ATO (screened through a 500 μm screen). A homogenous blend of about 1000 g was manufactured by dispensing about 889.0 g of bupropion HCl granules, about 81.0 g of escitalopram oxalate granules, and about 30.3 g of the screened Compritol 888 ATO. The material was added to a 8 qt. v-blender in the following order:

5. about half of the bupropion HCl granules

6. All of the escitalopram oxalate granules

7. All of the Compritol 888 ATO

8. The remaining bupropion HCl granules

The tablet core components were homogenously blended for about 10 minutes, with the v-shell speed set to 25 rpm. The homogenous blend was discharged from the v-shell and charged to a tablet press (Manesty Betapress 16 station) and compressed to a target tablet weight of about 349.5 mg and a target tablet hardness of about 130N using 9 mm round normal concave shaped tablet tooling. The resulting product comprises the homogenous tablet core, which at this point is an immediate release core having the following composition:

Homogenous IR Tablet Core Composition

| Component | mg/tablet | % w/w |
|---|---|---|
| Bupropion HCl | 300.0 | 85.96 |
| Escitalopram oxalate | 25.5 | 7.31 |
| Polyvinyl Alcohol | 10.6 | 3.03 |
| Butylated hydroxytoluene | 0.38 | 0.11 |
| Kollidon ® 90F | 1.91 | 0.55 |
| Compritol 888 ATO | 10.6 | 3.03 |

Tablet Coat Composition and Method of Manufacture

Tablet Control-releasing Coat Composition

| Component | % w/w |
|---|---|
| Ethocel 100 FP Premium | 4.61 |
| Kollidon ® 90F | 2.86 |
| Polyethylene Glyol 3350 | 1.03 |
| Dibutylsebacate | 0.50 |
| 2-Propanol | 8.68 |
| Absolute Ethanol | 81.86 |
| Purified water | 0.46 |

The homogenous IR tablet cores were coated with an ethylcellulose based film by preparing an organic solvent solution consisting of about 4.61% ethocel standard 100FP premium, about 2.86% Kollidon® 90F, about 1.03% carbowax sentry polyethylene glycol 3350 granular NF FCC grade, about 0.5% dibutyl sebacate NF, about 8.68% 2-propanol, about 81.86% absolute ethanol, and about 0.46% purified water. The plasticized polymer solution is applied to about 1.7 kg of the tablet cores, including about 0.2 kg of active tablet cores and about 1.5 kg placebo tablets (comprising 69% lactose monohydrate, 30% microcrystalline cellulose and 1% magnesium stearate), using an O'Hara Labcoat II-X tablet coater (15″ pan) until about a 10% weight gain is obtained. The product temperature is maintained between about 30-33° C., and the liquid spray rate is maintained between about 20-22 g/min throughout the coating process. The controlled release coated tablets are then cured for about 25 minutes (inlet air is set at about 50° C., pan speed set at 3 rpm).

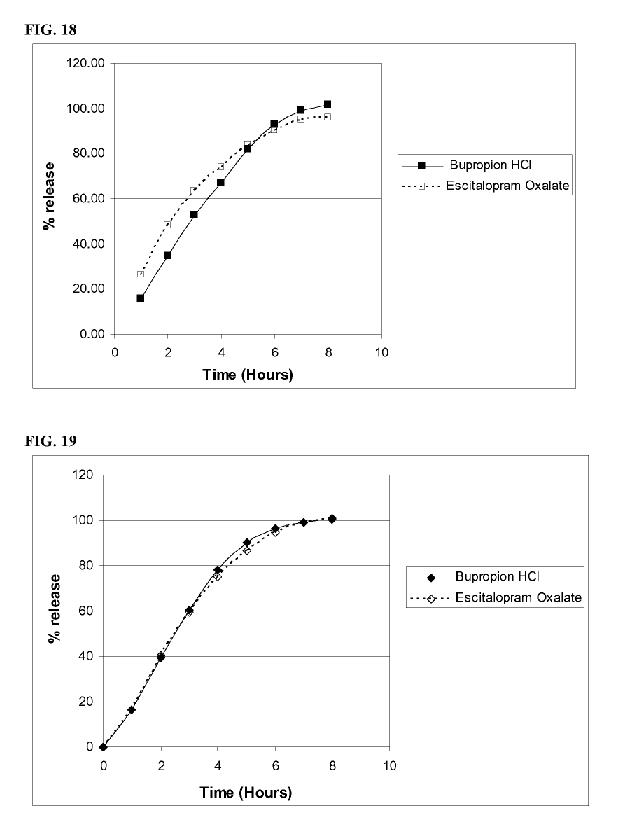

The dissolution results of the above coated talbets are presented as a % of the total bupropion HCl and escitalopram oxalate in the controlled release tablet (batch no. BUPHCL/ESC-300/25.5-02-07). The dissolution profile was determined under the dissolution conditions shown in Table 18 and is also depicted in FIG. 18.

TABLE 18
Dissolution Conditions: 900 ml 0.1N HCl,
USP Apparatus 1, 75 rpm, 37° C.

% Released

| | | Escitalopram |
|---|---|---|
| Time (hr) | Bupropion HCl | Oxalate |
| 1 | 15.77 | 26.30 |
| 2 | 34.68 | 48.19 |
| 3 | 52.34 | 63.33 |
| 4 | 67.21 | 74.10 |
| 5 | 81.76 | 83.56 |
| 6 | 92.94 | 90.30 |
| 7 | 99.19 | 95.04 |
| 8 | 101.69 | 95.74 |

### Example 19

Homogenous Tablet Core Composition and Method of Manufacture

The homogenous tablet core comprising about 225 mg of bupropion HCl (equivalent to about 195 mg of bupropion base) and about 75 mg of escitalopram oxalate (equivalent to about 58.8 mg escitalopram base) was prepared by blending and tabletting the following compositions: about 71.42% bupropion HCl granules (manufactured according to the separate granulation method)), and about 25.51% escitalopram oxalate granules (manufactured according to the separate granulation method), potency of the escitalopram oxalate granules is 90.1%), and about 3.07% Compritol 888 ATO (screened through a 500 μm screen). A homogenous blend of about 100 g was manufactured by dispensing about 71.4 g of bupropion HCl granules, about 25.5 g of escitalopram oxalate granules, and about 3.07 g of the screened Compritol 888 ATO, and manually bag mixing for 2 minutes.

The bulk blend was compressed using Natoli Single Station Press equipped with 9 mm round concave shaped tablet tooling to a target tablet weight of about 326 mg and a target tablet hardness of about 140N. 3 tons of compression force was applied. The resulting homogenous tablet core (batch no. BUPHCL/ESC-225-75-01-07) has the following composition.

Homogenous Tablet Core Composition

| Component | mg/tablet | % w/w |
|---|---|---|
| Bupropion HCl | 225.0 | 68.98 |
| Escitalopram oxalate | 75.0 | 23.0 |
| PVA | 7.94 | 2.43 |
| BHT | 1.37 | 0.42 |
| Kollidon ® 90F | 6.84 | 2.10 |
| Compritol 888 ATO | 10.0 | 3.07 |

Tablet Coat Composition and Method of Manufacture

Tablet Control-releasing Coat Composition

| Component | % w/w |
|---|---|
| Ethocel 100 FP Premium | 4.61 |
| Kollidon ® 90F | 2.86 |
| PEG 3350 | 1.03 |
| Dibutylsebacate | 0.50 |
| 2-Propanol | 8.68 |
| Absolute Ethanol | 81.86 |
| Purified water | 0.46 |

The homogenous tablet cores were coated separately with an ethylcellulose based film by preparing an organic solvent solution consisting of about 4.61% ethocel standard 100FP premium, about 2.86% Kollidon® 90F, about 1.03% carbowax sentry polyethylene glycol 3350 granular NF FCC grade, about 0.5% dibutyl sebacate NF, about 8.68% 2-propanol, about 81.86% absolute ethanol, and about 0.46% purified water. The plasticized polymer solution was applied to about 1.53 kg of the tablet cores, including about 30 g of active tablet cores and about 1.5 kg placebo tablets (comprising 69% lactose monohydrate, 30% microcrystalline cellulose and 1% magnesium stearate), using an O'Hara Labcoat II-X tablet coater (15″ pan) until about a 10% weight gain is obtained. The product temperature was maintained between about 30-33° C., and the liquid spray rate was maintained between about 20-22 g/min throughout the coating process. The controlled release coated tablets were then cured for about 25 minutes (inlet air is set at about 50° C., pan speed set at 3 rpm).

The dissolution results of the coated homogenous tablets (batch no. BUPHCL/ESC-225-75-02-07) are presented as a % of the total bupropion HCl and escitalopram oxalate released under the conditions described in Table 19. The dissolution profile is also depicted in FIG. 19.

TABLE 19
Dissolution Conditions: 900 ml 0.1N HCl,
USP Apparatus 1, 75 rpm, 37° C.

% Released (Homogenous Tablets)

| | | Escitalopram |
|---|---|---|
| Time (hr) | Bupropion HCl | Oxalate |
| 1 | 16.63 | 16.65 |
| 2 | 39.40 | 40.32 |
| 3 | 60.60 | 59.61 |
| 4 | 78.36 | 75.31 |
| 5 | 90.30 | 86.68 |
| 6 | 96.38 | 94.46 |
| 7 | 99.14 | 99.30 |
| 8 | 100.26 | 101.01 |

### Example 20

### Homogenous Tablet Core Composition and Method of Manufacture

To manufacture a homogenous tablet core comprising about 300 mg of tramadol HCl (equivalent to about 263.5 mg of tramadol base) and about 25.5 mg of escitalopram oxalate (equivalent to about 20 mg escitalopram base), a blend with the following composition was prepared: about 88.9% tramadol HCl powder, about 7.6% escitalopram oxalate powder, about 3.1% Compritol 888 ATO (screened through a 500 μm screen) and 0.5% magnesium stearate (screened through a 500 μm screen). A homogenous blend of about 93.4 g was manufactured by dispensing about 83.0 g of tramadol HCl powder, about 7.1 g of escitalopram oxalate, about 2.9 g of the screened Compritol 888 ATO, and about 0.47 g of magnesium stearate. The material was manually blended by bag mixing for 2 minutes.

The homogenous blend was compressed using Natoli Single Station Press equipped with 9 mm round concave shaped tablet tooling to a target tablet weight of about 337.5 mg and a target tablet hardness of about 90N. 1.75 tons of compression force is applied. The resulting homogenous tablet core (batch no. 08029T) has the following composition.

Homogenous Tablet Core Composition

| Component | mg/tablet | % w/w |
|---|---|---|
| Tramadol HCl | 300.00 | 88.89 |
| Escitalopram Oxalate | 25.50 | 7.56 |
| Compritol 888 ATO | 10.30 | 3.05 |
| Magnesium Stearate | 1.69 | 0.50 |

Tablet Coat Composition and Method of Manufacture

Tablet Control-releasing Coat Composition

| Component | % w/w |
|---|---|
| Ethocel 100 FP Premium | 4.61 |
| Kollidon ® 90F | 2.86 |
| PEG 3350 | 1.03 |
| Dibutylsebacate | 0.50 |
| 2-Propanol | 8.68 |
| Absolute Ethanol | 81.86 |
| Purified water | 0.46 |

The homogenous IR tablet cores were coated with an ethylcellulose based film by preparing an organic solvent solution consisting of about 4.61% ethocel standard 100FP premium, about 2.86% Kollidon® 90F, about 1.03% carbowax sentry polyethylene glycol 3350 granular NF FCC grade, about 0.5% dibutyl sebacate NF, about 8.68% 2-propanol, about 81.86% absolute ethanol, and about 0.46% purified water. The plasticized polymer solution was applied to about 1.84 kg of the tablet cores, including about 40.8 g of active tablet cores and about 1.8 kg placebo tablets (comprising 69% lactose monohydrate, 30% microcrystalline cellulose and 1% magnesium stearate), using an O'Hara Labcoat II-X tablet coater (15″ pan) until about a 10% weight gain is obtained. The product temperature was maintained between about 30-34° C., and the liquid spray rate was maintained between about 20-22 g/min throughout the coating process. The controlled release coated tablets were then cured for about 25 minutes (inlet air is set at about 48° C., pan speed set at 3 rpm).

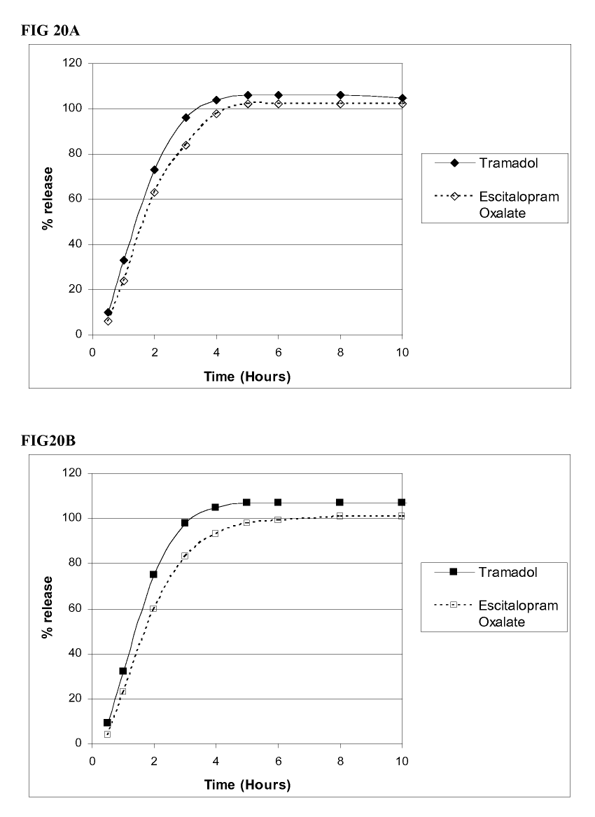

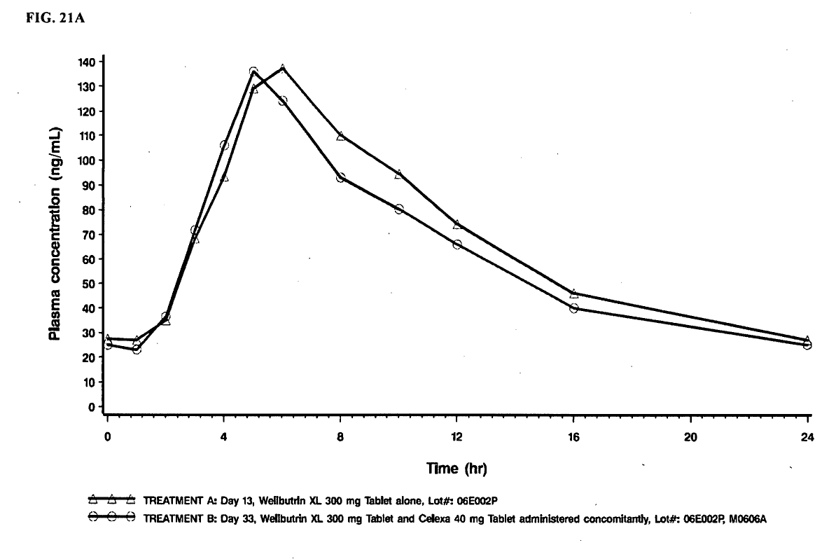

The dissolution profile of the above pharmaceutical composition was determined under the dissolution conditions described below in Table 20A and 20B. The results of the dissolution testing as presented as a % of the total tramadol HCl and escitalopram Ox in the controlled release tablet (batch no. 08029C) and is also depicted in FIGS. 20A and 20B.

The dissolution profile of the above pharmaceutical composition was determined under the dissolution conditions described below in Tables 3A, 3B, and 3C. The results of the dissolution testing are presented as a % of the total bupropion HBr and escitalopram Ox in the controlled release tablet (batch no. 0704028) and are also depicted in FIGS. 3A, 3B, and 3C.

TABLE 20A
Dissolution Conditions: 900 ml 0.1N HCl,
USP Apparatus 1, 75 rpm, 37° C.

% Released

| | | Escitalopram |
|---|---|---|
| Time (hr) | Tramadol HCl | Oxalate |
| 0.5 | 10 | 6 |
| 1 | 33 | 24 |
| 2 | 73 | 63 |
| 3 | 96 | 84 |
| 4 | 104 | 98 |
| 5 | 106 | 102 |
| 6 | 106 | 102 |
| 8 | 106 | 102 |
| 10 | 105 | 102 |
| 12 | 105 | 101 |

TABLE 20B
Dissolution Conditions: 900 ml pH 6.8 phosphate
buffer, USP Apparatus 1, 75 rpm, 37° C.

% Released

| | | Escitalopram |
|---|---|---|
| Time (hr) | Tramadol HCl | Oxalate |
| 0.5 | 9 | 4 |
| 1 | 32 | 23 |
| 2 | 75 | 60 |
| 3 | 98 | 83 |
| 4 | 105 | 93 |
| 5 | 107 | 98 |
| 6 | 107 | 99 |
| 8 | 107 | 101 |
| 10 | 107 | 101 |
| 12 | 107 | 101 |

### Example 21

### A Steady State Drug Interaction Study of Wellbutrin XL® Tablets and Celexa® Tablets in Healthy, Non-Smoking Male and Female Subjects

This study was a single period, 2 treatment, open-label, multiple-dose, drug interaction study under fed conditions. The objective of this study was to evaluate the potential drug interaction of citalopram (Celexa® 40 mg Tablets) on bupropion (Wellbutrin XL® 300 mg Tablets) under steady state conditions. Normal, healthy, non-smoking male and female subjects between the ages of 18 and 55 years were included in the study.

Following an overnight fast of at least 10 hours, and 1 hour after the start of a standard breakfast, subjects received 1 Wellbutrin XL® 150 mg Tablet (Lot #: 06E065P) daily on Days 1 to 3, 1 Wellbutrin XL® 300 mg Tablet (Lot #: 06E002P) daily on Days 4 to 13, 1 Wellbutrin XL® 300 mg Tablet (Lot #: 06E002P) and 1 Celexa® 20 mg Tablet (Lot #: M0512M) daily on Days 14 to 19, 1 Wellbutrin XL® 300 mg Tablet (Lot #: 06E002P) and 1 Celexa® 40 mg Tablet (Lot #: M0606A) daily on Days 20 to 33, and 1 Celexa®20 mg Tablet (Lot #: M0512M) on Days 34 to 36. All treatments were administered orally with 240 mL of ambient temperature water.

26 subjects were dosed in the study, 23 of whom completed the study. One subject was dismissed because of adverse events (AEs), two subjects withdrew for personal reasons. Pharmacokinetic and statistical analyses were performed on the 23 subjects who completed the study.

During the study, 31 blood samples were collected from each subject at the following time points:
Days 1, 10, 11, 12, 30, 31, and 32: 0.00 hour (pre-dose)
Day 13: 0.00 (pre-dose), 1.00, 2.00, 3.00, 4.00, 5.00, 6.00, 8.00, 10.00, 12.00, 16.00, and 24.00 hours post-dose.
Day 33: 0.00 (pre-dose), 1.00, 2.00, 3.00, 4.00, 5.00, 6.00, 8.00, 10.00, 12.00, 16.00, and 24.00 hours post-dose.

Bupropion and its metabolites—hydroxybupropion, bupropion erythroamino alcohol, and bupropion threoamino alcohol, and the internal standard, 1-(3-chlorophenyl)-piperazine, were extracted by solid phase extraction into an organic media from 0.50 mL of human plasma. An aliquot of this extract was injected into a High Performance Liquid Chromatography system and detected using a tandem mass spectrometer. The analytes were separated by reverse phase chromatography. Evaluation of the assay was carried out by the construction of an eight-point calibration curve (excluding zero concentration) covering the range of 1.000 ng/mL to 1023.900 ng/mL for bupropion, 3.907 ng/mL to 4001.200 ng/mL for hydroxybupropion, 1.000 ng/mL to 1024.310 ng/mL for bupropion erythroamino alcohol, and 1.000 ng/mL to 1023.850 ng/mL for bupropion threoamino alcohol in human plasma. The slope and intercept of the calibration curves were determined through weighted linear regression analysis (1/area ratio.²) Two calibration and duplicate QC samples (at three concentration levels) were analyzed along with each batch of the study samples. Peak area ratios were used to determine the concentration of the standards, quality control samples, and the unknown study samples from the calibration curves.

Citalopram and its metabolites—demethylcitalopram and didemethylcitalopram, and the internal standards, citalopram analog, demethylcitalopram analog, and didemethylcitalopram analog, were extracted by liquid-liquid extraction into an organic media from 1.00 mL of human plasma. An aliquot of this extract was injected into a High Performance Liquid Chromatography system and detected using a tandem mass spectrometer. The analytes were separated by reverse phase chromatography. Evaluation of the assay was carried out by the construction of an eight-point calibration curve (excluding zero concentration) covering the range of 0.250 ng/mL to 64.023 ng/mL for citalopram, 0.050 ng/mL to 12.812 ng/mL for demethylcitalopram, and 0.050 ng/mL to 12.796 ng/mL for didemethylcitalopram in human plasma. The slope and intercept of the calibration curves were determined through weighted linear regression analysis (1/conc.²). Two calibration curves and duplicate QC samples (at three concentration levels) were analyzed along with each batch of the study samples. Peak area ratios were used to determine the concentration of the standards, quality control samples, and the unknown study samples from the calibration curves.

The pharmacokinetic analysis was performed on 23 subjects who completed the study. The safety assessment was performed on all subjects who received at least 1 dose during the course of the study.

The following pharmacokinetic parameters for bupropion, bupropion erythroamino alcohol, bupropion threoamino alcohol, hydroxybupropion, and PAWC were calculated by standard non-compartmental methods: AUC_{τ}, Cₘₐₓ, Cₘᵢₙ, C_{avg}, Tₘₐₓ, % Fluctuation, % Swing, Mean Residence Time (MRT), CL/F, and (Metabolite/Parent) M/P ratio. Citalopram and its metabolites concentrations were taken to show that 1) Subjects did take the citalopram products, and 2) Systemic levels of citalopram and its metabolites were present due to Celexa® administration on Days 14-36.

Using General Linear Model (GLM) procedures in Statistical Analysis System (SAS), analysis of variance (ANOVA) was performed on ln-transformed AUC_{τ}, Cₘₐₓ, Cₘᵢₙ, and C_{avg} and on untransformed % Fluctuation, % Swing, MRT M/P ratio and CL/F at the significance level of 0.05. The intra-subject coefficient of variation (CV) was calculated using the Mean Square Error (MSE) from the ANOVA table. The ratio of geometric means and the 90% geometric confidence interval (90% C.I.) were calculated based on the difference in the Least Squares Means of the ln-transformed AUC_{τ}, Cₘₐₓ, and Cₘᵢₙ between the test and reference formulations. Tₘₐₓ was analyzed using nonparametric methods.

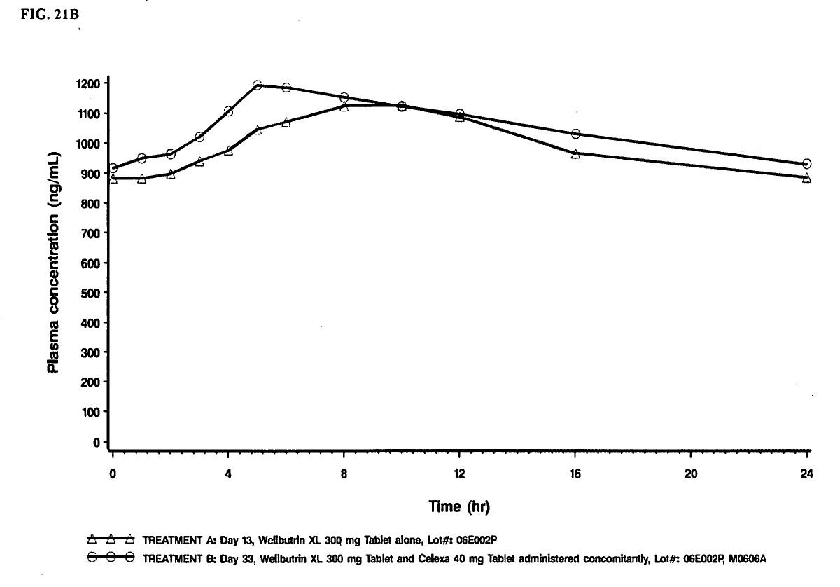

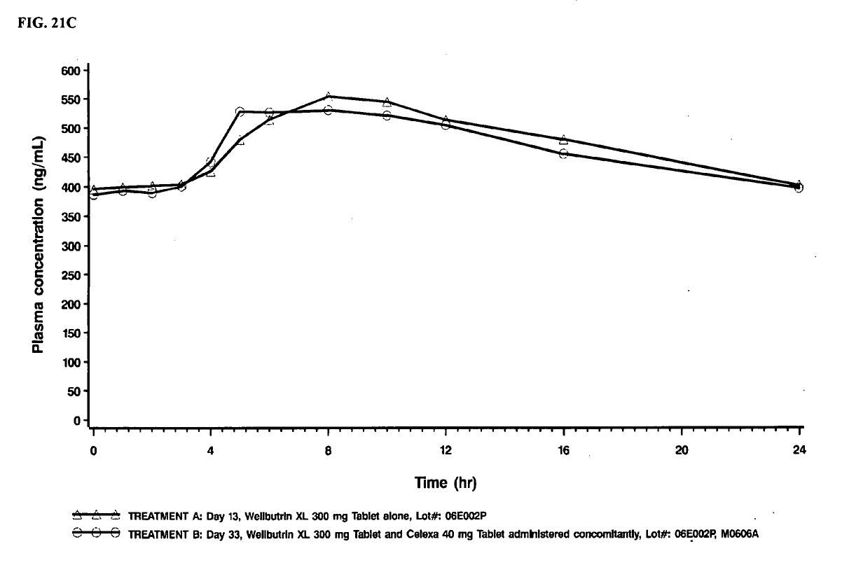

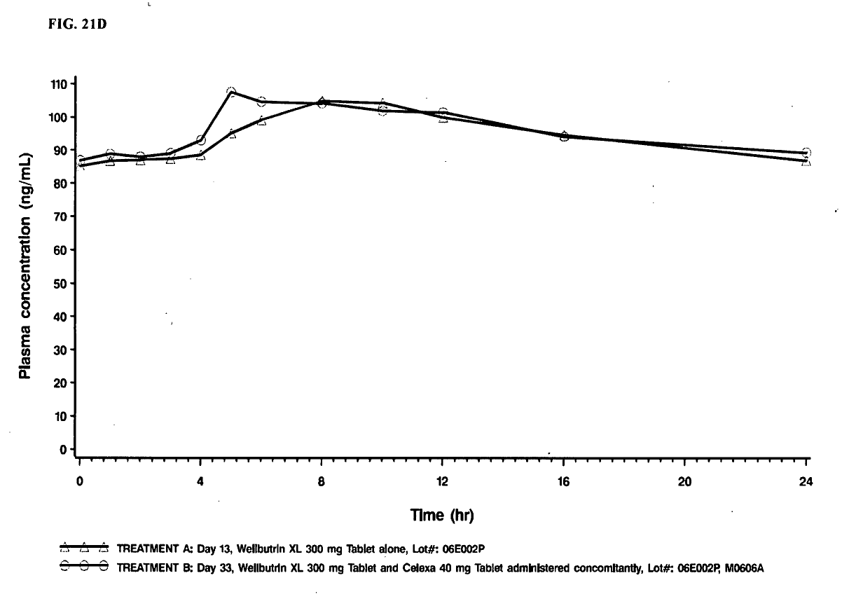

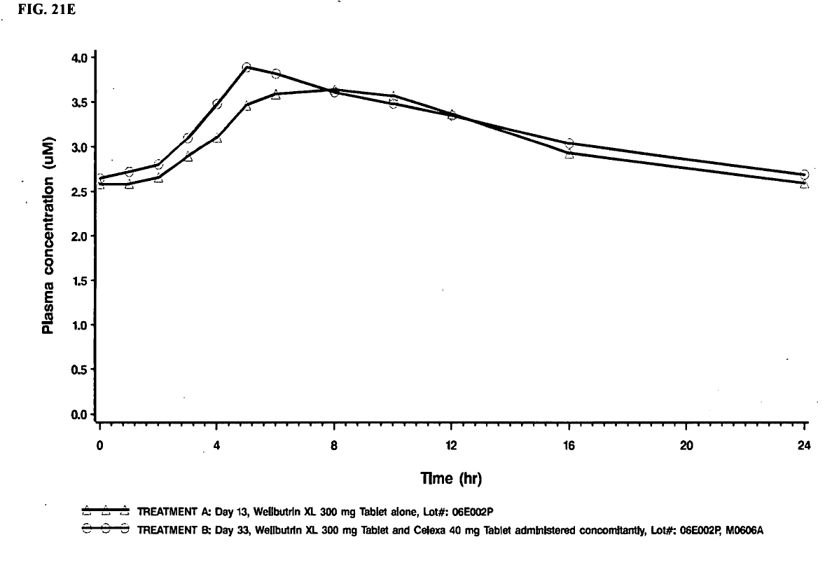

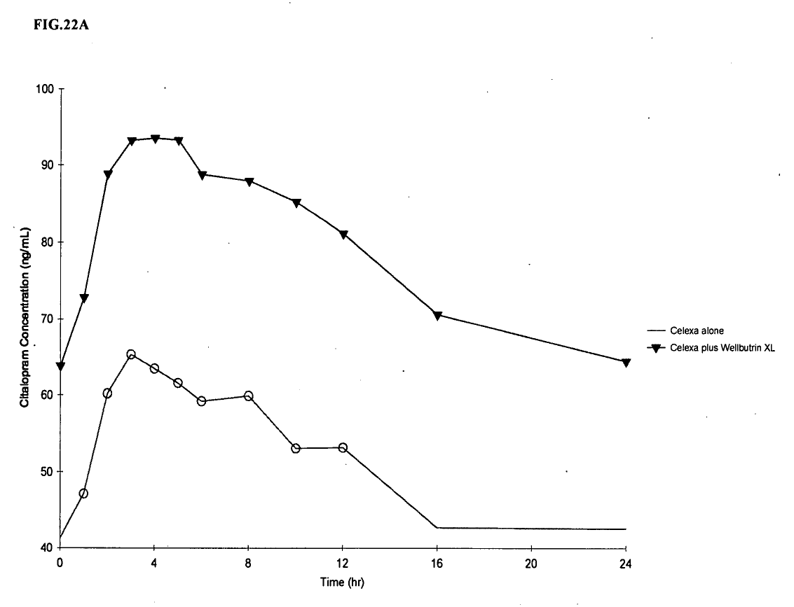

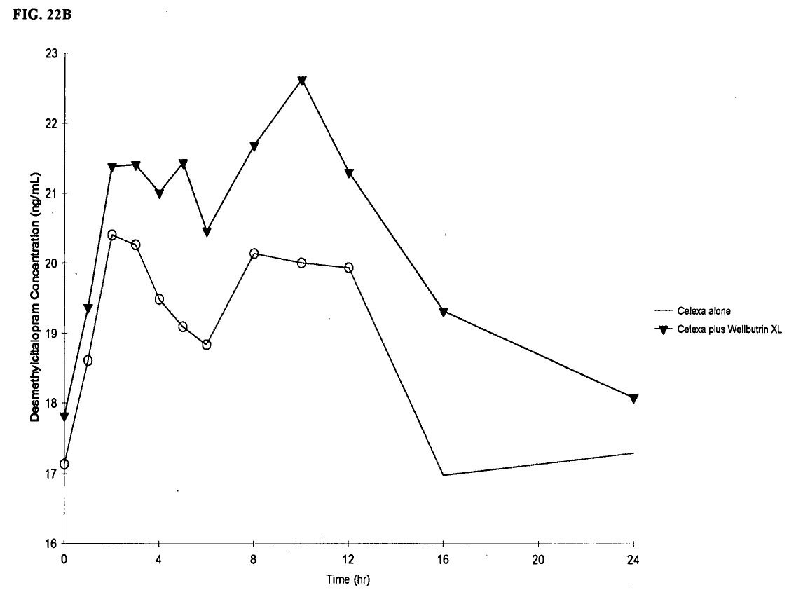

Data for the pharmacokinetic parameters for bupropion and citalopram and its metabolites is presented in the tables below and in FIGS. 21A-E.

Pharmacokinetic Parameters for Bupropion

| | Geometric Mean (% CV) | | | | |
|---|---|---|---|---|---|
| Pharmacokinetic | Arithmetic Mean ± SD | | | | |
| Parameters | Treatment A (n = 23) | | Treatment B (n = 23) | | p-Value |
| AUC_{τ} | 1551.73 | (22.74) | 1418.22 | (24.45) | 0.2095 |
| (ng · hr/mL) | 1591.23 ± 361.82 | | 1459.93 ± 357.01 | | |
| Cₘₐₓ (ng/mL) | 148.70 | (26.29) | 148.12 | (25.55) | 0.9604 |
| | 153.57 ± 40.38 | | 153.24 ± 39.15 | | |
| Cₘᵢₙ (ng/mL) | 25.84 | (32.17) | 24.13 | (31.75) | 0.4705 |
| | 27.17 ± 8.74 | | 25.27 ± 8.02 | | |
| C_{avg} (ng/mL) | 64.66 | (22.74) | 59.09 | (24.45) | 0.2095 |
| | 66.30 ± 15.08 | | 60.83 ± 14.88 | | |
| Tₘₐₓ (hr)* | 6.00 | (3.00-10.00) | 5.00 | (2.00-6.00) | 0.015 |

| | | | |
|---|---|---|---|
| Degree of | 191.16 ± 37.69 | 212.90 ± 50.88 | 0.1067 |
| Fluctuation (%) | | | |
| Degree of Swing (%) | 501.57 ± 180.74 | 548.70 ± 207.50 | 0.4159 |
| MRT (hr) | 10.00 ± 0.61 | 9.77 ± 0.43 | 0.1442 |
| CL/F | 26.45 ± 6.29 | 29.05 ± 7.26 | 0.2019 |

*median (min-max)
TREATMENT A: Day 13, Wellbutrin XL ® 300 mg Tablet alone
TREATMENT B: Day 33, Wellbutrin XL ® 300 mg Tablet and Celexa ® 40 mg Tablet administered concomitantly

Relative Bioavailability Assessments for Bupropion

| Parameter | 90% C.I. | Ratio of Means | Intra-Subject CV |
|---|---|---|---|
| AUC_{τ} | 81.17% to 102.92% | 91.40% | 24.31% |
| Cₘₐₓ | 87.28% to 113.68% | 99.61% | 27.15% |
| Cₘᵢₙ | 79.73% to 109.37% | 93.38% | 32.73% |

Pharmacokinetic Parameters for Hydroxybupropion

| | Geometric Mean (% CV) | | | | |
|---|---|---|---|---|---|
| Pharmacokinetic | Arithmetic Mean ± SD | | | | |
| Parameters | Treatment A (n = 23) | | Treatment B (n = 23) | | p-Value |
| AUC_{τ} | 23059.77 | (28.43) | 24303.78 | (28.58) | 0.5209 |
| (ng · hr/mL) | 23910.83 ± 6797.16 | | 25231.86 ± 7210.51 | | |
| Cₘₐₓ (ng/mL) | 1142.95 | (28.61) | 1202.83 | (27.82) | 0.5270 |
| | 1185.68 ± 339.19 | | 1245.98 ± 346.67 | | |
| Cₘᵢₙ (ng/mL) | 849.10 | (31.17) | 890.49 | (31.92) | 0.5952 |
| | 886.37 ± 276.27 | | 932.34 ± 297.61 | | |
| C_{avg} (ng/mL) | 960.82 | (28.43) | 1012.66 | (28.58) | 0.5209 |
| | 996.28 ± 283.21 | | 1051.33 ± 300.44 | | |
| Tₘₐₓ (hr)* | 10.00 | (1.00-12.00) | 6.00 | (3.00-12.00) | 0.004 |

| | | | |
|---|---|---|---|
| Degree of | 30.54 ± 9.83 | 30.80 ± 10.22 | 0.9301 |
| Fluctuation (%) | | | |
| Degree of Swing (%) | 35.20 ± 12.91 | 35.77 ± 14.27 | 0.8892 |
| MRT (hr) | 11.80 ± 0.23 | 11.72 ± 0.23 | 0.2276 |
| M/P Ratio | 14.99 ± 6.05 | 17.08 ± 5.95 | 0.2435 |

*median (min-max)
TREATMENT A: Day 13, Wellbutrin XL ® 300 mg Tablet alone
TREATMENT B: Day 33, Wellbutrin XL ® 300 mg Tablet and Celexa ® 40 mg Tablet administered concomitantly

Relative Bioavailability Assessments for Hydroxybupropion

| Parameter | 90% C.I. | Ratio of Means | Intra-Subject CV |
|---|---|---|---|
| AUC_{τ} | 91.96% to 120.80% | 105.39% | 28.06% |
| Cₘₐₓ | 91.99% to 120.40% | 105.24% | 27.66% |
| Cₘᵢₙ | 90.32% to 121.77% | 104.87% | 30.85% |

Pharmacokinetic Parameters for Bupropion Threoamino Alcohol

| | Geometric Mean (% CV) | | | | |
|---|---|---|---|---|---|
| Pharmacokinetic | Arithmetic Mean ± SD | | | | |
| Parameters | Treatment B (n = 23) | | Treatment B (n = 23) | | p-Value |
| AUC_{τ} | 10586.56 | (38.17) | 10277.50 | (41.17) | 0.7954 |
| (ng · hr/mL) | 11321.06 ± 4320.85 | | 11083.99 ± 4563.35 | | |
| Cₘₐₓ (ng/mL) | 540.16 | (34.92) | 529.62 | (35.49) | 0.8468 |
| | 571.88 ± 199.67 | | 560.62 ± 198.96 | | |
| Cₘᵢₙ (ng/mL) | 371.01 | (42.80) | 364.30 | (45.59) | 0.8843 |
| | 403.25 ± 172.60 | | 398.40 ± 181.63 | | |
| C_{avg} (ng/mL) | 441.11 | (38.17) | 428.23 | (41.17) | 0.7954 |
| | 471.71 ± 180.04 | | 461.83 ± 190.14 | | |
| Tₘₐₓ (hr)* | 8.00 | (5.00-16.00) | 6.00 | (5.00-12.00) | 0.048 |

| | | | |
|---|---|---|---|
| Degree of | 38.33 ± 11.96 | 38.78 ± 13.37 | 0.9050 |
| Fluctuation (%) | | | |
| Degree of Swing (%) | 46.55 ± 17.32 | 46.45 ± 18.39 | 0.9844 |
| MRT (hr) | 11.83 ± 0.29 | 11.74 ± 0.25 | 0.2713 |
| M/P Ratio | 7.14 ± 2.36 | 7.56 ± 2.54 | 0.5617 |

*median (min-max)
TREATMENT A: Day 13, Wellbutrin XL ® 300 mg Tablet alone
TREATMENT B: Day 33, Wellbutrin XL ® 300 mg Tablet and Celexa ® 40 mg Tablet administered concomitantly

Relative Bioavailability Assessments
for Bupropion Threoamino Alcohol

| Parameter | 90% C.I. | Ratio of Means | Intra-Subject CV |
|---|---|---|---|
| AUC_{τ} | 80.22% to 117.49% | 97.08% | 39.99% |
| Cₘₐₓ | 82.69% to 116.26% | 98.05% | 35.43% |
| Cₘᵢₙ | 79.64% to 121.06% | 98.19% | 44.21% |

Pharmacokinetic Parameters for Bupropion Erythoamino Alcohol

| | Geometric Mean (% CV) | | | | |
|---|---|---|---|---|---|
| Pharmacokinetic | Arithmetic Mean ± SD | | | | |
| Parameters | Treatment B (n = 23) | | Treatment B (n = 23) | | p-Value |
| AUC_{τ} | 2175.39 | (30.56) | 2194.09 | (32.58) | 0.9264 |
| (ng · hr/mL) | 2271.60 ± 694.27 | | 2306.73 ± 751.53 | | |
| Cₘₐₓ (ng/mL) | 104.88 | (30.07) | 107.19 | (30.59) | 0.8056 |
| | 109.43 ± 32.91 | | 111.94 ± 34.24 | | |
| Cₘᵢₙ (ng/mL) | 83.23 | (30.69) | 84.25 | (36.01) | 0.9017 |
| | 87.03 ± 26.71 | | 89.42 ± 32.20 | | |
| C_{avg} (ng/mL) | 90.64 | (30.56) | 91.42 | (32.58) | 0.9264 |
| | 94.65 ± 28.93 | | 96.11 ± 31.31 | | |
| Tₘₐₓ (hr)* | 10.00 | (2.00-16.00) | 6.00 | (5.00-12.00) | 0.112 |

| | | | |
|---|---|---|---|
| Degree of | 23.80 ± 8.64 | 25.05 ± 10.61 | 0.6633 |
| Fluctuation (%) | | | |
| Degree of Swing (%) | 26.45 ± 10.81 | 27.88 ± 13.19 | 0.6898 |
| MRT (hr) | 11.91 ± 0.23 | 11.84 ± 0.23 | 0.2821 |
| M/P Ratio | 1.46 ± 0.44 | 1.60 ± 0.44 | 0.2923 |

*median (min-max)
TREATMENT A: Day 13, Wellbutrin XL ® 300 mg Tablet alone
TREATMENT B: Day 33, Wellbutrin XL ® 300 mg Tablet and Celexa ® 40 mg Tablet administered concomitantly

Relative Bioavailability Assessments
for Bupropion Erythroamino Alcohol

| Parameter | 90% C.I. | Ratio of Means | Intra-Subject CV |
|---|---|---|---|
| AUC_{τ} | 86.38% to 117.76% | 100.86% | 32.05% |
| Cₘₐₓ | 88.14% to 118.52% | 102.21% | 30.56% |
| Cₘᵢₙ | 85.93% to 119.23% | 101.22% | 33.98% |

Pharmacokinetic Parameters for PAWC Data

| | Geometric Mean (% CV) | | | | |
|---|---|---|---|---|---|
| Pharmacokinetic | Arithmetic Mean ± SD | | | | |
| Parameters | Treatment B (n = 23) | | Treatment B (n = 23) | | p-Value |
| AUC_{τ} | 72.19 | (22.98) | 74.24 | (24.46) | 0.6877 |
| uM · hr | 73.98 ± 17.00 | | 76.37 ± 18.68 | | |
| Cₘₐₓ (uM) | 3.76 | (22.20) | 3.94 | (21.94) | 0.4614 |
| | 3.85 ± 0.85 | | 4.03 ± 0.89 | | |
| Cₘᵢₙ (uM) | 2.51 | (27.27) | 2.59 | (28.65) | 0.6868 |
| | 2.60 ± 0.71 | | 2.70 ± 0.77 | | |
| C_{avg} (uM) | 3.01 | (22.98) | 3.09 | (24.46) | 0.6877 |
| | 3.08 ± 0.71 | | 3.18 ± 0.78 | | |
| Tₘₐₓ (hr)* | 8.00 | (3.00-10.00) | 5.00 | (3.00-8.00) | 0.003 |

| | | | |
|---|---|---|---|
| Degree of | 41.68 ± 11.69 | 43.75 ± 12.47 | 0.5646 |
| Fluctuation (%) | | | |
| Degree of Swing (%) | 50.80 ± 16.63 | 53.05 ± 18.48 | 0.6657 |
| MRT (hr) | 11.64 ± 0.25 | 11.56 ± 0.21 | 0.2816 |

*median (min-max)
TREATMENT A: Day 13, Wellbutrin XL ® 300 mg Tablet alone
TREATMENT B: Day 33, Wellbutrin XL ® 300 mg Tablet and Celexa ® 40 mg Tablet administered concomitantly

Relative Bioavailability Assessments for PAWC Data

| Parameter | 90% C.I. | Ratio of Means | Intra-Subject CV |
|---|---|---|---|
| AUC_{τ} | 91.56% to 115.50% | 102.84% | 23.76% |
| Cₘₐₓ | 94.23% to 116.62% | 104.83% | 21.77% |
| Cₘᵢₙ | 90.15% to 118.51% | 103.36% | 28.14% |

### Example 22

### Affect of Bupropion HCl on Steady-State Pharmacokinetics of Citalopram HBr in Healthy Non-Smoking Volunteers

The intent of this steady-state drug interaction study was to determine if a 300 mg dose of bupropion (Wellbutrin XL® 300 mg Tablets, GlaxoSmithKline, USA) would affect the pharmacokinetics of a 20 mg dose of Citalopram (Celexa® 20 mg Tables, Forrest Laboratories, Inc. USA). This study followed a single period, 2 treatments with up- and down-titration phases, open-label, multiple-dose design pharmacokinetic design study in which 28 subjects were scheduled to receive multiple dose administrations of Bupropion HCl (Wellbutrin XL®) and Citalopram HBr (Celexa®).

Healthy adult male or female volunteers, 18-55 years of age, BMI greater than or equal to 18.5 kg/m² and less than or equal to 29.9 kg/m² were included in this study.

Duration, dose, and mode of administration was as follows: Daily oral dose of one Celexa® 20 mg Tablet (Lot: M0512) on Days 1-14; Daily oral dose of one Celexa® 20 mg tablet and one Wellbutrin XL® 150 mg tablet (Lot: 06E065P) on days 15-17; Daily oral dose of one Celexa® 20 mg tablet and one Wellbutrin XL® 300 mg tablet (Lot: 06E002P) on Days 18-27; and Daily oral dose of Celexa® 10 mg tablet (Lot: M0517 J) and Wellbutrin XL® 150 mg tablet on Days 28-30.

Data from the 26 subjects who completed the study were included in the pharmacokinetic and statistical analyses. The concentration-time data were transferred from Watson directly to WinNonlin Enterprise Edition (Version 4.0, Pharsight Corporation) using the Custom Query Builder option for analysis. Data were analyzed by noncompartmental methods in WinNonlin. In the pharmacokinetic analysis, BLQ concentrations were treated as zero from time-zero up to the time at which the first quantifiable concentration was observed; embedded and/or terminal BLQ concentrations were treated as “missing.” Full precision concentration data (not rounded to three significant figures) and actual sample times were used for all pharmacokinetic and statistical analyses. Bupropion, hydroxybupropion, erythro-hydrobupropion, threo-hydrobupropion, citalopram, desmethylcitalopram (demethycitalopram), and didesmethylcitalopram (didemethylcitaopram) were included in the analysis. PAWC (potency corrected molar concentration summed for bupropion and its metabolites) at each time-point was calculated by multiplying the molar concentrations of bupropion, hydroxybupropion, threo-hydrobupropion, and erythro-hydrobupropion by relative potency (1.0, 0.6, 0.2, and 0.2, respectively) and adding all four concentrations. PAWC data were also included in the pharmacokinetic analysis.

The following pharmacokinetic parameters for bupropion and its metabolites hydroxybupropion, bupropion erythroamino alcohol, and bupropion threoamino alcohol, including pharmacologic activity-weighted composite (PAWC), and citalopram and its metabolites, demethylcitalopram and didemethylcitalopram were calculated by standard non-compartmental methods: AUC₀₋ₜ, AUC_{0-inf}, Cₘₐₓ, Tₘₐₓ, Kₑₗ, t_{1/2}, MRT, and M/P ratio.

The following M/P ratios were considered: Bupropion Erythroamino Alcohol/Bupropion, Bupropion Threoamino Alcohol/Bupropion, Hydroxybupropion/Bupropion, Demethylcitalopram/Citalopram, Didemethylcitalopram/Citalopram.

To test for a potential drug interaction between Celexa® and Wellbutrin XL®, pharmacokinetic parameters of citalopram, desmethylcitalopram, and didemethylcitalopram were compared. Analysis of variance (ANOVA) and the Schuirmann's two one-sided t-test procedures at the 5% significance level were applied to the natural log-transformed pharmacokinetic exposure parameters Cₘₐₓ, Cₘᵢₙ, C_{avg}, and AUC_{inf} and on untransformed values of % fluctuation, % swing, MRT, and M/P ratio. Pharmacokinetic parameters were analyzed for differences between treatments using an ANOVA model with factors for sequence, subject within sequence, period, and treatment. The 90% confidence interval for the ratio of the geometric means (Celexa®+Wellbutrin XL®/Celexa® alone) was calculated, using Celexa® alone as a reference. A lack of significant drug interaction was demonstrated if the lower and upper confidence intervals of the log-transformed parameters were within 80% to 125%. Tₘₐₓ values of citalopram, demethylcitalopram, and didemethylcitalopram were compared (Celexa®+Wellbutrin XL® vs. Celexa® alone) using nonparametric methods; a significant difference was defined a priori as p<0.05.

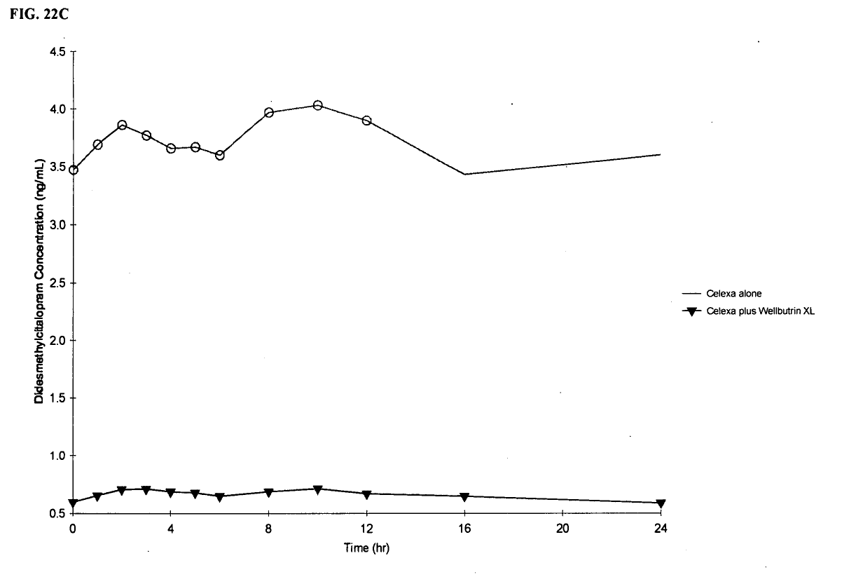

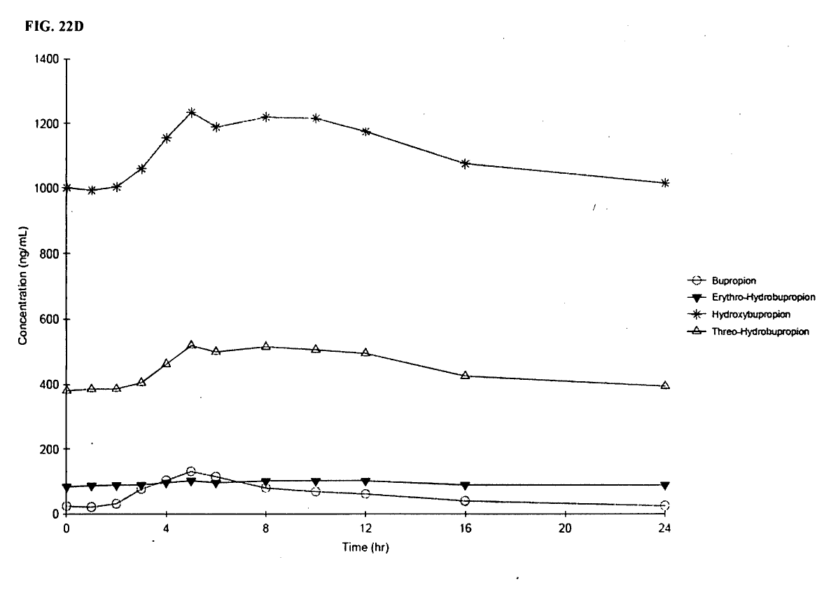

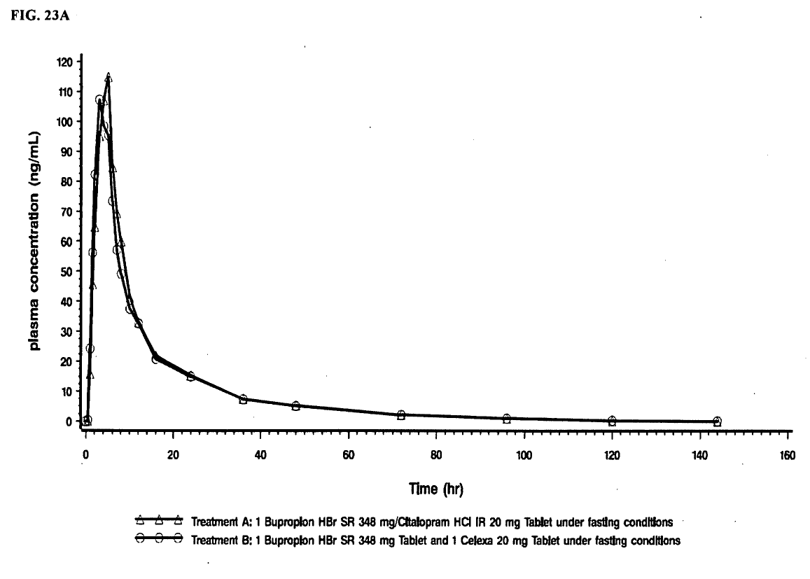

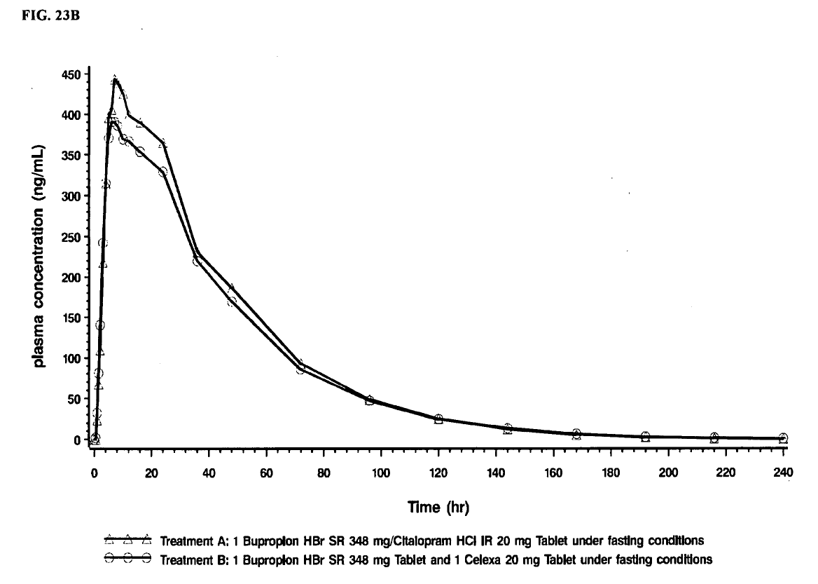

Data for the pharmacokinetic parameters for bupropion and citalopram and its metabolites is presented in the tables below and in FIGS. 22A-D.

Pharmacokinetic Parameters of Citalopram During Steady-State
Dosing of Celexa ® 20 mg Alone

| Parameter | n | Mean | SD | CV % |
|---|---|---|---|---|
| AUC_{0-τ} (hr*ng/mL) | 26 | 1219 | 321.6 | 26.38 |
| Cₘₐₓ (ng/mL) | 26 | 67.9 | 17.0 | 25.04 |
| Tₘₐₓ (hr) | 26 | 3.96 | 2.03 | 51.23 |
| Cₘᵢₙ (ng/mL) | 26 | 39.5 | 11.5 | 29.13 |
| C_{avg} (ng/mL) | 26 | 50.8 | 13.4 | 26.38 |
| Fluctuation (%) | 26 | 57.22 | 13.31 | 23.27 |
| Swing (%) | 26 | 75.55 | 23.10 | 30.58 |
| CL/F (L/hr) | 26 | 17.66 | 5.214 | 29.52 |
| Vss/F (L) | 26 | 749.6 | 172.0 | 22.95 |
| MRT (hr) | 26 | 48.62 | 13.96 | 28.71 |
| M/P Ratio | 26 | 1.000 | 0.000 | 0.00 |

Pharmacokinetic Parameters of Desmethylcitalopram During
Steady-State Dosing of Celexa ® 20 mg Alone

| Parameter | n | Mean | SD | CV % |
|---|---|---|---|---|
| AUC_{0-τ} (hr*ng/mL) | 26 | 445.9 | 112.9 | 25.31 |
| Cₘₐₓ (ng/mL) | 26 | 21.7 | 5.97 | 27.45 |
| Tₘₐₓ (hr) | 26 | 5.46 | 3.69 | 67.58 |
| Cₘᵢₙ (ng/mL) | 26 | 16.3 | 4.25 | 26.03 |
| C_{avg} (ng/mL) | 26 | 18.6 | 4.70 | 25.31 |
| Fluctuation (%) | 26 | 28.93 | 6.18 | 21.38 |
| Swing (%) | 26 | 33.09 | 7.53 | 22.75 |
| CL/F (L/hr) | — | — | — | — |
| Vss/F (L) | — | — | — | — |
| MRT (hr) | 26 | 98.54 | 41.21 | 41.82 |
| M/P Ratio | 26 | 0.4062 | 0.1330 | 32.75 |

Pharmacokinetic Parameters of Didesmethylcitalopram During
Steady-State Dosing of Celexa ® 20 mg Alone

| Parameter | n | Mean | SD | CV % |
|---|---|---|---|---|
| AUC_{0-τ} (hr*ng/mL) | 25 | 92.04 | 50.09 | 54.42 |
| Cₘₐₓ (ng/mL) | 25 | 4.40 | 2.44 | 55.35 |
| Tₘₐₓ (hr) | 25 | 6.41 | 4.30 | 67.17 |
| Cₘᵢₙ (ng/mL) | 25 | 3.42 | 1.91 | 55.73 |
| C_{avg} (ng/mL) | 25 | 3.84 | 2.09 | 54.42 |
| Fluctuation (%) | 25 | 26.43 | 14.71 | 55.64 |
| Swing (%) | 25 | 29.97 | 17.95 | 59.90 |
| CL/F (L/hr) | — | — | — | — |
| Vss/F (L) | — | — | — | — |
| MRT (hr) | 25 | 258.96 | 260.91 | 100.75 |
| M/P Ratio | 25 | 0.09587 | 0.07202 | 75.12 |

Pharmacokinetic Parameters of Citalopram ® During Steady-State
Dosing of Celexa 20 mg Plus Wellbutrin XL ® 300 mg

| Parameter | n | Mean | SD | CV % |
|---|---|---|---|---|
| AUC_{0-τ}(hr*ng/mL) | 26 | 1877 | 369.3 | 19.68 |
| Cₘₐₓ (ng/mL) | 26 | 99.4 | 20.1 | 20.23 |
| Tₘₐₓ (hr) | 26 | 3.77 | 1.37 | 36.23 |
| Cₘᵢₙ (ng/mL) | 26 | 61.6 | 12.5 | 20.32 |
| C_{avg} (ng/mL) | 26 | 78.2 | 15.4 | 19.68 |
| Fluctuation (%) | 26 | 48.46 | 9.08 | 18.73 |
| Swing (%) | 26 | 62.15 | 14.13 | 22.74 |
| CL/F (L/hr) | 26 | 11.04 | 2.097 | 18.98 |
| Vss/F (L) | 26 | 604.3 | 277.5 | 45.92 |
| MRT (hr) | 26 | 57.21 | 23.18 | 40.51 |
| M/P Ratio | 26 | 1.000 | 0.000 | 0.00 |

Pharmacokinetic Parameters of Desmethylcitalopram
During Steady-State Dosing of Celexa ® 20
mg Plus Wellbutrin XL ® 300 mg

| Parameter | n | Mean | SD | CV % |
|---|---|---|---|---|
| AUC_{0-τ} (hr*ng/mL) | 26 | 485.2 | 113.6 | 23.41 |
| Cₘₐₓ (ng/mL) | 26 | 23.6 | 6.69 | 28.39 |
| Tₘₐₓ (hr) | 26 | 7.12 | 3.47 | 48.70 |
| Cₘᵢₙ (ng/mL) | 26 | 17.3 | 4.15 | 24.06 |
| C_{avg} (ng/mL) | 26 | 20.2 | 4.73 | 23.41 |
| Fluctuation (%) | 26 | 30.39 | 8.73 | 28.72 |
| Swing (%) | 26 | 35.98 | 11.68 | 32.48 |
| CL/F (L/hr) | — | — | — | — |
| Vss/F (L) | — | — | — | — |
| MRT (hr) | 26 | 85.78 | 35.06 | 40.87 |
| M/P Ratio | 26 | 0.2814 | 0.09099 | 32.33 |

Pharmacokinetic Parameters of Didesmethylcitalopram
During Steady-State Dosing of Celexa ® 20
mg Plus Wellbutrin XL ® 300 mg

| Parameter | n | Mean | SD | CV % |
|---|---|---|---|---|
| AUC_{0-τ} (hr*ng/mL) | 25 | 16.39 | 8.055 | 49.15 |
| Cₘₐₓ (ng/mL) | 25 | 0.781 | 0.395 | 50.62 |
| Tₘₐₓ (hr) | 25 | 5.72 | 3.92 | 68.52 |
| Cₘᵢₙ (ng/mL) | 25 | 0.591 | 0.271 | 45.78 |
| C_{avg} (ng/mL) | 25 | 0.683 | 0.336 | 49.15 |
| Fluctuation (%) | 25 | 26.66 | 6.91 | 25.91 |
| Swing (%) | 25 | 30.90 | 9.25 | 29.94 |
| CL/F (L/hr) | — | — | — | — |
| Vss/F (L) | — | — | — | — |
| MRT (hr) | 24 | 110.87 | 72.21 | 65.13 |
| M/P Ratio | 25 | 0.01029 | 0.006047 | 58.77 |

Pharmacokinetic Parameters of Bupropion During Steady-State Dosing
of Celexa ® 20 mg Plus Wellbutrin XL ® 300 mg

| Parameter | n | Mean | SD | CV % |
|---|---|---|---|---|
| AUC_{0-τ} (hr*ng/mL) | 26 | 1356 | 311.1 | 22.93 |
| Cₘₐₓ (ng/mL) | 26 | 142 | 38.5 | 27.11 |
| Tₘₐₓ (hr) | 26 | 4.81 | 0.85 | 17.67 |
| Cₘᵢₙ (ng/mL) | 26 | 19.7 | 8.48 | 43.11 |
| C_{avg} (ng/mL) | 26 | 56.5 | 13.0 | 22.93 |
| Fluctuation (%) | 26 | 218.86 | 41.63 | 19.02 |
| Swing (%) | 26 | 795.19 | 622.18 | 78.24 |
| CL/F (L/hr) | 26 | 232.9 | 54.31 | 23.32 |
| Vss/F (L) | 26 | 3041 | 1065 | 35.03 |
| MRT (hr) | 26 | 15.83 | 3.04 | 19.19 |
| M/P Ratio | 26 | 1.000 | 0.000 | 0.00 |

Pharmacokinetic Parameters of Erythro-Hydrobupropion During
Steady-State Dosing of Celexa ® 20 mg Plus Wellbutrin XL ® 300 mg

| Parameter | n | Mean | SD | CV % |
|---|---|---|---|---|
| AUC_{0-τ} (hr*ng/mL) | 26 | 2239 | 889.3 | 39.73 |
| Cₘₐₓ (ng/mL) | 26 | 110 | 43.1 | 39.18 |
| Tₘₐₓ (hr) | 26 | 8.89 | 3.26 | 36.64 |
| Cₘᵢₙ (ng/mL) | 26 | 77.3 | 34.9 | 45.17 |
| C_{avg} (ng/mL) | 26 | 93.3 | 37.1 | 39.73 |
| Fluctuation (%) | 26 | 38.06 | 17.80 | 46.76 |
| Swing (%) | 26 | 51.68 | 42.25 | 81.75 |
| CL/F (L/hr) | — | — | — | — |
| Vss/F (L) | — | — | — | — |
| MRT (hr) | 25 | 97.66 | 70.54 | 72.22 |
| M/P Ratio | 26 | 1.622 | 0.4313 | 26.59 |

Pharmacokinetic Parameters of Hydroxybupropion
During Steady-State Dosing of Celexa ® 20
mg Plus Wellbutrin XL ® 300 mg

| Parameter | n | Mean | SD | CV % |
|---|---|---|---|---|
| AUC_{0-τ} (hr*ng/mL) | 26 | 26620 | 8713 | 32.74 |
| Cₘₐₓ (ng/mL) | 26 | 1320 | 448 | 33.99 |
| Tₘₐₓ (hr) | 26 | 7.42 | 2.83 | 38.14 |
| Cₘᵢₙ (ng/mL) | 26 | 918 | 357 | 38.85 |
| C_{avg} (ng/mL) | 26 | 1110 | 363 | 32.74 |
| Fluctuation (%) | 26 | 37.32 | 17.83 | 47.76 |
| Swing (%) | 26 | 50.52 | 42.97 | 85.05 |
| CL/F (L/hr) | — | — | — | — |
| Vss/F (L) | — | — | — | — |
| MRT (hr) | 26 | 89.99 | 48.99 | 54.44 |
| M/P Ratio | 26 | 19.06 | 6.515 | 34.18 |

Pharmacokinetic Parameters of Threo-Hydrobupropion
During Steady-State Dosing of Celexa ® 20
mg Plus Wellbutrin XL ® 300 mg

| Parameter | n | Mean | SD | CV % |
|---|---|---|---|---|
| AUC_{0-τ} (hr*ng/mL) | 26 | 10750 | 4719 | 43.90 |
| Cₘₐₓ (ng/mL) | 26 | 553 | 237 | 42.90 |
| Tₘₐₓ (hr) | 26 | 7.93 | 2.83 | 35.72 |
| Cₘᵢₙ (ng/mL) | 26 | 352 | 186 | 52.87 |
| C_{avg} (ng/mL) | 26 | 448 | 197 | 43.90 |
| Fluctuation (%) | 26 | 50.12 | 22.25 | 44.40 |
| Swing (%) | 26 | 75.49 | 68.58 | 90.86 |
| CL/F (L/hr) | — | — | — | — |
| Vss/F (L) | — | — | — | — |
| MRT (hr) | 25 | 56.01 | 21.70 | 38.74 |
| M/P Ratio | 26 | 7.856 | 2.969 | 37.79 |

Pharmacokinetic Parameters of PAWC During Steady-State Dosing of
Celexa ® 20 mg Plus Wellbutrin XL ® 300 mg

| Parameter | n | Mean | SD | CV % |
|---|---|---|---|---|
| AUC_{0-τ} (hr*nmol/L) | 26 | 78860 | 22910 | 29.05 |
| Cₘₐₓ (nmol/L) | 26 | 4130 | 1190 | 28.80 |
| Tₘₐₓ (hr) | 26 | 6.19 | 2.47 | 39.82 |
| Cₘᵢₙ (nmol/L) | 26 | 2620 | 966 | 36.82 |
| C_{avg} (nmol/L) | 26 | 3290 | 955 | 29.05 |
| Fluctuation (%) | 26 | 47.76 | 17.02 | 35.63 |
| Swing (%) | 26 | 66.14 | 45.71 | 69.11 |
| CL/F (L/hr) | — | — | — | — |
| Vss/F (L) | — | — | — | — |
| MRT (hr) | 26 | 65.23 | 28.69 | 43.98 |

Statistical Analysis of the Log-Transformed Systemic
Exposure Parameters of Citalopram

| | Geometric | | Ratio | | | |
|---|---|---|---|---|---|---|
| Dependent | Mean^{a} | | (%)^{b} | 90% CI^{c} | | |
| Variable | Test | Ref | (Test/Ref) | Lower | Upper | Power |
| ln(AUC_{0-τ}) | 1846.90 | 1175.60 | 157.10 | 140.27 | 175.95 | 0.9447 |
| ln(Cₘₐₓ) | 97.71 | 65.80 | 148.51 | 133.44 | 165.27 | 0.9626 |
| ln(Cₘᵢₙ) | 60.47 | 37.70 | 160.38 | 141.47 | 181.83 | 0.9009 |
| ln(C_{avg}) | 76.95 | 48.98 | 157.10 | 140.27 | 175.95 | 0.9447 |

^{a}Geometric Mean for Celexa ® 20 mg plus Wellbutrin XL ® 300 mg (Test) and Celexa ® 20 mg alone (Ref) based on Least Squares Mean of log-transformed parameter values
^{b}Ratio (%) = Geometric Mean (Test)/Geometric Mean (Ref)
^{c}90% Confidence Interval

Statistical Analysis of the Log-Transformed Systemic
Exposure Parameters of Desmethylcitalopram

| | Geometric | | | | | |
|---|---|---|---|---|---|---|
| Dependent | Mean^{a} | | Ratio (%)^{b} | 90% CI^{c} | | |
| Variable | Test | Ref | (Test/Ref) | Lower | Upper | Power |
| ln(AUC_{0-τ}) | 473.82 | 433.72 | 109.24 | 98.32 | 121.38 | 0.9667 |
| ln(Cₘₐₓ) | 22.83 | 21.05 | 108.50 | 96.55 | 121.93 | 0.9337 |
| ln(Cₘᵢₙ) | 16.84 | 15.84 | 106.29 | 95.36 | 118.46 | 0.9587 |
| ln(C_{avg}) | 19.74 | 18.07 | 109.24 | 98.32 | 121.38 | 0.9667 |

^{a}Geometric Mean for Celexa ® 20 mg plus Wellbutrin XL ® 300 mg (Test) and Celexa ® 20 mg alone (Ref) based on Least Squares Mean of log-transformed parameter values
^{b}Ratio (%) = Geometric Mean (Test)/Geometric Mean (Ref)
^{c}90% Confidence Interval

Statistical Analysis of the Log-Transformed Systemic
Exposure Parameters of Didesmethylcitalopram

| | Geometric | | | | | |
|---|---|---|---|---|---|---|
| Dependent | Mean^{a} | | Ratio (%)^{b} | 90% CI^{c} | | |
| Variable | Test | Ref | (Test/Ref) | Lower | Upper | Power |
| ln(AUC_{0-τ}) | 14.99 | 82.11 | 18.26 | 14.69 | 22.70 | 0.5179 |
| ln(Cₘₐₓ) | 0.71 | 3.93 | 18.08 | 14.55 | 22.46 | 0.5193 |
| ln(Cₘᵢₙ) | 0.54 | 3.05 | 17.89 | 14.42 | 22.19 | 0.5244 |
| ln(C_{avg}) | 0.62 | 3.42 | 18.26 | 14.69 | 22.70 | 0.5179 |

^{a}Geometric Mean for Celexa ® 20 mg plus Wellbutrin XL ® 300 mg (Test) and Celexa ® 20 mg alone (Ref) based on Least Squares Mean of log-transformed parameter values
^{b}Ratio (%) = Geometric Mean (Test)/Geometric Mean (Ref)
^{c}90% Confidence Interval

### Example 23

### A Two-Part, Two-Way Crossover, Open-Label, Single-Dose, Fasting and Food-Effect, Pharmacokinetic Study of Bupropion HBr XL 348 mg/Citalopram HCl IR 20 mg Tablets Versus Bupropion HBr XL 348 mg Tablets Given Concomitantly with Celexa™ (Citalopram HBr) 20 Mg Tablets in Normal, Healthy, Non-Smoking Male and Female Subjects

This study was a two-part, two-way crossover, randomized, open-label, single-dose, fasting and food-effect, Phase I study. The objectives of this study were: a) to determine and compare the rate and extent of absorption of bupropion and citalopram from a test fixed dose combination tablet formulation of Bupropion HBr XL (sustained release) 348 mg/Citalopram HCl Immediate Release (IR) 20 mg versus Bupropion HBr XL 348 mg tablets given concomitantly with Celexa™ 20 mg tablets under fasting conditions, (Group 1) and, b) to determine the effect of food on the rate and extent of absorption of a fixed dose combination tablet formulation of Bupropion HBr XL 348 mg/Citalopram HCl IR 20 mg (Group 2). The bupropion HBr XL (sustained release) tablet used in this study was manufactured as described in U.S. Pat. No. 7,241,805. To obtain the fixed dose combination tablet formulation of bupropion HBr XL (sustained release) 348 mg/citalopram HCl Immediate Release (IR) 20 mg, the bupropion HBr XL tablet is over coated with an immediate release coating comprising 20 mg citalopram HCl according to methods well known in the art. Normal, healthy, non-smoking male and female subjects between the ages of 18 and 55 years were included in the study.

Following an overnight fast of at least 10 hours, and 30 minutes after the start of a high fat content meal, 1 Bupropion HBr XL 348 mg/Citalopram HCl IR 20 mg Tablet, Lot #: 0608055 [potency value=citalopram (95.2%) and bupropion (98.8%) of label claim], administered orally with 240 mL of ambient temperature water. Following an overnight fast of at least 10 hours, 1 Bupropion HBr XL 348 mg Tablet, Lot #: 06C159P (potency value=97.8% of label claim) and 1 Celexa™ 20 mg Tablet, Lot #: M0512M (potency value=97.9% of label claim) administered orally with 240 mL of ambient temperature water.

There were 13 subjects dosed in Group 1, 11 of whom completed the study. One subject was dismissed after emesis within 24.00 hours of dosing, and 1 subject was dismissed because of administrative reasons. Pharmacokinetic and statistical analyses were performed on 11 subjects who completed the study.

There were 14 subjects dosed in Group II, 10 of whom completed the study. Two subjects were dismissed after emesis within 24.00 hours of dosing, and 2 subjects withdrew for personal reasons. Pharmacokinetic and statistical analyses were performed on 10 subjects who completed the study.

During each study period, 25 blood samples (10 mL each as 1×4 mL tube and 1×6 mL tube for each time-point) were collected from each subject at the following timepoints: 0.00 (pre-dose), 0.50, 1.00, 1.50, 2.00, 3.00, 4.00, 5.00, 6.00, 7.00, 8.00, 10.00, 12.00, 16.00, 24.00, 36.00, 48.00, 72.00, 96.00, 120.00, 144.00, 168.00, 192.00, 216.00, and 240.00 hours post-dose.

Bupropion, its metabolites—hydroxybupropion, bupropion erythroamino alcohol, and bupropion threoamino alcohol, and the internal standard, 1-(3-chlorophenyl)-piperazine, were extracted by solid phase extraction into an organic media from 0.50 mL of human plasma. An aliquot of this extract was injected into a High Performance Liquid Chromatography system and detected using a tandem mass spectrometer. The analytes were separated by reverse phase chromatography. Evaluation of the assay was carried out by the construction of an eight (8) point calibration curve (excluding zero concentration) covering the range of 1.000 ng/mL to 1023.900 ng/mL for bupropion, 3.907 ng/mL to 4001.200 ng/mL for hydroxybupropion, 1.000 ng/mL to 1024.310 ng/mL for bupropion erythroamino alcohol, and 1.000 ng/mL to 1023.850 ng/mL for bupropion threoamino alcohol in human plasma. The slope and intercept of the calibration curves were determined through weighted linear regression analysis (1/area ratio.²). Two calibration curves and duplicate QC samples (at three concentration levels) were analyzed along with each batch of the study samples. Peak area ratios were used to determine the concentration of the standards, quality control samples, and the unknown study samples from the calibration curves.

Citalopram, its metabolites—demethylcitalopram and didemethylcitalopram, and the internal standards, citalopram analog, demethylcitalopram analog, and didemethylcitalopram analog, were extracted by liquid-liquid extraction into an organic media from 1.00 mL of human plasma. An aliquot of this extract was injected into a High Performance Liquid Chromatography system and detected using a tandem mass spectrometer. The analytes were separated by reverse phase chromatography. Evaluation of the assay was carried out by the construction of an eight (8) point calibration curve (excluding zero concentration) covering the range of 0.250 ng/mL to 63.944 ng/mL for Citalopram, 0.050 ng/mL to 12.812 ng/mL for demethylcitalopram, and 0.050 ng/mL to 12.796 ng/mL for didemethylcitalopram in human plasma. The slope and intercept of the calibration curves were determined through weighted linear regression analysis (1/conc.²). Two calibration curves and duplicate QC samples (at three concentration levels) were analyzed along with each batch of the study samples. Peak area ratios were used to determine the concentration of the standards, quality control samples, and the unknown study samples from the calibration curves.

The pharmacokinetic analysis was performed on 21 subjects who completed the 2 study periods (11 subjects in Group 1 and 10 subjects in Group 2). The safety assessment was performed on all subjects who received at least 1 dose during the course of the study.

The following pharmacokinetic parameters for bupropion and its metabolites hydroxybupropion, bupropion erythroamino alcohol, and bupropion threoamino alcohol, including pharmacologic activity-weighted composite (PAWC), and citalopram and its metabolites, demethylcitalopram and didemethylcitalopram were calculated by standard non-compartmental methods: AUC₀₋ₜ, AUC_{0-inf}, Cₘₐₓ, Tₘₐₓ, Kₑₗ, t_{1/2}, MRT, and M/P ratio.

Statistical analysis was carried out using General Linear Model (GLM) procedures in Statistical Analysis System (SAS), analysis of variance (ANOVA) was performed on ln-transformed AUC₀₋ₜ, AUC_{0-inf}, and Cₘₐₓ and on untransformed Kₑₗ, t_{1/2}, MRT, and M/P ratio at the significance level of 0.05. The intra-subject coefficient of variation (CV) was calculated using the Mean Square Error (MSE) from the ANOVA table. The ratio of geometric means and the 90% geometric confidence interval (90% C.I.) were calculated based on the difference in the Least Squares Means of the ln-transformed AUC₀₋ₜ, AUC_{0-inf}, and Cₘₐₓ between the test and reference formulations. Tₘₐₓ was analyzed using nonparametric methods.

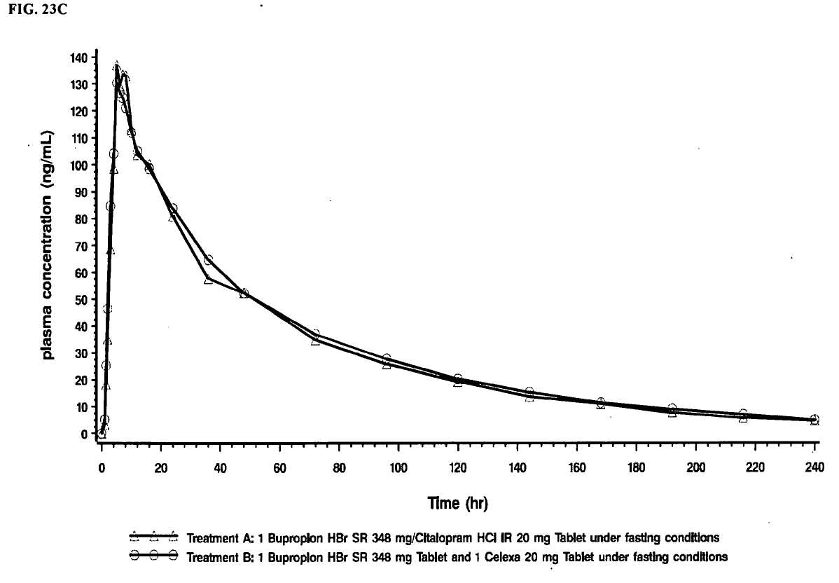

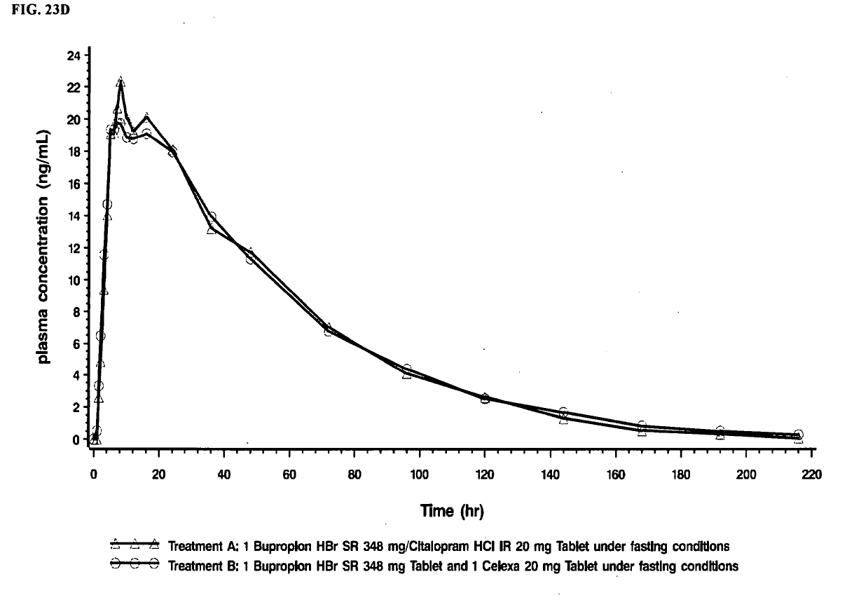

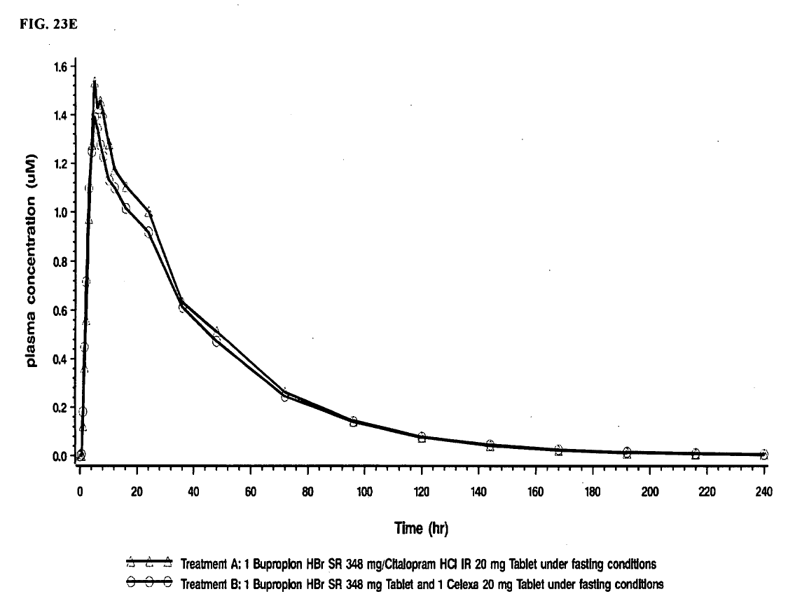

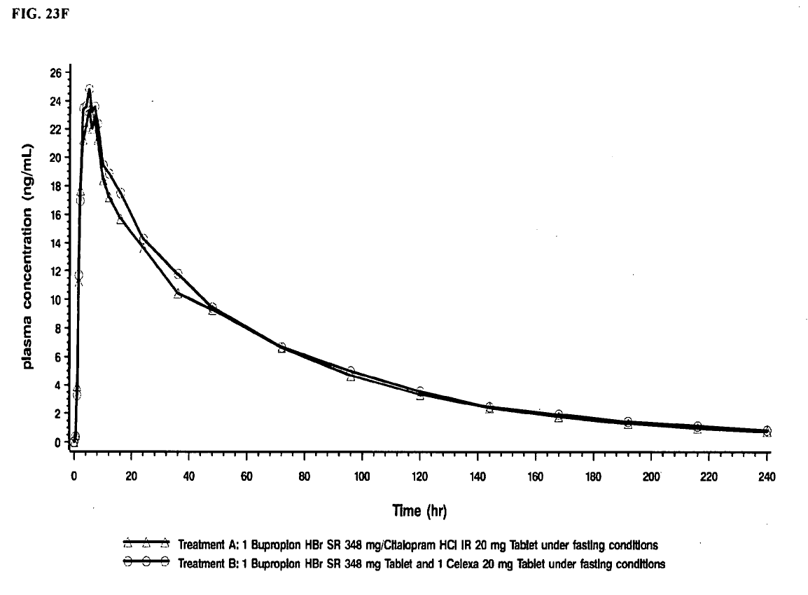

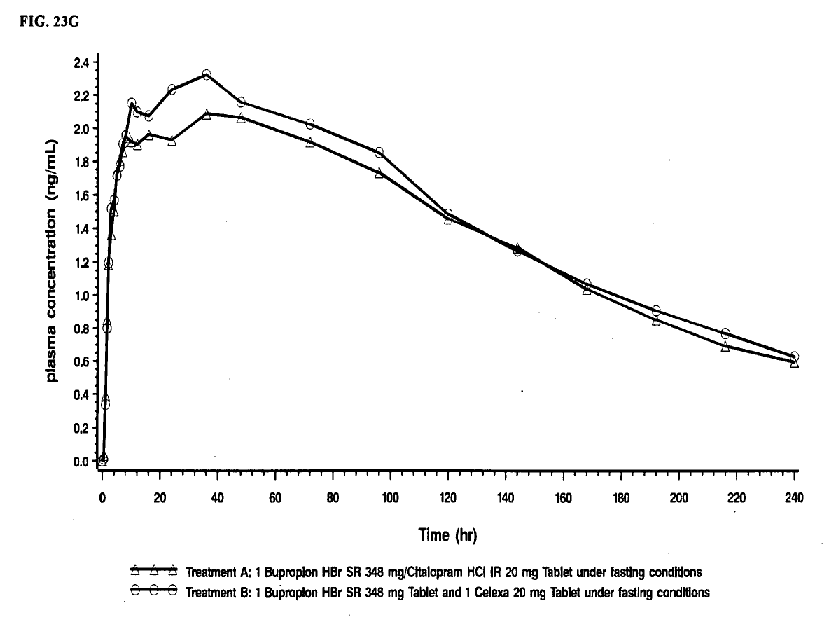

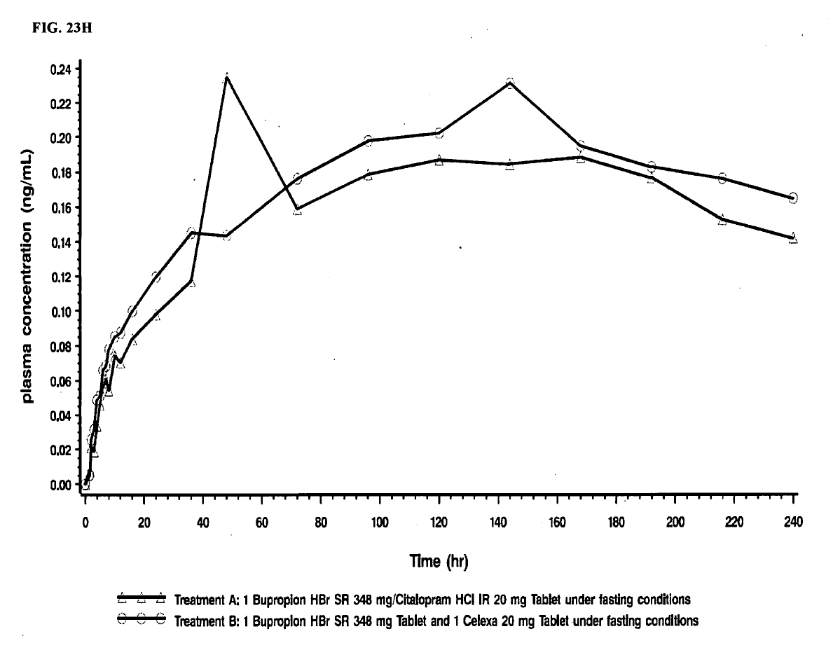

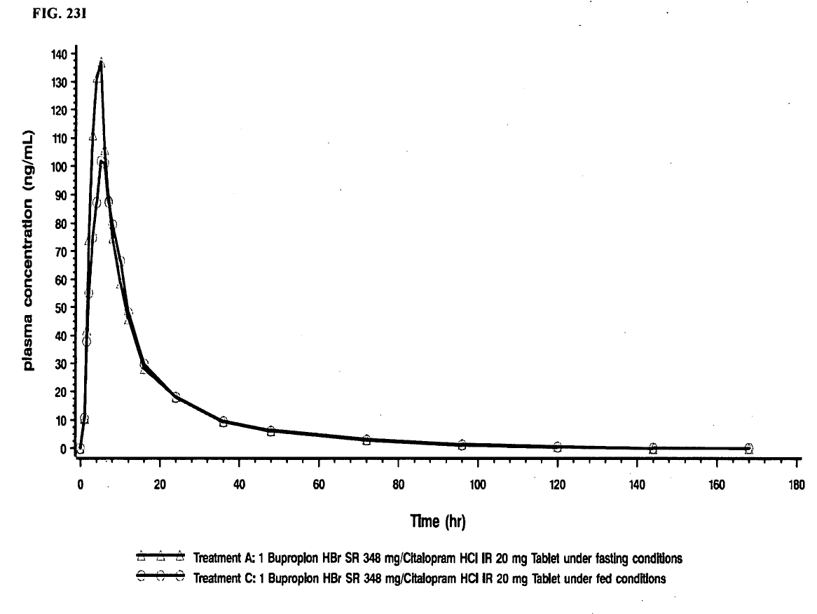

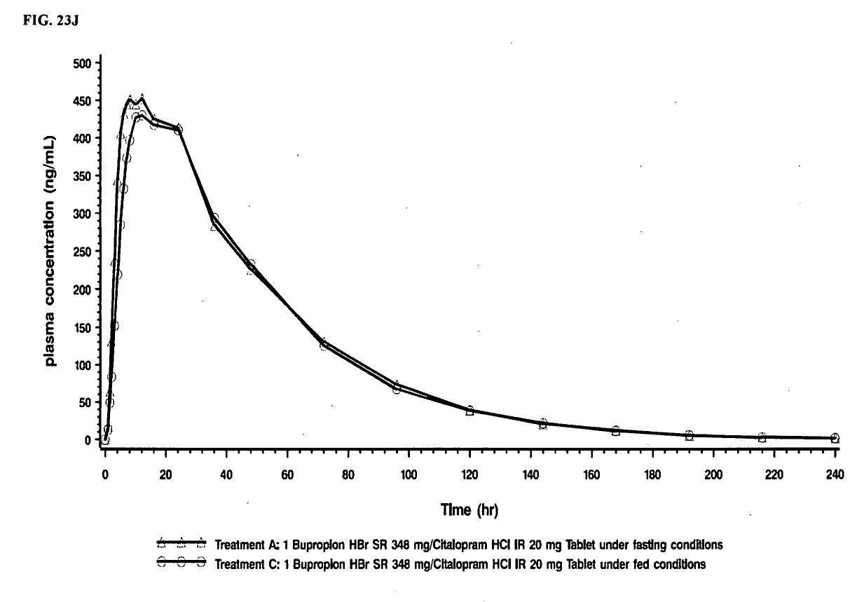

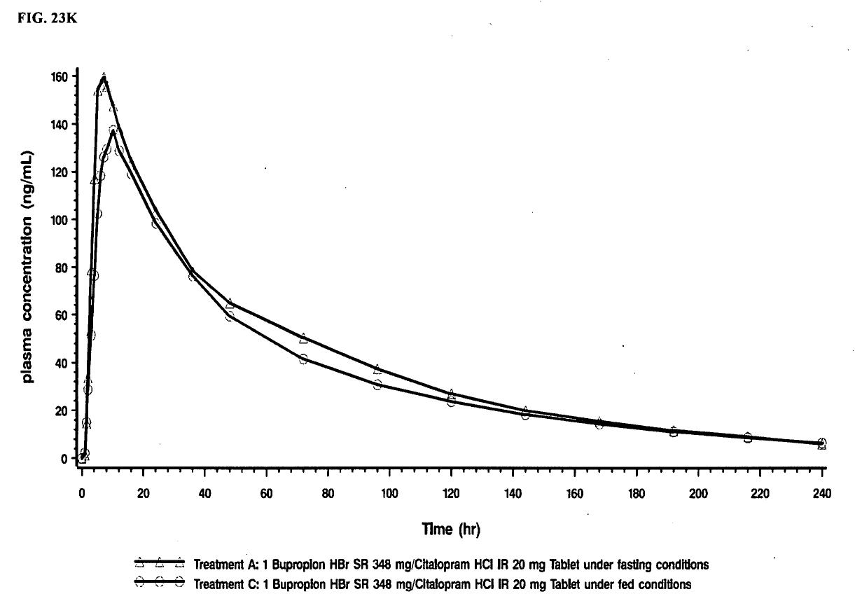

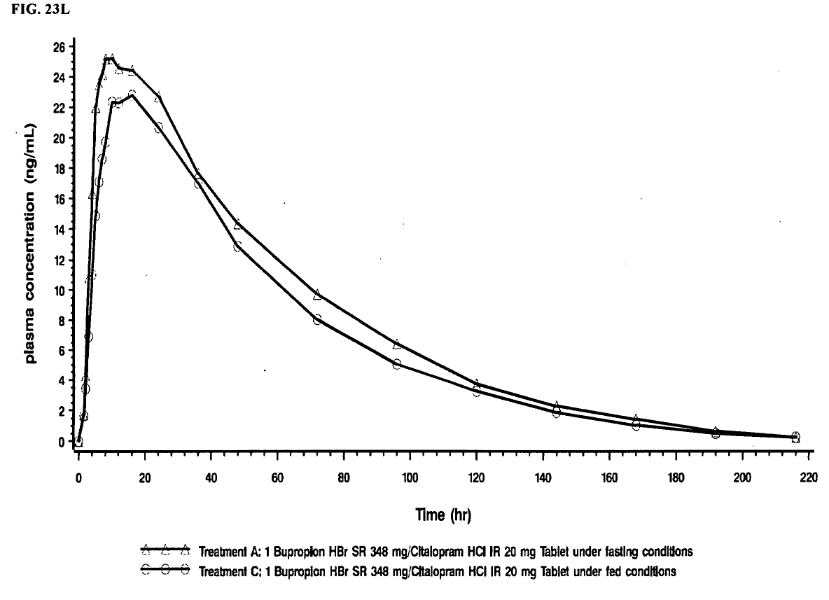

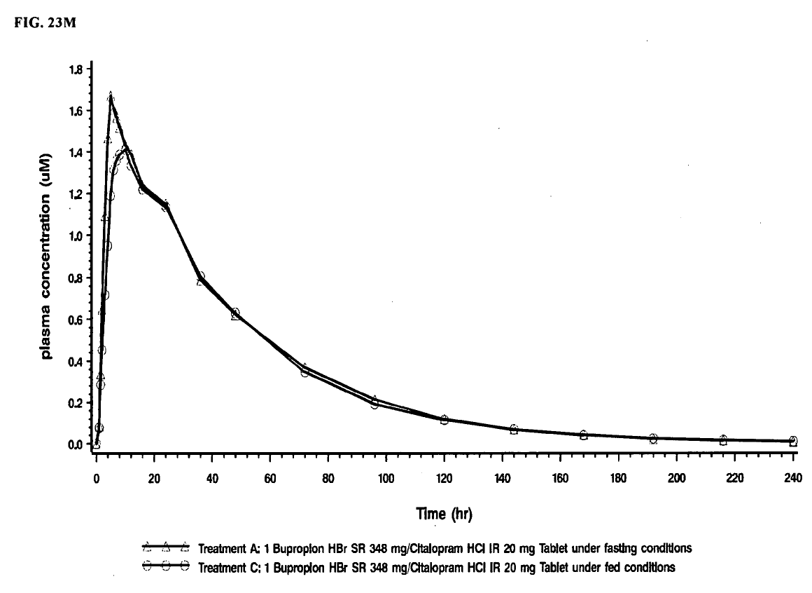

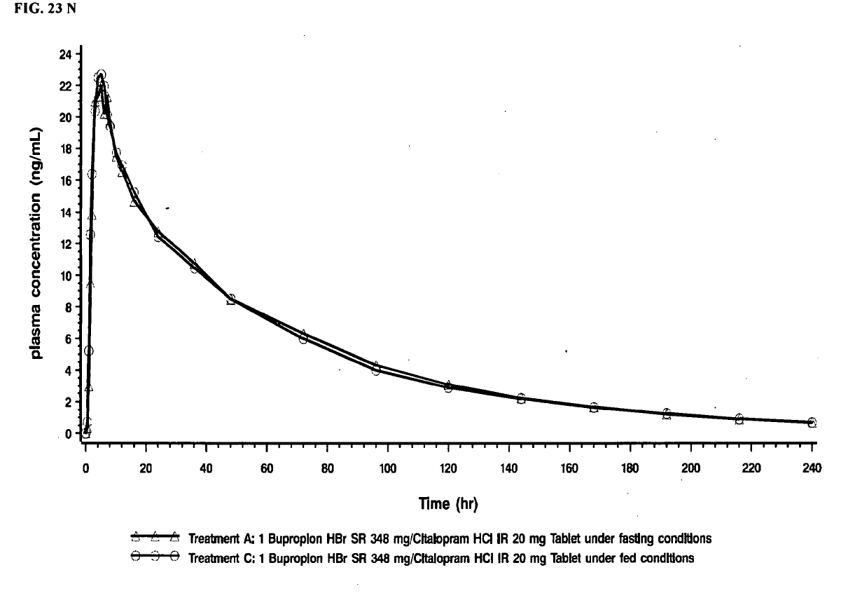

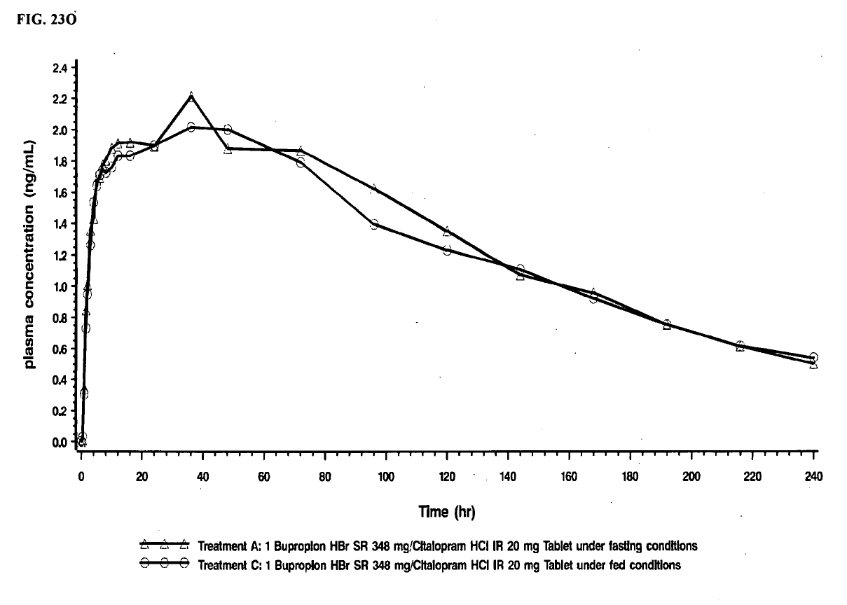

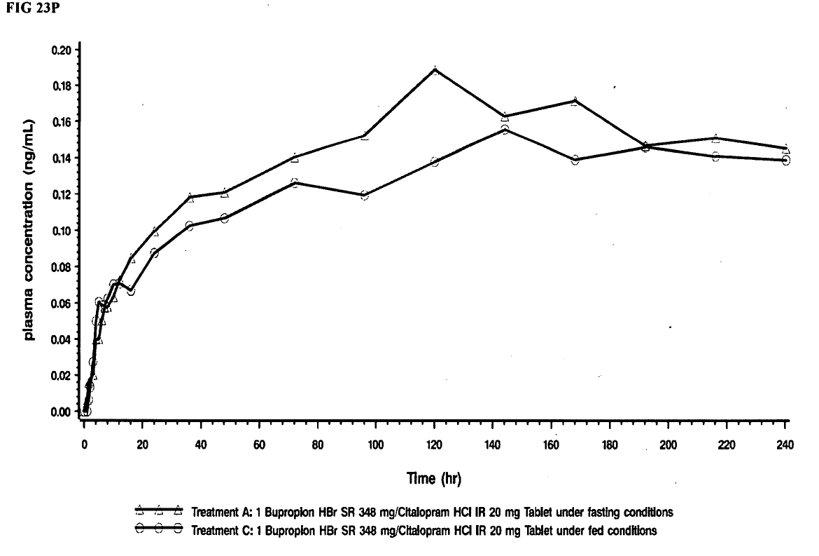

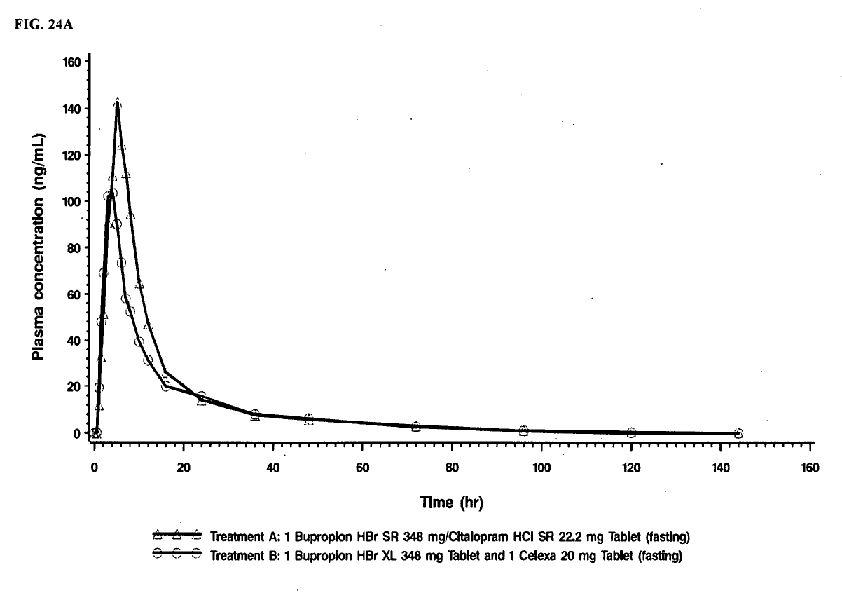

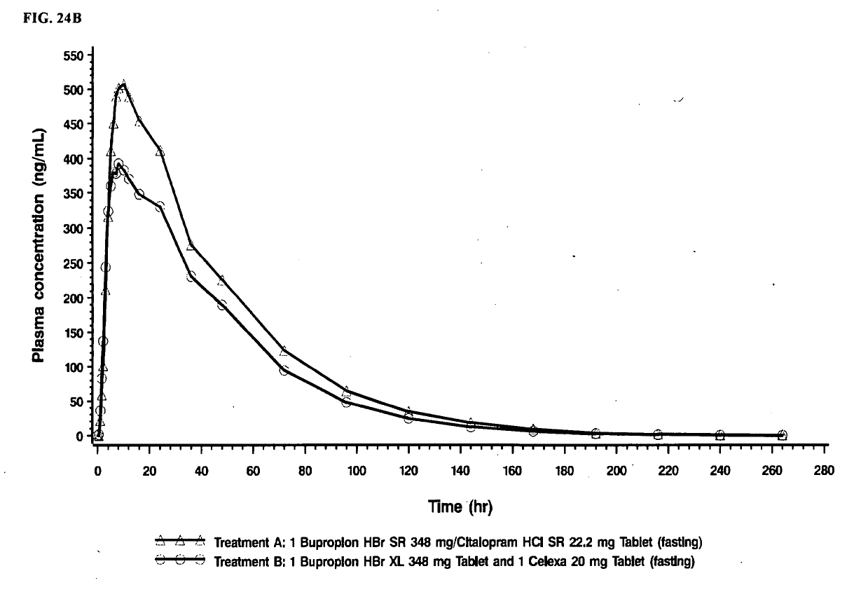

Data for the pharmacokinetic parameters for bupropion and citalopram and their metabolites is presented in the tables below and in FIGS. 23A-P.

Pharmacokinetic Parameters Bupropion (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 1306.88 | (28.24) | 1246.49 | (32.72) |
|---|---|---|---|---|
| (ng · hr/mL) | 1355.50 ± 382.79 | | 1301.77 ± 425.92 | |
| AUC_{0-inf} | 1352.18 | (27.47) | 1288.48 | (32.09) |
| (ng · hr/mL) | 1399.78 ± 384.47 | | 1343.41 ± 431.13 | |
| Cₘₐₓ (ng/mL) | 110.13 | (47.04) | 114.67 | (30.63) |
| | 124.00 ± 58.33 | | 119.58 ± 36.62 | |
| Tₘₐₓ (hr)* | 5.00 | (2.00-5.02) | 3.00 | (2.00-5.00) |

| | | |
|---|---|---|
| t_{1/2} (hr) | 21.41 ± 8.32 | 19.55 ± 7.39 |
| Kₑₗ (hr⁻¹) | 3.96E−02 ± 2.19E−02 | 4.16E−02 ± 1.90E−02 |
| MRT (hr) | 21.15 ± 6.19 | 20.36 ± 6.33 |

*median (min-max)

Relative Bioavailability Assessments for Bupropion (Group 1)

| | Potency Uncorrected Data | | |
|---|---|---|---|
| Parameter | 90% C.I. | Ratio of Means | Intra-Subject CV |
| AUC₀₋ₜ | 88.13% to 125.53% | 105.18% | 22.82% |
| AUC_{0-inf} | 88.82% to 124.82% | 105.29% | 21.93% |
| Cₘₐₓ | 70.81% to 133.09% | 97.08% | 41.88% |

Potency Corrected Data

| Parameter | 90% C.I. | Ratio of Means |
|---|---|---|
| AUC₀₋ₜ | 87.24% to 124.26% | 104.12% |
| AUC_{0-inf} | 87.92% to 123.56% | 104.23% |
| Cₘₐₓ | 70.10% to 131.74% | 96.10% |

Pharmacokinetic Parameters for Hydroxybupropion (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 19769.84 | (43.38) | 17749.81 | (52.24) |
|---|---|---|---|---|
| (ng · hr/mL) | 21212.19 ± 9202.72 | | 19742.73 ± 10313.94 | |
| AUC_{0-inf} | 19991.61 | (43.01) | 17973.86 | (51.79) |
| (ng · hr/mL) | 21424.72 ± 9214.70 | | 19951.68 ± 10332.15 | |
| Cₘₐₓ (ng/mL) | 450.03 | (27.74) | 393.57 | (33.64) |
| | 468.22 ± 129.87 | | 415.16 ± 139.64 | |
| Tₘₐₓ (hr)* | 8.00 | (7.00-24.00) | 8.00 | (5.00-24.00) |

| | | |
|---|---|---|
| t_{1/2} (hr) | 25.04 ± 6.12 | 24.56 ± 6.03 |
| Kₑₗ (hr⁻¹) | 2.91E−02 ± 6.63E−03 | 2.98E−02 ± 7.07E−03 |
| MRT (hr) | 39.38 ± 7.67 | 39.40 ± 7.84 |
| M/P Ratio | 14.82 ± 6.20 | 14.35 ± 7.97 |

*median (min-max)

Relative Bioavailability Assessments
for Hydroxybupropion (Group 1)

| Parameter | 90% C.I. | Ratio of Means | Intra-Subject CV |
|---|---|---|---|
| AUC₀₋ₜ | 97.13% to 130.18% | 112.45% | 18.82% |
| AUC_{0-inf} | 97.21% to 129.69% | 112.28% | 18.52% |
| Cₘₐₓ | 94.88% to 139.31% | 114.97% | 24.84% |

Pharmacokinetic Parameters for Bupropion Threoamino Alcohol (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 6781.91 | (39.37) | 6721.27 | (54.76) |
|---|---|---|---|---|
| (ng · hr/mL) | 7277.11 ± 2865.28 | | 7614.66 ± 4169.98 | |
| AUC_{0-inf} | 7379.72 | (40.50) | 7142.17 | (54.16) |
| (ng · hr/mL) | 8015.86 ± 3246.55^{†} | | 8106.20 ± 4390.12 | |
| Cₘₐₓ (ng/mL) | 138.27 | (39.58) | 128.32 | (40.64) |
| | 149.64 ± 59.23 | | 139.06 ± 56.51 | |
| Tₘₐₓ (hr)* | 7.00 | (5.00-24.00) | 6.00 | (3.00-12.00) |

| | | |
|---|---|---|
| t_{1/2} (hr) | 68.95 ± 50.06^{†} | 61.63 ± 12.49 |
| Kₑₗ (hr⁻¹) | 1.33E−02 ± 5.42E−03^{†} | 1.17E−02 ± 2.30E−03 |
| MRT (hr) | 79.83 ± 32.07^{†} | 78.61 ± 17.93 |
| M/P Ratio | 5.85 ± 2.53^{†} | 5.83 ± 2.38 |

*median (min-max)
^{†}n = 10

Relative Bioavailability Assessments for
Bupropion Threoamino Alcohol (Group 1)

| Parameter | 90% C.I. | Ratio of Means | Intra-Subject CV |
|---|---|---|---|
| AUC₀₋ₜ | 85.35% to 120.65% | 101.47% | 22.32% |
| AUC_{0-inf} | 83.93% to 124.82% | 102.36% | 24.20% |
| Cₘₐₓ | 84.68% to 142.18% | 109.73% | 33.93% |

Pharmacokinetic Parameters for Bupropion Eryhthroamino Alcohol (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 1202.67 | (32.03) | 1154.00 | (49.06) |
|---|---|---|---|---|
| (ng · hr/mL) | 1266.30 ± 405.65 | | 1276.47 ± 626.26 | |
| AUC_{0-inf} | 1269.58 | (30.77) | 1222.29 | (48.02) |
| (ng · hr/mL) | 1331.59 ± 409.70 | | 1344.73 ± 645.78 | |
| Cₘₐₓ (ng/mL) | 22.82 | (24.43) | 21.28 | (21.62) |
| | 23.44 ± 5.73 | | 21.76 ± 4.70 | |
| Tₘₐₓ (hr)* | 8.00 | (7.00-24.00) | 8.00 | (5.00-24.00) |

| | | |
|---|---|---|
| t_{1/2} (hr) | 31.24 ± 7.88 | 33.57 ± 9.70 |
| Kₑₗ (hr⁻¹) | 2.37E−02 ± 7.02E−03 | 2.24E−02 ± 6.94E−03 |
| MRT (hr) | 51.70 ± 12.47 | 53.60 ± 14.03 |
| M/P Ratio | 0.96 ± 0.25 | 0.97 ± 0.27 |

*median (min-max)

Relative Assessments for Bupropion
Erythroamino Alcohol (Group 1)

| Parameter | 90% C.I. | Ratio of Means | Intra-Subject CV |
|---|---|---|---|
| AUC₀₋ₜ | 87.85% to 124.74% | 104.68% | 22.61% |
| AUC_{0-inf} | 88.21% to 123.03% | 104.17% | 21.43% |
| Cₘₐₓ | 87.56% to 132.77% | 107.82% | 26.99% |

Pharmacokinetic Parameters for PAWC (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 59.69 | (36.13) | 54.79 | (43.74) |
|---|---|---|---|---|
| (uM · hr) | 62.72 ± 22.66 | | 59.34 ± 25.96 | |
| AUC_{0-inf} | 60.05 | (36.27) | 55.05 | (44.08) |
| (uM · hr) | 63.12 ± 22.90 | | 59.68 ± 26.30 | |
| Cₘₐₓ (uM) | 1.59 | (27.50) | 1.40 | (26.06) |
| | 1.65 ± 0.45 | | 1.45 ± 0.38 | |
| Tₘₐₓ (hr)* | 5.00 | (4.00-10.00) | 5.00 | (4.00-12.00) |

| | | |
|---|---|---|
| t_{1/2} (hr) | 43.90 ± 28.03 | 33.52 ± 5.25 |
| Kₑₗ (hr⁻¹) | 1.95E−02 ± 7.81E−03 | 2.12E−02 ± 3.49E−03 |
| MRT (hr) | 40.67 ± 7.48 | 40.58 ± 7.73 |

*median (min-max)

Phrmcokinetic Parameters for Citalopram (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 1243.68 | (29.90) | 1310.90 | (33.48) |
|---|---|---|---|---|
| (ng · hr/mL) | 1298.07 ± 388.17 | | 1376.28 ± 460.76 | |
| AUC_{0-inf} | 1308.68 | (31.80) | 1371.57 | (35.69) |
| (ng · hr/mL) | 1373.05 ± 436.59 | | 1448.18 ± 516.91 | |
| Cₘₐₓ (ng/mL) | 23.89 | (21.76) | 26.28 | (23.38) |
| | 24.47 ± 5.32 | | 26.91 ± 6.29 | |
| Tₘₐₓ (hr)* | 5.00 | (2.00-8.00) | 5.00 | (3.00-8.00) |

| | | |
|---|---|---|
| t_{1/2} (hr) | 58.25 ± 16.80 | 54.75 ± 9.44 |
| Kₑₗ (hr⁻¹) | 1.28E−02 ± 3.51E−03 | 1.30E−02 ± 2.46E−03 |
| MRT (hr) | 73.81 ± 14.08 | 71.88 ± 12.49 |

*median (min-max)

Relative Bioavailability Assessments for Citalopram (Group 1)

| | Potency Uncorrected Data | | |
|---|---|---|---|
| Parameter | 90% C.I. | Ratio of Means | Intra-Subject CV |
| AUC₀₋ₜ | 90.26% to 100.49% | 95.24% | 6.85% |
| AUC_{0-inf} | 90.42% to 101.39% | 95.75% | 7.31% |
| Cₘₐₓ | 83.61% to 99.39% | 91.16% | 11.04% |

Potency Corrected Data

| Parameter | 90% C.I. | Ratio of Means |
|---|---|---|
| AUC₀₋ₜ | 92.82% to 103.34% | 97.94% |
| AUC_{0-inf} | 92.98% to 104.27% | 98.47% |
| Cₘₐₓ | 85.98% to 102.20% | 93.74% |

Pharmacokinetic Parameters for Demetheylcitalopram (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 329.83 | (21.36) | 349.57 | (21.08) |
|---|---|---|---|---|
| (ng · hr/mL) | 337.60 ± 72.12 | | 358.21 ± 75.50 | |
| AUC_{0-inf} | 352.35 | (8.86) | 442.09 | (19.62) |
| (ng · hr/mL) | 353.53 ± 31.32^{‡} | | 448.82 ± 88.08^{‡} | |
| Cₘₐₓ (ng/mL) | 2.22 | (25.49) | 2.38 | (27.71) |
| | 2.30 ± 0.59 | | 2.49 ± 0.69 | |
| Tₘₐₓ (hr)* | 48.00 | (8.00-96.00) | 36.00 | (10.00-36.03) |

| | | |
|---|---|---|
| t_{1/2} (hr) | 59.70 ± 8.76^{‡} | 77.33 ± 15.85^{‡} |
| Kₑₗ (hr⁻¹) | 1.18E−02 ± 1.74E−03^{‡} | 9.29E−03 ± 1.89E−03^{‡} |
| MRT (hr) | 117.28 ± 18.17^{‡} | 128.01 ± 26.80^{‡} |
| M/P Ratio | 0.35 ± 0.13^{‡} | 0.39 ± 0.08^{‡} |

*median (min-max)
^{‡}n = 6

Relative Bioavailability Assessments
for Demethylcitalopram (Group 1)

| Parameter | 90% C.I. | Ratio of Means | Intra-Subject CV |
|---|---|---|---|
| AUC₀₋ₜ | 90.20% to 99.07% | 94.53% | 5.98% |
| AUC_{0-inf} | 55.06% to 152.04% | 91.50% | 9.31% |
| Cₘₐₓ | 87.90% to 97.97% | 92.80% | 6.92% |

Pharmacokinetic Parameters for Didemethylcitalopram (Group 1)

| | Geometric Mean (% CV) | | |
|---|---|---|---|
| | Arithmetic Mean ± SD | | |
| | | Bupropion HBr XL 348 | Bupropion HBr XL |
| | | mg/Citalopram HCl IR | 348 mg Tablets and |
| | | 20 mg Tablets (A) | Celexa ™ 20 mg Tablets (B) |
| Pharmacokinetic | | (fasting conditions) | (fasting conditions) |
| Parameters | | (n = 11) | (n = 11) |

| AUC₀₋ₜ | 27.72 | (84.96) | 31.08 | (75.70) |
|---|---|---|---|---|
| (ng · hr/mL) | 38.58 ± 32.78 | | 41.36 ± 31.31 | |
| AUC_{0-inf} | NC | | NC | |
| (ng · hr/mL) | | | | |
| Cₘₐₓ (ng/mL) | 0.25 | (128.93) | 0.24 | (62.84) |
| | 0.31 ± 0.40 | | 0.26 ± 0.16 | |
| Tₘₐₓ (hr)* | 144.00 | (48.00-216.00)^{§} | 132.10 | (96.00-216.00)^{†} |

| | | |
|---|---|---|
| t_{1/2} (hr) | NC | NC |
| Kₑₗ (hr⁻¹) | NC | NC |
| MRT (hr) | NC | NC |
| M/P Ratio | NC | NC |

*median (min-max)
^{§}n = 9
^{†}n = 10
NC = No Calculation

Relative Bioavailability Assessments
for Didemethylcitalopram (Group 1)

| Parameter | 90% C.I. | Ratio of Means | Intra-Subject CV |
|---|---|---|---|
| AUC₀₋ₜ | 36.51% to 98.89% | 60.09% | 56.44% |
| AUC_{0-inf} | NC | 60.09% | NC |
| Cₘₐₓ | 63.80% to 151.20% | 98.21% | 48.01% |

Pharmacokinetic Parameters for Bupropion (Group 2)

| | Geometric Mean (% CV) | | | |
|---|---|---|---|---|
| | Arithmetic Mean ± SD | | | |
| | Bupropion HBr XL 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 1683.03 | (24.53) | 1512.58 | (35.95) |
| (ng · hr/mL) | 1728.43 ± 424.01 | | 1599.22 ± 574.93 | |
| AUC_{0-inf} | 1729.27 | (24.35) | 1555.02 | (35.50) |
| (ng · hr/mL) | 1775.06 ± 432.18 | | 1640.96 ± 582.47 | |
| Cₘₐₓ (ng/mL) | 141.59 | (32.39) | 109.86 | (39.28) |
| | 149.11 ± 48.29 | | 119.88 ± 47.09 | |
| Tₘₐₓ (hr)* | 4.50 | (3.00-10.00) | 5.00 | (2.00-8.00) |

| | | |
|---|---|---|
| t_{1/2} (hr) | 23.70 ± 5.97 | 20.70 ± 7.63 |
| Kₑₗ (hr⁻¹) | 3.14E−02 ± 9.70E−03 | 3.73E−02 ± 1.20E−02 |
| MRT (hr) | 22.27 ± 6.15 | 22.09 ± 6.96 |

*median (min-max)

Relative Bioavailability Assessments for Bupropion (Group 2)

| | Potency Uncorrected Data | | |
|---|---|---|---|
| Parameter | 90% C.I. | Ratio of Means | Intra-Subject CV |
| AUC₀₋ₜ | 79.81% to 101.21% | 89.87% | 14.36% |
| AUC_{0-inf} | 80.17% to 100.86% | 89.92% | 13.87% |
| Cₘₐₓ | 63.13% to 95.36% | 77.59% | 25.19% |

Pharmacokinetic Parameters for Hydroxybupropion (Group 2)

| | Geometric Mean (% CV) | | | |
|---|---|---|---|---|
| | Arithmetic Mean ± SD | | | |
| | Bupropion HBr XL 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 24257.00 | (37.65) | 21904.96 | (53.59) |
| (ng · hr/mL) | 25952.95 ± 9771.48 | | 25096.58 ± 13449.54 | |
| AUC_{0-inf} | 24490.88 | (37.48) | 22142.07 | (53.35) |
| (ng · hr/mL) | 26183.71 ± 9812.76 | | 25322.65 ± 13510.55 | |
| Cₘₐₓ (ng/mL) | 466.81 | (33.07) | 421.37 | (45.63) |
| | 488.11 ± 161.41 | | 466.37 ± 212.79 | |
| Tₘₐₓ (hr)* | 9.00 | (7.00-24.00) | 12.00 | (8.00-36.00) |

| | | |
|---|---|---|
| t_{1/2} (hr) | 26.27 ± 6.08 | 27.45 ± 6.09 |
| Kₑₗ (hr⁻¹) | 2.79E−02 ± 7.15E−03 | 2.64E−02 ± 5.74E−03 |
| MRT (hr) | 44.92 ± 10.09 | 44.93 ± 8.66 |
| M/P Ratio | 14.06 ± 4.99 | 14.25 ± 5.61 |

*median (min-max)

Relative Bioavailability Assessments
for Hydroxybupropion (Group 2)

| Parameter | 90% C.I. | Ratio of Means | Intra-Subject CV |
|---|---|---|---|
| AUC₀₋ₜ | 79.80% to 102.19% | 90.30% | 14.95% |
| AUC_{0-inf} | 80.05% to 102.11% | 90.41% | 14.72% |
| Cₘₐₓ | 80.44% to 101.29% | 90.27% | 13.92% |

Pharmacokinetic Parameters for Bupropion
Threoamino Alcohol (Group 2)

| | Geometric Mean (% CV) | | | |
|---|---|---|---|---|
| | Arithmetic Mean ± SD | | | |
| | Bupropion HBr XL 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 9052.10 | (41.63) | 7946.73 | (45.82) |
| (ng · hr/mL) | 9695.24 ± 4035.97 | | 8695.70 ± 3984.62 | |
| AUC_{0-inf} | 9664.70 | (45.82) | 8489.87 | (48.32) |
| (ng · hr/mL) | 10511.83 ± 4816.41 | | 9395.31 ± 4540.05 | |
| Cₘₐₓ (ng/mL) | 168.34 | (25.85) | 134.83 | (40.31) |
| | 173.22 ± 44.78 | | 145.82 ± 58.78 | |
| Tₘₐₓ (hr)* | 7.00 | (5.00-10.00) | 10.00 | (6.00-36.00) |

| | | |
|---|---|---|
| t_{1/2} (hr) | 58.30 ± 21.53 | 62.13 ± 16.94 |
| Kₑₗ (hr⁻¹) | 1.39E−02 ± 6.46E−03 | 1.20E−02 ± 3.65E−03 |
| MRT (hr) | 80.09 ± 22.79 | 81.49 ± 20.47 |
| M/P Ratio | 5.80 ± 2.04 | 5.88 ± 2.98 |

*median (min-max)

Relative Bioavailability Assessments for
Bupropion Threoamino Alcohol (Group 2)

| Parameter | 90% C.I. | Ratio of Means | Intra-Subject CV |
|---|---|---|---|
| AUC₀₋ₜ | 75.33% to 102.30% | 87.79% | 18.56% |
| AUC_{0-inf} | 74.69% to 103.31% | 87.84% | 19.69% |
| Cₘₐₓ | 66.26% to 96.82% | 80.10% | 23.10% |

Pharmacokinetic Parameters for Bupropion
Erythroamino Alcohol (Group 2)

| | Geometric Mean (% CV) | | | |
|---|---|---|---|---|
| | Arithmetic Mean ± SD | | | |
| | Bupropion HBr XL 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 1597.56 | (33.52) | 1380.30 | (36.03) |
| (ng · hr/mL) | 1680.63 ± 563.35 | | 1457.66 ± 525.14 | |
| AUC_{0-inf} | 1664.27 | (32.70) | 1453.53 | (34.45) |
| (ng · hr/mL) | 1746.86 ± 571.29 | | 1528.45 ± 526.55 | |
| Cₘₐₓ (ng/mL) | 26.81 | (22.05) | 23.80 | (26.45) |
| | 27.30 ± 6.02 | | 24.61 ± 6.51 | |
| Tₘₐₓ (hr)* | 8.00 | (6.00-24.00) | 11.00 | (10.00-36.00) |

| | | |
|---|---|---|
| t_{1/2} (hr) | 33.17 ± 7.58 | 34.67 ± 10.38 |
| Kₑₗ (hr⁻¹) | 2.20E−02 ± 5.48E−03 | 2.18E−02 ± 7.23E−03 |
| MRT (hr) | 56.09 ± 12.79 | 56.55 ± 13.91 |
| M/P Ratio | 0.98 ± 0.22 | 0.95 ± 0.20 |

*median (min-max)

Relative Bioavailability for Bupropion
Erythoamino Alcohol (Group 2)

| Parameter | 90% C.I. | Ratio of Means | Intra-Subject CV |
|---|---|---|---|
| AUC₀₋ₜ | 73.23% to 101.93% | 86.40% | 20.08% |
| AUC_{0-inf} | 74.38% to 102.56% | 87.34% | 19.50% |
| Cₘₐₓ | 76.27% to 103.33% | 88.78% | 18.41% |

Pharmacokinetic Parameters for PAWC (Group 2)

| | Geometric Mean (% CV) | | | |
|---|---|---|---|---|
| | Arithmetic Mean ± SD | | | |
| | Bupropion HBr XL 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 74.24 | (31.23) | 66.95 | (47.43) |
| (uM · hr) | 77.70 ± 24.27 | | 74.13 ± 35.16 | |
| AUC_{0-inf} | 74.73 | (31.51) | 67.40 | (47.71) |
| (uM · hr) | 78.27 ± 24.67 | | 74.70 ± 35.64 | |
| Cₘₐₓ (uM) | 1.73 | (25.00) | 1.42 | (39.90) |
| | 1.78 ± 0.44 | | 1.53 ± 0.61 | |
| Tₘₐₓ (hr)* | 5.50 | (4.00-12.00) | 10.00 | (4.00-36.00) |

| | | |
|---|---|---|
| t_{1/2} (hr) | 30.34 ± 10.99 | 34.85 ± 17.95 |
| Kₑₗ (hr⁻¹) | 2.63E−02 ± 1.11E−02 | 2.36E−02 ± 9.23E−03 |
| MRT (hr) | 45.14 ± 10.49 | 45.44 ± 9.04 |

*median (min-max)

Relative Bioavailability Assessments for PAWC (Group 2)

| Parameter | 90% C.I. | Ratio of Means | Intra-Subject CV |
|---|---|---|---|
| AUC₀₋ₜ | 79.87% to 101.84% | 90.19% | 14.69% |
| AUC_{0-inf} | 79.77% to 101.99% | 90.20% | 14.85% |
| Cₘₐₓ | 72.53% to 92.60% | 81.95% | 14.77% |

Pharmacokinetic Parameters for Citalopram (Group 2)

| | Geometric Mean (% CV) | | | |
|---|---|---|---|---|
| | Arithmetic Mean ± SD | | | |
| | Bupropion HBr XL 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 1204.03 | (15.08) | 1185.17 | (15.02) |
| (ng · hr/mL) | 1216.86 ± 183.46 | | 1197.82 ± 179.96 | |
| AUC_{0-inf} | 1261.16 | (15.59) | 1249.49 | (15.23) |
| (ng · hr/mL) | 1275.36 ± 198.81 | | 1263.33 ± 192.37 | |
| Cₘₐₓ (ng/mL) | 22.41 | (20.18) | 23.80 | (18.23) |
| | 22.80 ± 4.60 | | 24.13 ± 4.40 | |
| Tₘₐₓ (hr)* | 5.00 | (3.00-7.00) | 4.00 | (2.00-6.00) |

| | | |
|---|---|---|
| t_{1/2} (hr) | 58.24 ± 9.70 | 61.99 ± 13.28 |
| Kₑₗ (hr⁻¹) | 1.22E−02 ± 2.09E−03 | 1.16E−02 ± 2.36E−03 |
| MRT (hr) | 73.00 ± 9.19 | 73.59 ± 8.44 |

*median (min-max)

Relative Bioavailability Assessments for Citalopram (Group 2)

| Parameter | 90% C.I. | Ratio of Means | Intra-Subject CV |
|---|---|---|---|
| AUC₀₋ₜ | 95.60% to 101.35% | 98.43% | 3.51% |
| AUC_{0-inf} | 96.24% to 102.00% | 99.07% | 3.50% |
| Cₘₐₓ | 99.30% to 113.59% | 106.21% | 8.09% |

Pharmacokinetic Parameters for Demethylcitalopram (Group 2)

| | Geometric Mean (% CV) | | | |
|---|---|---|---|---|
| | Arithmetic Mean ± SD | | | |
| | Bupropion HBr Xl 348 | | Bupropion HBr XL 348 | |
| | mg/Citalopram HCl IR | | mg/Citalopram HCl IR | |
| | 20 mg Tablets (A) | | 20 mg Tablets (C) | |
| Pharmacokinetic | (fasting conditions) | | (fed conditions) | |
| Parameters | (n = 10) | | (n = 10) | |
| AUC₀₋ₜ | 310.98 | (20.51) | 297.01 | (22.54) |
| (ng · hr/mL) | 316.79 ± 64.99 | | 304.48 ± 68.63 | |
| AUC_{0-inf} | 350.62 | (22.44) | 365.75 | (22.53) |
| (ng · hr/mL) | 357.86 ± 80.30^{Δ} | | 374.08 ± 84.27^{‡} | |
| Cₘₐₓ (ng/mL) | 2.25 | (21.56) | 2.04 | (24.70) |
| | 2.30 ± 0.50 | | 2.10 ± 0.52 | |
| Tₘₐₓ (hr)* | 36.00 | (7.00-96.00) | 24.01 | (5.00-48.30) |

| | | |
|---|---|---|
| t_{1/2} (hr) | 79.40 ± 11.89^{Δ} | 76.85 ± 13.06^{‡} |
| Kₑₗ (hr⁻¹) | 8.92E−03 ± 1.46E−03^{Δ} | 9.25E−03 ± 1.65E−03^{‡} |
| MRT (hr) | 131.77 ± 17.94^{Δ} | 132.77 ± 18.04^{‡} |
| M/P Ratio | 0.29 ± 0.09^{Δ} | 0.31 ± 0.06^{‡} |

*median (min-max)
^{‡}n = 6
^{Δ}n = 8

Relative Bioavailability Assessments
for Demethylcitalopram (Group 2)

| Parameter | 90% C.I. | Ratio of Means | Intra-Subject CV |
|---|---|---|---|
| AUC₀₋ₜ | 87.01% to 104.84% | 95.51% | 11.24% |
| AUC_{0-inf} | 81.31% to 132.92% | 103.96% | 16.28% |
| Cₘₐₓ | 83.45% to 98.85% | 90.83% | 10.21% |

Pharmacokinetic Parameters for Didemethylcitalopram (Group 2)

| | Geometric Mean (% CV) | | |
|---|---|---|---|
| | Arithmetic Mean ± SD | | |
| | | Bupropion HBr Xl 348 | Bupropion HBr XL 348 |
| | | mg/Citalopram HCl IR | mg/Citalopram HCl IR |
| | | 20 mg Tablets (A) | 20 mg Tablets (C) |
| Pharmacokinetic | | (fasting conditions) | (fed conditions) |
| Parameters | | (n = 10) | (n = 10) |

| AUC₀₋ₜ | 38.65 | (75.94) | 33.81 | (74.08) |
|---|---|---|---|---|
| (ng · hr/mL) | 34.29 ± 26.04 | | 29.76 ± 22.04 | |
| AUC_{0-inf} | NC | | NC | |
| (ng · hr/mL) | | | | |
| Cₘₐₓ (ng/mL) | 0.22 | (77.48) | 0.19 | (74.14) |
| | 0.20 ± 0.15 | | 0.17 ± 0.12 | |
| Tₘₐₓ (hr)* | 120.00 | (96.00-216.75)^{Δ} | 144.00 | (72.03-192.00)^{Δ} |

| | | |
|---|---|---|
| t_{1/2} (hr) | NC | NC |
| Kₑₗ (hr⁻¹) | NC | NC |
| MRT (hr) | NC | NC |
| M/P Ratio | NC | NC |

*median (min-max)
^{Δ}n = 8
NC = No Calculation

Relative Bioavailability Assessments
for Didemethylcitalopram (Group 2)

| Parameter | 90% C.I. | Ratio of Means | Intra-Subject CV |
|---|---|---|---|
| AUC₀₋ₜ | 75.91% to 98.91% | 86.65% | 13.24% |
| AUC_{0-inf} | NC | 86.65% | NC |
| Cₘₐₓ | 77.81% to 92.95% | 85.04% | 8.88% |

### Example 24

A Pilot, Single-Dose, Pharmacokinetic Study of a controlled-Release Pharmaceutical Composition of the Invention Comprising Bupropion HBr 348 mg/CITALOPRAM HCl 22.2 mg Tablets in Healthy, Non-Smoking Male and Female Subjects

This was a two-way crossover, randomized, open-label, single-dose, fasting and food-effect, Phase I study. The objectives of this study were a) to determine and compare the rate and extent of absorption of bupropion and citalopram from a test pharmaceutical composition manufactured according to Example 1 versus Bupropion HBr (XL) 348 mg Tablets described in U.S. Pat. No. 7,241,805 co-administered with Celexa® 20 mg Tablets under fasting conditions, and b) to determine the effect of food on the rate and extent of absorption of a fixed dose combination pharmaceutical composition of Example 1. Normal, healthy, non-smoking male and female subjects between the ages of 18 and 55 years were included in the study.

The test formulation used was: Treatment A: One (1) Pharmaceutical Composition of Example 1, Lot #: 0612087, administered orally under fasting conditions, and Treatment C: One (1) Pharmaceutical Composition of Example 1, Lot #: 0612087, administered orally under fed conditions.

The reference formulation used was: Treatment B: One (1) Bupropion HBr XL 348 mg Tablet as described in U.S. Pat. No. 7,241,805, Lot #: 06M064P and 1 Celexa® 20 mg Tablet, Lot #: M0512M administered orally under fasting conditions.

There were 14 subjects dosed in Group 1, 13 of whom completed the study. One subject was dismissed after emesis within 24.00 hours of dosing. Pharmacokinetic and statistical analyses were performed on 13 subjects who completed the study. There were 14 subjects dosed in Group II, 13 of whom completed the study. One subject withdrew for personal reasons. Pharmacokinetic and statistical analyses were performed on 13 subjects who completed the study.

During each study period, 27 blood samples were collected from each subject at the following time points: 0.00 (pre-dose), 0.50, 1.00, 1.50, 2.00, 3.00, 4.00, 5.00, 6.00, 7.00, 8.00, 10.00, 12.00, 16.00, 24.00, 36.00, 48.00, 72.00, 96.00, 120.00, 144.00, 168.00, 192.00, 216.00, 240.00, 264.00 and 288.00 hours post-dose.

The pharmacokinetic analysis was performed on 26 subjects who completed the 2 study periods. The safety assessment was performed on all subjects who received at least 1 dose during the course of the study.

The following pharmacokinetic parameters for bupropion and its metabolites hydroxybupropion, bupropion erythroamino alcohol, and bupropion threoamino alcohol, including pharmacologic activity-weighted composite (PAWC), and citalopram and its metabolites, demethylcitalopram and didemethylcitalopram were calculated by standard non-compartmental methods: AUC₀₋ₜ, AUC_{0-inf}, Cₘₐₓ, Tₘₐₓ, Kₑₗ, t_{1/2}, Mean Residence Time (MRT), and Metabolite/Parent (M/P) ratio.

Statistical methods used General Linear Model (GLM) procedures in Statistical Analysis System (SAS), analysis of variance (ANOVA) was performed on ln-transformed AUC₀₋ₜ, AUC_{0-inf}, and Cₘₐₓ and on untransformed Kₑₗ, t_{1/2}, MRT, and M/P ratio at the significance level of 0.05. The intra-subject coefficient of variation (CV) was calculated using the Mean Square Error (MSE) from the ANOVA table. The ratio of geometric means and the 90% geometric confidence interval (90% C.I.) were calculated based on the difference in the Least Squares Means of the ln-transformed AUC₀₋ₜ, AUC_{0-inf}, and Cₘₐₓ between the test and reference formulations. Tₘₐₓ was analyzed using nonparametric methods.

Bupropion and its metabolites, hydroxybupropion, bupropion erythroamino alcohol, bupropion threoamino alcohol and the internal standard, 1-(3-chlorophenyl)-piperazine, were extracted by solid phase extraction into an organic media from 0.50 mL of human plasma. An aliquot of this extract was injected into a High Performance Liquid Chromatography system and detected using a tandem mass spectrometer. Evaluation of the assay was carried out by the construction of an eight (8) point calibration curve (excluding zero concentration) covering the range of 1.000 ng/mL to 1023.900 ng/mL for bupropion, 3.905 ng/mL to 3998.970 ng/mL for hydroxybupropion, 1.000 ng/mL to 1024.000 ng/mL for bupropion erythroamino alcohol, and 1.000 ng/mL to 1024.000 ng/mL for bupropion threoamino alcohol in human plasma. The slope and intercept of the calibration curves were determined through weighted linear regression analysis (1/area ratio.²). Two calibration curves and duplicate QC samples (at three concentration levels) were analyzed along with each batch of the study samples. Peak area ratios were used to determine the concentration of the standards, quality control samples, and the unknown study samples from the calibration curves.

Citalopram, its metabolites—demethylcitalopram and didemethylcitalopram, and the internal standards, citalopram analog, demethylcitalopram analog, and didemethylcitalopram analog, were extracted by liquid-liquid extraction into an organic media from 1.00 mL of human plasma. An aliquot of this extract was injected into a High Performance Liquid Chromatography system and detected using a tandem mass spectrometer. The analytes were separated by reverse phase chromatography. Evaluation of the assay was carried out by the construction of an eight (8) point calibration curve (excluding zero concentration) covering the range of 0.250 ng/mL to 64.034 ng/mL for citalopram, 0.050 ng/mL to 12.810 ng/mL for demethylcitalopram, and 0.050 ng/mL to 12.791 ng/mL for didemethylcitalopram in human plasma. The slope and intercept of the calibration curves were determined through weighted linear regression analysis (1/conc.²). Two calibration curves and duplicate QC samples (at three concentration levels) were analyzed along with each batch of the study samples. Peak area ratios were used to determine the concentration of the standards, quality control samples, and the unknown study samples from the calibration curves.

Data for the pharmacokinetic parameters for bupropion and citalopram and their metabolites is presented in the tables below and in FIGS. 24A-P.

Pharmacokinetic Parameters for Bupropion (Group 1)

| | Geometric Mean (% CV) | | |
|---|---|---|---|
| | Arithmetic Mean ± SD | | |
| | | Pharmaceutical | Bupropion HBr XL |
| | | Composition according | 348 mg Tablets and |
| | | to Example 1 (A) | Celexa ® 20 mg Tablets (B) |
| Pharmacokinetic | | [fasting conditions] | [fasting conditions] |
| Parameters | | (n = 13) | (n = 13) |

| AUC₀₋ₜ | 1570.10 | (33.74) | 1264.46 | (32.84) |
|---|---|---|---|---|
| (ng · hr/mL) | 1660.28 ± 560.15 | | 1331.74 ± 437.31 | |
| AUC_{0-inf} | 1628.89 | (32.43) | 1315.88 | (31.87) |
| (ng · hr/mL) | 1713.92 ± 555.80 | | 1381.81 ± 440.42 | |
| Cₘₐₓ (ng/mL) | 141.29 | (24.80) | 107.70 | (30.78) |
| | 145.74 ± 36.15 | | 112.77 ± 34.71 | |
| Tₘₐₓ (hr)* | 5.00 | (4.00-7.00) | 4.00 | (2.00-5.00) |

| | | |
|---|---|---|
| t_{1/2} (hr) | 25.03 ± 5.56 | 21.85 ± 5.41 |
| Kₑₗ (hr⁻¹) | 2.91E−02 ± 7.08E−03 | 3.37E−02 ± 9.18E−03 |
| MRT (hr) | 21.45 ± 4.47 | 22.14 ± 5.33 |

*median (min-max)

Relative Bioavailability Assessments
(A vs B) for Bupropion (Group 1)

| | Potency Uncorrected Data | | |
|---|---|---|---|
| Parameter | 90% C.I. | Ratio of Means | Intra-Subject CV |
| AUC₀₋ₜ | 99.67% to 152.59% | 123.32% | 30.84% |
| AUC_{0-inf} | 100.10% to 151.00% | 122.95% | 29.72% |
| Cₘₐₓ | 109.43% to 153.88% | 129.76% | 24.48% |

Potency Corrected Data

| Parameter | 90% C.I. | Ratio of Means |
|---|---|---|
| AUC₀₋ₜ | 96.91% to 148.36% | 119.91% |
| AUC_{0-inf} | 97.33% to 146.82% | 119.54% |
| Cₘₐₓ | 106.40% to 149.62% | 126.17% |

Pharmacokinetic Parameters for Hydroxybupropion (Group 1)

| | Geometric Mean (% CV) | | |
|---|---|---|---|
| | Arithmetic Mean ± SD | | |
| | | Pharmaceutical | Bupropion HBr XL |
| | | Composition according | 348 mg Tablets and |
| | | to Example 1 (A) | Celexa ® 20 mg Tablets (B) |
| Pharmacokinetic | | [fasting conditions] | [fasting conditions] |
| Parameters | | (n = 13) | (n = 13) |

| AUC₀₋ₜ | 23529.49 | (41.42) | 18413.09 | (46.25) |
|---|---|---|---|---|
| (ng · hr/mL) | 25590.82 ± 10600.46 | | 20410.99 ± 9439.43 | |
| AUC_{0-inf} | 23753.82 | (41.18) | 18617.18 | (45.86) |
| (ng · hr/mL) | 25803.36 ± 10626.20 | | 20596.42 ± 9445.45 | |
| Cₘₐₓ (ng/mL) | 510.83 | (27.27) | 375.83 | (39.88) |
| | 531.47 ± 144.94 | | 403.42 ± 160.90 | |
| Tₘₐₓ (hr)* | 10.00 | (5.03-12.00) | 8.00 | (5.00-12.00) |

| | | |
|---|---|---|
| t_{1/2} (hr) | 25.58 ± 7.44 | 24.47 ± 4.56 |
| Kₑₗ (hr⁻¹) | 2.89E−02 ± 6.95E−03 | 2.93E−02 ± 5.60E−03 |
| MRT (hr) | 41.71 ± 8.13 | 40.87 ± 7.13 |
| M/P Ratio | 14.30 ± 4.15 | 13.90 ± 4.23 |

*median (min-max)

Relative Bioavailability Assessments (A
vs B) for Hydroxybupropion (Group 1)

| | Potency Uncorrected Data | | |
|---|---|---|---|
| Parameter | 90% C.I. | Ratio of Means | Intra-Subject CV |
| AUC₀₋ₜ | 101.24% to 158.47% | 126.67% | 32.52% |
| AUC_{0-inf} | 101.23% to 158.07% | 126.50% | 32.34% |
| Cₘₐₓ | 114.13% to 160.12% | 135.18% | 24.31% |

Pharmacokinetic Parameters for Bupropion Threoamino Alcohol (Group 1)

| | Geometric Mean (% CV) | | |
|---|---|---|---|
| | Arithmetic Mean ± SD | | |
| | | Pharmaceutical | Bupropion HBr XL |
| | | Composition according | 348 mg Tablets and |
| | | to Example 1 (A) | Celexa ® 20 mg Tablets (B) |
| Pharmacokinetic | | [fasting conditions] | [fasting conditions] |
| Parameters | | (n = 13) | (n = 13) |

| AUC₀₋ₜ | 7551.59 | (71.70) | 5908.31 | (55.22) |
|---|---|---|---|---|
| (ng · hr/mL) | 8885.47 ± 6370.75 | | 6697.79 ± 3698.44 | |
| AUC_{0-inf} | 6936.64 | (39.91)^{†} | 6106.99 | (59.82) |
| (ng · hr/mL) | 7471.68 ± 2982.15 | | 7024.33 ± 4202.27 | |
| Cₘₐₓ (ng/mL) | 160.20 | (49.22) | 111.90 | (31.85) |
| | 177.47 ± 87.35 | | 118.65 ± 37.79 | |
| Tₘₐₓ (hr)* | 7.00 | (5.00-10.00) | 6.00 | (5.00-8.00) |

| | | |
|---|---|---|
| t_{1/2} (hr) | 48.99 ± 17.20^{†} | 51.54 ± 16.00 |
| Kₑₗ (hr⁻¹) | 1.57E−02 ± 4.97E−03^{†} | 1.45E−02 ± 3.72E−03 |
| MRT (hr) | 67.81 ± 18.80^{†} | 73.63 ± 26.80 |
| M/P Ratio | 4.62 ± 1.60^{†} | 5.01 ± 2.36 |

*median (min-max);
^{†}n = 12

Relative Bioavailability Assessments (A vs B)
for Bupropion Threoamino Alcohol (Group 1)

| | Potency Uncorrected Data | | |
|---|---|---|---|
| Parameter | 90% C.I. | Ratio of Means | Intra-Subject CV |
| AUC₀₋ₜ | 101.81% to 158.62% | 127.08% | 32.17% |
| AUC_{0-inf} | 96.53% to 152.17% | 121.20% | 31.04% |
| Cₘₐₓ | 115.35% to 176.72% | 142.77% | 30.89% |

Pharmacokinetic Parameters for Bupropion
Erythroamino Alcohol (Group 1)

| | Geometric Mean (% CV) | | | |
|---|---|---|---|---|
| | Arithmetic Mean ± SD | | | |
| | Pharmaceutical | | Bupropion HBr XL | |
| | Composition according | | 348 mg Tablets and | |
| | to Example 1 (A) | | Celexa ® 20 mg Tablets (B) | |
| Pharmacokinetic | [fasting conditions] | | [fasting conditions] | |
| Parameters | (n = 13) | | (n = 13) | |
| AUC₀₋ₜ | 1240.23 | (72.45) | 962.02 | (51.81) |
| (ng · hr/mL) | 1519.16 ± 1100.65 | | 1103.04 ± 571.47 | |
| AUC_{0-inf} | 1319.17 | (70.35) | 1027.55 | (49.98) |
| (ng · hr/mL) | 1591.01 ± 1119.21 | | 1163.50 ± 581.51 | |
| Cₘₐₓ (ng/mL) | 25.84 | (38.01) | 18.75 | (28.79) |
| | 27.46 ± 10.44 | | 19.57 ± 5.63 | |
| Tₘₐₓ (hr)* | 10.00 | (5.03-16.00) | 7.00 | (5.00-16.00) |

| | | |
|---|---|---|
| t_{1/2} (hr) | 31.68 ± 10.63 | 29.30 ± 6.44 |
| Kₑₗ (hr⁻¹) | 2.36E−02 ± 5.90E−03 | 2.45E−02 ± 4.41E−03 |
| MRT (hr) | 49.91 ± 12.19 | 48.65 ± 12.48 |
| M/P Ratio | 0.88 ± 0.39 | 0.84 ± 0.33 |

*median (min-max)

Relative Bioavailability Assessments (A vs B)
for Bupropion Erythroamino Alcohol (Group 1)

| | Potency Uncorrected Data | | |
|---|---|---|---|
| Parameter | 90% C.I. | Ratio of Means | Intra-Subject CV |
| AUC₀₋ₜ | 99.18% to 165.51% | 128.12% | 37.47% |
| AUC_{0-inf} | 100.68% to 161.99% | 127.71% | 34.64% |
| Cₘₐₓ | 113.39% to 167.08% | 137.64% | 27.96% |

Pharmacokinetic Parameters for PAWC (Group 1)

| | Geometric Mean (% CV) | | | |
|---|---|---|---|---|
| | Arithmetic Mean ± SD | | | |
| | Pharmaceutical | | Bupropion HBr XL | |
| | Composition according | | 348 mg Tablets and | |
| | to Example 1 (A) | | Celexa ® 20 mg Tablets (B) | |
| Pharmacokinetic | [fasting conditions] | | [fasting conditions] | |
| Parameters | (n = 13) | | (n = 13) | |
| AUC₀₋ₜ | 70.42 | (39.85) | 55.31 | (41.83) |
| (uM · hr) | 75.85 ± 30.23 | | 60.12 ± 25.15 | |
| AUC_{0-inf} | 70.70 | (40.25) | 55.46 | (41.82) |
| (uM · hr) | 76.22 ± 30.68 | | 60.29 ± 25.21 | |
| Cₘₐₓ (uM) | 1.84 | (22.53) | 1.34 | (31.92) |
| | 1.89 ± 0.43 | | 1.40 ± 0.45 | |
| Tₘₐₓ (hr)* | 7.00 | (5.00-8.00) | 5.00 | (4.00-8.00) |

| | | |
|---|---|---|
| t_{1/2} (hr) | 39.22 ± 24.12 | 32.28 ± 15.17 |
| Kₑₗ (hr⁻¹) | 2.48E−02 ± 1.42E−02 | 2.61E−02 ± 1.13E−02 |
| MRT (hr) | 42.42 ± 9.64 | 41.36 ± 8.78 |

*median (min-max)

Relative Bioavailability Assessments
(A vs B) for PAWC (Group 1)

| Parameter | 90% C.I. | Ratio of Means | Intra-Subject CV |
|---|---|---|---|
| AUC₀₋ₜ | 101.38% to 157.32% | 126.29% | 31.86% |
| AUC_{0-inf} | 101.52% to 157.50% | 126.45% | 31.85% |
| Cₘₐₓ | 115.68% to 161.47% | 136.67% | 23.94% |

Pharmacokinetic Parameters for Citalopram (Group 1)

| | Geometric Mean (% CV) | | | |
|---|---|---|---|---|
| | Arithmetic Mean ± SD | | | |
| | Pharmaceutical | | Bupropion HBr XL | |
| | Composition according | | 348 mg Tablets and | |
| | to Example 1 (A) | | Celexa ® 20 mg Tablets (B) | |
| Pharmacokinetic | [fasting conditions] | | [fasting conditions] | |
| Parameters | (n = 13) | | (n = 13) | |
| AUC₀₋ₜ | 909.34 | (44.20) | 1024.06 | (27.26) |
| (ng · hr/mL) | 992.44 ± 438.68 | | 1060.55 ± 289.09 | |
| AUC_{0-inf} | 942.09 | (45.70) | 1052.26 | (28.16) |
| (ng · hr/mL) | 1029.79 ± 470.62 | | 1091.04 ± 307.26 | |
| Cₘₐₓ (ng/mL) | 15.21 | (20.97) | 20.05 | (14.19) |
| | 15.53 ± 3.26 | | 20.24 ± 2.87 | |
| Tₘₐₓ (hr)* | 12.00 | (5.00-16.00) | 5.00 | (4.00-6.00) |

| | | |
|---|---|---|
| t_{1/2} (hr) | 54.95 ± 13.02 | 52.50 ± 12.07 |
| Kₑₗ (hr⁻¹) | 1.32E−02 ± 2.60E−03 | 1.38E−02 ± 2.85E−03 |
| MRT (hr) | 71.76 ± 13.63 | 67.16 ± 12.09 |

*median (min-max)

Relative Bioavailability Assessments
(A vs B) for Citalopram (Group 1)

| | Potency Uncorrected Data | | |
|---|---|---|---|
| Parameter | 90% C.I. | Ratio of Means | Intra-Subject CV |
| AUC₀₋ₜ | 72.72% to 108.10% | 88.66% | 28.62% |
| AUC_{0-inf} | 73.58% to 108.59% | 89.39% | 28.08% |
| Cₘₐₓ | 68.03% to 84.62% | 75.87% | 15.53% |

Pharmacokinetic Parameters for Demethylcitalopram (Group 1)

| | Geometric Mean (% CV) | | |
|---|---|---|---|
| | Arithmetic Mean ± SD | | |
| | | Pharmaceutical | Bupropion HBr XL |
| | | Composition according | 348 mg Tablets and |
| | | to Example 1 (A) | Celexa ® 20 mg Tablets (B) |
| Pharmacokinetic | | [fasting conditions] | [fasting conditions] |
| Parameters | | (n = 13) | (n = 13) |

| AUC₀₋ₜ | 242.98 | (30.83) | 293.58 | (24.56) |
|---|---|---|---|---|
| (ng · hr/mL) | 253.57 ± 78.17 | | 302.30 ± 74.26 | |
| AUC_{0-inf} | 255.23 | (28.48)^{†} | 318.43 | (25.71) |
| (ng · hr/mL) | 264.42 ± 75.31 | | 328.57 ± 84.48 | |
| Cₘₐₓ (ng/mL) | 1.62 | (35.69) | 2.01 | (28.37) |
| | 1.71 ± 0.61 | | 2.09 ± 0.59 | |
| Tmax (hr)* | 36.50 | (36.00-72.10) | 36.00 | (8.00-72.00) |

| | | |
|---|---|---|
| t_{1/2} (hr) | 72.22 ± 15.19^{†} | 69.25 ± 13.88 |
| Kₑₗ (hr⁻¹) | 1.01E−02 ± 2.48E−03^{†} | 1.03E−02 ± 1.62E−03 |
| MRT (hr) | 128.38 ± 14.12^{†} | 123.03 ± 18.91 |
| M/P Ratio | 0.32 ± 0.12^{†} | 0.33 ± 0.12 |

*median (min-max);
^{†}n = 12

Relative Bioavailability Assessments (A
vs B) for Demethylcitalopram (Group 1)

| | Potency Uncorrected Data | | |
|---|---|---|---|
| Parameter | 90% C.I. | Ratio of Means | Intra-Subject CV |
| AUC₀₋ₜ | 70.35% to 96.71% | 82.48% | 22.81% |
| AUC_{0-inf} | 68.33% to 96.92% | 81.38% | 23.61% |
| Cₘₐₓ | 69.21% to 93.34% | 80.38% | 21.41% |

Pharmacokinetic Parameters for Didemetheylcitalopram (Group 1)

| | Geometric Mean (% CV) | | |
|---|---|---|---|
| | Arithmetic Mean ± SD | | |
| | | Pharmaceutical | Bupropion HBr XL |
| | | Composition according | 348 mg Tablets and |
| | | to Example 1 (A) | Celexa ® 20 mg Tablets (B) |
| Pharmacokinetic | | [fasting conditions] | [fasting conditions] |
| Parameters | | (n = 13) | (n = 13) |

| AUC₀₋ₜ | 31.09 | (47.73) | 40.92 | (38.77) |
|---|---|---|---|---|
| (ng · hr/mL) | 34.01 ± 16.24 | | 44.00 ± 17.06 | |
| AUC_{0-inf} | 57.29 | (NC)^{†} | 76.64 | (NC)^{†} |
| (ng · hr/mL) | 57.29 ± NC | | 76.64 ± NC | |
| Cₘₐₓ (ng/mL) | 0.16 | (42.87) | 0.20 | (39.39) |
| | 0.17 ± 0.07 | | 0.22 ± 0.08 | |
| Tₘₐₓ (hr)* | 144.00 | (72.00-240.00) | 120.03 | (72.07-240.00) |

| | | |
|---|---|---|
| t_{1/2} (hr) | 88.31 ± NC^{†} | 87.50 ± NC^{†} |
| Kₑₗ (hr⁻¹) | 7.85E−03 ± NC^{†} | 7.92E−03 ± NC^{†} |
| MRT (hr) | 189.53 ± NC^{†} | 175.85 ± NC^{†} |
| M/P Ratio | 0.10 ± NC^{†} | 0.11 ± NC^{†} |

*median (min-max);
^{†}n = 1;
NC = No Calculation

Relative Bioavailability Assessments
for Didemethylcitalopram (Group 1)

| | Potency Uncorrected Data | | |
|---|---|---|---|
| Parameter | 90% C.I. | Ratio of Means | Intra-Subject CV |
| AUC₀₋ₜ | 68.47% to 84.12% | 75.89% | 14.65% |
| AUC_{0-inf} | NC | NC | NC |
| Cₘₐₓ | 71.29% to 88.24% | 79.31% | 15.18% |

NC = No Calculation

Pharmacokinetic Parameters for Bupropion (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 1430.25 | (29.21) | 1793.34 | (19.73) |
| (ng · hr/mL) | 1488.54 ± 434.88 | | 1827.81 ± 360.54 | |
| AUC_{0-inf} | 1485.63 | (28.48) | 1845.33 | (19.58) |
| (ng · hr/mL) | 1542.82 ± 439.38 | | 1880.14 ± 368.17 | |
| Cₘₐₓ (ng/mL) | 112.22 | (34.48) | 144.31 | (17.58) |
| | 117.99 ± 40.69 | | 146.37 ± 25.73 | |
| Tₘₐₓ (hr)* | 5.00 | (4.00-12.00) | 5.00 | (5.00-7.00) |

| | | |
|---|---|---|
| t_{1/2} (hr) | 23.70 ± 5.98 | 24.62 ± 4.82 |
| Kₑₗ (hr⁻¹) | 3.14E−02 ± 9.65E−03 | 2.92E−02 ± 5.88E−03 |
| MRT (hr) | 21.65 ± 4.41 | 21.57 ± 3.87 |

*median (min-max)

Relative Bioavailability Assessments (Fed vs
Fasting (C vs A)) for Bupropion (Group 2)

| | Potency Uncorrected Data | | |
|---|---|---|---|
| Parameter | 90% C.I. | Ratio of Means | Intra-Subject CV |
| AUC₀₋ₜ | 109.66% to 141.16% | 124.42% | 18.01% |
| AUC_{0-inf} | 109.16% to 139.23% | 123.28% | 17.35% |
| Cₘₐₓ | 103.65% to 155.19% | 126.83% | 29.16% |

Pharmacokinetic Parameters for Hydroxybupropion (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 19905.35 | (55.57) | 21620.85 | (35.78) |
| (ng · hr/mL) | 23223.14 ± 12904.38 | | 23226.52 ± 8310.59 | |
| AUC_{0-inf} | 20166.01 | (55.02) | 21871.10 | (35.47) |
| (ng · hr/mL) | 23431.66 ± 12892.50 | | 23456.76 ± 8319.25 | |
| Cₘₐₓ (ng/mL) | 417.94 | (48.54) | 454.81 | (34.81) |
| | 476.51 ± 231.30 | | 492.18 ± 171.35 | |
| Tₘₐₓ (hr)* | 12.00 | (8.00-16.00) | 12.00 | (8.00-24.00) |

| | | |
|---|---|---|
| t_{1/2} (hr) | 26.75 ± 5.12 | 26.63 ± 5.08 |
| Kₑₗ (hr⁻¹) | 2.68E−02 ± 5.15E−03 | 2.68E−02 ± 4.42E−03 |
| MRT (hr) | 41.65 ± 6.53 | 42.83 ± 6.01 |
| M/P Ratio | 14.44 ± 6.48 | 12.28 ± 4.92 |

*median (min-max)

Relative Bioavailability Assessments(Fed vs Fasting
(C vs A)) for Hydroxybupropion (Group 2)

| | Potency Uncorrected Data | | |
|---|---|---|---|
| Parameter | 90% C.I. | Ratio of Means | Intra-Subject CV |
| AUC₀₋ₜ | 93.72% to 123.00% | 107.36% | 19.42% |
| AUC_{0-inf} | 93.85% to 122.52% | 107.23% | 19.04% |
| Cₘₐₓ | 94.59% to 122.05% | 107.44% | 18.19% |

Pharmacokinetic Parameters for Bupropion
Threoamino Alcohol (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 7522.59 | (47.43) | 8692.42 | (31.12) |
| (ng · hr/mL) | 8176.10 ± 3877.66 | | 9046.50 ± 2814.88 | |
| AUC_{0-inf} | 7767.17 | (50.35) | 8996.27 | (32.35) |
| (ng · hr/mL) | 8496.38 ± 4277.79 | | 9396.06 ± 3040.00 | |
| Cₘₐₓ (ng/mL) | 136.93 | (36.47) | 163.81 | (25.26) |
| | 145.99 ± 53.24 | | 168.28 ± 42.51 | |
| Tₘₐₓ (hr)* | 10.00 | (5.00-12.00) | 8.00 | (5.00-12.00) |

| | | |
|---|---|---|
| t_{1/2} (hr) | 53.77 ± 13.61 | 61.46 ± 19.76 |
| Kₑₗ (hr⁻¹) | 1.37E−02 ± 3.50E−03 | 1.25E−02 ± 4.62E−03 |
| MRT (hr) | 76.86 ± 18.02 | 77.31 ± 17.82 |
| M/P Ratio | 5.38 ± 1.55 | 5.01 ± 1.47 |

*median (min-max)

Relative Bioavailability Assessments (Fed vs Fasting
(C vs A)) for Bupropion Threoamino Alcohol (Group 2)

| | Potency Uncorrected Data | | |
|---|---|---|---|
| Parameter | 90% C.I. | Ratio of Means | Intra-Subject CV |
| AUC₀₋ₜ | 101.77% to 129.31% | 114.72% | 17.07% |
| AUC_{0-inf} | 102.12% to 129.52% | 115.01% | 16.94% |
| Cₘₐₓ | 101.80% to 137.59% | 118.35% | 21.56% |

Pharmacokinetic Parameters for Bupropion
Erythroamino Alcohol (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 1277.83 | (61.07) | 1493.34 | (43.69) |
| (ng · hr/mL) | 1451.80 ± 886.65 | | 1597.68 ± 698.05 | |
| AUC_{0-inf} | 1343.22 | (59.16) | 1561.88 | (42.33) |
| (ng · hr/mL) | 1516.13 ± 896.90 | | 1665.50 ± 704.99 | |
| Cₘₐₓ (ng/mL) | 25.11 | (27.00) | 28.53 | (20.72) |
| | 26.18 ± 7.07 | | 29.06 ± 6.02 | |
| Tₘₐₓ (hr)* | 12.00 | (8.00-16.03) | 12.00 | (8.00-16.00) |

| | | |
|---|---|---|
| t_{1/2} (hr) | 30.55 ± 7.65 | 32.09 ± 7.62 |
| Kₑₗ (hr⁻¹) | 2.40E−02 ± 5.70E−03 | 2.28E−02 ± 5.55E−03 |
| MRT (hr) | 50.16 ± 13.62 | 51.48 ± 10.29 |
| M/P Ratio | 0.95 ± 0.34 | 0.89 ± 0.34 |

*median (min-max)

Relative Bioavailability Assessments (Fed vs Fasting (C
vs A)) for Bupropion Erythroamino Alcohol (Group 2)

| | Potency Uncorrected Data | | |
|---|---|---|---|
| Parameter | 90% C.I. | Ratio of Means | Intra-Subject CV |
| AUC₀₋ₜ | 99.90% to 134.28% | 115.83% | 21.16% |
| AUC_{0-inf} | 100.32% to 132.61% | 115.34% | 19.94% |
| Cₘₐₓ | 98.96% to 127.47% | 112.32% | 18.06% |

Pharmacokinetic Parameters for PAWC (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 61.80 | (49.03) | 68.49 | (27.96) |
| (uM · hr) | 68.91 ± 33.79 | | 71.18 ± 19.91 | |
| AUC_{0-inf} | 62.01 | (49.01) | 68.75 | (27.86) |
| (uM · hr) | 69.11 ± 33.87 | | 71.42 ± 19.90 | |
| Cₘₐₓ (uM) | 1.52 | (34.30) | 1.67 | (25.47) |
| | 1.61 ± 0.55 | | 1.74 ± 0.44 | |
| Tₘₐₓ (hr)* | 8.00 | (5.00-12.00) | 8.00 | (5.00-12.00) |

| | | |
|---|---|---|
| t_{1/2} (hr) | 46.97 ± 20.35 | 52.22 ± 24.12 |
| Kₑₗ (hr⁻¹) | 1.81E−02 ± 9.36E−03 | 1.69E−02 ± 1.01E−02 |
| MRT (hr) | 42.32 ± 7.31 | 43.19 ± 6.67 |

*median (min-max)

Relative Bioavailability Assessments (Fed
vs Fasting (C vs A)) for PAWC (Group 2)

| | Potency Uncorrected Data | | |
|---|---|---|---|
| Parameter | 90% C.I. | Ratio of Means | Intra-Subject CV |
| AUC₀₋ₜ | 96.52% to 124.43% | 109.59% | 18.12% |
| AUC_{0-inf} | 96.62% to 124.35% | 109.61% | 18.00% |
| Cₘₐₓ | 94.39% to 124.73% | 108.50% | 19.92% |

Pharmacokinetic Parameters for Citalopram (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 880.17 | (41.69) | 1026.46 | (25.16) |
| (ng · hr/mL) | 951.81 ± 396.81 | | 1052.52 ± 264.82 | |
| AUC_{0-inf} | 909.29 | (42.25) | 1057.35 | (26.24) |
| (ng · hr/mL) | 983.67 ± 415.60 | | 1085.96 ± 284.96 | |
| Cₘₐₓ (ng/mL) | 13.67 | (28.83) | 16.15 | (11.95) |
| | 14.23 ± 4.10 | | 16.25 ± 1.94 | |
| Tₘₐₓ (hr)* | 12.00 | (6.00-16.00) | 12.00 | (6.00-16.00) |

| | | |
|---|---|---|
| t_{1/2} (hr) | 51.00 ± 12.09 | 53.86 ± 14.81 |
| Kₑₗ (hr⁻¹) | 1.43E−02 ± 3.30E−03 | 1.38E−02 ± 3.78E−03 |
| MRT (hr) | 73.11 ± 12.92 | 73.48 ± 13.08 |

*median (min-max);

Relative Bioavailability Assessments (Fed vs
Fasting (C vs A)) for Citalopram (Group 2)

| | Potency Uncorrected Data | | |
|---|---|---|---|
| Parameter | 90% C.I. | Ratio of Means | Intra-Subject CV |
| AUC₀₋ₜ | 101.45% to 133.49% | 116.37% | 19.61% |
| AUC_{0-inf} | 101.53% to 132.67% | 116.06% | 19.10% |
| Cₘₐₓ | 102.57% to 133.54% | 117.03% | 18.84% |

Pharmacokinetic Parameters for Demethylcitalopram (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 13) | | (n = 13) | |
| AUC₀₋ₜ | 266.04 | (36.92) | 296.07 | (34.31) |
| (ng · hr/mL) | 284.13 ± 104.90 | | 311.92 ± 107.00 | |
| AUC_{0-inf} | 307.20 | (31.27)^{†} | 318.99 | (35.69)^{†} |
| (ng · hr/mL) | 320.62 ± 100.26 | | 337.06 ± 120.28 | |
| Cₘₐₓ (ng/mL) | 1.71 | (34.71) | 1.89 | (33.51) |
| | 1.80 ± 0.62 | | 1.98 ± 0.66 | |
| Tₘₐₓ (hr)* | 36.07 | (24.00-72.02) | 36.00 | (16.00-96.05) |

| | | |
|---|---|---|
| t_{1/2} (hr) | 73.60 ± 22.83^{†} | 70.51 ± 21.22^{†} |
| Kₑₗ (hr⁻¹) | 1.03E−02 ± 3.36E−03^{†} | 1.06E−02 ± 3.05E−03^{†} |
| MRT (hr) | 136.19 ± 27.81^{†} | 131.71 ± 25.36^{†} |
| M/P Ratio | 0.37 ± 0.13^{†} | 0.34 ± 0.12^{†} |

*median (min-max);
^{†}n = 11

Relative Bioavailability Assessments (Fed vs Fasting
(C vs A)) for Demethylcitalopram (Group 2)

| | Potency Uncorrected Data | | |
|---|---|---|---|
| Parameter | 90% C.I. | Ratio of Means | Intra-Subject CV |
| AUC₀₋ₜ | 99.59% to 123.94% | 111.10% | 15.57% |
| AUC_{0-inf} | 99.77% to 118.41% | 108.69% | 10.12% |
| Cₘₐₓ | 99.19% to 123.23% | 110.56% | 15.44% |

Pharmacokinetic Parameters for Didemethylcitalopram (Group 2)

| | Geometric Mean (% CV) | | | |
|---|---|---|---|---|
| | Arithmetic Mean ± SD | | | |
| | Pharmaceutical | | Pharmaceutical | |
| | Composition according | | Composition according | |
| | to Example 1 (A) | | to Example 1 (C) | |
| Pharmacokinetic | [fasting conditions] | | [fed conditions] | |
| Parameters | (n = 11) | | (n = 11) | |
| AUC₀₋ₜ | 43.47 | (51.49) | 45.45 | (49.86) |
| (ng · hr/mL) | 49.19 ± 25.33 | | 52.15 ± 26.00 | |
| AUC_{0-inf} | 89.06 | (NC)^{†} | 51.20 | (NC)^{†} |
| (ng · hr/mL) | 89.06 ± NC | | 51.20 ± NC | |
| Cₘₐₓ (ng/mL) | 0.22 | (56.74) | 0.23 | (53.18) |
| | 0.25 ± 0.14 | | 0.27 ± 0.14 | |
| Tₘₐₓ (hr)* | 144.00 | (72.00-192.17) | 144.00 | (72.00-192.00) |

| | | |
|---|---|---|
| t_{1/2} (hr) | 73.00 ± NC^{†} | 84.53 ± NC^{†} |
| Kₑₗ (hr⁻¹) | 9.49E−03 ± NC^{†} | 8.20E−03 ± NC^{†} |
| MRT (hr) | 192.53 ± NC^{†} | 188.05 ± NC^{†} |
| M/P Ratio | 0.09 ± NC^{†} | 0.07 ± NC |

*median (min-max);
^{†}n = 1;
NC = No Calculation

Relative Bioavailability Assessments (Fed vs Fasting
(C vs A)) for Didemethylcitalopram (Group 2)

| | Potency Uncorrected Data | | |
|---|---|---|---|
| Parameter | 90% C.I. | Ratio of Means | Intra-Subject CV |
| AUC₀₋ₜ | 88.70% to 124.37% | 105.03% | 21.03% |
| AUC_{0-inf} | NC | NC | NC |
| Cₘₐₓ | 92.92% to 128.57% | 109.30% | 20.18% |

### Example 25

A Four-Way, Pilot, Single Dose, Fasting Study of a Controlled-Release Pharmaceutical Composition of the Invention Comprising Bupropion HBr (348 mg)/Escitalopram Oxalate (25.5 Mg).

This was a 4-way crossover, randomized, open-label, single-dose, fasting, pilot, phase I (comparative bioavailability) study. The objective of this study was to evaluate the relative peak and systemic exposure of 2 controlled-release pharmaceutical compositions of the invention comprising Bupropion HBr (348 mg)/Escitalopram Oxalate (25.5 mg (Formulations A (manufactured according to Example 3 (Batch No. 0704028) and B (manufactured according to Example 2 (Batch No. 0705037)) to Bupropion HBr XL 348 mg Tablets manufactured according to U.S. Pat. No. 7,241,805 (Lot # 07D042P) and Lexapro® 20 mg Tablets (Lot M0603M) given concomitantly (i.e., co-administered) under single dose fasting conditions. The secondary objective was to determine whether there was a drug-by-formulation (i.e., different method of manufacture (separate granulation vs co-granulation) interaction with respect to the escitalopram component. Normal, healthy, non-smoking male and female subjects between the ages of 18 and 55 years were included in the study.

The following treatments were administered in this study:
Treatment A: One (1) Bupropion HBr (348 mg)/Escitalopram Oxalate (25.5 mg) SR Tablet [Formulation A], Lot #: 0704028, administered orally;
Treatment B: One (1) Bupropion HBr (348 mg)/Escitalopram Oxalate (25.5 mg) SR Tablet [Formulation B], Lot #: 0705037, administered orally;
Treatment C: One (1) Bupropion HBr XL 349 mg Tablet, Lot # 07D042P and one (1) Lexapro® 20 mg Tablet, Lot#: M0603M, administered orally; AND
Treatment D: One (1) Lexapro® 20 mg Tablet, Lot M0603M, administered orally

There were 24 subjects dosed in Period I, 13 of whom completed the study. Two subjects withdrew because of adverse events (AEs) (Subject #007, who experienced dizziness, nausea, sweating, shakiness and weakness; Subject #013, who experienced a laceration on his right hand). One subject (Subject #010) was dismissed due to an AE (low hemoglobin levels). Two subjects (Subjects #002 and #006) were dismissed after emesis within 24.00 hours of dosing. Two subjects were dismissed because of administrative reasons (Subject #014, whose personal residence was infested with bedbugs, and Subject #024 who tested positive for benzodiazepines at checking for Period III). Four subjects (Subjects #008, 009, #020 and #022) withdrew for personal reasons.

During each study period, 23 blood samples were collected from each subject at the following timepoints: 0.00 (pre-dose), 1.00, 2.00, 3.00, 4.00, 6.00, 8.00, 10.00, 12.00, 16.00, 24.00, 36.00, 48.00, 72.00, 96.00, 120.00, 144.00, 168.00, 192.00, 216.00, 240.00, 264.00 and 288.00 hours post-dose.

The following pharmacokinetic parameters for bupropion and its metabolites hydroxybupropion, bupropion threoamino alcohol, bupropion erythroamino alcohol, and PAWC as well as escitalopram and its metabolites S-demethylcitalopram and S-didemethylcitalopram were calculated by standard non-compartmental methods: AUC₀₋ₜ, AUC_{0-inf}, Cₘₐₓ, Tₘₐₓ, t_{1/2}, Kₑₗ, MRT, and M/P ratio.

Using General Linear Model (GLM) procedures in Statistical Analysis System (SAS), analysis of variance (ANOVA) was performed on ln-transformed AUC₀₋ₜ, AUC_{0-inf}, and Cₘₐₓ and on untransformed t_{1/2}, Kₑₗ, MRT, and M/P ratio at the significance level of 0.05. The intra-subject coefficient of variation (CV) was calculated using the Mean Square Error (MSE) from the ANOVA table. The ratio of geometric means and the 90% geometric confidence interval (90% C.I.) were calculated based on the difference in the Least Squares Means of the ln-transformed AUC₀₋ₜ, AUC_{0-inf}, and Cₘₐₓ between the test and reference formulations. Tₘₐₓ was analyzed using nonparametric methods.

Bupropion and its metabolites and citalopram and its metabolites were assayed as follows. Bupropion, hydroxybupropion, bupropion erythroamino alcohol, bupropion threoamino alcohol, and the internal standard, 1-(3-chlorophenyl)-piperazine, were extracted from human plasma (0.50 mL), by solid phase extraction (SPE) into an organic medium. The analytes were separated by High Performance Liquid Chromatography (HPLC) system using reverse phase chromatography conditions, detected using an API 3000 tandem mass spectrometer. Method sensitivity and selectivity were achieved by detecting distinct precursor to production mass transitions for bupropion (240.3→184.0), hydroxybupropion (256.3→238.0), bupropion erythroamino alcohol (242.4→168.1), bupropion threoamino alcohol (242.4→168.1) and the internal standard, 1-(3-chlorophenyl)-piperazine (197.3→153.8), at defined retention time. Evaluation of the assay, using defined acceptance criteria, was carried out by the construction of an eight (8) point calibration curve (excluding zero concentration) covering the range of 1.000 ng/ml to 1023.900 ng/ml for bupropion, 3.907 ng/ml to 4000.320 ng/ml for hydroxybupropion, 1.000 ng/ml to 1024.000 ng/ml for bupropion erythroamino alcohol, and 1.000 ng/ml to 1024.000 ng/ml for bupropion threoamino alcohol in human plasma. the slope and intercept of the calibration curves were determined through weighted linear regression analysis (1/peak area ratio²). Two calibration curves and duplicate QC samples (at three or five concentration levels) were analyzed along with each batch of the study samples. Peak area ratios were used to determine the concentration of the standards, quality control samples, and the unknown study samples from the calibration curves. The concentrations of escitalopram, S-demethylcitalopram and S-didemthylcitalopram were measured using an achrial LC-MS/MS method for citalopram, demethylcitalopram and didemethylcitalopram. Citalopram, demethylcitalopram, didemethylcitalopram and the internal standards, citalopram analog, demethylcitalopram analog, and didemethylcitalopram analog, were extracted from human plasma (0.75 mL), using sodium heparin as an anticoagulant, by liquid-liquid extraction into an organic medium followed by back extraction into a dilute acid. An aliquot of this extract was injected into a High Performance Liquid Chromatography system and detected using a TSQ Quantum tandem mass spectrometer. The analytes were separated by reverse phase chromatography. Method sensitivity and selectivity were achieved by detecting distinct precursor to product ion mass transitions for citalopram (325.1→109.0), demethylcitalopram (311.1→109.0), didemethylcitalopram (297.1→109.0 and 297.1→260.0), and the internal standards, citalopram analog (341.1→125.0), demethylcitalopram analog (327.1→125.0), and didemethylcitalopram analog (313.1→125.0), at defined retention time. Evaluation of the assay, using defined acceptance criteria, was carried out by the construction of an eight (8) point calibration curve (excluding zero concentration) covering the range of 0.251 ng/ml to 64.020 ng/ml for citalopram, 0.126 ng/ml to 32.013 ng/ml for demethylcitalopram, and 0.025 ng/ml to 6.397 ng/ml for didemethylcitalopram in human plasma. The slope and intercept of the calibration curves were determined through weighted linear regression analysis (1/conc.²). Two calibration curves and duplicate QC samples (at three concentration levels) were analyzed along with each batch of the study samples. Peak area ratios were used to determine the concentration of the standards, quality control samples, and the unknown study samples from the calibration curves.

The pharmacokinetic and statistical analyses were performed on data for bupropion and its metabolites from 14 subjects, 13 of who completed the 4 study periods and 1 for whom there were sufficient data in at least 2 periods to potentially allow for a meaningful analysis. The pharmacokinetic and statistical analysis was performed data for citalopram and its metabolites on 13 subjects who completed the 4 study periods.

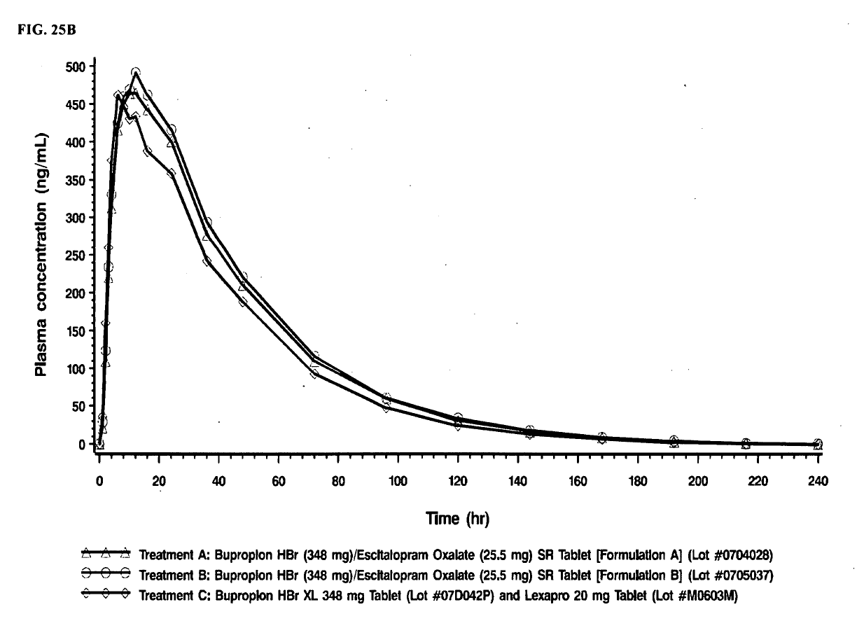

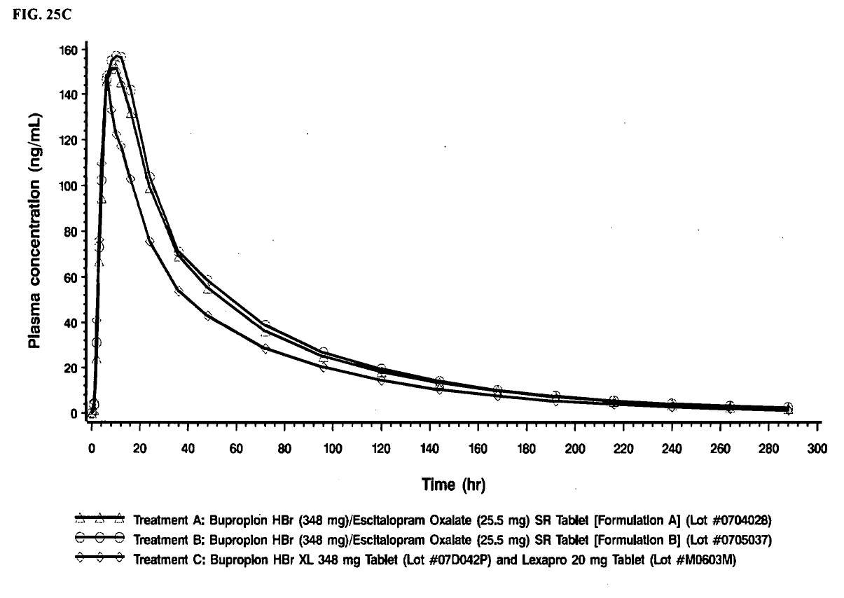

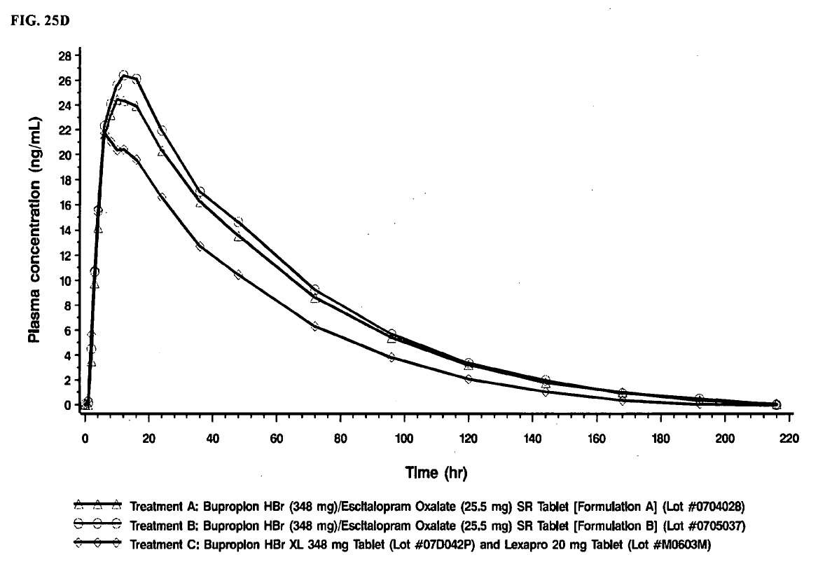

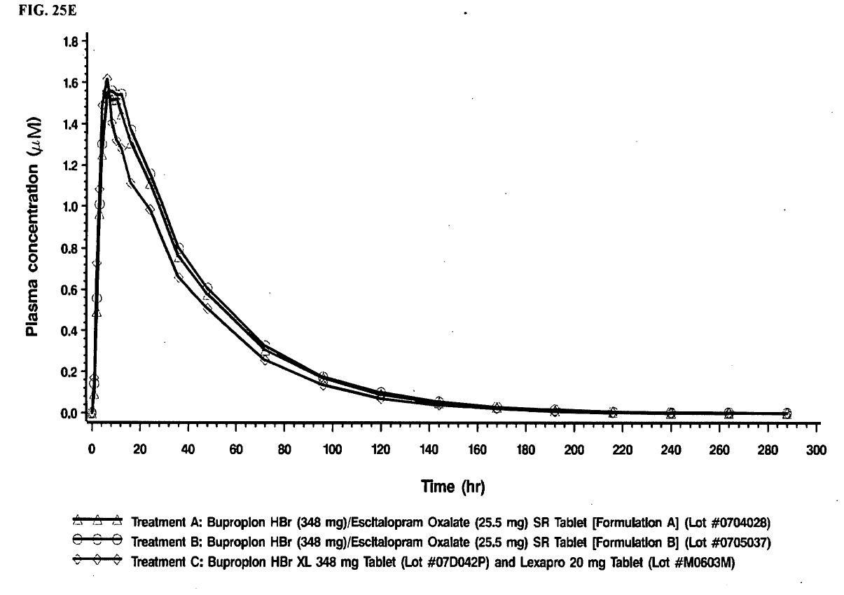

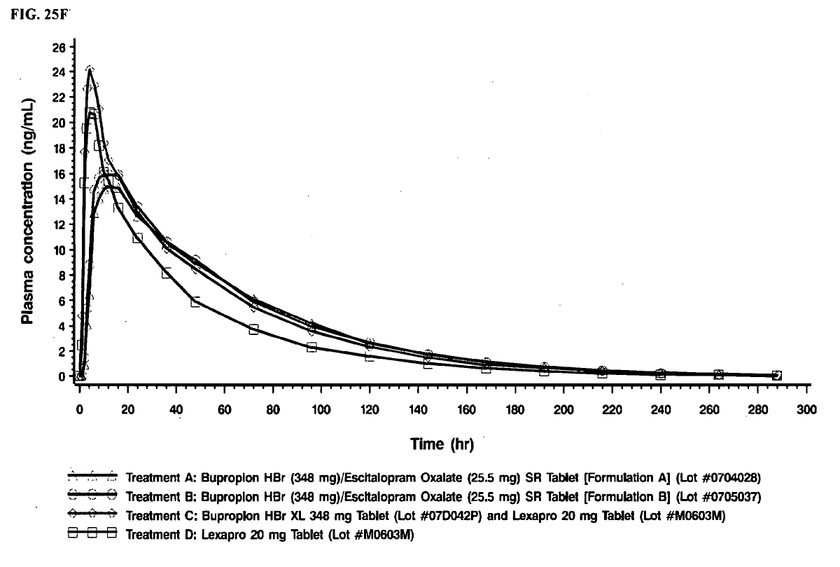

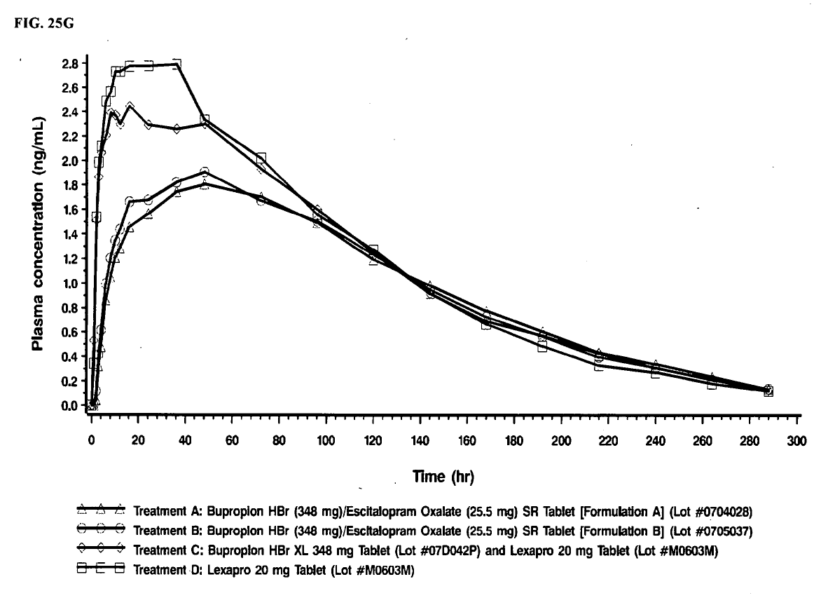

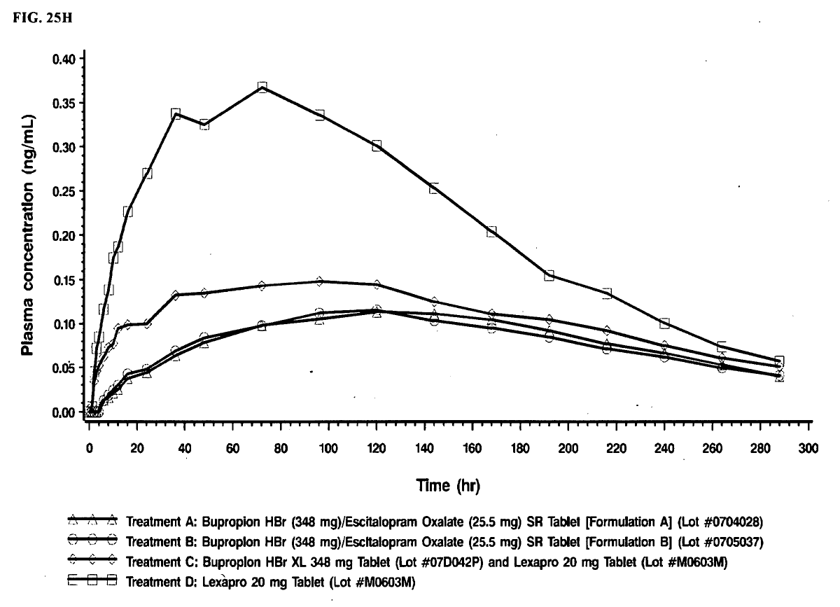

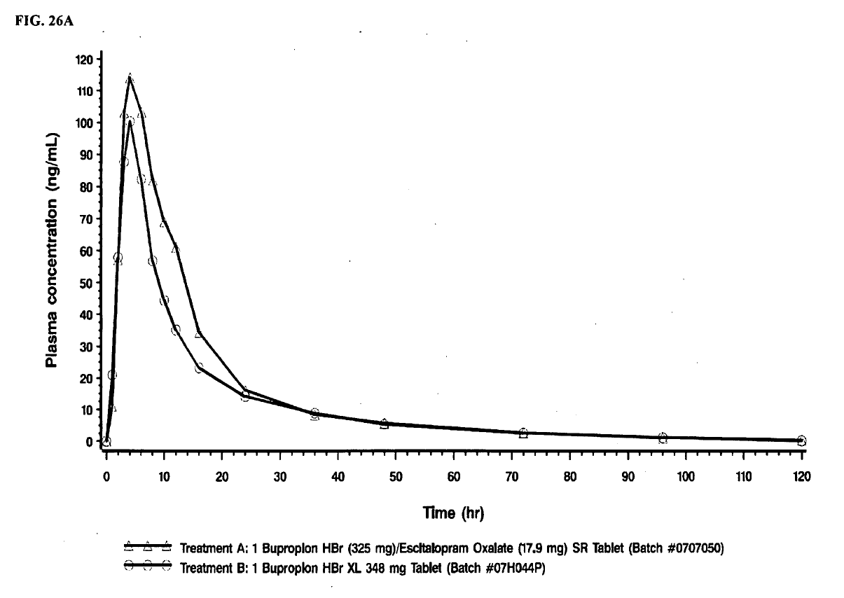

Data for the pharmacokinetic parameters for bupropion and citalopram and its metabolites is presented in the tables below and in FIGS. 25A-H.

Pharmacokinetic Parameters for Bupropion

| | Geometric Mean (% CV) | | | | | |
|---|---|---|---|---|---|---|
| | Arithmetic Mean ± SD | | | | | |
| | Pharmaceutical | | Pharmaceutical | | Bupropion HBr XL | |
| | Composition according | | Composition according | | 348 mg Tablets | |
| | to Example 3 | | to Example 2 | | and Lexapro ® | |
| Pharmacokinetic | [Formulation A] | | [Formulation B] | | 20 mg Tablets | |
| Parameters | (A) (n = 14) | | (B) (n = 14) | | (C) (n = 14) | |
| AUC₀₋ₜ | 1592.27 | (29.72) | 1674.66 | (28.35) | 1401.39 | (25.50) |
| (ng · hr/mL) | 1665.89 ± 495.03 | | 1733.70 ± 491.44 | | 1444.53 ± 368.32 | |
| AUC_{0-inf} | 1646.47 | (28.85) | 1726.12 | (27.75) | 1448.54 | (24.96) |
| (ng · hr/mL) | 1718.79 ± 495.92 | | 1784.69 ± 495.25 | | 1491.40 ± 372.29 | |
| Cₘₐₓ (ng/mL) | 108.41 | (22.58) | 108.97 | (25.09) | 121.53 | (33.25) |
| | 111.42 ± 25.16 | | 112.38 ± 28.20 | | 127.76 ± 42.47 | |
| Tₘₐₓ (hr)* | 6.00 | (3.00-10.00) | 4.00 | (3.02-8.00) | 4.00 | (2.00-6.00) |

| | | | |
|---|---|---|---|
| t_{1/2} (hr) | 24.59 ± 6.00 | 24.99 ± 6.13 | 23.43 ± 5.74 |
| Kₑₗ (hr⁻¹) | 3.07E−02 ± 1.20E−02 | 2.97E−02 ± 8.87E−03 | 3.14E−02 ± 8.04E−03 |
| MRT (hr) | 22.63 ± 3.42 | 23.01 ± 4.15 | 21.53 ± 4.36 |

*median (min-max)

Relative Bioavailability Assessments For Bupropion (A vs C)

| Parameter | 90% C.I. | Ratio of Means | Intra-Subject CV |
|---|---|---|---|
| AUC₀₋ₜ | 103.98% to 140.41% | 120.83% | 18.06% |
| AUC_{0-inf} | 104.59% to 139.92% | 120.97% | 17.49% |
| Cₘₐₓ | 78.28% to 110.88% | 93.17% | 20.99% |

Relative Bioavailability Assessments For Bupropion (B vs C)

| Parameter | 90% C.I. | Ratio of Means | Intra-Subject CV |
|---|---|---|---|
| AUC₀₋ₜ | 108.81% to 140.84% | 123.79% | 18.06% |
| AUC_{0-inf} | 108.98% to 139.95% | 123.50% | 17.49% |
| Cₘₐₓ | 79.11% to 106.70% | 91.87% | 20.99% |

Relative Bioavailability Assessments For Bupropion (B vs A)

| Parameter | Ratio of Means |
|---|---|
| AUC₀₋ₜ | 102.45% |
| AUC_{0-inf} | 102.09% |
| Cₘₐₓ | 98.61% |

Pharmacokinetic Parameters for Hydroxybupropion

| | Geometric Mean (% CV) | | | | | |
|---|---|---|---|---|---|---|
| | Arithmetic Mean ± SD | | | | | |
| | Pharmaceutical | | Pharmaceutical | | Bupropion HBr XL | |
| | Composition of | | Composition of | | 348 mg Tablets | |
| | Example 3 | | Example 2 | | and Lexapro ® | |
| Pharmacokinetic | [Formulation A] | | [Formulation B] | | 20 mg Tablets | |
| Parameters | (A) (n = 14) | | (B) (n = 14) | | (C) (n = 14) | |
| AUC₀₋ₜ | 22649.81 | (36.33) | 24259.45 | (28.61) | 20951.94 | (24.98) |
| (ng · hr/mL) | 24149.14 ± 8773.57 | | 25264.47 ± 7227.97 | | 21698.92 ± 5420.94 | |
| AUC_{0-inf} | 22862.02 | (36.11) | 24462.97 | (28.44) | 21163.98 | (24.80) |
| (ng · hr/mL) | 24358.34 ± 8795.64 | | 25464.13 ± 7243.12 | | 21902.02 ± 5432.04 | |
| Cₘₐₓ (ng/mL) | 479.62 | (29.20) | 492.08 | (22.35) | 459.12 | (22.85) |
| | 501.27 ± 146.38 | | 506.09 ± 113.13 | | 472.84 ± 108.06 | |
| Tₘₐₓ (hr)* | 11.00 | (8.00-24.00) | 12.00 | (8.00-16.05) | 6.00 | (4.00-24.00) |

| | | | |
|---|---|---|---|
| t_{1/2} (hr) | 25.61 ± 3.60 | 25.86 ± 3.08 | 25.07 ± 3.41 |
| Kₑₗ (hr⁻¹) | 2.76E−02 ± 3.91E−03 | 2.72E−02 ± 3.56E−03 | 2.81E−02 ± 3.60E−03 |
| MRT (hr) | 42.00 ± 5.20 | 42.39 ± 6.22 | 40.13 ± 5.15 |
| M/P Ratio | 13.28 ± 2.79 | 13.60 ± 3.02 | 14.09 ± 3.51 |

*median (min-max)

Relative Bioavailability Assessments
For Hydroxybupropion (A vs C)

| Parameter | 90% C.I. | Ratio of Means | Intra-Subject CV |
|---|---|---|---|
| AUC₀₋ₜ | 95.69% to 136.26% | 114.19% | 21.31% |
| AUC_{0-inf} | 95.78% to 136.09% | 114.17% | 21.18% |
| Cₘₐₓ | 96.30% to 125.86% | 110.09% | 16.07% |

Relative Bioavailability Assessments
For Hydroxybupropion (B vs C)

| Parameter | 90% C.I. | Ratio of Means | Intra-Subject CV |
|---|---|---|---|
| AUC₀₋ₜ | 102.58% to 138.99% | 119.41% | 21.31% |
| AUC_{0-inf} | 102.54% to 138.67% | 119.24% | 21.18% |
| Cₘₐₓ | 98.42% to 123.88% | 110.41% | 16.07% |

Relative Bioavailability Assessments
For Hydroxybupropion (B vs A)

| Parameter | Ratio of Means |
|---|---|
| AUC₀₋ₜ | 104.57% |
| AUC_{0-inf} | 104.44% |
| Cₘₐₓ | 100.29% |

Pharmacokinetic Parameters for Bupropion Threoamino Alcohol

| | Geometric Mean (% CV) | | | | | |
|---|---|---|---|---|---|---|
| | Arithmetic Mean ± SD | | | | | |
| | Pharmaceutical | | | | Bupropion HBr XL | |
| | Composition according | | Pharmaceutical | | 348 mg Tablets | |
| | to Example 3 | | Composition according | | and Lexapro ® | |
| Pharmacokinetic | [Formulation A] | | to [Formulation B] | | 20 mg Tablets | |
| Parameters | (A) (n = 14) | | (B) (n = 14) | | (C) (n = 14) | |
| AUC₀₋ₜ | 7554.14 | (39.64) | 8053.81 | (35.18) | 6090.33 | (38.15) |
| (ng · hr/mL) | 8137.89 ± 3225.77 | | 8584.81 ± 3020.41 | | 6547.74 ± 2498.29 | |
| AUC_{0-inf} | 7744.52 | (39.30) | 8326.17 | (34.81) | 6228.78 | (37.98) |
| (ng · hr/mL) | 8333.68 ± 3275.03 | | 8874.78 ± 3089.23 | | 6692.84 ± 2542.17 | |
| Cₘₐₓ (ng/mL) | 154.51 | (40.88) | 154.75 | (38.37) | 137.07 | (45.52) |
| | 167.29 ± 68.40 | | 165.90 ± 63.66 | | 150.98 ± 68.73 | |
| Tₘₐₓ (hr)* | 8.02 | (6.00-24.00) | 10.00 | (6.00-16.05) | 6.00 | (4.00-16.07) |

| | | | |
|---|---|---|---|
| t_{1/2} (hr) | 56.32 ± 15.32 | 63.55 ± 28.76 | 51.21 ± 13.10 |
| Kₑₗ (hr⁻¹) | 1.32E−02 ± 3.89E−03 | 1.21E−02 ± 3.18E−03 | 1.43E−02 ± 3.41E−03 |
| MRT (hr) | 68.01 ± 13.43 | 72.06 ± 19.85 | 64.70 ± 11.64 |
| M/P Ratio | 4.83 ± 1.29 | 4.99 ± 1.44 | 4.47 ± 1.39 |

*median (min-max)

Relative Bioavailability Assessments For
Bupropion Threoamino Alcohol (A vs C)

| Parameter | 90% C.I. | Ratio of Means | Intra-Subject CV |
|---|---|---|---|
| AUC₀₋ₜ | 105.72% to 159.52% | 129.86% | 24.91% |
| AUC_{0-inf} | 105.55% to 159.53% | 129.76% | 25.02% |
| Cₘₐₓ | 98.32% to 144.38% | 119.14% | 23.22% |

Relative Bioavailability Assessments For
Bupropion Threoamino Alcohol (B vs C)

| Parameter | 90% C.I. | Ratio of Means | Intra-Subject CV |
|---|---|---|---|
| AUC₀₋ₜ | 113.69% to 161.90% | 135.67% | 24.91% |
| AUC_{0-inf} | 114.61% to 163.45% | 136.87% | 25.02% |
| Cₘₐₓ | 98.72% to 137.35% | 116.45% | 23.22% |

Relative Bioavailability Assessments For
Bupropion Threoamino Alcohol (B vs A)

| Parameter | Ratio of Means |
|---|---|
| AUC₀₋ₜ | 104.47% |
| AUC_{0-inf} | 105.48% |
| Cₘₐₓ | 97.74% |

Pharmacokinetic Parameters for Bupropion Erythroamino Alcohol

| | Geometric Mean (% CV) | | | | | |
|---|---|---|---|---|---|---|
| | Arithmetic Mean ± SD | | | | | |
| | Pharmaceutical | | Pharmaceutical | | Bupropion HBr XL | |
| | Composition according | | Composition according | | 348 mg Tablets | |
| | to Example 3 | | to Example 2 | | and Lexapro ® | |
| Pharmacokinetic | [Formulation A] | | [Formulation B] | | 20 mg Tablets | |
| Parameters | (A) (n = 14) | | (B) (n = 14) | | (C) (n = 14) | |
| AUC₀₋ₜ | 1399.37 | (39.81) | 1505.89 | (34.93) | 1088.96 | (36.24) |
| (ng · hr/mL) | 1509.83 ± 601.09 | | 1609.12 ± 562.08 | | 1164.97 ± 422.23 | |
| AUC_{0-inf} | 1468.79 | (37.99) | 1574.95 | (33.82) | 1162.77 | (34.29) |
| (ng · hr/mL) | 1573.77 ± 597.87 | | 1674.54 ± 566.25 | | 1233.20 ± 422.86 | |
| Cₘₐₓ (ng/mL) | 25.82 | (26.80) | 26.94 | (23.10) | 21.56 | (28.77) |
| | 26.73 ± 7.16 | | 27.68 ± 6.39 | | 22.43 ± 6.45 | |
| Tₘₐₓ (hr)* | 10.00 | (8.00-24.00) | 12.00 | (10.00-16.05) | 8.00 | (4.00-16.07) |

| | | | |
|---|---|---|---|
| t_{1/2} (hr) | 33.08 ± 4.13 | 31.82 ± 4.10 | 31.40 ± 4.26 |
| Kₑₗ (hr⁻¹) | 2.12E−02 ± 2.56E−03 | 2.21E−02 ± 3.03E−03 | 2.25E−02 ± 3.13E−03 |
| MRT (hr) | 53.61 ± 7.20 | 53.12 ± 7.36 | 50.14 ± 6.28 |
| M/P Ratio | 0.92 ± 0.24 | 0.95 ± 0.28 | 0.83 ± 0.26 |

*median (min-max)

Relative Bioavailability Assessments For
Bupropion Erythroamino Alcohol (A vs C)

| Parameter | 90% C.I. | Ratio of Means | Intra-Subject CV |
|---|---|---|---|
| AUC₀₋ₜ | 108.01% to 168.46% | 134.89% | 26.98% |
| AUC_{0-inf} | 107.24% to 163.72% | 132.50% | 25.64% |
| Cₘₐₓ | 105.93% to 145.48% | 124.14% | 19.09% |

Relative Bioavailability Assessments For
Bupropion Erythroamino Alcohol (B vs C)

| Parameter | 90% C.I. | Ratio of Means | Intra-Subject CV |
|---|---|---|---|
| AUC₀₋ₜ | 117.39% to 171.99% | 142.09% | 26.98% |
| AUC_{0-inf} | 116.01% to 166.87% | 139.13% | 25.64% |
| Cₘₐₓ | 111.22% to 146.08% | 127.46% | 19.09% |

Relative Bioavailability Assessments For
Bupropion Erythroamino Alcohol (B vs A)

| Parameter | Ratio of Means |
|---|---|
| AUC₀₋ₜ | 105.34% |
| AUC_{0-inf} | 105.00% |
| Cₘₐₓ | 102.67% |

Pharmacokinteic Parameters for PAWC

| | Geometric Mean (% CV) | | | | | |
|---|---|---|---|---|---|---|
| | Arithmetic Mean ± SD | | | | | |
| | Pharmaceutical | | Pharmaceutical | | Bupropion HBr XL | |
| | Composition of | | Composition of | | 348 mg Tablets | |
| | Example 3 | | Example 2 Tablets | | and Lexapro ® | |
| Pharmacokinetic | [Formulation A] | | [Formulation B] | | 20 mg Tablets | |
| Parameters | (A) (n = 14) | | (B) (n = 14) | | (C) (n = 14) | |
| AUC₀₋ₜ | 67.86 | (34.18) | 72.59 | (26.77) | 61.56 | (23.84) |
| (μM · hr) | 71.85 ± 24.56 | | 75.17 ± 20.13 | | 63.56 ± 15.15 | |
| AUC_{0-inf} | 67.99 | (34.11) | 72.80 | (26.60) | 61.65 | (23.84) |
| (μM · hr) | 71.96 ± 24.55 | | 75.35 ± 20.04 | | 63.64 ± 15.17 | |
| Cₘₐₓ (μM) | 1.59 | (25.87) | 1.61 | (21.37) | 1.60 | (24.85) |
| | 1.65 ± 0.43 | | 1.65 ± 0.35 | | 1.66 ± 0.41 | |
| Tₘₐₓ (hr)* | 8.02 | (4.00-24.00) | 8.00 | (4.12-12.09) | 6.00 | (4.00-10.00) |

| | | | |
|---|---|---|---|
| t_{1/2} (hr) | 43.58 ± 23.09 | 47.19 ± 35.90 | 38.34 ± 21.55 |
| Kₑₗ (hr⁻¹) | 2.06E−02 ± 1.06E−02 | 2.07E−02 ± 9.86E−03 | 2.29E−02 ± 1.03E−02 |
| MRT (hr) | 41.30 ± 5.02 | 42.27 ± 5.73 | 39.08 ± 5.09 |

*median (min-max)

Relative Bioavailability Assessments For PAWC (A vs C)

| Parameter | 90% C.I. | Ratio of Means | Intra-Subject CV |
|---|---|---|---|
| AUC₀₋ₜ | 98.11% to 137.79% | 116.27% | 20.47% |
| AUC_{0-inf} | 98.17% to 137.83% | 116.33% | 20.45% |
| Cₘₐₓ | 92.17% to 118.78% | 104.63% | 15.21% |

Relative Bioavailability Assessments For PAWC (B vs C)

| Parameter | 90% C.I. | Ratio of Means | Intra-Subject CV |
|---|---|---|---|
| AUC₀₋ₜ | 105.00% to 140.60% | 121.50% | 20.47% |
| AUC_{0-inf} | 105.17% to 140.77% | 121.67% | 20.45% |
| Cₘₐₓ | 92.74% to 115.31% | 103.41% | 15.21% |

Relative Bioavailability Assessments For PAWC (B vs A)

| Parameter | Ratio of Means |
|---|---|
| AUC₀₋ₜ | 104.50% |
| AUC_{0-inf} | 104.60% |
| Cₘₐₓ | 98.83% |

Pharmacokinetic Parameters for Escitalopram

| | Geometric Mean (% CV) | | | | | | | |
|---|---|---|---|---|---|---|---|---|
| | Arithmetic Mean ± SD | | | | | | | |
| | Pharmaceutical | | Pharmaceutical | | Bupropion HBr XL | | | |
| | Composition according | | Composition according | | 348 mg Tablets | | | |
| | to Example 3 | | to Example 2 | | and Lexapro ® | | Lexapro ® | |
| Pharmacokinetic | [Formulation A] | | [Formulation B] | | 20 mg Tablets | | (20 mg) | |
| Parameters | (A) (n = 13) | | (B) (n = 13) | | (C) (n = 13) | | (D) (n = 13) | |
| AUC₀₋ₜ | 1007.64 | (33.18) | 1036.88 | (32.87) | 1083.76 | (26.12) | 809.59 | (35.50) |
| (ng · hr/mL) | 1062.98 ± 352.70 | | 1087.81 ± 357.52 | | 1120.42 ± 292.62 | | 858.14 ± 304.67 | |
| AUC_{0-inf} | 1025.87 | (32.88) | 1055.23 | (32.55) | 1101.96 | (25.84) | 828.03 | (35.21) |
| (ng · hr/mL) | 1080.89 ± 355.43 | | 1106.25 ± 360.14 | | 1138.28 ± 294.09 | | 876.81 ± 308.72 | |
| Cₘₐₓ (ng/mL) | 16.11 | (24.35) | 16.87 | (20.54) | 25.13 | (21.81) | 22.06 | (25.13) |
| | 16.57 ± 4.03 | | 17.18 ± 3.53 | | 25.68 ± 5.60 | | 22.68 ± 5.70 | |
| Tₘₐₓ (hr)* | 12.00 | (6.00-24.00) | 16.00 | (6.00-24.00) | 4.00 | (2.00-6.00) | 4.00 | (2.00-6.00) |

| | | | | |
|---|---|---|---|---|
| t_{1/2} (hr) | 35.81 ± 6.28 | 36.58 ± 5.81 | 37.33 ± 6.15 | 37.90 ± 10.63 |
| Kₑₗ (hr⁻¹) | 1.98E−02 ± 3.02E−03 | 1.94E−02 ± 3.08E−03 | 1.90E−02 ± 3.22E−03 | 1.93E−02 ± 4.30E−03 |
| MRT (hr) | 61.61 ± 9.50 | 60.09 ± 9.16 | 53.90 ± 8.73 | 48.79 ± 10.27 |

*median (max-min)

Relative Bioavailability Assessments For Escitalopram (A vs C)

| Parameter | 90% C.I. | Ratio of Means | Intra-Subject CV |
|---|---|---|---|
| AUC₀₋ₜ | 86.89% to 100.30% | 93.35% | 10.68% |
| AUC_{0-inf} | 87.07% to 100.31% | 93.45% | 10.53% |
| Cₘₐₓ | 58.98% to 69.86% | 64.19% | 12.62% |

Relative Bioavailability Assessments For Escitalopram (A vs D)

| Parameter | 90% C.I. | Ratio of Means | Intra-Subject CV |
|---|---|---|---|
| AUC₀₋ₜ | 110.27% to 127.91% | 118.76% | 10.68% |
| AUC_{0-inf} | 109.85% to 127.17% | 118.19% | 10.53% |
| Cₘₐₓ | 65.45% to 77.98% | 71.44% | 12.62% |

Relative Bioavailability Assessments For Escitalopram (B vs C)

| Parameter | 90% C.I. | Ratio of Means | Intra-Subject CV |
|---|---|---|---|
| AUC₀₋ₜ | 93.50% to 108.46% | 100.70% | 10.68% |
| AUC_{0-inf} | 93.65% to 108.42% | 100.76% | 10.53% |
| Cₘₐₓ | 64.37% to 76.69% | 70.26% | 12.62% |

Relative Bioavailability Assessments For Escitalopram (B vs D)

| Parameter | 90% C.I. | Ratio of Means | Intra-Subject CV |
|---|---|---|---|
| AUC₀₋ₜ | 119.24% to 137.64% | 128.11% | 10.68% |
| AUC_{0-inf} | 118.73% to 136.78% | 127.43% | 10.53% |
| Cₘₐₓ | 71.85% to 85.11% | 78.20% | 12.62% |

Relative Bioavailability Assessments For Escitalopram (C vs D)

| Parameter | 90% C.I. | Ratio of Means | Intra-Subject CV |
|---|---|---|---|
| AUC₀₋ₜ | 118.12% to 137.02% | 127.22% | 10.68% |
| AUC_{0-inf} | 117.54% to 136.07% | 126.47% | 10.53% |
| Cₘₐₓ | 101.97% to 121.48% | 111.30% | 12.62% |

Relative Bioavailability Assessments For Escitalopram (B vs A)

| Parameter | Ratio of Means |
|---|---|
| AUC₀₋ₜ | 107.87% |
| AUC_{0-inf} | 107.82% |
| Cₘₐₓ | 109.47% |

Pharmacokinetic Parameters for S-Demethylcitalopram

| | Geometric Mean (% CV) | | | | | | | |
|---|---|---|---|---|---|---|---|---|
| | Arithmetic Mean ± SD | | | | | | | |
| | Pharmaceutical | | Pharmaceutical | | Bupropion HBr XL | | | |
| | Composition according | | Composition according | | 348 mg Tablets | | | |
| | to Example 3 | | to Example 2 | | and Lexapro ® | | Lexapro ® | |
| Pharmacokinetic | [Formulation A] | | [Formulation B] | | 20 mg Tablets | | (20 mg) | |
| Parameters | (A) (n = 13) | | (B) (n = 13) | | (C) (n = 13) | | (D) (n = 13) | |
| AUC₀₋ₜ | 264.27 | (35.79) | 271.43 | (25.68) | 314.78 | (30.02) | 330.98 | (22.37) |
| (ng · hr/mL) | 276.25 ± 98.88 | | 278.68 ± 71.55 | | 325.97 ± 97.86 | | 337.85 ± 75.58 | |
| AUC_{0-inf} | 279.36 | (36.24) | 286.48 | (25.13) | 329.65 | (29.24) | 345.20 | (22.67) |
| (ng · hr/mL) | 292.24 ± 105.91 | | 293.73 ± 73.81 | | 340.74 ± 99.64 | | 352.43 ± 79.88 | |
| Cₘₐₓ (ng/mL) | 1.82 | (34.15) | 1.93 | (31.30) | 2.53 | (30.27) | 2.96 | (24.35) |
| | 1.91 ± 0.65 | | 2.01 ± 0.63 | | 2.64 ± 0.80 | | 3.04 ± 0.74 | |
| Tₘₐₓ (hr)* | 36.00 | (16.00-72.03) | 48.00 | (16.00-96.00) | 10.00 | (3.00-48.02) | 16.00 | (2.00-36.32) |

| | | | | |
|---|---|---|---|---|
| t_{1/2} (hr) | 54.73 ± 8.35 | 55.43 ± 7.00 | 54.29 ± 8.75 | 54.14 ± 12.30 |
| Kₑₗ (hr⁻¹) | 1.30E−02 ± 2.19E−03 | 1.27E−02 ± 1.60E−03 | 1.31E−02 ± 2.21E−03 | 1.34E−02 ± 2.83E−03 |
| MRT (hr) | 114.39 ± 13.77 | 112.08 ± 12.71 | 100.93 ± 13.69 | 93.96 ± 16.50 |
| M/P ratio | 0.30 ± 0.11 | 0.30 ± 0.12 | 0.33 ± 0.12 | 0.47 ± 0.18 |

*median (max-min)

Relative Bioavailability Assessments
For S-demethylcitalopram (A vs C)

| Parameter | 90% C.I. | Ratio of Means | Intra-Subject CV |
|---|---|---|---|
| AUC₀₋ₜ | 79.52% to 90.65% | 84.90% | 9.75% |
| AUC_{0-inf} | 80.15% to 91.57% | 85.67% | 9.91% |
| Cₘₐₓ | 68.89% to 78.81% | 73.68% | 10.01% |

Relative Bioavailability Assessments
For S-demethylcitalopram (A vs D)

| Parameter | 90% C.I. | Ratio of Means | Intra-Subject CV |
|---|---|---|---|
| AUC₀₋ₜ | 73.81% to 84.53% | 78.99% | 9.75% |
| AUC_{0-inf} | 74.61% to 85.64% | 79.93% | 9.91% |
| Cₘₐₓ | 57.37% to 65.93% | 61.50% | 10.01% |

Relative Bioavailability Assessments
For S-demethylcitalopram (B vs C)

| Parameter | 90% C.I. | Ratio of Means | Intra-Subject CV |
|---|---|---|---|
| AUC₀₋ₜ | 82.77% to 94.78% | 88.57% | 9.75% |
| AUC_{0-inf} | 83.29% to 95.60% | 89.23% | 9.91% |
| Cₘₐₓ | 73.35% to 84.30% | 78.64% | 10.01% |

Relative Bioavailability Assessments
For S-demethylcitalopram (B vs D)

| Parameter | 90% C.I. | Ratio of Means | Intra-Subject CV |
|---|---|---|---|
| AUC₀₋ₜ | 77.17% to 87.98% | 82.40% | 9.75% |
| AUC_{0-inf} | 77.89% to 88.99% | 83.26% | 9.91% |
| Cₘₐₓ | 61.37% to 70.20% | 65.64% | 10.01% |

Relative Bioavailability Assessments
For S-demethylcitalopram (C vs D)

| Parameter | 90% C.I. | Ratio of Means | Intra-Subject CV |
|---|---|---|---|
| AUC₀₋ₜ | 86.94% to 99.55% | 93.03% | 9.75% |
| AUC_{0-inf} | 87.09% to 99.96% | 93.30% | 9.91% |
| Cₘₐₓ | 77.86% to 89.48% | 83.47% | 10.01% |

Relative Bioavailability Assessments
For S-demethylcitalopram (B vs A)

| Parameter | Ratio of Means |
|---|---|
| AUC₀₋ₜ | 104.32% |
| AUC_{0-inf} | 104.16% |
| Cₘₐₓ | 106.73% |

Pharmacokinetic Parameters for S-Didemethylcitalopram

| | Geometric Mean (% CV) | | | | | | | |
|---|---|---|---|---|---|---|---|---|
| | Arithmetic Mean ± SD | | | | | | | |
| | Pharmaceutical | | Pharmaceutical | | Bupropion HBr XL | | | |
| | Composition according | | Composition according | | 348 mg Tablets | | | |
| | to Example 3 | | to Example 2 | | and Lexapro ® | | Lexapro ® | |
| Pharmacokinetic | [Formulation A] | | [Formulation B] | | 20 mg Tablets | | (20 mg) | |
| Parameters | (A) (n = 13) | | (B) (n = 13) | | (C) (n = 13) | | (D) (n = 13) | |
| AUC₀₋ₜ | 17.86 | (67.01) | 18.83 | (58.38) | 25.50 | (68.89) | 47.34 | (69.19) |
| (ng · hr/mL) | 23.37 ± 15.66 | | 22.83 ± 13.33 | | 31.22 ± 21.51 | | 62.42 ± 43.19 | |
| AUC_{0-inf} | 27.65 | (33.72)† | 35.84 | (38.51)‡ | 35.91 | (69.32)$ | 66.16 | (58.94) |
| (ng · hr/mL) | 29.01 ± 9.78 | | 37.83 ± 14.57 | | 42.67 ± 29.57 | | 76.22 ± 44.92 [symbol] | |
| Cₘₐₓ (ng/mL) | 0.10 | (61.69) | 0.11 | (52.10) | 0.14 | (62.79) | 0.30 | (68.55) |
| | 0.12 ± 0.08 | | 0.12 ± 0.06 | | 0.17 ± 0.10 | | 0.38 ± 0.26 | |
| Tₘₐₓ (hr)* | 120.00 | (72.00-168.00) | 120.02 | (72.00-168.10) | 96.00 | (12.02-168.00) | 72.00 | (36.00-96.00) |

| | | | | |
|---|---|---|---|---|
| t_{1/2} (hr) | 69.57 ± 2.11† | 84.44 ± 11.68‡ | 74.45 ± 14.23$ | 63.52 ± 10.01 [symbol] |
| Kₑₗ (hr⁻¹) | 9.97E−03 ± 3.11E−04† | 8.35E−03 ± 1.22E−03‡ | 9.65E−03 ± 2.08E−03$ | 1.12E−02 ± 1.87E−03 [symbol] |
| MRT (hr) | 171.76 ± 14.40† | 187.03 ± 12.54‡ | 166.30 ± 19.71$ | 135.38 ± 9.77 [symbol] |
| M/P ratio | 0.04 ± 0.01† | 0.05 ± 0.02‡ | 0.05 ± 0.03$ | 0.12 ± 0.08 [symbol] |

*median (max-min);
†n = 4;
‡n = 6;
$n = 8; [symbol]
n = 11

Relative Bioavailability Assessments
For S-didemethylcitalopram (A vs C)

| Parameter | 90% C.I. | Ratio of Means | Intra-Subject CV |
|---|---|---|---|
| AUC₀₋ₜ | 64.15% to 81.95% | 72.50% | 18.33% |
| AUC_{0-inf} | 64.25% to 98.83% | 79.68% | 14.60% |
| Cₘₐₓ | 61.74% to 90.43% | 74.72% | 28.89% |

Relative Bioavailability Assessments
For S-didemethylcitalopram (A vs D)

| | Potency Uncorrected Data | | |
|---|---|---|---|
| Parameter | 90% C.I. | Ratio of Means | Intra-Subject CV |
| AUC₀₋ₜ | 34.86% to 44.91% | 39.57% | 18.33% |
| AUC_{0-inf} | 39.46% to 58.94% | 48.23% | 14.60% |
| Cₘₐₓ | 31.31% to 46.46% | 38.14% | 28.89% |

Relative Bioavailability Assessments
For S-didemethylcitalopram (B vs C)

| Parameter | 90% C.I. | Ratio of Means | Intra-Subject CV |
|---|---|---|---|
| AUC₀₋ₜ | 65.92% to 84.92% | 74.82% | 18.33% |
| AUC_{0-inf} | 60.78% to 87.61% | 72.97% | 14.60% |
| Cₘₐₓ | 60.68% to 90.05% | 73.92% | 28.89% |

Relative Bioavailability Assessments
For S-didemethylcitalopram (B vs D)

| Parameter | 90% C.I. | Ratio of Means | Intra-Subject CV |
|---|---|---|---|
| AUC₀₋ₜ | 36.12% to 46.15% | 40.83% | 18.33% |
| AUC_{0-inf} | 38.05% to 51.26% | 44.16% | 14.60% |
| Cₘₐₓ | 31.18% to 45.67% | 37.73% | 28.89% |

Relative Bioavailability Assessments
For S-didemethylcitalopram (C vs D)

| Parameter | 90% C.I. | Ratio of Means | Intra-Subject CV |
|---|---|---|---|
| AUC₀₋ₜ | 48.08% to 61.94% | 54.57% | 18.33% |
| AUC_{0-inf} | 52.67% to 69.55% | 60.52% | 14.60% |
| Cₘₐₓ | 41.91% to 62.18% | 51.05% | 28.89% |

Relative Bioavailability Assessments
For S-didemethylcitalopram (B vs A)

| Parameter | Ratio of Means |
|---|---|
| AUC₀₋ₜ | 103.20% |
| AUC_{0-inf} | 91.57% |
| Cₘₐₓ | 98.93% |

### Example 26

### A Pilot Three-Way, Single-Dose, Fasting Study of a Pharmaceutical Composition of the Invention Comprising Bupropion HBr (325 Mg)/Escitalopram Oxalate (17.9 mg)

This was a 3-way crossover, randomized, open-label, single-dose, fasting, pilot, Phase I (comparative bioavailability) study. The objective of this study was to evaluate the relative peak and systemic exposure of a test formulation of the pharmaceutical composition manufactured according to Example 10 to Bupropion HBr Extended Release (XL) 348 mg Tablets described in U.S. Pat. No. 7,241,805 and Lexapro® 20 mg Tablets when co-admninistered under fasting conditions. Normal, healthy, non-smoking male and female subjects between the ages of 18 and 55 years were included in the study.

The test formulation used was one (1) pharmaceutical composition of Example 10, Batch #: 0707050, administered orally. The reference formulation was One (1) Bupropion HBr XL 348 mg Tablet as described in U.S. Pat. No. 7,241,805 Batch #: 07H044P, administered orally and one (1) Lexapro® 20 mg Tablet, Lot #: M0603M, administered orally.

There were 18 subjects dosed in Period I, 13 of whom completed the study. One subject was dismissed because of an adverse event (AE, decreased hemoglobin), 1 subject was dismissed after emesis within 24.00 hours of dosing with Treatment B, and 3 subjects withdrew for personal reasons. Pharmacokinetic and statistical analyses were performed on 13 subjects who completed the study for bupropion and its metabolites. Pharmacokinetic and statistical analyses were performed on 14 subjects (13 subjects who completed the study and 1 subject for whom there were sufficient data for a meaningful analysis) for escitalopram and its metabolite.

During each study period, 23 blood samples were collected from each subject at the following timepoints: 0.00 (pre-dose), and at 1.00, 2.00, 3.00, 4.00, 6.00, 8.00, 10.00, 12.00, 16.00, 24.00, 36.00, 48.00, 72.00, 96.00, 120.00, 144.00, 168.00, 192.00, 216.00, 240.00, 264.00 and 288.00 hours post-dose.

Pharmacokinetic and statistical analyses were performed on 13 subjects who completed the study for bupropion and its metabolites. Pharmacokinetic and statistical analyses were performed on 14 subjects (13 subjects who completed the study and 1 subject for whom there were sufficient data for a meaningful analysis) for escitalopram and its metabolites. The safety assessment was performed on all subjects who received at least 1 dose during the course of the study.

The following pharmacokinetic parameters for bupropion, hydroxybupropion, bupropion threoamino alcohol, bupropion erythroamino alcohol, Pharmacologic Activity-Weighted Composite (PAWC), escitalopram, S-demethylcitalopram, and S-didemethylcitalopram were calculated by standard non-compartmental methods: AUC₀₋ₜ, AUC_{0-inf}, Cₘₐₓ, Tₘₐₓ, t_{1/2}, Kₑₗ, MRT, and M/P ratio.

Bupropion, hydroxybupropion, bupropion erythroamino alcohol, bupropion threoamino alcohol, and the internal standard, 1-(3-chlorophenyl)-piperazine, were extracted from human plasma (0.50 mL), by solid phase extraction (SPE) into an organic medium. The analytes were separated by High Performance Liquid Chromatography (HPLC) system using reverse phase chromatography conditions, detected using an API 3000 tandem mass spectrometer. Method sensitivity and selectivity were achieved by detecting distinct precursor to production mass transitions for bupropion (240.3→184.0), hydroxybupropion (256.3→238.0), bupropion erythroamino alcohol (242.4→168.1), bupropion threoamino alcohol (242.4→168.1) and the internal standard, 1-(3-chlorophenyl)-piperazine (197.3→153.8), at defined retention time.

For standards prepared on Jul. 25, 2007, evaluation of the assay, using defined acceptance criteria, was carried out by the construction of an eight (8) point calibration curve (excluding zero concentration) covering the range of 1.000 ng/mL to 1023.900 ng/mL for bupropion, 3.905 ng/mL to 3998.970 ng/mL for hydroxybupropion, 1.000 ng/mL to 1024.000 ng/mL for bupropion erythroamino alcohol, and 1.000 ng/mL to 1024.000 ng/mL for bupropion threoamino alcohol in human plasma. For standards prepared on Sep. 27, 2007, evaluation of the assay, using defined acceptance criteria, was carried out by the construction of an eight (8) point calibration curve (excluding zero concentration) covering the range of 1.000 ng/mL to 1023.900 ng/mL for bupropion, 3.907 ng/mL to 4000.320 ng/mL for hydroxybupropion, 1.000 ng/mL to 1024.000 ng/mL for bupropion erythroamino alcohol, and 1.000 ng/mL to 1024.000 ng/mL for bupropion threoamino alcohol in human plasma. The slope and intercept of the calibration curves were determined through weighted linear regression analysis (1/peak area ratio²). Two calibration curves and duplicate QC samples (at three or five concentration levels) were analyzed along with each batch of the study samples. Peak area ratios were used to determine the concentration of the standards, quality control samples, and the unknown study samples from the calibration curves. The Concentration of escitalopram, S-demethylcitalopram and S-didemethylcitalopram were measured using an archiral LC-MS/MS method for citalopram, demethylcitalopram, didemethylcitalopram. citalopram, demethylcitalopram, didemethylcitalopram and the internal standards, citalopram analog, demethylcitalopram analog, and didemethylcitalopram analog, were extracted from human plasma (0.75 mL), using sodium heparin as an anticoagulant, by liquid-liquid extraction into an organic medium followed by back extraction into a dilute acid. An aliquot of this extract was injected into a High Performance Liquid Chromatography system and detected using a TSQ Quantum tandem mass spectrometer. The analytes were separated by reverse phase chromatography. Method sensitivity and selectivity were achieved by detecting distinct precursor to production mass transitions for citalopram (325.1→109.0), demethylcitalopram (311.1→109.0), didemethylcitalopram (297.1→109.0 and 297.1→260.0), and the internal standards, citalopram analog (341.1→125.0), demethylcitalopram analog (327.1→125.0), and didemethylcitalopram analog (313.1→125.0), at defined retention time.

Evaluation of the assay, using defined acceptance criteria, was carried out by the construction of an eight (8) point calibration curve (excluding zero concentration) covering the range of 0.251 ng/mL to 64.020 ng/mL for citalopram, 0.126 ng/mL to 32.013 ng/mL for demethylcitalopram, and 0.025 ng/mL to 6.397 ng/mL for didemethylcitalopram in human plasma. The slope and intercept of the calibration curves were determined through weighted linear regression analysis (1/conc.²). Two calibration curves and duplicate QC samples (at three or four concentration levels) were analyzed along with each batch of the study samples. Peak area ratios were used to determine the concentration of the standards, quality control samples, and the unknown study samples from the calibration curves.

Pharmacokinetic and statistical analyses were performed on 13 subjects who completed the study for bupropion and its metabolites. Pharmacokinetic and statistical analyses were performed on 14 subjects (13 subjects who completed the study and 1 subject for whom there were sufficient data for a meaningful analysis) for escitalopram and its metabolites. The safety assessment was performed on all subjects who received at least 1 dose during the course of the study.

The following pharmacokinetic parameters for bupropion, hydroxybupropion, bupropion threoamino alcohol, bupropion erythroamino alcohol, Pharmacologic Activity-Weighted Composite (PAWC), escitalopram, S-demethylcitalopram, and S-didemethylcitalopram were calculated by standard non-compartmental methods: AUC₀₋ₜ, AUC_{0-inf}, Cₘₐₓ, Tₘₐₓ, t_{1/2}, Kₑₗ, MRT, and M/P ratio.

Data for the pharmcokinetic parameters for bupropion and citalopram and their metabolites is presented in the tables below and in FIGS. 26A-H.

Pharmacokinetic Parameters for Bupropion

| | Geometric Mean (% CV) | | | |
|---|---|---|---|---|
| | Arithmetic Mean ± SD | | | |
| | Pharmaceutical | | | |
| | Composition according | | Bupropion HBr XL | |
| | to Example 10 (A) | | 348 mg Tablets (B) | |
| Pharmacokinetic | (n = 13) | | (n = 13) | |
| Parameters | Batch #: 0707050 | | Batch #: 07H044P | |
| AUC₀₋ₜ | 1540.44 | (42.08) | 1306.14 | (24.03) |
| (ng · hr/mL) | 1682.56 ± 708.04 | | 1342.18 ± 322.57 | |
| AUC_{0-inf} | 1588.39 | (40.98) | 1363.51 | (22.80) |
| (ng · hr/mL) | 1726.27 ± 707.35 | | 1396.89 ± 318.46 | |
| Cₘₐₓ (ng/mL) | 114.98 | (30.47) | 103.18 | (29.69) |
| | 119.83 ± 36.51 | | 108.30 ± 32.16 | |
| Tₘₐₓ (hr)* | 4.00 | (3.00-6.02) | 4.00 | (3.00-6.00) |

| | | |
|---|---|---|
| t_{1/2} (hr) | 21.71 ± 6.75 | 23.24 ± 7.87 |
| Kₑₗ (hr⁻¹) | 3.46E−02 ± 9.52E−03 | 3.29E−02 ± 1.03E−02 |
| MRT (hr) | 19.98 ± 4.42 | 23.13 ± 5.50 |

*median (min-max)

Relative Bioavailability Assessments for Bupropion (A vs B)

| Parameter | 90% C.I. | Ratio of Means | Intra-Subject CV |
|---|---|---|---|
| AUC₀₋ₜ | 99.28% to 136.83% | 116.55% | 22.43% |
| AUC_{0-inf} | 98.58% to 134.69% | 115.23% | 21.81% |
| Cₘₐₓ | 96.59% to 124.44% | 109.63% | 17.63% |

Pharmacokinetic Parameters for Hydroxybupropion

| | Geometric Mean (% CV) | | | |
|---|---|---|---|---|
| | Arithmetic Mean ± SD | | | |
| | Pharmaceutical | | | |
| | Composition According | | Bupropion HBr XL | |
| | to Example 10 (A) | | 348 mg Tablets (B) | |
| Pharmacokinetic | (n = 13) | | (n = 13) | |
| Parameters | Batch #: 0707050 | | Batch #: 07H044P | |
| AUC₀₋ₜ | 25490.93 | (43.68) | 23053.82 | (36.28) |
| (ng · hr/mL) | 28065.86 ± 12259.79 | | 24480.15 ± 8881.54 | |
| AUC_{0-inf} | 25732.33 | (43.33) | 23288.15 | (36.18) |
| (ng · hr/mL) | 28286.16 ± 12257.45 | | 24717.94 ± 8943.32 | |
| Cₘₐₓ (ng/mL) | 531.88 | (30.91) | 443.18 | (33.63) |
| | 556.01 ± 171.88 | | 464.00 ± 156.03 | |
| Tₘₐₓ (hr)* | 10.00 | (6.00-24.00) | 10.05 | (6.00-16.00) |

| | | |
|---|---|---|
| t_{1/2} (hr) | 26.74 ± 5.19 | 26.26 ± 5.38 |
| Kₑₗ (hr⁻¹) | 2.68E−02 ± 4.79E−03 | 2.73E−02 ± 4.83E−03 |
| MRT (hr) | 42.91 ± 5.69 | 43.87 ± 6.50 |
| M/P Ratio | 15.47 ± 3.03 | 16.47 ± 3.85 |

*median (min-max)

Relative Bioavailability Assessments
For Hydroxybupropion (A vs B)

| Parameter | 90% C.I. | Ratio of Means | Intra-Subject CV |
|---|---|---|---|
| AUC₀₋ₜ | 93.58% to 127.93% | 109.41% | 21.85% |
| AUC_{0-inf} | 93.52% to 127.76% | 109.31% | 21.80% |
| Cₘₐₓ | 105.36% to 136.24% | 119.81% | 17.90% |

Pharmacokinetic Parameters for Bupropion Threoamino Alcohol

| | Geometric Mean (% CV) | | | |
|---|---|---|---|---|
| | Arithmetic Mean ± SD | | | |
| | Pharmaceutical | | | |
| | Composition According | | Bupropion HBr XL | |
| | to Example 10 (A) | | 348 mg Tablets (B) | |
| Pharmacokinetic | (n = 13) | | (n = 13) | |
| Parameters | Batch #: 0707050 | | Batch #: 07H044P | |
| AUC₀₋ₜ | 7079.68 | (53.12) | 6911.03 | (22.19) |
| (ng · hr/mL) | 8074.61 ± 4289.60 | | 7074.21 ± 1569.85 | |
| AUC_{0-inf} | 7350.60 | (52.90) | 7106.82 | (22.14) |
| (ng · hr/mL) | 8387.70 ± 4437.17 | | 7273.18 ± 1610.27 | |
| Cₘₐₓ (ng/mL) | 145.38 | (45.89) | 128.08 | (28.91) |
| | 159.05 ± 72.99 | | 133.02 ± 38.46 | |
| Tₘₐₓ (hr)* | 10.00 | (6.00-12.02) | 8.00 | (3.00-10.00) |

| | | |
|---|---|---|
| t_{1/2} (hr) | 60.92 ± 19.38 | 56.08 ± 13.20 |
| Kₑₗ (hr⁻¹) | 1.27E−02 ± 4.82E−03 | 1.31E−02 ± 3.53E−03 |
| MRT (hr) | 74.50 ± 18.13 | 72.24 ± 16.32 |
| M/P Ratio | 4.77 ± 1.40 | 5.28 ± 1.14 |

*median (min-max)

Relative Bioavailability Assessments For
Bupropion Threoamino Alcohol (A vs B)

| Parameter | 90% C.I. | Ratio of Means | Intra-Subject CV |
|---|---|---|---|
| AUC₀₋ₜ | 79.64% to 127.13% | 100.62% | 33.16% |
| AUC_{0-inf} | 80.41% to 127.84% | 101.39% | 32.86% |
| Cₘₐₓ | 89.70% to 138.99% | 111.66% | 30.95% |

Pharmacokinetic Parameters for Bupropion Erythroamino Alcohol

| | Geometric Mean (% CV) | | | |
|---|---|---|---|---|
| | Arithmetic Mean ± SD | | | |
| | Bupropion HBr (325 | | | |
| | mg)/Escitalopram Oxalate | | Bupropion HBr XL | |
| | (17.9 mg) SR Tablets (A) | | 348 mg Tablets (B) | |
| Pharmacokinetic | (n = 13) | | (n = 13) | |
| Parameters | Batch #: 0707050 | | Batch #: 07H044P | |
| AUC₀₋ₜ | 1299.62 | (48.00) | 1313.17 | (22.29) |
| (ng · hr/mL) | 1449.54 ± 695.84 | | 1343.05 ± 299.34 | |
| AUC_{0-inf} | 1370.88 | (46.20) | 1369.92 | (22.09) |
| (ng · hr/mL) | 1517.67 ± 701.16 | | 1400.60 ± 309.40 | |
| Cₘₐₓ (ng/mL) | 25.29 | (33.84) | 23.76 | (22.13) |
| | 26.67 ± 9.03 | | 24.30 ± 5.38 | |
| Tₘₐₓ (hr)* | 10.00 | (6.00-24.00) | 10.00 | (6.00-16.00) |

| | | |
|---|---|---|
| t_{1/2} (hr) | 32.39 ± 5.61 | 29.71 ± 5.49 |
| Kₑₗ (hr⁻¹) | 2.21E−02 ± 4.21E−03 | 2.40E−02 ± 4.13E−03 |
| MRT (hr) | 52.35 ± 8.97 | 51.51 ± 9.53 |
| M/P Ratio | 0.88 ± 0.20 | 1.02 ± 0.22 |

*median (min-max)

Relative Bioavailability Assessments For
Bupropion Erythroamino Alcohol (A vs B)

| Parameter | 90% C.I. | Ratio of Means | Intra-Subject CV |
|---|---|---|---|
| AUC₀₋ₜ | 77.31% to 123.34% | 97.65% | 33.12% |
| AUC_{0-inf} | 78.57% to 123.77% | 98.61% | 32.17% |
| Cₘₐₓ | 86.21% to 126.76% | 104.54% | 27.10% |

Pharmacokinetic Parameters for PAWC

| | Geometric Mean (% CV) | | | |
|---|---|---|---|---|
| | Arithmetic Mean ± SD | | | |
| | Pharmaceutical | | | |
| | Composition According | | Bupropion HBr XL | |
| | to Example 10 (A) | | 348 mg Tablets (B) | |
| Pharmacokinetic | (n = 13) | | (n = 13) | |
| Parameters | Batch #: 0707050 | | Batch #: 07H044P | |
| AUC₀₋ₜ | 73.87 | (42.70) | 67.15 | (31.81) |
| (μM · hr) | 80.98 ± 34.58 | | 70.32 ± 22.37 | |
| AUC_{0-inf} | 74.05 | (42.61) | 67.26 | (31.75) |
| (μM · hr) | 81.16 ± 34.58 | | 70.43 ± 22.36 | |
| Cₘₐₓ (μM) | 1.70 | (29.51) | 1.45 | (34.69) |
| | 1.76 ± 0.52 | | 1.52 ± 0.53 | |
| Tₘₐₓ (hr)* | 10.00 | (4.00-12.02) | 6.00 | (4.00-10.05) |

| | | |
|---|---|---|
| t_{1/2} (hr) | 37.98 ± 23.54 | 35.58 ± 17.38 |
| Kₑₗ (hr⁻¹) | 2.48E−02 ± 1.26E−02 | 2.41E−02 ± 1.09E−02 |
| MRT (hr) | 42.31 ± 5.69 | 43.35 ± 5.86 |

*median (min-max)

Relative Bioavailability Assessments For PAWC (A vs B)

| Parameter | 90% C.I. | Ratio of Means | Intra-Subject CV |
|---|---|---|---|
| AUC₀₋ₜ | 92.92% to 127.38% | 108.79% | 22.05% |
| AUC_{0-inf} | 92.98% to 127.47% | 108.87% | 22.05% |
| Cₘₐₓ | 101.40% to 134.02% | 116.57% | 19.44% |

Pharmacokinetic Parameters For Escitalopram

| | Geometric Mean (% CV) | | | |
|---|---|---|---|---|
| | Arithmetic Mean ± SD | | | |
| | Pharmaceutical | | | |
| | Composition According | | Lexapro ® | |
| | to Examle 10 (A) | | 20 mg Tablets (C) | |
| Pharmacokinetic | (n = 14) | | (n = 14) | |
| Parameters | Batch #: 0707050 | | Lot #: M0603M | |
| AUC₀₋ₜ | 606.10 | (41.90) | 751.78 | (25.80) |
| (ng · hr/mL) | 661.94 ± 277.35 | | 774.39 ± 199.81 | |
| AUC_{0-inf} | 625.05 | (41.30) | 767.40 | (25.59) |
| (ng · hr/mL) | 680.63 ± 281.08 | | 790.08 ± 202.21 | |
| Cₘₐₓ (ng/mL) | 10.79 | (24.93) | | 21.56 (22.86) |
| | 11.14 ± 2.78 | | 22.04 ± 5.04 | |
| Tₘₐₓ (hr)* | 12.00 | (6.00-16.00) | 6.00 | (2.02-8.00) |

| | | |
|---|---|---|
| t_{1/2} (hr) | 36.15 ± 8.36 | 31.85 ± 7.08 |
| Kₑₗ (hr⁻¹) | 2.01E−02 ± 4.45E−03 | 2.29E−02 ± 5.42E−03 |
| MRT (hr) | 56.89 ± 10.51 | 42.51 ± 8.99 |

*median (min-max)

Relative Bioavailability Assessments For Escitalopram (A vs C)

| Parameter | 90% C.I. | Ratio of Means | Intra-Subject CV |
|---|---|---|---|
| AUC₀₋ₜ | 69.49% to 103.47% | 84.80% | 27.18% |
| AUC_{0-inf} | 70.27% to 103.82% | 85.42% | 26.63% |
| Cₘₐₓ | 45.12% to 59.43% | 51.78% | 18.63% |

Pharmacokinetic Parameters For S-demethylcitalopram

| | Geometric Mean (% CV) | | | |
|---|---|---|---|---|
| | Arithmetic Mean ± SD | | | |
| | Bupropion HBr (325 | | | |
| | mg)/Escitalopram Oxalate | | Lexapro ® | |
| | (17.9 mg) SR Tablets (A) | | 20 mg Tablets (C) | |
| Pharmacokinetic | (n = 14) | | (n = 14) | |
| Parameters | Batch #: 0707050 | | Lot #: M0603M | |
| AUC₀₋ₜ | 184.49 | (25.46) | 346.32 | (13.42) |
| (ng · hr/mL) | 191.48 ± 48.75 | | 349.33 ± 46.88 | |
| AUC_{0-inf} | 198.12 | (24.54) | 358.80 | (12.90) |
| (ng · hr/mL) | 204.94 ± 50.29 | | 361.61 ± 46.63 | |
| Cₘₐₓ (ng/mL) | 1.39 | (23.98) | 3.23 | (17.15) |
| | 1.44 ± 0.34 | | 3.27 ± 0.56 | |
| Tₘₐₓ (hr)* | 42.00 | (24.02-72.00) | 16.00 | (8.00-48.00) |

| | | |
|---|---|---|
| t_{1/2} (hr) | 56.20 ± 16.03 | 46.57 ± 8.83 |
| Kₑₗ (hr⁻¹) | 1.33E−02 ± 3.88E−03 | 1.55E−02 ± 3.56E−03 |
| MRT (hr) | 109.23 ± 19.06 | 84.48 ± 15.83 |
| M/P Ratio | 0.34 ± 0.10 | 0.50 ± 0.13 |

*median (min-max)

Relative Bioavailability Assessments For S-demethylcitalopram

| Parameter | 90% C.I. | Ratio of Means | Intra-Subject CV |
|---|---|---|---|
| AUC₀₋ₜ | 48.67% to 61.57% | 54.74% | 15.86% |
| AUC_{0-inf} | 50.64% to 64.01% | 56.93% | 15.81% |
| Cₘₐₓ | 39.85% to 48.34% | 43.89% | 13.00% |

Pharmacokinetic Parameters For S-didemethylcitalopram

| | Geometric Mean (% CV) | | | |
|---|---|---|---|---|
| | Arithmetic Mean ± SD | | | |
| | Pharmaceutical | | | |
| | Composition According | | Lexapro ® | |
| | to Example 10 (A) | | 20 mg Tablets (C) | |
| Pharmacokinetic | (n = 14) | | (n = 14) | |
| Parameters | Batch #: 0707050 | | Lot #: M0603M | |
| AUC₀₋ₜ | 17.80 | (42.15) | 73.99 | (34.64) |
| (ng · hr/mL) | 19.62 ± 8.27 | | 79.34 ± 27.49 | |
| AUC_{0-inf} | 27.31 | (31.63)^{†} | 80.34 | (33.33) |
| (ng · hr/mL) | 28.40 ± 8.98 | | 85.66 ± 28.55 | |
| Cₘₐₓ (ng/mL) | 0.10 | (36.95) | 0.46 | (40.41) |
| | 0.11 ± 0.04 | | 0.51 ± 0.20 | |
| Tₘₐₓ (hr)* | 120.00 | (72.00-144.00) | 72.00 | (36.00-120.00) |

| | | |
|---|---|---|
| t_{1/2} (hr) | 68.43 ± 7.71^{†} | 62.45 ± 14.59 |
| Kₑₗ (hr⁻¹) | 1.03E−02 ± 1.26E−03^{†} | 1.18E−02 ± 3.44E−03 |
| MRT (hr) | 160.29 ± 20.47^{†} | 132.86 ± 22.77 |
| M/P Ratio | 0.06 ± 0.02^{†} | 0.13 ± 0.06 |

*median (min-max);
^{†}n = 7

Relative Bioavailability Assessments for S-demethylcitalopram

| Parameter | 90% C.I. | Ratio of Means | Intra-Subject CV |
|---|---|---|---|
| AUC₀₋ₜ | 21.07% to 26.80% | 23.76% | 16.24% |
| AUC_{0-inf} | 23.62% to 32.01% | 27.49% | 12.80% |
| Cₘₐₓ | 17.91% to 24.71% | 21.04% | 21.84% |

### Comparative Examples

### Example 27

### Homogenous Tablet Core Composition and Method of Manufacture-Separate Granulation Method

A. Tramadol HCl Granulation and Bulk Blend

| | | Tramadol HCl ER Bulk Blend |
|---|---|---|
| Tramadol HCl Granules Composition | | Composition |
| Component | % w/w | % w/w |
| Tramadol HCl | 97.09 | 96.16 |
| Aerosil 200 (Colloidal | 0.97 | 0.96 |
| Silicone Dioxide) | | |
| Polyvinyl alcohol (PVA) | 1.94 | 1.92 |
| Pruv (Sodium Searyl | — | 0.96 |
| Fumarate) | | |

The pharmaceutical active, tramadol HCl, was top-spray granulated using Aeromatic 4/5 Fluid Bed granulator. The theoretical batch size was 66.950 kg. An aqueous (purified water) solution of polyvinyl alcohol (PVA, 4.59% solution) was sprayed onto 65.000 kg of tramadol HCl and 0.650 kg of Aerosil 2000 (colloidal silicone dioxide) to a weight gain of 1.98% to produce granules comprising 97.09% of tramadol HCL, 0.97% Aerosil 200 and 1.94% PVA. The powder bed temperature was maintained between 25-35° C., and the liquid spray was maintained between 150-300 g/min throughout the granulation process. When spraying of the granulation solution was completed, the granules were fluid bed dried to an LOD of <1%. The granules were then sieved through 1.5 mm screen using Sweco sifter. The oversized granules were passed at 1000 rpm through a Comill fitted with 1.57 mm screen. The resulting granules were added to the sifted granules to make one granule batch. These granules were then blended for 10 minutes with 0.635 kg of screened Pruv (sodium stearyl fumarate screened through 0.590 mm size) using a V blender. The total blend batch size was 65.995 kg.

B. Meloxicam Granulation

Meloxicam Granule Composition

| Component | % w/w |
|---|---|
| Meloxicam | 98.74 |
| Polyvinyl alcohol (PVA) | 1.26 |

The pharmaceutical active, meloxicam, was granulated using high shear mixer. The theoretical batch size after granulation was 303.9 g. An aqueous solution of polyvinyl alcohol (4.6% solution) was sprayed onto about 300 g of meloxicam to a weight gain of about 1.3% weight gain to produce granules comprising about 98.72% meloxicam and 1.28% polyvinyl alcohol. The granules were tray dried at 45 C to an LOD of 0.1%. The resulting granules were co-milled through 1143 μm screen for manufacture of the homogenous tablet core.

C. Homogenous Tablet Core Composition and Method of Manufacture

To manufacture the homogenous tablet core comprising of about 120 mg of tramadol and about 6 mg of meloxicam, a blend with the following composition was prepared: about 95.36% tramadol HCl granules (manufactured as described above, but milled again through a 991 μm comill screen to obtain comparable particle size granules as meloxicam), and 4.64% meloxicam granules (manufactured as described above). A homogenous blend of about 2000 g was manufactured by dispensing about 1907.2 g of tramadol HCL granules, and about 92.8 g of meloxicam granules. The material was added to a v-blender (4 quart shell) in the following order:

1. About half of the tramadol HCl granules

2. All of the meloxicam granules

3. The remaining of tramadol HCl granules

The tablet core components were homogenously blended for about 10 minutes, with the intensifier bar turnedd off. The homogenous blend was discharged from the shell and charged onto a tablet press (Piccola 10 station rotary press) and compressed to a target weight of about 131 mg and a target hardness of about 60N using 6.5 mm round standard concave tablet tooling. The resulting product comprises the homogenous tablet core, which at this point is an immediate release (IR) having the following composition:

Homogenous Tablet Core Composition

| Component | Mg/tablet | % w/w |
|---|---|---|
| Tramadol HCl | 120.11 | 91.69 |
| Meloxicam | 6.08 | 4.64 |
| Aerosil 2000 (Colloidal Silicone | 1.21 | 0.92 |
| Dioxide) | | |
| Polyvinyl alcohol (PVA) | 2.40 | 1.83 |
| Pruv (Sodium Stearyl Fumarate) | 1.21 | 0.92 |

D. Tablet Coating Composition and Method of Manufacture

Tablet Control-Release Coating Composition

| Component | % w/w |
|---|---|
| Ethocel 100 FP Premium | 4.61 |
| Kollidon ® 90F | 2.86 |
| Polyethylene Glycol 3350 (PEG) | 1.03 |
| Dibutyl sebacate (DBS) | 0.50 |
| 2-Propanol | 8.68 |
| Absolute Ethanol | 81.86 |
| Purified Water | 0.46 |

The homogenous IR tablet core was coated with an ethylcellulose based system by preparing an organic solvent solution consisting of about 4.61% ethocel standard 100FP premium, 2.86% Kollidon 90F, 1.03% carbowax sentry polyethylen glycol 3350 granular NF/FCC grade, 0.5% dibutyl sebacate NF, 8.68% 2-propanol, 81.86% absolute ethanol, and 0.46% purified water. The plasticized polymer solution was applied to 700 g of tablet core using O'Hara Labcoat 1 fitted with 12″ pan until about 17% weight gain was obtained. During spraying, the product temperature was maintained between 39-44° C., and the liquid spray rate was maintained between 10-22 g/min. The controlled release coated tablets were then dried for a further 30 minutes (inlet air set to 43 C, pan speed set at 5 rpm jog mode).

The dissolution profile of the above pharmaceutical composition was determined under the dissolution conditions described below in Table 27. The result of the dissolution testing is presented as a % of the total tramadol HCl and meloxicam in the controlled release tablet (batch 1258-171) and also depicted in FIG. 27

TABLE 27
Dissolution Conditions: 900 ml Phosphate Buffer,
pH 7.5, Apparatus 1, 75 rpm, 37 C.

% Release

| Time (hrs) | Tramadol HCl | Meloxicam |
|---|---|---|
| 2 | 51 | 10 |
| 4 | 70 | 20 |
| 8 | 96 | 39 |
| 10 | 100 | 50 |
| 16 | 100 | 74 |

### Example 28

A homogenous tablet core was prepared according to the method and composition described in Example 27. The homogenous cores thus obtained were coated according to the following method and composition:

Control-Release Coating Composition (Kollicoat SR30D based)

| Component | % w/w |
|---|---|
| Kollicoat SR 30D (30% dispersion) | 36.97 |
| Triethylcitrate | 0.55 |
| Pharmacoat 606 (Hydroxypropylmethyl cellulose) | 2.77 |
| Talc | 3.59 |
| Purified Water | 56.12 |

The homogenous IR tablet core was coated wih Kollicoat SR 30D based system by preparing an aqueous coating suspension consisting of about 36.97% Kollicoat SR 30D (30% dispersion), 055% triethylcitrate (TEC), 2.77% pharmacot 606, 3.59% talc, and 56.12% purified water. The plasticized polymer suspension was applied to 700 g of tablet core using O'Hara Labcoat 1 fitted with 12″ pan until about 40% weight gain was obtained. During spraying, the product temperature was maintained between 35-41 C, and the liquid spray rate maintained between 9-17 g/min. The controlled release coated tablets were then cured for 3 hours (inlet air set to 67 C, pan speed set at 3.3 rpm jog mode).

The dissolution profile of the above pharmaceutical composition was determined under the dissolution conditions described below in Table 28. The result of the dissolution testing is presented as a % of the total tramadol HCl and meloxicam in the controlled release tablet (batch 1258-175) and also depicted in FIG. 28.

TABLE 28
Dissolution Conditions: 900 ml Phosphate Buffer,
pH 7.5, Apparatus 1, 75 rpm, 37 C.

% Release

| Time (hrs) | Tramadol HCl | Meloxicam |
|---|---|---|
| 2 | 0 | 0 |
| 4 | 7 | 0 |
| 8 | 61 | 18 |
| 10 | 74 | 21 |
| 16 | 84 | 23 |

### Example 29

### Homogenous CR Tablet Core Composition and Method of Manufacture

The homogenous CR tablets comprising about 300 mg of tramadol HCl (equivalent to about 263.5 mg of tramadol base) and about 20 mg of meloxicam were prepared by direct blending of about 40.4% tramadol HCl powder, about 2.7% meloxicam powder, about 26.9% hydroxypropyl cellulose, about 16.8% lactose, about 9.7% microcrystalline cellulose, about 0.3% silicon dioxide, and about 3.0% magnesium stearate.

A homogenous blend of about 150 g was manufactured by dispensing about 60.63 g of tramadol HCl, about 4.04 g of meloxicam, about 40.42 of hydroxypropyl cellulose, about 25.26 g of lactose, about 14.59 g of microcrystalline cellulose, about 0.51 g of silicon dioxide, and about 4.55 g of magnesium stearate. The silicon dioxide and magnesium stearate were pre-screened through the 30 mesh screen prior to dispensing. Manual bag mixing was applied for 2 minutes.

The homogenous blend was further compressed using Natoli Single Station Press equipped with 0.706″×0.329″ capsule shaped tablet tooling. The target tablet weight was 742.2 mg. The hardness of the table was about 160 N. 1.75 tons of compression force was applied. The resulting product comprises the homogenous tablet core, which at this point is an controlled release core having the following composition:

Homogenous CR Tablet Core Composition

| Component | mg/tablet | % w/w |
|---|---|---|
| Tramadol HCl | 300.0 | 40.42 |
| Meloxicam | 20.0 | 2.69 |
| Hydroxypropyl | 200.0 | 26.95 |
| cellulose | | |
| Lactose DT | 125.0 | 16.84 |
| Microcrystalline | 72.2 | 9.73 |
| cellulose | | |
| Silicon dioxide | 2.5 | 0.34 |
| Magnesium stearate | 22.5 | 3.03 |

The dissolution profile of the above pharmaceutical composition was determined under the dissolution conditions described below in Table 29A and 29B. The result of the dissolution testing is presented as a % of the total tramadol HCl and meloxicam in the controlled release tablet (batch no. 08070T) and is also depicted in FIGS. 29A and 29B.

TABLE 29A
Dissolution Conditions: 900 ml 0.1N HCl,
USP Apparatus 1, 75 rpm, 37° C.

% Released

| Time (hr) | Tramadol HCl | Meloxicam |
|---|---|---|
| 0.5 | 18 | 0 |
| 1 | 28 | 0 |
| 2 | 42 | 0 |
| 3 | 53 | 0 |
| 4 | 62 | 0 |
| 5 | 70 | 0 |
| 6 | 76 | 0 |
| 8 | 86 | 0 |
| 10 | 93 | 0 |
| 12 | 97 | 0 |

TABLE 29B
Dissolution Conditions: 900 ml pH 6.8 phosphate
buffer, USP Apparatus 1, 75 rpm, 37° C.

% Released

| Time (hr) | Tramadol HCl | Meloxicam |
|---|---|---|
| 0.5 | 19 | 2 |
| 1 | 29 | 3 |
| 2 | 44 | 7 |
| 3 | 55 | 11 |
| 4 | 65 | 14 |
| 5 | 72 | 18 |
| 6 | 79 | 22 |
| 8 | 90 | 29 |
| 10 | 98 | 37 |
| 12 | 102 | 44 |

1. A pharmaceutical composition comprising:
a) a homogenous core comprising a therapeutically effective combination of active agents selected from the group consisting of bupropion hydrochloride and escitalopram oxalate, bupropion hydrobromide and citalopram hydrochloride, and bupropion hydrobromide and escitalopram oxalate, at least one stabilizer in an effective stabilizing amount, and at least one pharmaceutically acceptable excipient, and
b) a control-releasing coating surrounding said core, said coating comprising a water-insoluble water-permeable film-forming polymer, a water-soluble polymer and optionally at least one plasticizer.

2. The pharmaceutical composition of claim 1 wherein said pharmaceutical composition provides for a synchronous release of the combination of actives independent of pH of dissolution media.

3. The pharmaceutical composition of claim 1 wherein said combination of actives is bupropion hydrobromide and escitalopram oxalate.

4. The pharmaceutical composition of claim 1, wherein said stabilizer is selected from the group consisting at least one suitable pharmaceutically acceptable inorganic acid, at least one suitable pharmaceutically acceptable organic acid, at least one suitable pharmaceutically acceptable salt of an organic base, at least one suitable pharmaceutically acceptable salt of an inorganic acid, at least one suitable pharmaceutically acceptable acid salt of an amino acid, potassium metabisulfite, sodium bisulfite, or at least one suitable pharmaceutically acceptable phenylated antioxidant, or any combination thereof.

5. The pharmaceutical composition of claim 1 wherein said at least one stabilizer comprises at least one suitable inorganic acid, which at a concentration of about 0.31% w/w/ forms an aqueous solution having a pH of from about 0.5 to about 0.4.

6. The pharmaceutical composition of claim 1 wherein said at least one stabilizer comprises at least one suitable organic acid that has a solubility in water at 20° C. of less than about 10 g/100 g water and that at a concentration of about 60% w/w forms an aqueous suspension having a pH of from about 0.9 to about 4.0.

7. The pharmaceutical composition of claim 1 wherein said at least one stabilizer comprises at least one suitable dicarboxylic acid that has a solubility in water at 20° C. of less than about 10 g/100 g water and that at a concentration of about 60% w/w forms an aqueous suspension having a pH of from about 0.9 to about 4.0.

8. The pharmaceutical composition of claim 1 wherein said stabilizer comprises at least one suitable pharmaceutically acceptable salt of an organic base having an aqueous pH of from about 2.70 to about 3.10 at a concentration of about 10% w/w.

9. The pharmaceutical composition of claim 1 wherein said at least one stabilizer comprises at least one suitable pharmaceutically acceptable salt of an organic base having an aqueous pH of from about 2.95 to about 3.05, at a concentration of about 20% w/w.

10. The pharmaceutical composition of claim 1 wherein said at least one stabilizer comprises at least one salt of an organic base having an aqueous pH of from about 2.70 to about 2.72, at a concentration of about 20% w/w.

11. The pharmaceutical composition of claim 1 wherein said at least one stabilizer comprises at least one suitable pharmaceutically acceptable salt of an inorganic acid having an aqueous pH of from about 4.20 to about 4.30 at a concentration of about 10 w/w.

12. The pharmaceutical composition of claim 1 wherein said at least one stabilizer comprises at least one suitable pharmaceutically acceptable acid salt of an amino acid.

13. The pharmaceutical composition of claim 1 wherein said at least one stabilizer is selected from the group consisting of potassium metabisulfite, sodium bisulfite, and any combination thereof.

14. The pharmaceutical composition of claim 1 wherein said at least one stabilizer comprises at least one suitable pharmaceutically acceptable phenylated antioxidant.

15. The pharmaceutical composition of claim 14 wherein said at least one stabilizer is selected from the group consisting of butlylated hydroxytoluene (BHT), butylated hydroxyanisole (BHA), and any combination thereof.

16. The pharmaceutical composition of claim 1 wherein said at least one stabilizer comprises butlylated hydroxytoluene.

17. The pharmaceutical composition of claim 1 wherein said at least one stabilizer comprises citric acid.

18. The pharmaceutical composition of claim 1 wherein said at least one stabilizer comprises a combination of citric acid and butylayed hydroxytoluene.

19. The pharmaceutical composition of claim 1, wherein said at least one pharmaceutically acceptable excipient is selected from the group consisting of a binder, a lubricant, a filler, a glidant, and any combinations thereof.

20. The pharmaceutical composition of claim 1, wherein said water-insoluble water-permeable film-forming polymer is selected from the group consisting of cellulose ethers, cellulose esters, methacrylic acid derivatives, aqueous ethylcellulose dispersions, aqueous acrylic enteric systems, polyvinyl derivatives, and any combination thereof.

21. The pharmaceutical composition of claim 1, wherein said water-soluble polymer is selected from the group consisting of methylcellulose, hydroxypropylcellulose, hydroxypropylmethylcellulose, hydroxyethylcellulose, polyvinyl alcohol, polyvinylpyrrolidone, and any combination thereof.

22. The pharmaceutical composition of claim 1, wherein said at least one plasticizer, when said plasticizer is present, is selected from the group consisting of an ester, a polyalkylene glycol and any combination thereof.

23. The pharmaceutical composition of claim 1, wherein said at least one plasticizer, when said plasticizer is present, comprises a combination of two plasticizers.

24. The pharmaceutical composition of claim 1, wherein said composition is in the form of a tablet.

25. The pharmaceutical composition of claim 1, when administered to a subject in need of such administration provides a about 15-25% increase in the bioavailability of bupropion when compared to co-administration of single active agent pharmaceutical compositions of bupropion hydrobromide and citalopram hydrochloride or bupropion hydrobromide and escitalopram oxalate.

26. The pharmaceutical composition of claim 3 wherein said bupropion hydrobromide is present at an amount at least about 10% less than a single active agent pharmaceutical composition comprising 348 mg bupropion hydrobromide.
